(19) United States
(12) Patent Application Publication
(10) Pub. No.: US 2008/0131920 A1
(43) Pub. Date: Jun. 5, 2008

(54) Ion flux in biological processes, and methods related thereto

(75) Inventor: Michael Levin, Swampscott, MA (US)

Correspondence Address: ROPES & GRAY LLP, PATENT DOCKETING 39/41, ONE INTERNATIONAL PLACE, BOSTON, MA, 02110-2624, US

(73) Assignee: The Forsyth Institute, Boston, MA (US)

(21) Appl. No.: 11/543,469

(22) Filed: Oct. 4, 2006

Related U.S. Application Data
(60) Provisional application No. 60/723,414, filed on Oct. 4, 2005.
(60) Provisional application No. 60/841,777, filed on Aug. 31, 2006.

(51) Int. Cl. C12N 5/00; C12N 5/02; C12Q 1/20; C12N 5/04

(52) U.S. Cl. 435/29; 435/375; 435/377; 435/378

(57) ABSTRACT

The present invention provides methods for promoting dedifferentiation and/or regeneration by modulating membrane potential and/or intracellular pH in non-naturally regenerating cells.

### RELATED APPLICATIONS

This application claims the benefit of priority to U.S. provisional application Ser. No. 60/841,777, filed Aug. 31, 2006, and U.S. provisional application Ser. No. 60/723,414, filed Oct. 4, 2005. The foregoing disclosures are hereby incorporated by reference in their entirety.

### FUNDING

The invention described herein was supported, in whole or in part, by the National Center for Research Resources, National Institute of Health, Research Facilities Improvement Grant Number grant no. CO6RR11244, the National Science Foundation Career grant IBN #0347295, National Institutes of Health training grant 1-T32-DE-08327, National Institute of Health grant 1-R01-GM-06227, and the American Cancer Society Research Scholar Grant RSG-02-046-01-DDC. The United States government has certain rights in the invention.

### BACKGROUND OF THE INVENTION

In the past several decades, scientific advances have enhanced our understanding of the molecular and cell biological basis for embryonic and adult development. However, the biophysics of development has received less attention. As such, a complete understanding of the role of biophysics, and the integration of biophysical, molecular, and cell biological events during development and disease remains lacking.

Cells are bounded by cell membranes. One important biophysical event that occurs throughout the development and life of cells results from the function of ion transporter proteins. Ion flows set up by ion transporter proteins such as ion channels, ion pumps, and gap junctions produce pH and voltage gradients within cells and across cell fields. The membrane potential and ion flux across cell membranes is crucial not only as a means of regulating cellular homeostasis, but also to help mediate specific biological processes.

Despite the importance of ion transporter proteins in development, the prior art fails to provide methods for effectively identifying what, if any, role ion transporters play in particular biological processes. As such, the prior art fails to provide guidance to move beyond a general appreciation that ion transporters may be important. The present invention provides methods for efficiently and effectively identifying particular roles for ion transporter proteins during embryonic and adult development or disease.

Once a role during a particular biological process is established, modulation of ion flux and/or expression of ion transporter proteins can be used to specifically regulate that particular biological process. The present invention provides examples whereby ion flux can be used to promote or inhibit a particular biological process.

### BRIEF SUMMARY OF THE INVENTION

The present invention provides methods for promoting dedifferentiation and/or regeneration by modulating membrane potential and/or intracellular pH in cells.

In a first aspect, the invention provides a method of promoting regeneration, comprising providing a population of naturally regenerating cells; determining the membrane potential or pH range permissive for regeneration in said population of regenerating cells; providing a population of non-regenerating cells; determining the membrane potential or pH range of said population of non-regenerating cells; and contacting the population of non-regenerating cells with an agent that modulates ion flux mediated by a class of ion transporter proteins. The agent modifies the membrane potential or pH of one or more cells in the population of non-regenerating cells to the range permissive for regeneration as determined in the second step of the method; thereby promoting regeneration of one or more cells in the population of cells.

In a second aspect, the invention provides a method of promoting regeneration, comprising determining the membrane potential or pH range permissive for regeneration in a population of regenerating cells; providing a population of non-regenerating cells; determining the membrane potential or pH range of said population of non-regenerating cells; and contacting the population of non-regenerating cells with an agent that modulates ion flux mediated by a class of ion transporter proteins. The agent modifies the membrane potential or pH of one or more cells in the population of non-regenerating cells to the range permissive for regeneration as determined in the second step of the method; thereby promoting regeneration of one or more cells in the population of cells.

In one embodiment of any of the foregoing, determining the membrane potential or pH range permissive for regeneration in said population of regenerating cells comprises providing a population of regenerating cells; contacting said population of cells with an agent which is a voltage sensitive agent that produces a detectable signal; and measuring the detectable signal to calculate an average membrane potential or pH of said population of cells during regeneration. In another embodiment of any of the foregoing, the first two steps are repeated for multiple types of naturally regenerating cells and the average membrane potential or pH range are used in the final step. In another embodiment of any of the foregoing, determining the membrane potential or pH range of said population of non-regenerating cells comprises providing a population of non-regenerating cells; contacting said population of cells with an agent which is a voltage sensitive agent that produces a detectable signal; and measuring the detectable signal to calculate an average membrane potential or pH of said population of cells.

In one embodiment of any of the foregoing, the method is an in vitro method and the population of non-regenerating cells are in culture. In another embodiment of any of the foregoing, the method is an in vitro method and the population of non-regenerating cells are in a preparation of tissue in culture. In another embodiment of any of the foregoing, the method is an in vivo method and the population of non-regenerating cells are in an animal.

In one embodiment of any of the foregoing, the population of non-regenerating cells are derived from or resident in an animal, and the animal is a flatworm, an amphibian, a fish, a reptile, a bird, or a mammal. In another embodiment of any of the foregoing, the mammal is a mouse, rat, cat, dog, rabbit, goat, hamster, pig, sheep, non-human primate, or human. In another embodiment of any of the foregoing, the mammal is a human. In another embodiment of any of the foregoing, the human is a patient in need of regeneration. In another embodiment of any of the foregoing, the population of non-regenerating cells are embryonic, fetal, larval, juvenile, or adult cells. In another embodiment of any of the foregoing, the cells are adult cells.

In one embodiment of any of the foregoing, the agent inhibits ion flux mediated by the class of transporter proteins. In another embodiment of any of the foregoing, the agent promotes ion flux mediated by the class of transporter proteins. In another embodiment of any of the foregoing, the agent is selected from a nucleic acid, a peptide, a protein, a small organic molecule, a small inorganic molecule, an antisense oligonucleotide, an RNAi construct, or an antibody.

In one embodiment of any of the foregoing, the agent that inhibits ion flux mediated by a class of ion transporters is an ion channel protein or a nucleotide construct that encodes an ion channel protein. In another embodiment of any of the foregoing, the ion transporter protein is a hyperpolarizing transporter. In another embodiment of any of the foregoing, the ion transporter protein is a depolarizing transporter. In another embodiment of any of the foregoing, the ion transporter protein is an H⁺ pump. In another embodiment of any of the foregoing, the H⁺ pump is a V-ATPase H⁺ pump. In another embodiment of any of the foregoing, the H⁺ pump is a P-type H⁺ ATPase pump. In another embodiment of any of the foregoing, the H⁺ pump is a yeast PMA1.2H⁺ pump. In another embodiment of any of the foregoing, the ion transporter protein is a K⁺ channel. In another embodiment of any of the foregoing, the K⁺ channel is a ROMK K⁺ channel. In another embodiment of any of the foregoing, the K⁺ channel is an ERG K⁺ channel. In another embodiment of any of the foregoing, the ion transporter protein is an Na⁺ channel. Note, however, that these particular ion transporter proteins are merely exemplary of particular proteins that can be manipulated to modulate membrane potential. One of skill in the art can readily select an ion transporter protein and manipulate the activity of that transporter protein (using an agent) to depolarize or hyperpolarize cell membranes, thereby shifting membrane potential of cells or cells in a tissue into a range permissive for regeneration. In certain embodiments, the particular ion transporter protein is chosen because it is endogenously expressed in the particular cells or tissues being studied. In certain embodiments, the particular ion transporter protein is chosen because it is not endogenously expressed in the particular cells or tissues being studied.

In a third aspect, the invention provides a method of promoting regeneration, comprising providing a population of cells of known membrane potential or pH; and contacting the population of cells with an agent that modulates ion flux mediated by a class of ion transporter proteins. The agent modifies the membrane potential or pH of one or more cells in the population of cells to a range permissive for regeneration; thereby promoting regeneration of one or more cells in the population of cells.

In one embodiment of any of the foregoing, the membrane potential range permissive for regeneration is between −70 mV and 30 mV. In another embodiment, the membrane potential range permissive for regeneration is between −40 mV and 20 mV. In still another embodiment, the lower range of the membrane potential is −70 mV, −60 mV, −50 mV, −40 mV, −35 mV, −30 mV, −25 mV, −20 mV, −15 mV, −10 mV, −5 mV, or 0 mV and the upper range of the membrane potential is −30 mV, −20 mV, −10 mV, −5 mV, 0 mV, 5 mV, 10 mV, 15 mV, 20, mV, 25 mV, or 30 mV.

In one embodiment of any of the foregoing, the pH range permissive for regeneration is less than or equal to about 7.0, 6.9, 6.8, 6.7, 6.6, 6.5, 6.4, 6.3, 6.2, 6.1, or 6.0. In another embodiment of any of the foregoing, both the membrane potential and the pH range permissive for regeneration may be used in any of the steps of the methods of the invention.

In a fourth aspect, the invention provides a method of promoting regeneration, comprising providing a population of cells; and contacting the population of cells with an agent that increases ion flux mediated by a V-ATPase H⁺ pump. The agent promotes relative hyperpolarization of cell membranes of one or more cells in the population of cells, thereby promoting regeneration of one or more cells in the population of cells.

In one embodiment of any of the foregoing, the agent is a nucleotide construct encoding the V-ATPase H⁺ pump.

In a fifth aspect, the invention provides an agent that modulates activity of a H⁺ pump in the manufacture of a pharmaceutical composition for promoting regeneration.

In one embodiment of any of the foregoing, the H⁺ pump is a P-type H⁺ ATPase pump. In another embodiment of any of the foregoing, the H⁺ pump is a V-ATPase H⁺ pump.

In a sixth aspect, the invention provides an agent that modulates the activity of a K⁺ channel in the manufacture of a pharmaceutical composition for promoting regeneration.

In one embodiment of any of the foregoing, the channel is a ROMK K⁺ channel. In another embodiment of any of the foregoing, the channel is an ERG K⁺ channel. In another embodiment of any of the foregoing, the channel is an KATP channel. In another embodiment of any of the foregoing, the channel is an KCNK1 channel. In another embodiment of any of the foregoing, the channel is an KCNQ1 channel.

In a seventh aspect, the invention provides an agent that activates an voltage-dependent sodium channel (NaV) in the manufacture of a pharmaceutical composition for promoting regeneration.

Note, that although certain embodiments and aspects of the invention involve manipulating particular ion transporter proteins, these particular ion transporter proteins are merely exemplary of particular proteins that can be manipulated to modulate membrane potential. One of skill in the art can readily select an ion transporter protein and manipulate the activity of that transporter protein (using an agent) to depolarize or hyperpolarize cell membranes, thereby shifting membrane potential of cells or cells in a tissue into a range permissive for regeneration. In certain embodiments, the particular ion transporter protein is chosen because it is endogenously expressed in the particular cells or tissues being studied. In certain embodiments, the particular ion transporter protein is chosen because it is not endogenously expressed in the particular cells or tissues being studied.

In an eighth aspect, the invention provides a method of screening for compounds that promote dedifferentiation of cells. The method comprises contacting a population of cells in culture with one or more compounds, and contacting the population of cells with a voltage sensitive agent that produces a detectable signal in response to a depolarized cell membrane. The detectable signal is measured and the average membrane potential of said population of cells is calculated. The average membrane potential of said population of cells cultured in the presence of the compounds is compared to that of a control population of cells cultured in the absence of the compounds. Compounds that increase the average membrane potential in said population of cells are identified as candidate compounds for promoting dedifferentiation of cells.

In a ninth aspect, the invention provides a method of screening for compounds that promote dedifferentiation of cells. The method comprises contacting an animal or tissue with one or more compounds, and contacting said animal or tissue with a voltage sensitive agent that produces a detectable signal in response to a depolarized cell membrane. The detectable signal is measured and the average membrane potential of one or more populations of cells in the animal or tissue is calculated. The average membrane potential of said population of cells cultured in the presence of the compounds is compared to that of a control population of cells cultured in the absence of the compounds. Compounds that increase the average membrane potential in said population of cells are identified as candidate compounds for promoting dedifferentiation of cells.

In a tenth aspect, the invention provides a method of screening for compounds that promote dedifferentiation of cells. The method comprises producing an injury in an animal or tissue, contacting said injured animal or tissue with one or more compounds, and contacting said injured animal or tissue with a voltage sensitive agent that produces a detectable signal in response to a depolarized cell membrane. The detectable signal is measured and the average membrane potential of one or more populations of cells proximate to the injury in the animal or tissue is calculated. The average membrane potential of said population of cells cultured in the presence of the compounds is compared to that of a control population of cells cultured in the absence of the compounds. Compounds that increase the average membrane potential in said one or more populations of cells proximate to the injury are identified as candidate compounds for promoting dedifferentiation of cells.

In one embodiment of any of the foregoing, the one or more compounds are independently selected from nucleic acids, peptides, proteins, small organic molecules, small inorganic molecules, antisense oligonucleotides, RNAi constructs, and antibodies.

In an eleventh aspect, the invention provides a method for promoting dedifferentiation. The method comprises providing a population of cells. The cells are contacted with a compound that modulates ion flux mediated by a class of ion transporter proteins. The compound that modulates ion flux mediated by a class of ion transporter proteins promotes depolarization of cell membranes of one or more cells in the population of cells, thereby promoting dedifferentiation of one or more cells in the population of cells.

In one embodiment, the method further comprises culturing the population of cells which includes one or more dedifferentiated cells, wherein said culturing promotes cellular regeneration.

In a twelfth aspect, the invention provides a method for inhibiting dedifferentiation. The method comprises providing a population of cells. The cells are contacted with a compound that modulates ion flux mediated by a class of ion transporter proteins. The compound that modulates ion flux mediated by a class of ion transporter proteins inhibits depolarization of cell membranes of one or more cells in the population of cells, thereby inhibiting dedifferentiation of one or more cells in the population of cells.

In one embodiment, the method is an in vitro method and the population of cells is in culture. In another embodiment, the method is an in vitro method and the population of cells is in a preparation of tissue in culture. In another embodiment, the method is an in vivo method and the population of cells is resident in an animal. In one embodiment, when the population of cells is resident in an animal, the animal includes an injury. In another embodiment, when the population of cells is resident in an animal, the animal is a fragment of an animal.

The above methods can be used in cells that are resident in or derived from any species or organism. Exemplary organisms are animals, although the method can similarly be used to access the role of ion flux in plants, bacteria and other prokaryotes, and fungi. In one embodiment, the cells are derived from or resident in an animal selected from a flatworm, an amphibian, a fish, a reptile, a bird, or a mammal. In one embodiment, the animal is a flatworm and the flatworm is a planarian of the class Turbellaria. In another embodiment, the animal is an amphibian and the amphibian is Xenopus laevis or Xenopus tropicalis. In yet another embodiment, the animal is a mammal selected from a mouse, rat, cat, dog, rabbit, goat, hamster, pig, sheep, non-human primate, or primate.

In any of the foregoing, the invention contemplates that the cells or animals, regardless of species, can be at any developmental stage. In one embodiment, the population of cells comprises embryonic, fetal, larval, juvenile, or adult cells. In another embodiment, the population of cells is resident in animal, and the animal is an embryonic, fetal, larval, juvenile, or adult stage animal. In another embodiment, the population of cells comprises fertilized or unfertilized oocytes.

In one embodiment of any of the foregoing, the compound inhibits ion flux mediated by the class of transporter proteins. In another embodiment, the compound promotes ion flux mediated by the class of transporter proteins.

In one embodiment of any of the foregoing, the compound is selected from a nucleic acid, a peptide, a protein, a small organic molecule, a small inorganic molecule, an antisense oligonucleotide, an RNAi construct, or an antibody.

In a thirteenth aspect, the invention provides pharmaceutical preparations of one or more compounds identified by the methods of the present invention.

In a fourteenth aspect, the invention provides pharmaceutical preparations for promoting dedifferentiation and/or regeneration in one or more cells in a population of cells.

In a fifteenth aspect, the invention provides pharmaceutical preparations for inhibiting dedifferentiation and/or regeneration in one or more cells in a population of cells.

In a sixteenth aspect, the invention provides use of a compound that modulates ion flux and/or membrane potential in the manufacture of a medicament for promoting dedifferentiation and/or regeneration.

In one embodiment, the compound inhibits ion flux mediated by a class of ion transporter proteins, thereby modulating ion flux and/or membrane potential. In another embodiment, the compound promotes ion flux mediated by a class of ion transporter proteins, thereby modulating ion flux and/or membrane potential.

In a seventeenth aspect, the invention provides use of a compound that modulates ion flux and/or membrane potential in the manufacture of a medicament for inhibiting dedifferentiation and/or regeneration.

In one embodiment, the compound inhibits ion flux mediated by a class of ion transporter proteins, thereby modulating ion flux and/or membrane potential. In another embodiment, the compound promotes ion flux mediated by a class of ion transporter proteins, thereby modulating ion flux and/or membrane potential.

In an eighteenth aspect, the invention provides a method for identifying progenitor cells. This aspect of the invention is based on the appreciation of a correlation between sternness and membrane potential. Given this correlation, methods of identifying depolarized cells can be used to identify and/or separate progenitor cells from amongst a population of cells, thereby facilitating further culture, purification, and analysis of progenitor cells. In one embodiment, a method for identifying progenitor cells comprises contacting a population of cells with a voltage sensitive agent that produces a detectable signal in response to a depolarized cell membrane. One or more cells in the population of cells which have a depolarized cell membrane with a membrane potential of greater than or equal to −20 mV are identified, thereby identifying candidate progenitor cells.

In a nineteenth aspect, a method for identifying progenitor cells comprises contacting a population of cells with a pH sensitive agent that produces a detectable signal. One or more cells in the population of cells which have an intracellular pH of less than or equal to 6.7 are identified, thereby identifying candidate progenitor cells.

In any of the foregoing, the method is an in vitro method and the population of cells is in culture. In another embodiment, the method is an in vitro method and the population of cells is in a preparation of tissue in culture. In another embodiment, the method is an in vivo method and the population of cells is resident in an animal.

The above methods can be used in cells that are resident in or derived from any species or organism. Exemplary organisms are animals, although the method can similarly be used to access the role of ion flux in plants, bacteria and other prokaryotes, and fungi. In one embodiment, the cells are derived from or resident in an animal selected from a flatworm, an amphibian, a fish, a reptile, a bird, or a mammal. In one embodiment, the animal is a flatworm and the flatworm is a planarian of the class Turbellaria. In another embodiment, the animal is an amphibian and the amphibian is Xenopus laevis or Xenopus tropicalis. In yet another embodiment, the animal is a mammal selected from a mouse, rat, cat, dog, rabbit, goat, hamster, pig, sheep, non-human primate, or primate.

In any of the foregoing, the invention contemplates that the cells or animals, regardless of species, can be at any developmental stage. In one embodiment, the population of cells comprises embryonic, fetal, larval, juvenile, or adult cells. In another embodiment, the population of cells is resident in animal, and the animal is an embryonic, fetal, larval, juvenile, or adult stage animal. In another embodiment, the population of cells comprises fertilized or unfertilized oocytes.

In one embodiment, the method comprises identifying one or more cells having a membrane potential greater than or equal to −20 mV and less than or equal to 30 mV. In another embodiment, the method comprises identifying one or more cells having a membrane potential of greater than or equal to −15 mV. In another embodiment, the method comprises identifying one or more cells having a membrane potential of greater than or equal to −10 mV. In still another embodiment, the method comprises identifying one or more cells having a membrane potential of greater than or equal to −5 mV. In yet another embodiment, the method comprises identifying one or more cells having a membrane potential of greater than or equal to 0 mV, 5 mV, 10 mV, 15 mV, or 20 mV.

In another embodiment, the method comprises contacting the population of cells with both a pH sensitive agent and a voltage sensitive agent. One or more cells having both an intracellular pH of less than or equal to 6.7 and a membrane potential of greater than or equal to −20 mV are identified, thereby identifying candidate progenitor cells.

In one embodiment of any of the foregoing, the method may further comprise separating candidate progenitor cells (e.g., all of the identified candidate progenitor cells or a subset of the identified candidate progenitor cells) from the population of the cells. When progenitor cells are separated from the population of cells, the separated progenitor cells may be cultured to produce a population of cells enriched in progenitor cells.

In one embodiment of any of the foregoing, the detectable signal produced by the agents (e.g., the voltage sensitive agent or the pH sensitive agent) is a fluorescent signal. Exemplary voltage sensitive agents include, but are not limited to, bis-(1,3-dibutylbarbituric acid)pentamethine oxonol (DiBAC₄(5)); bis-(1,3-dibutylbarbituric acid)trimethine oxonol (DiBAC₄(3)); bis-(1,3-diethylthiobarbituric acid)trimethine oxonol (DiSBAC₂(3)); 3,3′-diethyloxacarbocyanine iodide (DiOC₂(3)); 3,3′-diheptyloxacarbocyanine iodide (DiOC₇(3)); 3,3′-dihexyloxacarbocyanine iodide (DiOC₆(3)); 3,3′-dipentyloxacarbocyanine iodide (DiOC₅(3)); 1,1′,3,3,3′,3′-hexamethylindodicarbocyanine iodide (DiIC₁(5)); a structural variant thereof; or a functional variant thereof. Exemplary pH sensitive agents include, but are not limited to, 2′,7′-bis-(2-carboxyethyl)-5-(and-6)-carboxyfluorescein (BCECF); 5-(and-6)-carboxy SNARF®-1; LysoTracker® Blue DND-22; LysoTracker® Green DND-26; LysoTracker® Red DND-99; LysoTracker® Yellow HCK-123; a structural variant thereof; or a functional variant thereof.

In certain embodiment, the method comprises contacting cells with both an voltage sensitive agent and a pH sensitive agent. In one embodiment, the cells are contacted with the two agents simultaneously. In another embodiment, the cells are contacted with the two agents sequentially.

In a related embodiment of the foregoing aspects and embodiments of the invention, the invention provides a method for separating progenitor cells from an animal or tissue. The method comprises contacting an animal or tissue with a voltage sensitive agent that produces a detectable signal in cells in response to a depolarized cell membrane. One or more cells in the animal or tissue having a depolarized membrane potential of greater than or equal to −20 mV are identified, thereby identifying candidate progenitor cells. The identified progenitor cells are then removed from the animal or tissue, thereby separating the progenitor cells from the remainder of the animal or tissue.

In another related embodiment of the foregoing aspects and embodiments of the invention, the invention provides a method for separating progenitor cells from an animal or tissue. The method comprises contacting an animal or tissue with a pH sensitive agent that produces a detectable signal. One or more cells in the animal or tissue having an intracellular pH of less than or equal to 6.7 are identified, thereby identifying candidate progenitor cells. The identified progenitor cells are then removed from the animal or tissue, thereby separating the progenitor cells from the remainder of the animal or tissue.

In yet another related embodiment, the invention provides a method for separating progenitor cells from an animal or tissue. The method comprises contacting an animal or tissue with both a pH sensitive agent that produces a detectable signal and a voltage sensitive agent that produces a detectable signal in response to a depolarized cell membrane. One or more cells in the animal or tissue having both a depolarized membrane potential of greater than or equal to −20 mV and an intracellular pH of less than or equal to 6.7 are identified, thereby identifying candidate progenitor cells. The identified progenitor cells are then removed from the animal or tissue, thereby separating the progenitor cells from the remainder of the animal or tissue.

In one embodiment removing identified progenitor cells comprises dissecting out candidate progenitor cells, thereby removing candidate progenitor cells from the animal or tissue. In another embodiment, removing identified candidate progenitor cells comprises dissociating the animal or tissue; and sorting candidate progenitor cells, thereby separating candidate progenitor cells from the animal or tissue. When progenitor cells are sorted, the method of sorting can comprise sorting based on the detectable signal (e.g., the detectable signal provided by the pH sensitive agent or the voltage sensitive agent). An exemplary method of cell sorting based on a detectable signal fluorescence activated cell sorting (FACS) analysis.

The present invention also provides methods for identifying whether ion flux is involved in a particular biological process, and if so, which class of ion transporter proteins may mediate that biological process. The present invention further provides a variety of methods based on the role of ion transporter proteins and ion flux during cellular dedifferentiation and/or regeneration.

In a twentieth aspect, the invention provides a method for determining whether ion flux is involved in a particular biological process, and if so, identifying a class of ion transporter proteins that mediate ion flux during the particular biological process. A method for identifying a class of ion transporter proteins which mediate ion flux during a particular biological process comprises providing a population of cells that can be used to measure a particular biological process. The population of cells is contacted with a compound that modulates ion flux mediated by a class of ion transporter proteins. Following administration of a compound that modulates ion flux, the particular biological process is measured or otherwise assayed, and compared in the presence versus the absence of the compound. If there is a change in the particular biological process in the presence versus the absence of the compound (e.g., the compound that modulates ion flux mediated by a class of ion transporters), then that class of ion transporter proteins is identified as a candidate for mediating (in whole or in part) ion flux during that particular biological process in the population of cells.

In a twenty first aspect, the invention provides a method for identifying a class of ion transporter proteins that mediate ion flux during a particular biological process. The method is a reiterative method comprising assessing the effects of compounds that are increasingly specific. In other words, in each successive round of screening, the cells are contacted with a compound that modulates the activity of an increasingly specific/defined class of ion transporter proteins. By way of example, a method for identifying a class of ion transporter proteins which mediate ion flux during a particular biological process comprises providing a population of cells that can be used to measure a particular biological process. The population of cells is contacted with a first compound that modulates ion flux mediated by a first class of ion transporter proteins. Following administration of the first compound that modulates ion flux, the particular biological process is measured or otherwise assayed, and compared in the presence versus the absence of the compound. If there is a change in the particular biological process in the presence versus the absence of the compound (e.g., the compound that modulates ion flux mediated by a class of ion transporters), then that class of ion transporter proteins is identified as a candidate for mediating (in whole or in part) ion flux during that particular biological process in the population of cells. A second population of the equivalent cells is then contacted with a second compound that modulates ion flux mediated by a second class of ion transporter proteins. This second class of ion transporter proteins comprises a subset of the first class of ion transporter proteins, and thus the second compound serves to further narrow/specify the candidate class of ion transporter proteins that mediate ion flux and thus mediate the particular biological process. Following contacting the population of cells with the second compound, the method comprises measuring the particular biological process in the population of cells in the presence of the second compound versus the absence of the second compound, and determining whether the second compound that modulates ion flux mediated by the second class of ion transporter proteins changes the particular biological process in the population of cells. This method facilitates identification of a candidate class of ion transporter proteins that is more specific than that identified following the use of a single round of screening with a single compound.

In any of the foregoing, the method may comprise a reiterative method where the screening steps are repeated multiple times. In certain embodiments, a compound that modulates an increasingly specific class of ion transporter proteins is used in each successive round of screening. In this way, each successive round of screening serves to further define and narrow the candidate class of ion transporter proteins that may play a role in the particular biological process being studies. In this way, the invention provides an ordered, hierarchical screening approach for (i) identifying whether ion transport is involved in a particular biological process, (ii) identifying, through one or more rounds of successive screening, one or more candidate classes of ion transporter proteins that may mediate ion transport in a particular biological process, and in certain embodiments (iii) identifying one or more members of a particular class of ion transporter proteins capable of mediating ion flux involved in a particular biological process.

In one embodiment, the method is an in vitro method and the population of cells is in culture. In another embodiment, the method is an in vitro method and the population of cells is in a preparation of tissue in culture. In another embodiment, the method is an in vivo method and the population of cells is resident in an animal.

The above methods can be used in cells that are resident in or derived from any species or organism. Exemplary organisms are animals, although the method can similarly be used to access the role of ion flux in plants, bacteria and other prokaryotes, and fungi. In one embodiment, the cells are derived from or resident in an animal selected from a flatworm, an amphibian, a fish, a reptile, a bird, or a mammal. In one embodiment, the animal is a flatworm and the flatworm is a planarian of the class Turbellaria. In another embodiment, the animal is an amphibian and the amphibian is Xenopus laevis or Xenopus tropicalis. In yet another embodiment, the animal is a mammal selected from a mouse, rat, cat, dog, rabbit, goat, hamster, pig, sheep, non-human primate, or primate.

In any of the foregoing, the invention contemplates that the cells or animals, regardless of species, can be at any developmental stage. In one embodiment, the population of cells comprises embryonic, fetal, larval, juvenile, or adult cells. In another embodiment, the population of cells is resident in an animal, and the animal is an embryonic, fetal, larval, juvenile, or adult stage animal. In another embodiment, the population of cells comprises fertilized or unfertilized oocytes.

The foregoing methods comprise contacting cells with a compound that modulates ion flux mediated by the class of ion transporter proteins. In one embodiment, the compound inhibits ion flux mediated by the class of ion transporter proteins. In another embodiment, the compound promotes ion flux mediated by the class of transporter proteins. An inhibitor or promoter of ion flux may, for example, modulate the expression and/or activity of one or more ion transporters within a class of ion transporters. Compounds (e.g., inhibitors or promoters) may act directly upon one or more ion transporter protein (e.g., by directly binding to ion transporter proteins) or compounds may act indirectly (e.g., via a necessary cofactor, by influencing transcription or translation of ion transporter proteins, etc).

The foregoing methods comprise contacting cells with a compound that modulates ion flux mediated by a class of ion transporter proteins. In one embodiment, the compound is selected from nucleic acids, peptides, proteins, small organic molecules, small inorganic molecules, antisense oligonucleotides, RNAi constructs, or antibodies. When the methods comprise a reiterative method involving multiple rounds of screening using compounds that are increasing selective with respect to the class of ion transporter proteins, the one or more compounds used are independently selected from nucleic acids, peptides, proteins, small organic molecules, small inorganic molecules, antisense oligonucleotides, RNAi constructs, or antibodies.

The foregoing methods can be used to study any of a range of biological processes. The biological process can be defined generally, as well as specifically based on the model organism, developmental stage, and cell type under investigation. In one embodiment, the biological process is a general biological process selected from cell proliferation, cell differentiation, apoptosis, cell survival, cell migration, regeneration, or dedifferentiation. In certain embodiments, the particular biological process can be further described based on the cell type, organism, or stage of development being evaluated.

Numerous methods in cell and developmental biology can be used to assay the particular biological process. In one embodiment, measuring the particular biological process comprises measuring a change in gene expression, a change in protein expression, or a change in morphology. In another embodiment, measuring the particular biological process comprises measuring a change in the rate or extent of cell proliferation, a change in cell differentiation, a change in the rate of tissue regeneration, or a change in the rate or extent of apoptosis.

The foregoing provide a method for identifying a candidate class of ion transporter proteins that may mediate ion flux, thereby modulating a particular biological process. Identification of a candidate class of ion transporter proteins provides a list of candidate ion transporter proteins that may be involved in a particular biological process. In certain embodiments, the method further comprises evaluating one or more individual ion transporter proteins within the identified class to identify a particular ion transporter protein involved in modulating the particular biological process.

In one embodiment, evaluating one or more individual ion transporter proteins (e.g., individual proteins that are members of an identified class of transporters) comprises examining gene or protein expression of one or more individual ion transporter proteins.

In another embodiment, evaluating one or more individual ion transporter proteins comprises providing a population of cells that can be used to measure a particular biological process, and inhibiting expression or activity of one or more individual ion transporter proteins, which ion transporter proteins are members of the candidate class of ion transporter proteins. In another embodiment, evaluating one or more individual ion transporter proteins comprises providing a population of cells that can be used to measure a particular biological process, and promoting expression or activity of one or more individual ion transporter proteins, which ion transporter proteins are members of the candidate class of ion transporter proteins.

Following inhibiting or promoting expression or activity of one or more individual ion transporter proteins, the particular biological process can be assayed in the cells to determine whether inhibition or promotion of the expression or activity of said one or more ion transporter proteins changes the particular biological process in the population of cells. The invention contemplates a combinatorial strategy in which both expression and function of the particular one or more ion transporter proteins is evaluated.

In one embodiment of methods used to further identify one or more particular ion transporter proteins, inhibiting the expression or activity of the ion transporter protein may comprise contacting the population of cells with an agent that specifically inhibits the expression or activity of the ion transporter protein and does not substantially inhibit the expression or activity of other ion transporter proteins that are a member of the candidate class of ion transporter proteins. In one embodiment, the agent that inhibits expression or activity of an ion transporter protein is an RNAi construct that specifically inhibits the expression or activity of the ion transporter protein, and the method comprises contacting the population of cells with the RNAi construct. In another embodiment, the agent that inhibits expression or activity of an ion transporter protein is an antibody that is immunoreactive with and specifically inhibits the activity of the ion transporter protein, and the method comprises contacting the population of cells with the antibody.

In one embodiment, inhibiting the expression or activity of an ion transporter protein comprises contacting the population of cells with any of a nucleic acid, peptide, protein, small organic molecule, small inorganic molecule, antisense oligonucleotide, RNAi construct, or antibody, thereby specifically inhibiting the expression or activity of the ion transporter protein. In one embodiment, the agent is a nucleic acid and the nucleic acid encodes a dominant negative form of the ion transporter protein. In another embodiment, the agent is a protein, and the protein is a dominant negative form of the ion transporter protein.

In one embodiment, promoting the expression or activity of an ion transporter protein comprises contacting the population of cells with any of a nucleic acid, peptide, protein, small organic molecule, or small inorganic molecule, thereby specifically inhibiting the expression or activity of the ion transporter protein. In one embodiment, the agent is a nucleic acid and the nucleic acid encodes the candidate ion transporter protein. In another embodiment, the agent is a protein, and the protein is the ion transporter protein. Ectopic expression of the candidate ion transporter protein (e.g., using a nucleic acid or protein corresponding to the candidate ion transporter protein) can be used to promote expression of an ion transporter protein in a population of cells.

In any of the foregoing embodiments of this aspect of the invention, the invention further contemplates assaying the population of cells to determine whether a compound that modulates ion flux via a class of ion transporter proteins or via a particular candidate ion transporter protein also alters the membrane potential of cells within the population of cells. In one embodiment, the membrane potential is assessed using a vibrating probe. In another embodiment, the membrane potential is assessed using a fluorescent agent that emits a detectable signal indicative of the membrane potential.

In any of the foregoing embodiments of this aspect of the invention which call for multiple rounds of screening and analysis, the invention contemplates using the same or equivalent population of cells (e.g., populations that are derived from the same species, tissue type, and are of the same stage of developmental). Unless specifically stated, reference to the population of cells is not meant to imply that the identical cells are used in subsequent rounds of study. Rather, the term is meant to indicate that equivalent cells are used when conducting experiments that require multiple rounds of screening. However, the invention contemplates that it may sometimes be advantageous to conduct various rounds of screening using different populations of cells (e.g., populations of cells derived from a different species, tissue type, or stage of development).

In any of the foregoing embodiments of this aspect of the invention, the invention further contemplates screening methods for other families of proteins susceptible to hierarchical screening. For example, other susceptible families of proteins include neurotransmitters and molecular motors.

The invention contemplates combinations of any of the foregoing aspects and embodiments of the invention. Furthermore, in any of the foregoing aspects and embodiments of the invention, the invention contemplates the use of compounds to specifically modulate a particular biological process (e.g., to modulate a biological process directly rather than by generally perturbing viability of the organism).

### BRIEF DESCRIPTION OF THE DRAWINGS

The invention may be better understood by referring to the following description of illustrative embodiments, taken in conjunction with the accompanying drawings, in which like reference designations refer to like components and depicted components are not necessarily drawn to scale.

FIG. 1 illustrates a multi-step approach for evaluating a role for calcium ion flux in a biological process.

FIG. 2 illustrates a multi-step approach for evaluating a role for potassium ion flux in a biological process.

FIG. 3 illustrates a multi-step approach for evaluating a role for hydrogen ion flux in a biological process.

FIG. 4 illustrates a multi-step approach for evaluating a role for sodium ion flux in a biological process.

FIG. 5 illustrates a multi-step approach for evaluating a role for chloride ion flux in a biological process.

FIG. 6 illustrates a method for evaluating a role for ion flux in a biological process.

FIG. 7 illustrates a specific example of a multi-step approach for determining a class of ion transporter proteins that mediate calcium ion flux, and thereby modulate a particular biological process.

FIGS. 8A-8F illustrate standard curves used to interpret and utilize fluorescent dyes.

FIG. 9 summarizes the results of a candidate screen to identify ion transporter proteins involved in regeneration in Xenopus tails.

FIG. 10A-10F illustrate the results of manipulation of ion flux on regeneration in Xenopus tails.

FIG. 11A-11F depict membrane voltage and pH in regenerating tails in the presence and absence of compound that modulate ion flux mediated by ion transporter proteins.

FIG. 12A-12J show that the V-ATPase is required for tail regeneration in Xenopus.

FIG. 13A-13M′ show the characterization of expression of V-ATPase and physiology in the regeneration bud.

FIG. 14A-14N show V-ATPase function is required for the up-regulation of cell proliferation and axonal patterning.

FIG. 15 summarizes a step-wise model of tail regeneration consisting of physiological, gene expression, and morphogenetic modules.

FIG. 16 illustrates one embodiment of a method of promoting regeneration.

### DETAILED DESCRIPTION OF THE INVENTION

### (i) Overview

The present invention is based on our appreciation of the important role of ion flux and membrane potential in a range of biological processes. Ion flows set up by channels, pumps, and other ion transporter proteins produce pH and voltage gradients within cells and across cells fields. Ion flow and the regulation of ion flow plays an important role in a range of biological processes, and thus methods of identifying ion transporter proteins involved in particular biological processes are critical in understanding and manipulating a variety of physiological phenomenon in cells, tissues, and organisms.

We have uncovered a role for ion flux in a biological process of tremendous interest to scientists and physicians alike: cellular dedifferentiation and regeneration. In light of the role of ion flux in dedifferentiation and regeneration, compounds that modulate ion flux may be used to promote dedifferentiation and/or regeneration, thereby promoting regeneration of cells and tissues in vitro or in vivo. Furthermore, compounds that modulate ion flux may be used to inhibit dedifferentiation and/or regeneration, thereby inhibiting regeneration of cells and tissues in vitro or in vivo. For any of the foregoing, these compounds may be compounds already known to influence ion flux and membrane potential. Known modulators of ion flux and/or of the expression or activity of ion transporter proteins can be tested in a particular cell type or system to identify which of the known compounds modulate dedifferentiation and/or regeneration. Alternatively, compounds previously unknown to influence ion flux and membrane potential can be tested to identify compounds that both influence ion flux and membrane potential and modulate dedifferentiation and/or regeneration. In yet another alternative, novel compounds can be generated and tested for the ability to (i) modulate ion flux and membrane potential (e.g., by influencing the expression or activity of one or more ion transporter proteins) and (ii) modulate dedifferentiation and/or regeneration in one or more model systems.

The present invention also provides additional methods based on an appreciation for the importance of ion flux in a range of biological process. In one aspect, the invention provides methods for identifying and/or isolating progenitor cells based on membrane potential or pH characteristics typically associated with progenitor cells. In another aspect, the invention provides a hierarchical screening approach that allows ordered identification and characterization for whether a particular biological process is mediated, in whole or in part, by modulation of membrane potential. This ordered approach is unique to previous candidate screening approaches and permits efficient identification of (i) whether a particular process is mediated by modulation of membrane potential and (ii) what class or subclass of ion transporter protein mediates membrane potential in that particular system or process.

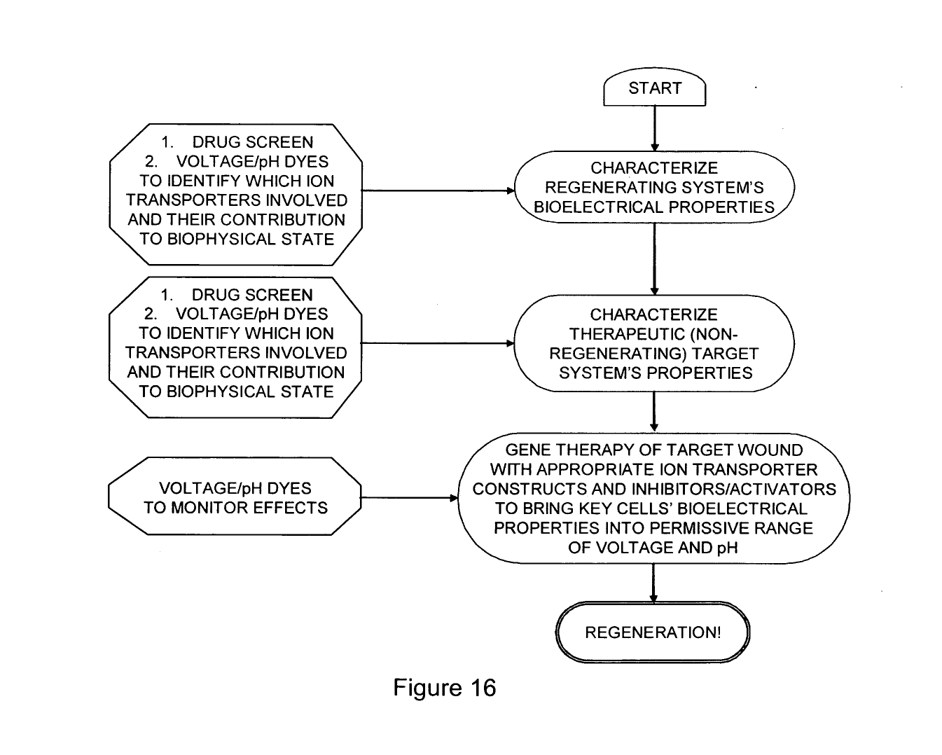

Finally, the present invention contemplates that the various screening methods of the present invention can be used in combination as part of an ordered approach for studying a process in a given biological system (e.g., regeneration) and selecting appropriate agents that modulate ion flux in that system. Use of a combination of the methods described herein to study a biological process is depicted in FIG. 16.

### (ii) Definitions

The articles “a” and “an” are used herein to refer to one or to more than one (i.e., to at least one) of the grammatical object of the article. By way of example, “an element” means one element or more than one element.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The term “wild type” refers to the naturally-occurring polynucleotide sequence encoding a protein, or a portion thereof, or protein sequence, or portion thereof, respectively, as it normally exists in vivo. The term “wild type” also refers to a phenotypically and genotypically normal organism.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent). The term “mutant” also refers to an organism with one or more phenotypic or genotypic alterations in comparison to a wild type organism of the same species.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The terms “compound” and “agent” are used interchangeably to refer to nucleic acids, peptides, polypeptides, or small molecules. In the context of the present invention, compounds or agents may modulate ion flux, for example, by inhibiting or promoting ion flux mediated by a particular ion transporter protein or class of ion transporter proteins. Exemplary nucleic acid agents include, but are not limited to, sense or antisense nucleic acids, sense or antisense oligonucleotides, ribozymes, and RNAi constructs. Exemplary peptide and polypeptide agents include growth factors, transcription factors, peptidomimetics, and antibodies, as well as particular ion transporter proteins or subunits thereof. Exemplary small molecules include small organic or inorganic molecules, e.g., with molecular weights less than 7500 amu, preferably less than 5000 amu, and even more preferably less than 2000, 1500, 1000, or 500 amu. One class of small organic or inorganic molecules is non-peptidyl, e.g., containing 2, 1, or no peptide and/or saccharide linkages. The term agent is also used, when specified, to refer to compounds that produce a detectable signal in response to pH or membrane voltage. In this context, the agent serves as an indicator of intracellular pH or membrane voltage. Exemplary agents include fluorescent dyes that provide a detectable signal indicative of membrane voltage or pH.

For purposes of this invention, the chemical elements are identified in accordance with the Periodic Table of the Elements, CAS version, Handbook of Chemistry and Physics, 67th Ed., 1986-87, inside cover.

A “marker” is used to determine the state of a cell. Markers are characteristics, whether morphological or biochemical (enzymatic), particular to a cell type, or molecules expressed by the cell type. A marker may be a protein marker, such as a protein marker possessing an epitope for antibodies or other binding molecules available in the art. A marker may also consist of any molecule found in a cell, including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells typically lose or greatly restrict their capacity to proliferate and such cells are commonly referred to as being terminally differentiated.

The term regeneration refers to the restoration of cells, tissues, or structures following injury, ablation, loss, or disease. Regeneration involves an interplay of proliferation, differentiation, sometimes dedifferentiation. In some instances, regeneration refers to individual cells or groups of cells. In other instances, regeneration comprises restoration of all or a portion of a tissue or organ. The invention provides methods of promoting or enhancing regeneration. In some embodiments, the method of promoting or enhancing regeneration includes modulating one or more of proliferation, differentiation, dedifferentiation, survival, or migration.

As used herein, the term “non-regenerating cells” refers to non-naturally regenerating cells from a non-regenerating organism or cells from a regenerating organism in a refractory period. The term “naturally regenerating cells” refers to cells from a regenerating organism that are typically capable of regenerating. In certain embodiments naturally regenerating cells may be in a regenerating state.

As used herein, the term “population of cells” refers to one or more cells in a tissue or organ. A tissue or organ of the invention may be part of an organism or cultured in vitro.

As used herein, the term “effective amount” means the total amount of the active component(s) of a composition or compound that is sufficient to cause a statistically significant change on a detectable biochemical or phenotypic characteristic. When applied to an individual active ingredient, administered alone, the term refers to that ingredient alone. When applied to a combination, the term refers to combined amounts of the active ingredients that result in the effect, whether administered in combination, serially or simultaneously.

The term “membrane” refers to phospholipid bilayers.

The term “ion flux” refers to the movement of ions through an area/unit time. The term does not imply anything about the mechanism of ion movement. The term includes ion flux mediated by any ion transporter protein regardless of whether the transporter protein actively or passively shuttles ions. The term ion flux includes movement of ions into a cell or movement of ions out of a cells (e.g., efflux or influx).

As used herein, the terms “ion transporter proteins” and “transporter proteins” are used interchangeably and include proteins that mediate ion flux regardless of the particular ion species transported or the particular mechanism of action. The term includes proteins that are passive transporters, as well as proteins that are active transporters. “Class of ion transporter proteins” refers to categories of transporter proteins organized based on similar functional characteristics. For examples, a class of ion transporter proteins may include transporter proteins that transport a particular ion species (e.g., Ca, Na, H) or transporter proteins that transport a particular ion species using a particular mechanism of action.

### DETAILED DESCRIPTION OF ILLUSTRATIVE EMBODIMENTS

### (i) Detailed Description of Methods and Apparatuses

(a) Methods of Modulating Dedifferentiation and/or Regeneration

The regeneration of complex tissues and organ systems lost to injury, senescence, or disease is a key goal of biomedicine. In addition to its clinical applications, the regeneration of organs is fascinating because it represents one of the most fundamental properties of most living things: recognition of damage, and self-repair. Currently, substantial efforts are being invested in academia and industry to understand the fundamental principles influencing dedifferentiation and regeneration so that the powers of these processes can be harnessed and used to treat degenerative diseases and injuries.

Animal regeneration can be conceptually divided into four phases: 1) injury processes which initiate regeneration, 2) formation of a blastema including in some cases the de-differentiation of local cells, 3) the differentiation of blastema components, and 4) morphogenesis of appropriate structures. Additionally, it has long been recognized that certain species appear to have a more robust regenerative capacity (e.g., planaria, amphibians, fish) while other species appear to have a less robust regenerative capacity. Thus, one goal of scientists studying regeneration has been to understand the principles and processes that modulate de-differentiation and/or regeneration in species capable of mounting robust regenerative responses, so that these principles and processes can be applied to increase the regenerative capacity of other organisms.

This aspect of the present invention is based, in part, on our findings indicating that modulation of membrane potential and/or pH can be used to reset differentiated cells. Without being bound by theory, the resetting of cells may promote their dedifferentiation. Once dedifferentiated, these cells are responsive to endogenous or exogenously supplied cues that drive proliferation and other processes. As a result, regeneration of tissues can occur. Accordingly, by modulating membrane potential and/or pH, cells, tissues, and organs can be regenerated. As outlined further below, studies performed in organisms and tissues that naturally regenerate indicates that there is a window of membrane potential and/or pH permissive for regeneration. Thus, by modulating membrane potential and/or pH to within this regeneration permissive range, the present invention provides methods for promoting regeneration in any of a variety of species—including species and tissues that do not have a naturally high regenerative potential.

In certain aspects, the present invention provides a method of promoting regeneration by modifying the membrane potential and/or pH of one or more cells. Promoting regeneration in non-regenerating cells may be accomplished by first determining the membrane potential and/or pH range permissive for regeneration in naturally regenerating cells. This membrane potential and/or pH range may be determined experimentally or by referring to known values. The membrane potential and/or pH range is determined in the population of non-regenerating cells of interest. Again, this membrane potential and/or pH range may be determined experimentally or by referring to known values. In certain embodiments, the permissive membrane potential and/or pH range will be the same in naturally regenerating cells and non-regenerating cells and will not need to be determined for each tissue or organ or organism. The population of non-regenerating cells is contacted with an agent that modulates ion flux mediated by a class of ion transporter proteins. The agent modifies the membrane potential and/or pH of one or more cells in the population of non-regenerating cells to the range permissive for regeneration as determined experimentally or by referring to known values; thereby promoting regeneration of one or more cells in the population of cells. Accordingly, compounds that modulate ion flux (e.g., inhibitor/promoters of the expression or activity of an ion transporter protein or a class of ion transporter proteins) can be used in methods for promoting dedifferentiation and/or regeneration. Thus, the present invention provides methods, compositions, and pharmaceutical compositions that can be used to promote cell dedifferentiation and/or regeneration. The methods and compositions of the invention can be used to increase our understanding of the principles underlying dedifferentiation and regeneration. The methods and compositions of the invention can be used to promote dedifferentiation and regeneration in vivo or in vitro. The methods and compositions of the invention can be used as a basis for the development of therapeutic methods for treating degenerative diseases and injuries that could be ameliorated using methods that enhance regenerative capacity.

In certain aspects, promotion of regeneration occurs through a biological program. In other words, modulation of membrane potential or pH resets cells to a state permissive for regeneration. The reset cells are activated to carryout the endogenously present developmental pathways that lead to coordinated proliferation, differentiation, and migration of the multiple cell types that constitute complex tissues and organs. Without being bound by theory and in this embodiment of the invention, once the membrane potential of cells is modulated into the regeneration permissive range and the cells are developmentally reset, further signaling cascades and proteins needed to promote coordinated differentiation need not be exogenously supplied, but rather, the endogenous programs that exist during normal development and during regeneration in regenerating organisms are activated within the reset cells or populations of cells.

In certain aspects, modulation of ion flux in a small number of cells is sufficient to promote dedifferentiation and/or regeneration of an entire tissue or organ. In certain embodiments, the cells that are modulated to promote dedifferentiation and/or regeneration are progenitor cells. In certain embodiments, the cells that are modulated to promote dedifferentiation and/or regeneration are a single cell type. In certain embodiments, the cell type or types that are modulated in order to promote dedifferentiation and/or regeneration are not specifically determined.

Illustrative examples whereby ion flux can be manipulated to promote dedifferentiation and/or regeneration are provided in the examples. Briefly, compounds that specifically modulate (inhibit or promote) ion flux, membrane potential, and/or pH mediated by a specific ion transporter protein or a class of ion transporter proteins can be administered to cells, tissues, or organisms. In one embodiment, the method comprising modulating regeneration in a system with enhanced regenerative capacity (e.g., planaria, Xenopus tail, Xenopus limb, zebrafish tail). In another embodiment, the method comprises promoting regeneration (e.g., enhancing regenerative capacity) in a system that does not endogenously have a robust regenerative capacity (FIG. 16).

Compounds identified as specifically modulating ion flux during regeneration can be formulated and administered as part of a method for promoting dedifferentiation and/or regeneration in vitro or in vivo. Such compounds can be administered as a composition or a pharmaceutical preparation. Such compounds can be developed and used as part of a therapeutic regimen.

In certain embodiments, any ion channel or pump may be expressed or modulated modulate membrane potential, thereby promoting the combination of proliferation, differentiation, and/or dedifferentiation needed to regenerate damaged, diseased, or injured tissue. In certain embodiments, the ion channel or pump may come from any organism and may originate from a different organism than the one being treated. Some representative channels and pumps are listed in Table 5 (the nucleic acid and amino acid sequences disclosed at the representative accession numbers are hereby incorporated by reference).

The present invention provide methods for identifying membrane potential and/or pH ranges permissive for regeneration, as well as methods for modulating membrane potential or pH into a regeneration permissive range. Based on these methods, the regenerative ability in tissues, organs, and organisms that are naturally refractory to regeneration can be enhanced.

In certain embodiments, membrane potential or pH can be modulated using any transporter protein (e.g., channel or pump) capable of modulating membrane potential into the permissive range (e.g., either hyperpolarizing or depolarizing membrane potential depending on the membrane potential of a give cell relative to the permissive range). Without being bound by theory, in such embodiments, the membrane potential itself, rather than the particular ion(s) being transporter, is critical for promoting regeneration. In other embodiments, membrane potential can be modulated using specific ion transporter proteins or classes of ion transporter proteins (e.g., channel or pump). Without being bound by theory, in such embodiments, both the membrane potential itself and the particular ion(s) being transporter may be important for regeneration.

In certain embodiments, the V-ATPase H⁺ pump, as well as compounds that promote the activity of the V-ATPase H⁺ pump, may be expressed to promote the combination of proliferation, differentiation, and/or dedifferentiation needed to regenerate damaged, diseased, or injured tissue. In certain embodiments, the P-type H⁺ATPase, as well as compounds that promote the activity of the P-type H⁺ ATPase, may be expressed to promote the combination of proliferation, differentiation, and/or dedifferentiation needed to regenerate damaged, diseased, or injured tissue. In certain embodiments, K⁺ channels, as well as compounds that promote the activity of K⁺ channels, may be expressed to promote the combination of proliferation, differentiation, and/or dedifferentiation needed to regenerate damaged, diseased, or injured tissue. In certain embodiments, the ROMK K⁺ channel may be expressed to promote the combination of proliferation, differentiation, and/or dedifferentiation needed to regenerate damaged, diseased, or injured tissue. In certain embodiments, the ERG K⁺ channel may be expressed to promote the combination of proliferation, differentiation, and/or dedifferentiation needed to regenerate damaged, diseased, or injured tissue. In certain embodiments, voltage-gated Na⁺ channels, as well as compounds that promote the activity of voltage-gated Na⁺ channels, may be expressed to promote the combination of proliferation, differentiation, and/or dedifferentiation needed to regenerate damaged, diseased, or injured tissue. In certain embodiments, more than one pump or channel may be expressed to promote the combination of proliferation, differentiation, and/or dedifferentiation needed to regenerate damaged, diseased, or injured tissue.

Note that particular cell and tissue types for which promoting regeneration is desirable may vary with respect to their membrane potential and pH characteristics. These characteristics may vary based on species, cell type, age, health, and other factors. Accordingly, modulating membrane potential or pH into a regeneration permissive range sometimes comprises hyperpolarizing the cell membrane and sometimes comprises depolarizing the cell membrane. However, given the extensive knowledge of ion transporters (e.g., hyperpolarizing and depolarizing ion transporters) and the existence of inhibitors and agonists of ion transporter function, one of skill in the art can readily select agents that hyperpolarize or depolarize—depending on the membrane potential relative to the permissive range.

Further embodiments of the foregoing methods are described in sections ii-v. Combinations of any of the foregoing and following embodiments are contemplated. Additionally, further discussions of ion transporter proteins and agents that modulate membrane potential are found throughout the specification.

(b) Ion Transporters in Biological Processes: An Efficient Screening Method

To generate an electric field, cell membranes provide a power source and generate a voltage potential by segregating ions across the barrier (resulting in membrane voltage). Because ions are charged, they cannot cross membranes by themselves. By establishing membrane bound compartments, cells use the movement of ions to establish ion concentration differences or gradients. Cells use stored energy in the form of these gradients (by carefully regulating the flow of ions across membranes) to accomplish everything, either by coupling local ion flux directly to local physiology (e.g. Ca²⁺ regulation of secretion) or by siphoning energy from the gradient and storing it as ATP.

Generally, the myriad of mechanisms by which ion transporter proteins shuttle ions and establish membrane potential are described using four characteristics: (a) the ion or ions that move; (b) the number of ions moved, and the relative direction of the ions when a transporter moves multiple ions; (c) whether the ion transporter protein undergoes a conformational change during transport; and (d) whether any of the ions move up its concentration gradient. This fourth characteristic determines whether movement of the ion requires energy (e.g., generated by ATP hydrolysis, generated via coupling the energy requiring process of moving up a gradient to the energy releasing process of moving down a gradient).

The first characteristic for describing an ion transporter protein is according to the particular ion or ions it translocates. The following ions generally move through biological membranes: H⁺, Cl⁻, Na⁺, K⁺, and Ca²⁺, and to a lesser degree, Fe²⁺, Cu²⁺, and Zn²⁺. Additionally, charged species such as HCO₃⁻ and OH⁻ can move via ion transporter proteins across cell membranes.

Most transporter proteins such as channels and pumps are highly specific with respect to the ion species translocated. However, gap junctions are ion transporter proteins that permit non-specific (with respect to ion species) ion flux (Bruzzone et al., 1996, Bioessays 18: 709-718; Nicholson, 2003, J Cell Sci 116: 4479-4481).

The second characteristic is the number of ion species shuttled by a particular transporter. When a transporter translocates a single ion species (e.g., only Cl⁻ ions or only Ca²⁺ ions), it is often referred to as a uniporter. When a transporter translocates two or more ion species and shuttles the two or more species in the same direction, the transporter is often referred to as a symporter or a cotransporter. When a transporter translocates two or more ion species but shuttles species in opposite directions relative to one another, the transporter is often referred to as an antiport or an exchanger.

The third characteristic deals with whether the transporter undergoes a conformational change during transit of the ion species. Ion transporter proteins that remain open at both ends during ion translocation (e.g., at both their intracellular and extracellular end), are often referred to as channels. Ion transporter proteins that undergo a conformational change during translocation so that one end of the transporter open at a time are often referred to as transporters. Ion transporter proteins that physically travel from one side of the membrane to the other during translocation of an ion species are often referred to as either transporters, carriers, or ionophores.

We note that ion transporter proteins can also be described based on whether the ends of the protein are opened or closed in the absence of active ion translocation. Proteins that remain closed (e.g., the intra and extracellular openings are closed) in the absence of ion translocation are often termed gated proteins. The foregoing terminology is often used in the art to help readily categorize various transporter proteins. Throughout the application however, unless reference to a particular protein is being made, we will use the term “ion transporter protein” generically to refer to any transporter protein regardless of mechanism of action.

The fourth characteristic involves whether movement of ion species requires energy input. Ion movement down a gradient does not require the input of energy, and is referred to as passive transport or facilitated diffusion. Ion movement up a concentration gradient requires energy, and is referred to as active transport. The energy for active transport may come from a variety of sources. For example, the energy may come from the hydrolysis of ATP. Alternatively, the energy may come from coupling ion movement requiring energy to ion movement that releases energy.

In addition to the wide variety of channels, pumps, and carriers, gap junctions (GJs) also modulate ion flux across membranes. Gap junctions are plasma membrane protein complexes that directly connect the cytoplasm of neighboring cells, allowing for cell-cell exchange known as gap junctional communication (GJC). GJs can be opened or closed post-translationally (in response to changes in local Ca²⁺ concentration, pH, and/or membrane voltage) and provide for fairly complex gating of the flow of ions and small molecules (<1 kD) directly between cells (Bruzzone and Giaume, 1999, Advances in Experimental Medicine and Biology 468: 321-337; Goldberg et al., 1999, Nature Cell Biology 1: 457-459; Jalife et al., 1999, J of Cardiovascular Electrophysiology 10: 1649-1663; Lampe and Lau, 2000, Archives of Biochemistry and Biophysics 384: 205-215; and White et al., 1994, Nature 371: 208-209). If the GJs connecting two cells are opened, any ion gradient that previously existed will quickly, although not instantaneously, dissipate. Thus, ion flux through GJs can be an important determinant of ion distribution in a tissue, and expression patterns of GJs can establish isopotential cell fields.

The above characteristics are useful for mechanistically describing how particular ion transporter proteins and classes of ion transporter proteins function to shuttle ion species and regulate ion flux across cell membranes. Many of the identified ion transporter proteins were named based on one or more of their functional characteristics. Throughout this application, however, the generic term ion transporter protein encompasses any of the foregoing categories of proteins (e.g., GJs, channels, pumps, etc) that function to shuttle one or more ion species across cell membranes to mediate ion flux, membrane potential, and/or pH. Other specific terminology is used, as necessary, to refer to a particular class of ion transporter proteins or to a particular ion transporter protein.

In addition to the many ion-specific concentration differences (the chemical gradients) that exist as a consequence of membrane flux, there is also a single, all-inclusive charge difference (the electrical gradient) across the membrane. An electrical gradient is called a voltage, and cellular membranes are described as having a membrane voltage (Vₘ). Many cells, for example differentiated cells, have resting potentials on the order of −60 mV.

Vₘ is determined by the gradients, and therefore the concentrations, of all the ions that move across cell membranes. Thus, changes in ion flux via a particular ion transporter protein may alter both the relative concentration of the particular ion in the cell and the membrane potential of the cell. Accordingly, the methods of the present invention can be used to assess not only whether modulation of a particular class of ion transporter proteins modulates ion flux, thereby mediating a particular biological process, but also whether modulation of a particular class of ion transporter proteins modulates membrane voltage, thereby mediating a particular biological process.

The appreciation that ion flux and membrane potential serve not only a general house keeping function, but also help mediate and/or initiate complex biological events is a recent phenomenon. To illustrate, ion transporter proteins can act to directly regulate the function of non-ion transporter proteins. Alternatively, ion transporter proteins can mediate the flux of an ion species that in turn interacts with an oppositely charged point on a peptide resident within a cell, thereby changing the charge distribution and/or structure of the protein. By way of another example, ion flux can influence downstream events by essentially transforming a cell or tissue into an electrophoresis apparatus (Levin and Mercola, 1998, Developmental Biology 203: 90-105; Levin and Mercola, 1999, Development 126: 4703-4714; Levin et al., 2002, Cell 111: 77-89, Fukumoto, T., Kema, I., and Levin, M., 2005, Current Biology, 15: 794-803; Adams D. S., Robinson K. R., Fukumoto T., Yuan S., Yelick P., Kuo L., McSweeney M., Levin M., 2006, Development, 133: 1657-1671; Hicks, C., Sorocco, D., and Levin, M., 2006, Journal of Neurobiology, in press; Esser, A. T., Smith, K. C., Weaver, J. C., and Levin, M., 2006, Developmental Dynamics, in press; Levin, M., Lauder, J., and Buznikov, G., 2006, Developmental Neuroscience, 28:171-185).

The invention provides a reiterative method for systematically (i) evaluating whether ion flux is involved in a particular biological process in a particular model system, and, if so, (ii) identifying a broad class of ion transporter proteins that mediate ion flux during the particular biological process and (iii) systematically and progressively narrowing the class of ion transporter proteins that mediate ion flux during the particular biological process. In this way, the method provides a systematic approach for identifying a subset of ion transporter proteins that modulate ion flux during a particular biological process. This subset of ion transporter proteins can then be individually studied to understand the role of individual ion transporter proteins during a particular biological process in a particular model system.

The logical structure of the screen is as follows. The first compound evaluated for an effect on a particular biological process is a compound with low specificity. In other words, the first compound modulates the activity of a large family or multiple families of ion transporter proteins (Step 1). We note that at this and any subsequent steps in the assay, compounds are applied in an amount effective to penetrate cells and to modulate ion flux without producing toxic effects. One of skill in the art can readily titrate compounds to select dosages of compounds appropriate for the particular model system.

If no effect on the particular biological process being assayed is observed after applying the step 1 compound, the entire broad class of ion transporter proteins is eliminated as a candidate for having a role in that particular biological process or model system. We note that, as confirmation of this result, one may optionally repeat the step 1 experiment using a different compound that also modulates the activity of the same broad class of ion transporter proteins. Failure of either compound to produce a detectable change in your biological process provides increased confidence that the broad class of ion transporter proteins is not involved in that biological process or model system.

If, however, a phenotype (e.g., a change in the particular biological process being observed) is detected following administration of the step 1 compound, the broad class of ion transporter proteins modulated by the step 1 compound is identified as a candidate class of ion transporter proteins that may be involved in mediating the particular biological process. In a second step, a population of cells is contacted with a second compound that is more specific. In other words, the second compound modulates ion flux mediated by a narrower class of ion transporter proteins. This narrower class of ion transporter proteins is a subset of the first, broad class of ion transporter proteins (step 2).

The method can continue using the same logic. At each subsequent round of screening (step 1, 2, 3, 4, etc), test cells are contacted with an increasingly specific compound. In other words, in each subsequent step, the cells are contacted with a compound that modulates ion flux mediated by an increasingly defined class of ion transporter proteins.

The number of steps of screening will depend on the system, and on the particular class of ion transporter proteins implicated in the previous step. 2, 3, 4, or more than 4 screening steps may suffice. Once the identified class of ion transporter proteins is sufficiently well defined and of reasonable size, one can begin analyzing individual ion transporter proteins. The number of ion transporter proteins within a class that can be reasonably analyzed individually will vary depending on the resources of the investigator, and the particular model system being used.

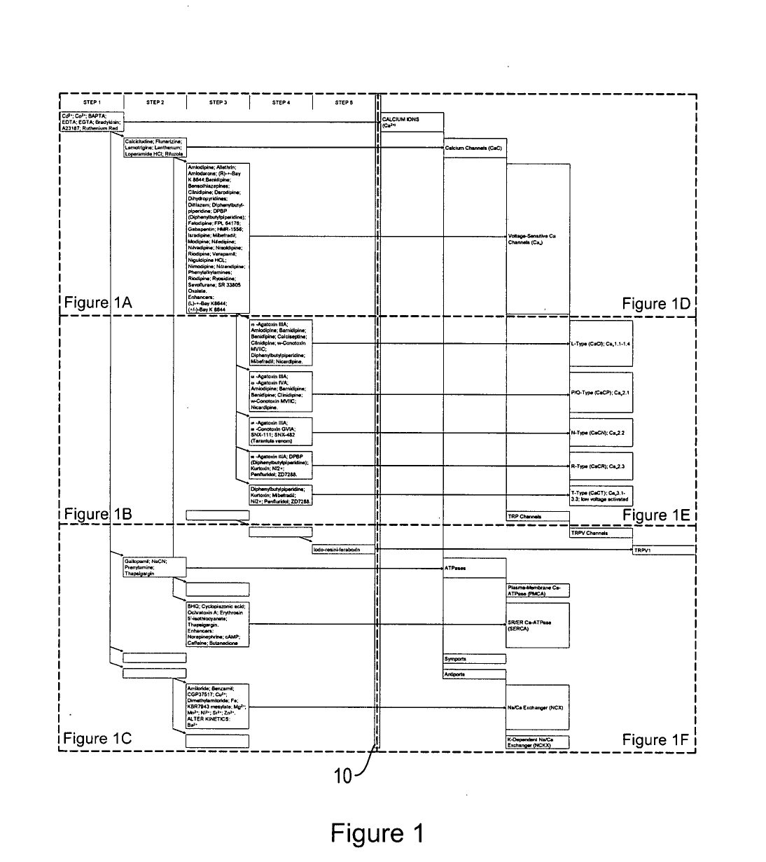

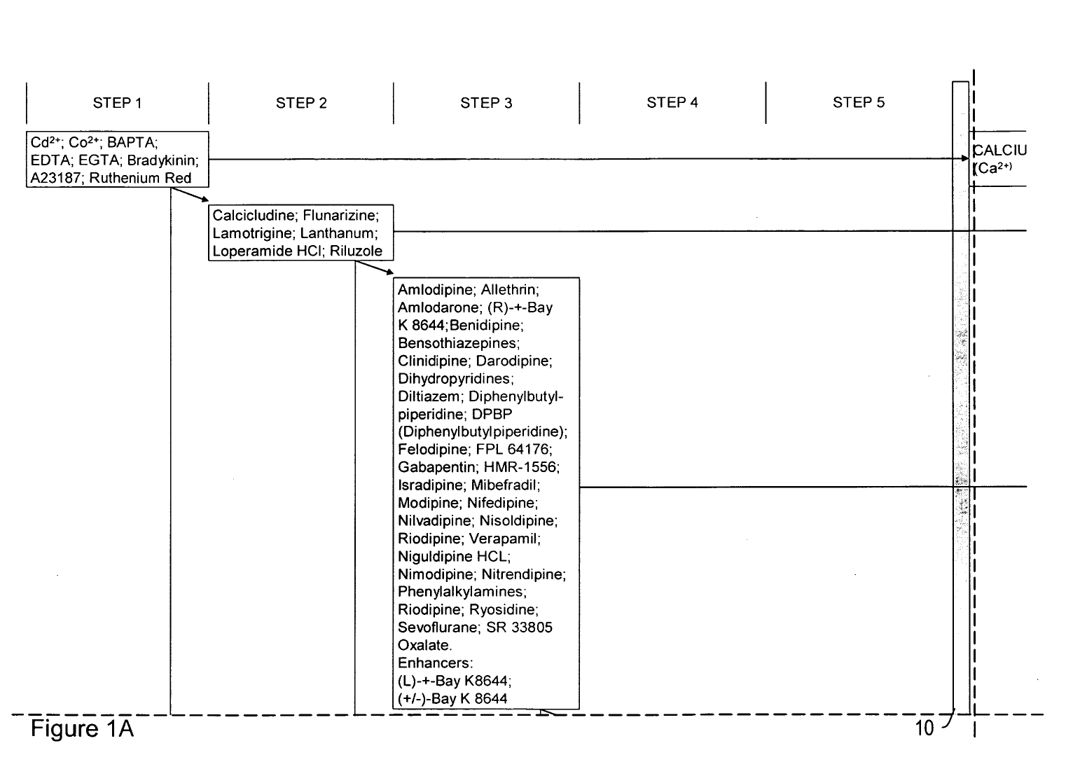

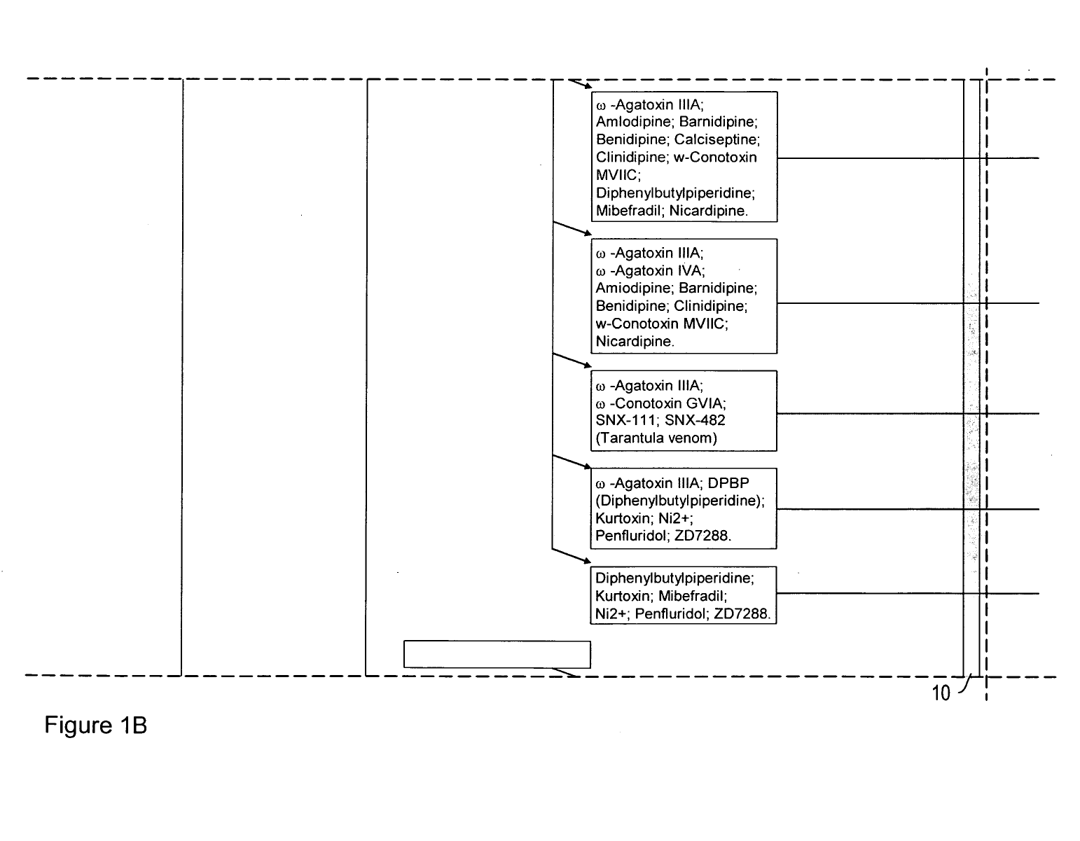

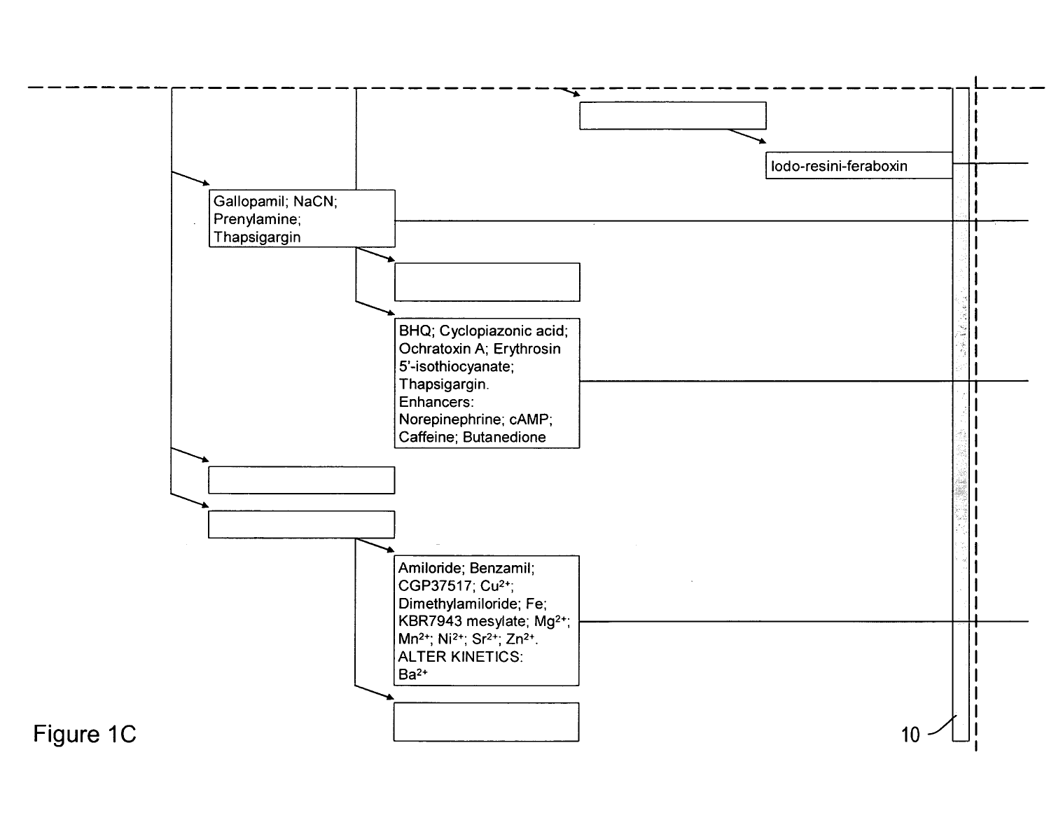

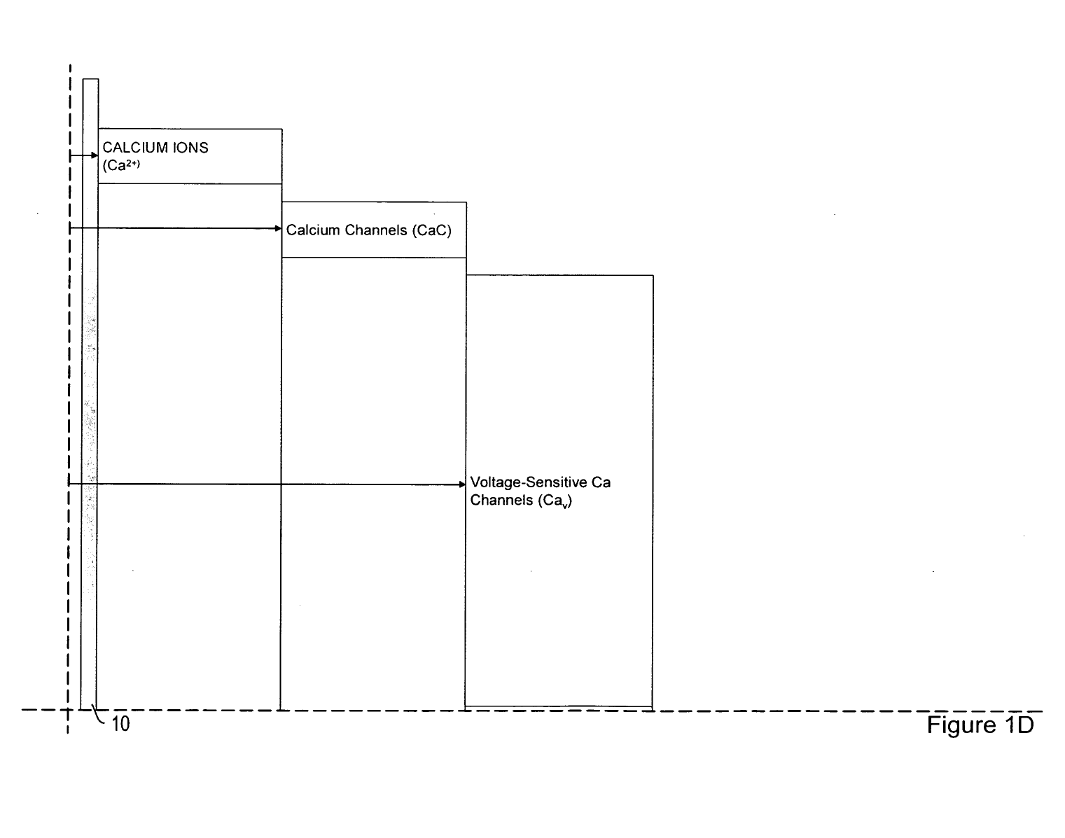

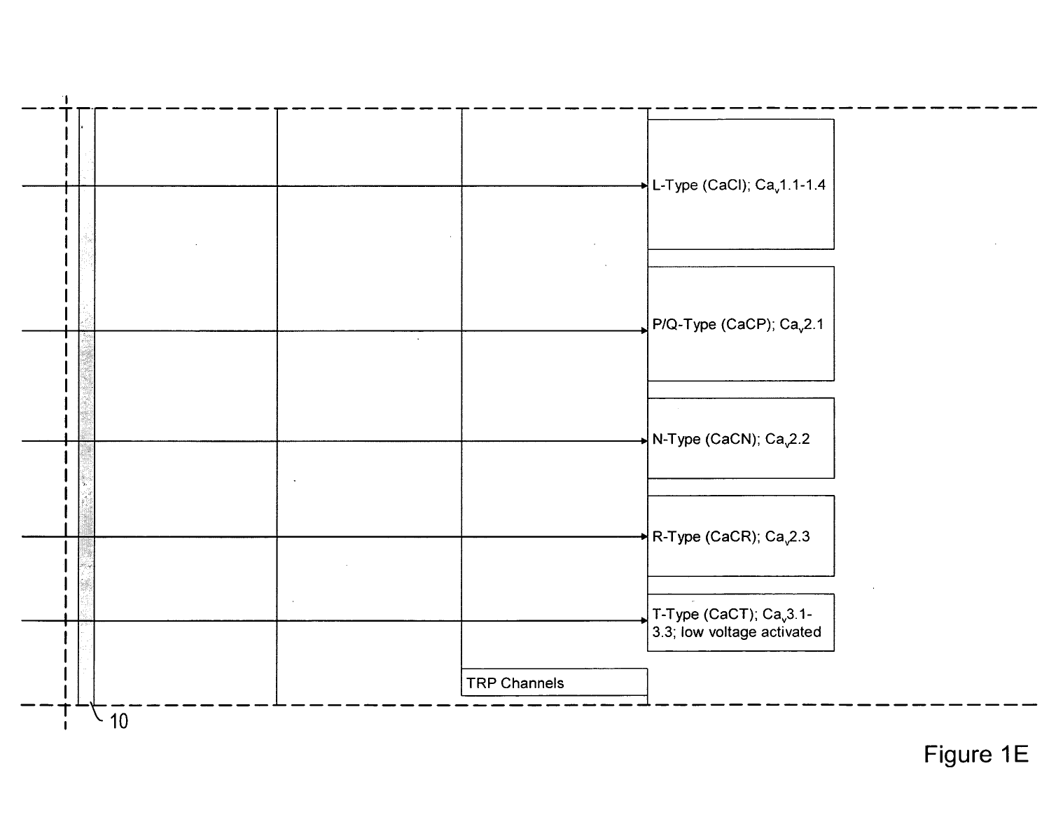

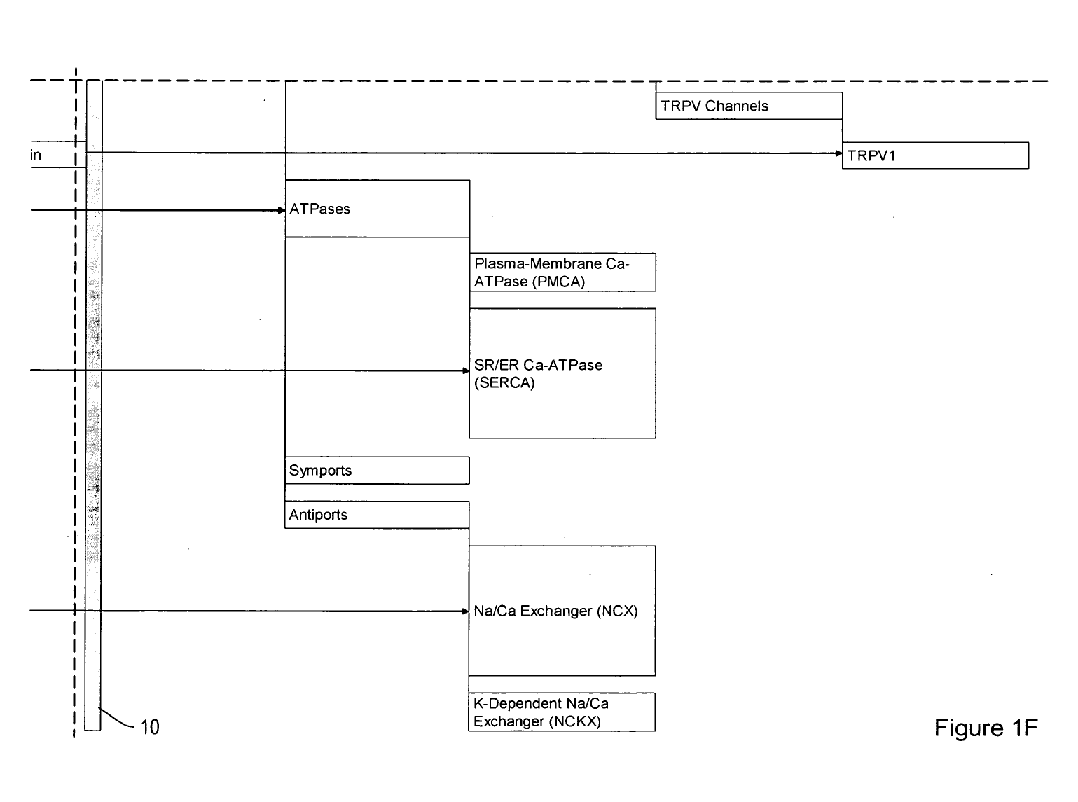

FIG. 1 provides an example of how this methodology can be used to systematically evaluate a role for calcium flux in a particular biological process and/or system. FIG. 1 summarizes a screening assay that includes up to five steps. Note that one can stop screening at any stage and begin analyzing the particular class of ion transporter proteins so identified. Alternatively, additional rounds of screening can be conducted.

At each step, a compound that modulates ion flux mediated by a class of ion transporter proteins is selected, and cells are contacted with said compound. Exemplary compounds that modulate the activity of a particular class of ion transporter proteins are depicted in the boxes shown to the left of the divider 10. The compounds have increasing specificity for a class of ion transporter proteins as the method progresses through steps 1, 2, 3, 4, and 5. Thus, a step 3 compound modulates a more narrowly defined class of ion transporter proteins then a class 2 compound, and a class 4 compound modulates a more narrowly defined class of ion transporter proteins than a class 3 compound.

To illustrate, one selects from amongst the compounds depicted in the box under step 1. If one of these compounds produces a phenotype in your particular system, calcium ion flux is identified as involved in your particular system. Thus, in step 1, one has identified an involvement for a broad class of ion transporter proteins that mediate calcium ion flux.

In step 2, one selects from amongst the compounds depicted in either of the boxes under step 2. If, for example, calcicludine, flunarizine, lamatrigine, lanthanum, riluzole, loperamide hcl, or a functionally similar compound produces a phenotype in your system, the broad class of ion transporter proteins that mediate calcium flux in your system has been narrowed to the calcium channel (CaC) class of transporter proteins. If, on the other hand, gallopamil, NaCN, prenylamine, or thapsigargin produces a phenotype in your system, the broad class of ion transporter proteins that mediate calcium flux in your system has been narrowed to the ATPase class of transporter proteins.

In each subsequent step, increasingly specific compounds can be used to identify an increasingly more define class of ion transporter proteins that mediate calcium flux, thereby producing a phenotype in your particular biological system.

We note that, in FIG. 1, the compounds listed to the left of divider 10 are ion transporter inhibitors unless otherwise indicated. Compounds are shown to the left of divider 10 and targets (e.g., class of ion transporter proteins) are represented to the right of divider 10. We also note that the listed compounds are exemplary. Additionally, we note that an investigator may chose to conduct each step of the screen in parallel and test a different compound that functions to inhibit the same class of compounds in parallel populations of cells. Observing the same or a similar phenotype using multiple compounds that modulate the same class of ion transporter proteins can be used to confirm the result and helps control for non-specific effects of the compounds.

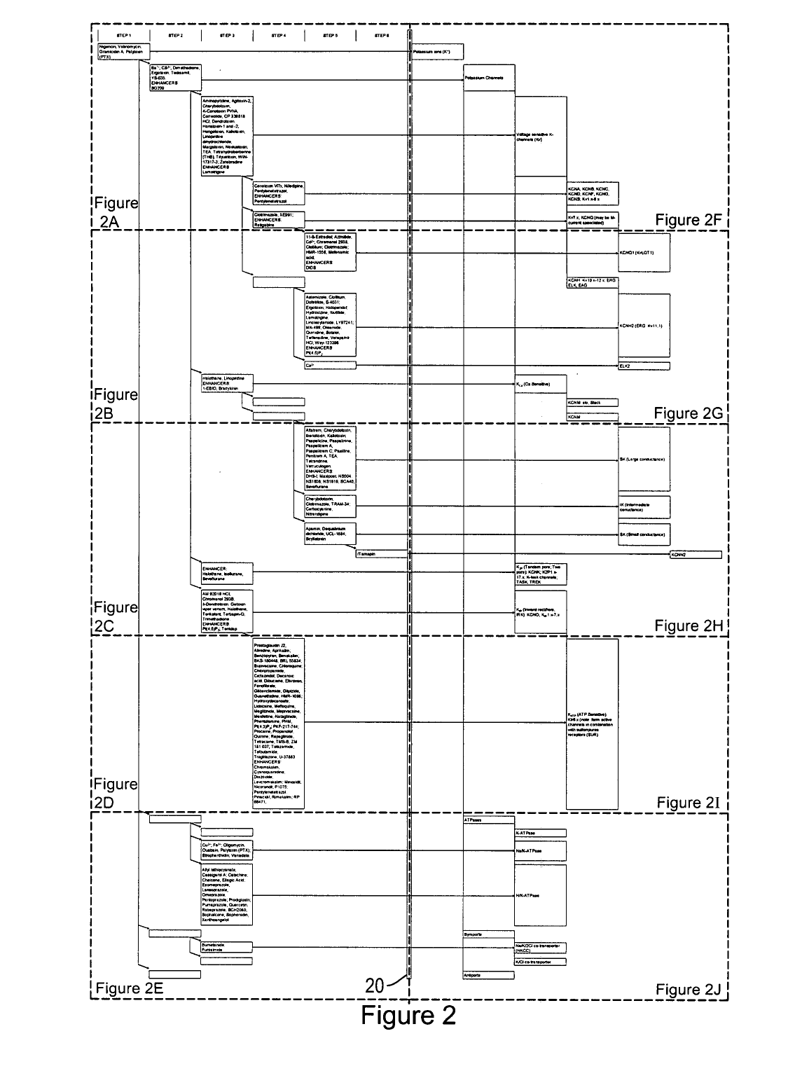

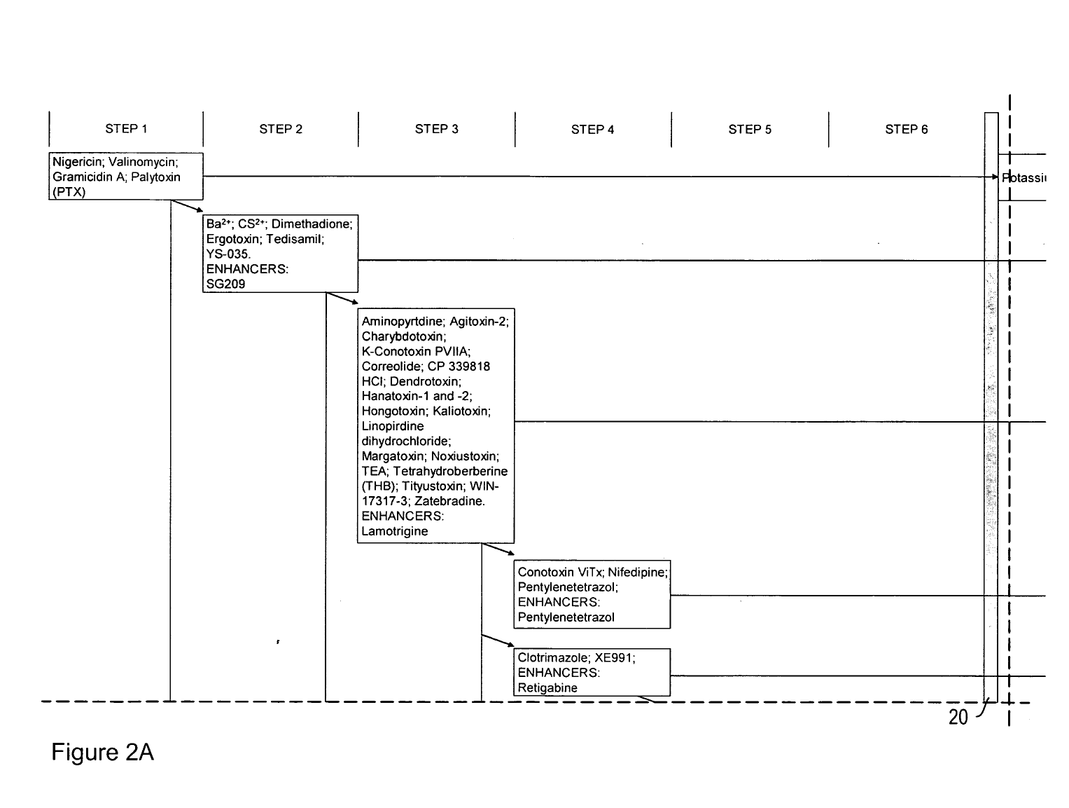

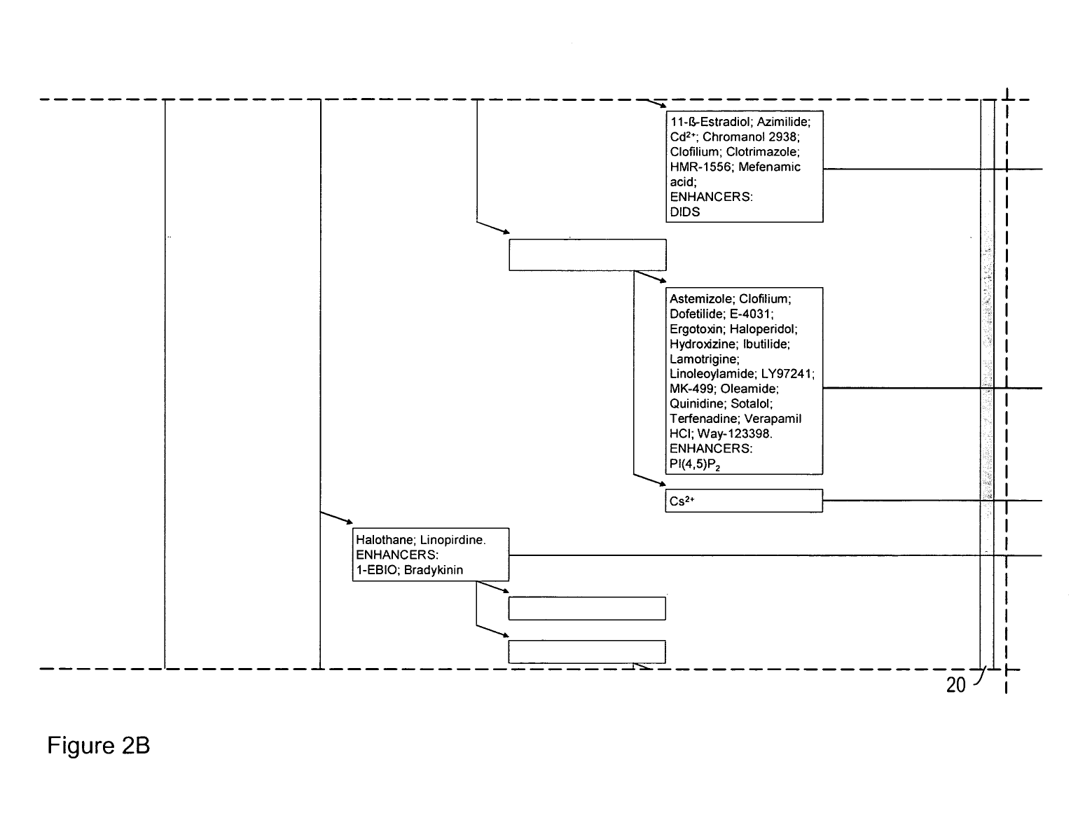

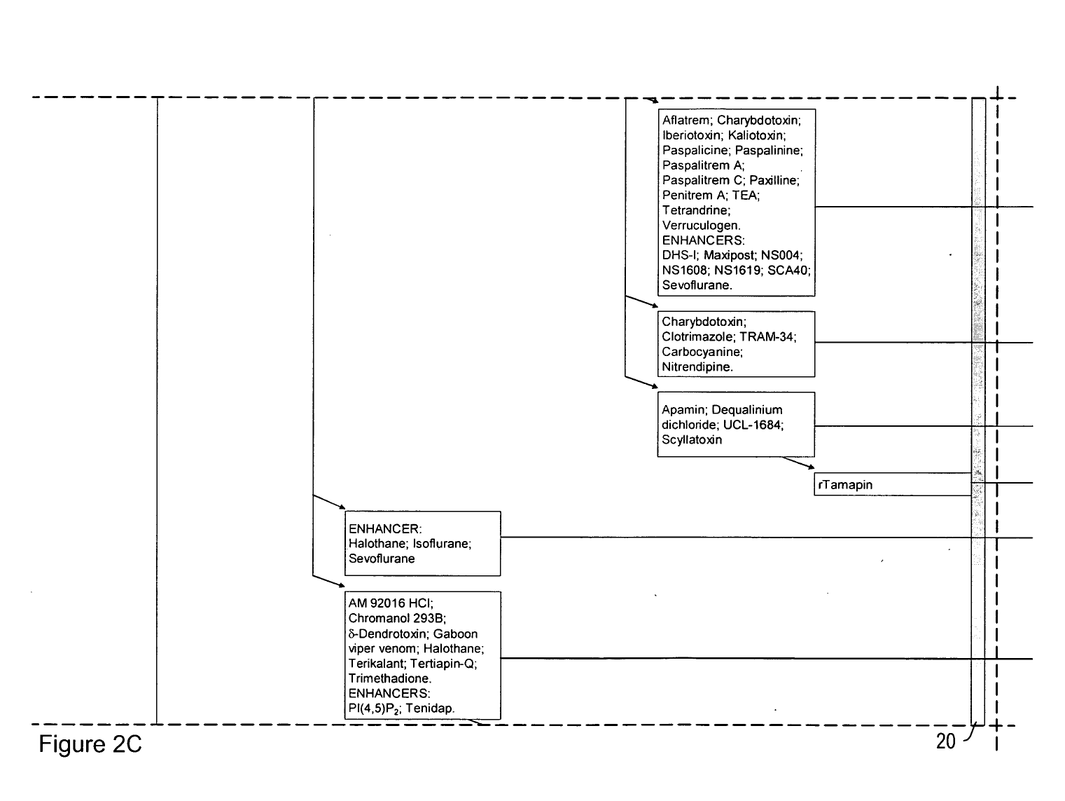

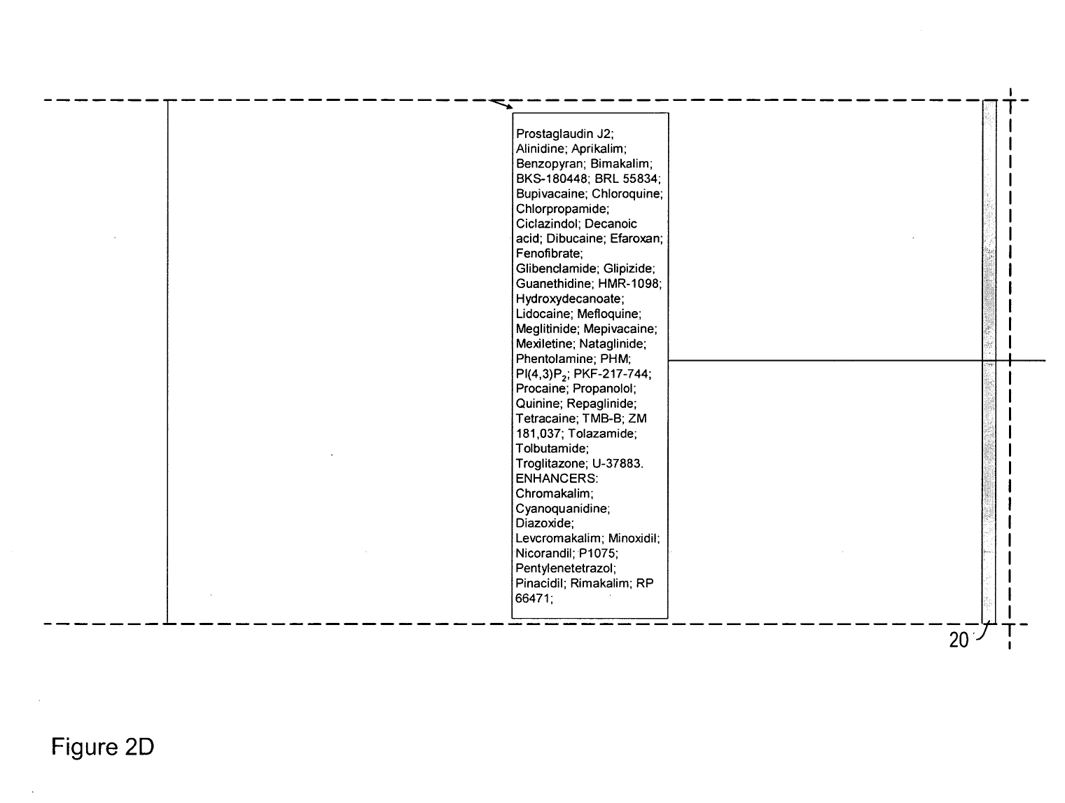

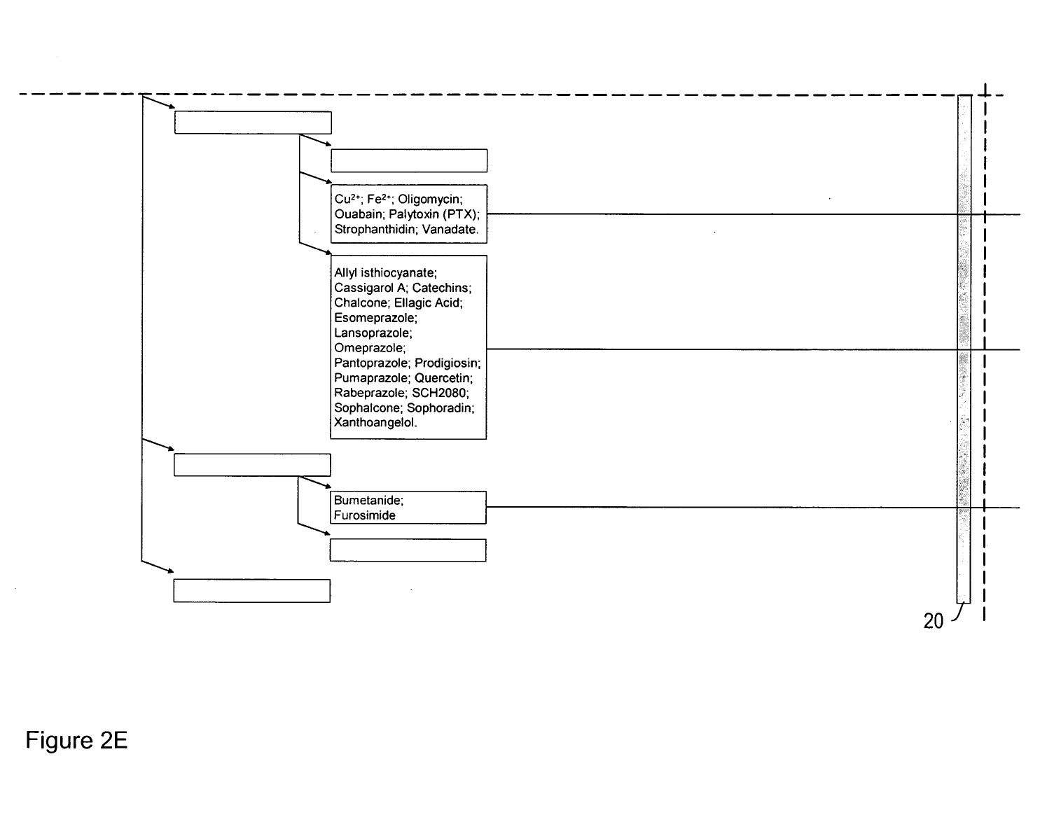

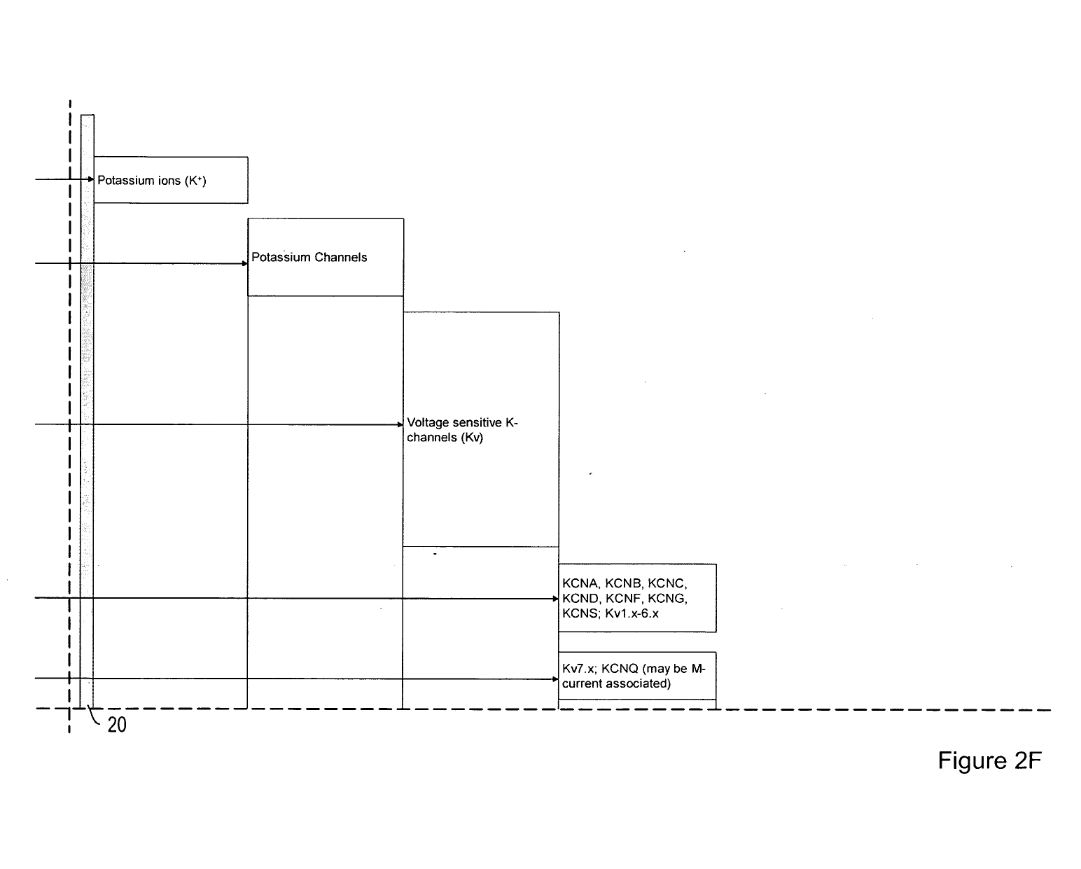

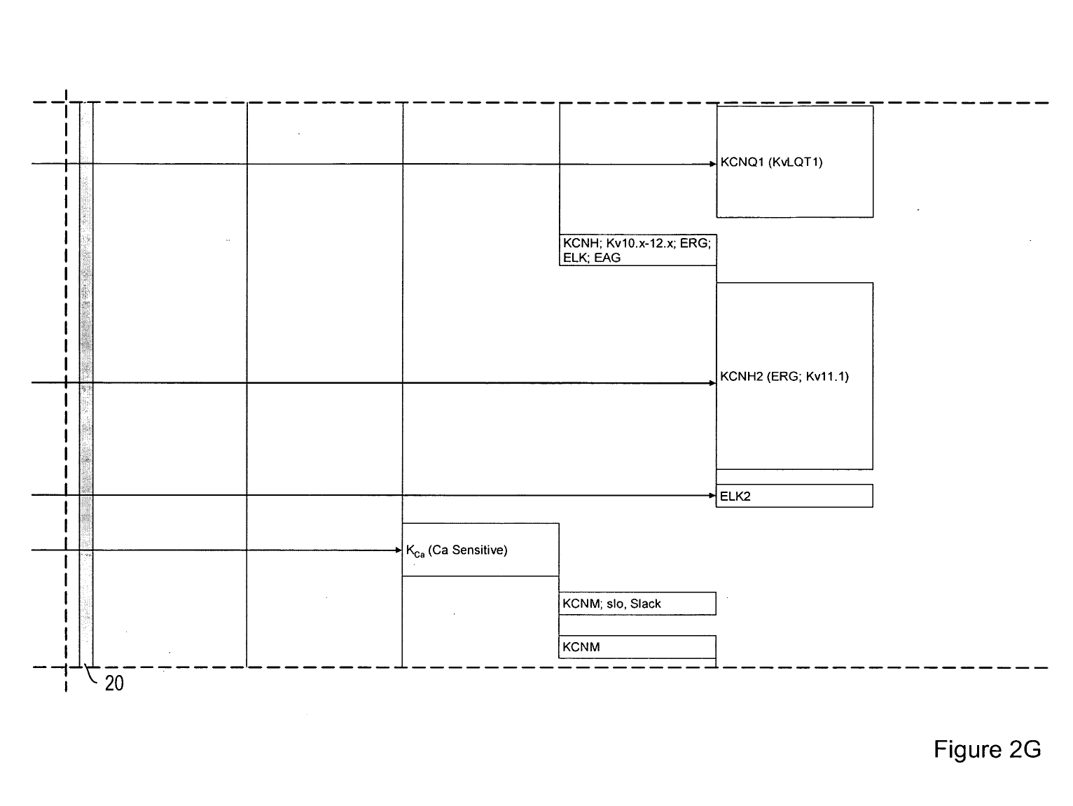

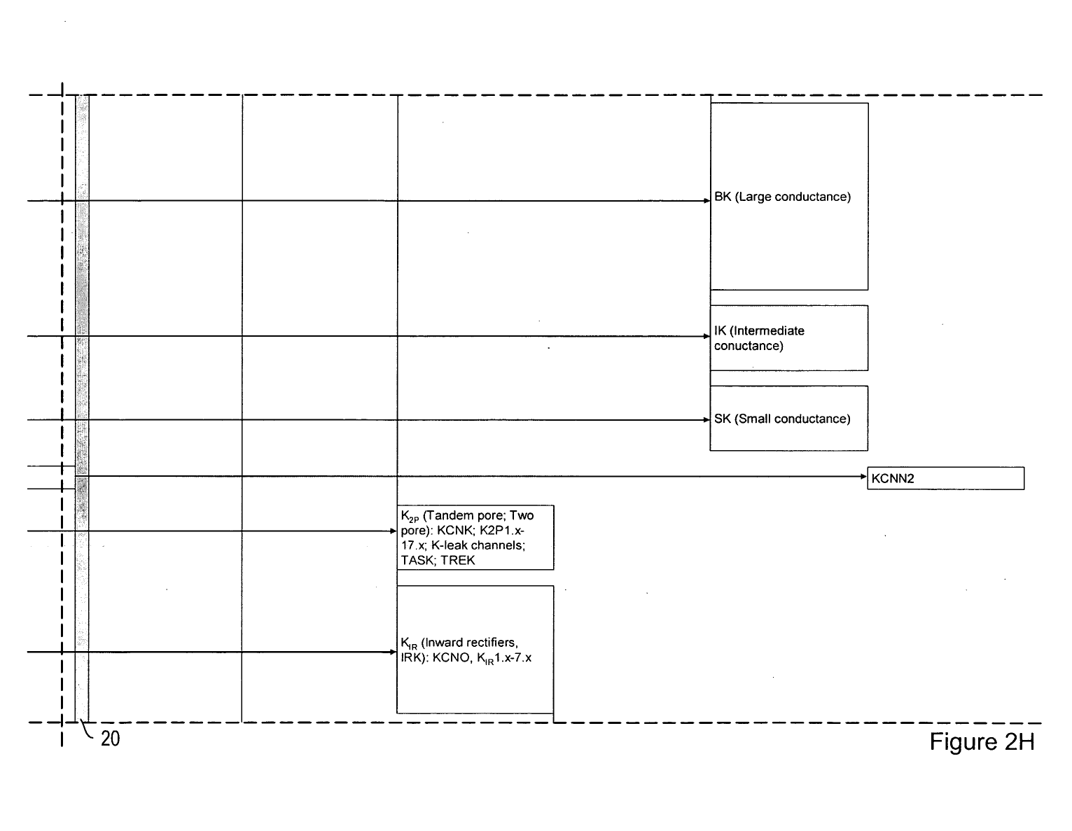

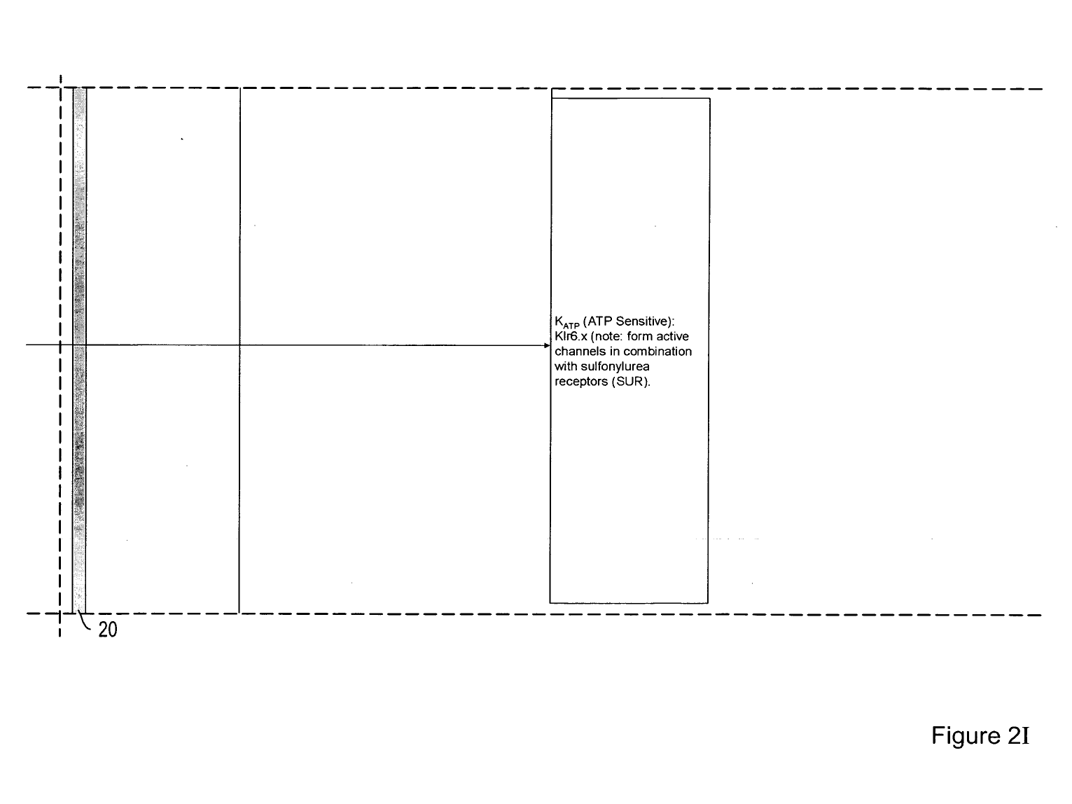

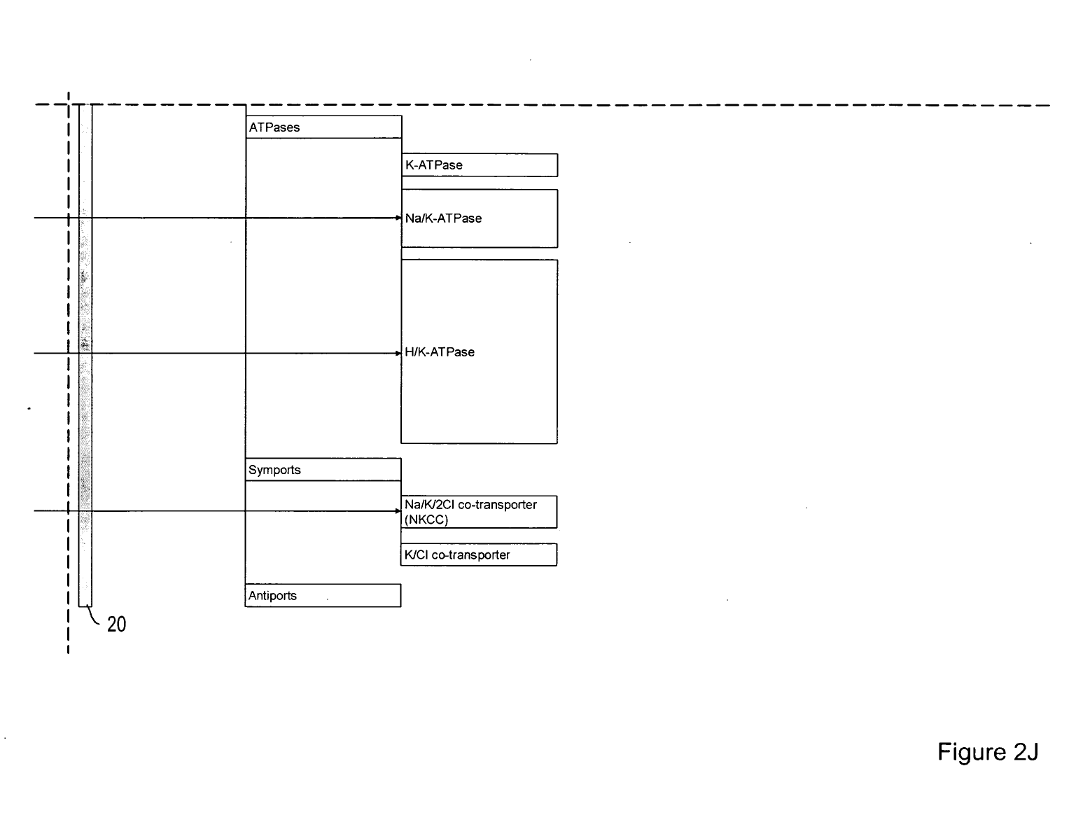

FIG. 2 provides an example of how this methodology can be used to systematically evaluate a role for potassium flux in a particular biological process and/or system. FIG. 2 summarizes a screening assay that may include up to five steps. Note that one can stop screening at any stage and begin analyzing the particular class of ion transporter proteins so identified. Alternatively, additional rounds of screening can be conducted.

At each step, a compound that modulates ion flux mediated by a class of ion transporter proteins is selected, and cells are contacted with said compound. Exemplary compounds that modulate the activity of a particular class of ion transporter proteins are depicted in the boxes shown to the left of the divider 20. The compounds have increasing specificity for a class of ion transporter proteins as the method progresses through steps 1, 2, 3, 4, and 5. Thus, a step 3 compound modulates a more narrowly defined class of ion transporter proteins then a class 2 compound, and a class 4 compound modulates a more narrowly defined class of ion transporter proteins than a class 3 compound.

To illustrate, one selects from amongst the compounds depicted in the box under step 1. If one of these compounds produces a phenotype in your particular system, potassium ion flux is identified as involved in your particular system. Thus, in step 1, one has identified an involvement for a broad class of ion transporter proteins that mediate potassium ion flux.

In step 2, one selects from amongst the compounds depicted in either of the boxes under step 2. If, for example, Ba, Cs, dimethadione, ergotoxin, tedisamil, YS035, SG209, or a functionally similar compound produces a phenotype in your system, the broad class of ion transporter proteins that mediate potassium flux in your system has been narrowed to the potassium channel class of transporter proteins.

In each subsequent step, increasingly specific compounds can be used to identify an increasingly more define class of ion transporter proteins that mediate potassium flux, thereby producing a phenotype in your particular biological system.

We note that, in FIG. 2, the compounds listed to the left of divider 20 are ion transporter inhibitors unless otherwise indicated. Compounds are shown to the left of divider 20 and targets (e.g., class of ion transporter proteins) are represented to the right of divider 20. We also note that the listed compounds are exemplary. Additionally, we note that an investigator may chose to conduct each step of the screen in parallel and test a different compound that functions to inhibit the same class of compounds in parallel populations of cells. Observing the same or a similar phenotype using multiple compounds that modulate the same class of ion transporter proteins can be used to confirm the result and helps control for non-specific effects of the compounds.

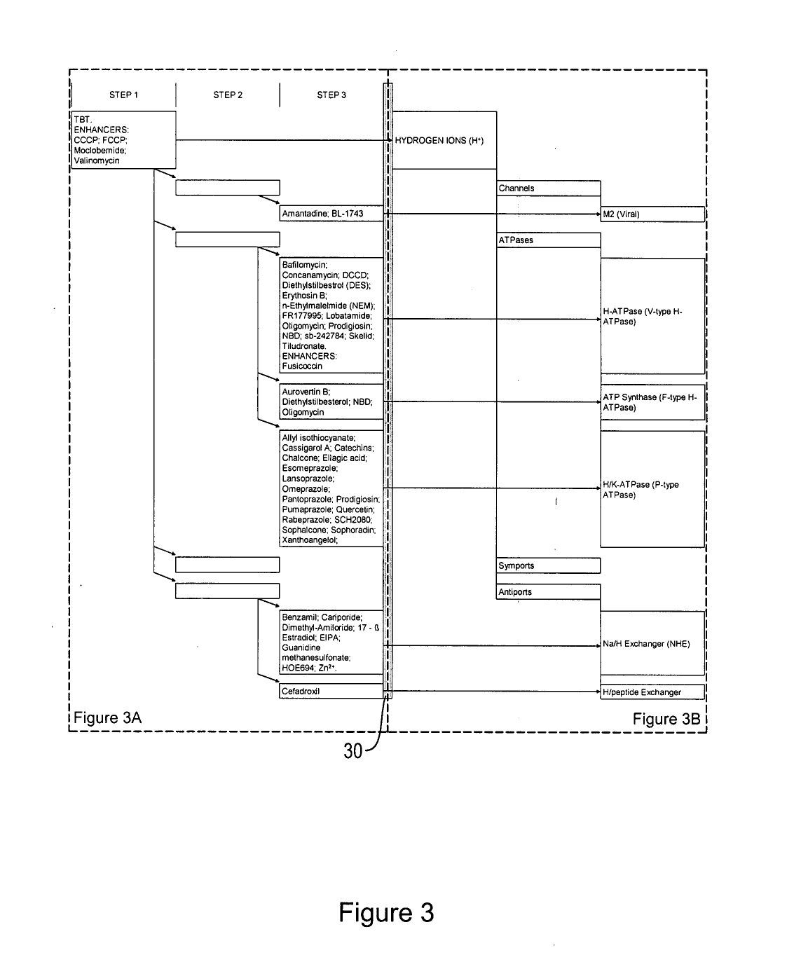

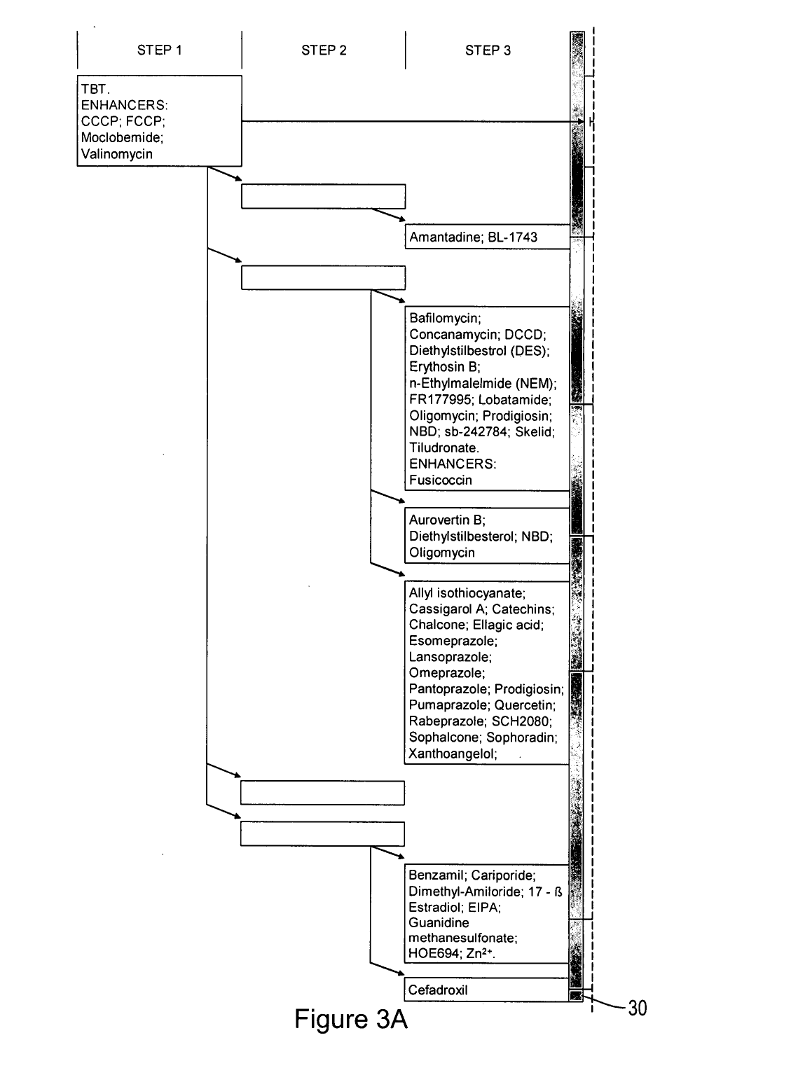

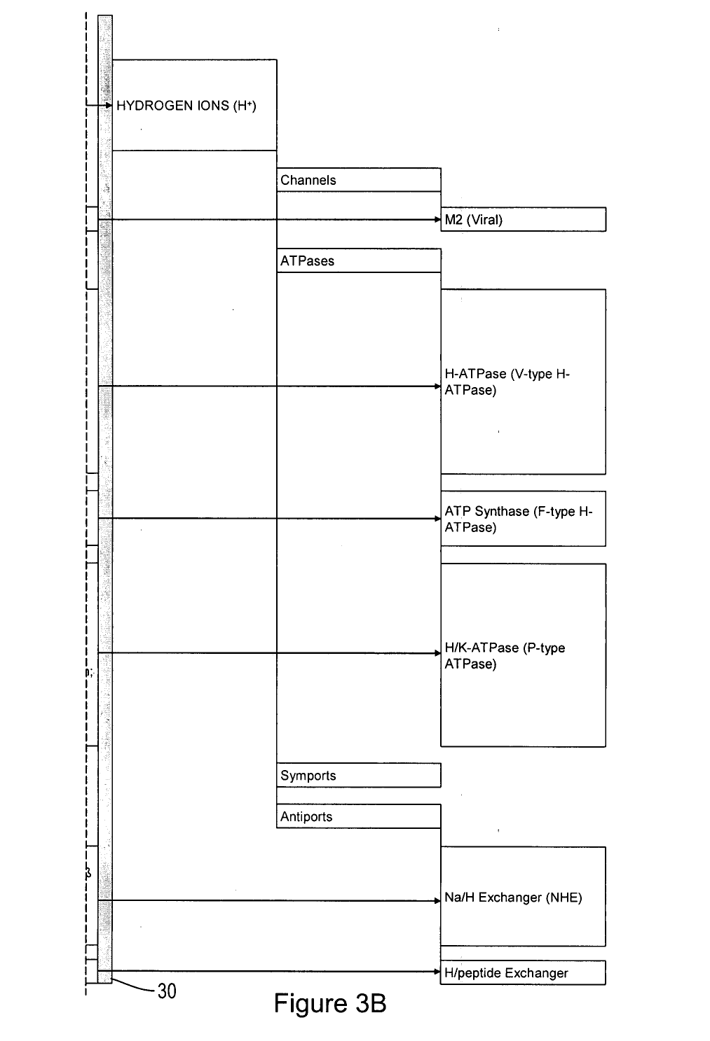

FIG. 3 provides an example of how this methodology can be used to systematically evaluate a role for hydrogen ion flux in a particular biological process and/or system. FIG. 3 summarizes a screening assay that include up to three steps. Note that one can stop screening at any stage and begin analyzing the particular class of ion transporter proteins so identified. Furthermore, one can conduct additional rounds of screening, if desired.

At each step, a compound that modulates ion flux mediated by a class of ion transporter proteins is selected, and cells are contacted with said compound. At each step, one can select a compound. Exemplary compounds that modulate the activity of a particular class of ion transporter proteins are depicted in the boxes shown to the left of the divider 30. The compounds have increasing specificity for a class of ion transporter proteins as the method progresses through steps 1, 2, 3. Thus, a step 3 compound modulates a more narrowly defined class of ion transporter proteins then a class 2 compound.

To illustrate, one selects from amongst the compounds depicted in the box under step 1. If one of these compounds produces a phenotype in your particular system, hydrogen ion flux is identified as involved in your particular system. Thus, in step 1, one has identified an involvement for a broad class of ion transporter proteins that mediate hydrogen ion flux.

In steps 2 and 3, one selects from amongst the compounds to distinct, for example, whether hydrogen ion flux in this particular system is modulated by one of the following classes of ion transporter proteins: V-type H-ATPase, ATP synthase, H/K-ATPase, Na/H exchanger, or H/peptide exchanger.

In each subsequent step, increasingly specific compounds can be used to identify an increasingly more define class of ion transporter proteins that mediate hydrogen flux, thereby producing a phenotype in your particular biological system.

We note that, in FIG. 3, the compounds listed to the left of divider 30 are ion transporter inhibitors unless otherwise indicated. Compounds are shown to the left of divider 30 and targets (e.g., class of ion transporter proteins) are represented to the right of divider 30. We also note that the listed compounds are exemplary. Additionally, we note that an investigator may chose to conduct each step of the screen in parallel and test a different compound that functions to inhibit the same class of compounds in parallel populations of cells. Observing the same or a similar phenotype using multiple compounds that modulate the same class of ion transporter proteins can be used to confirm the result and helps control for non-specific effects of the compounds.

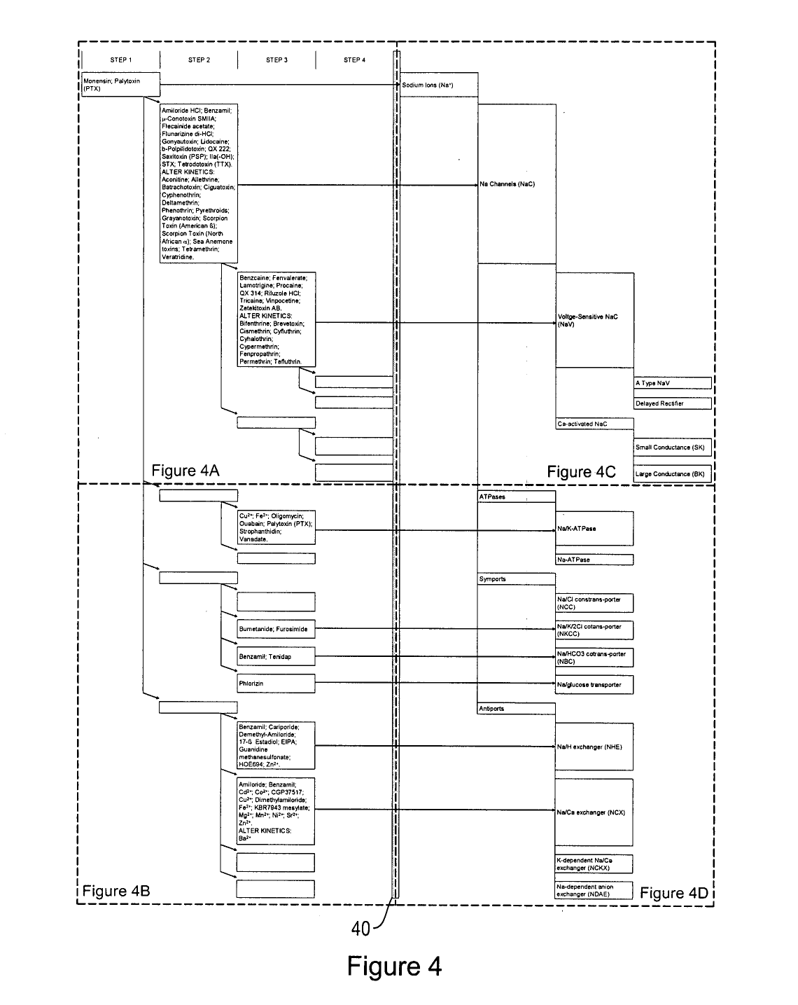

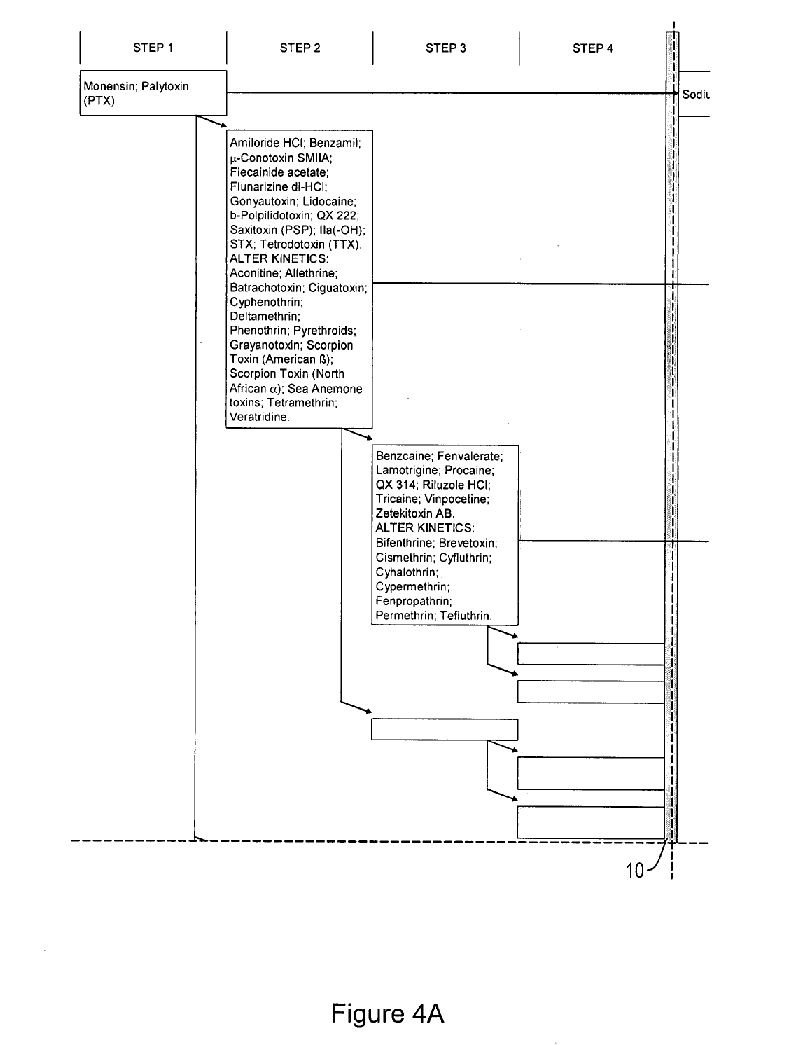

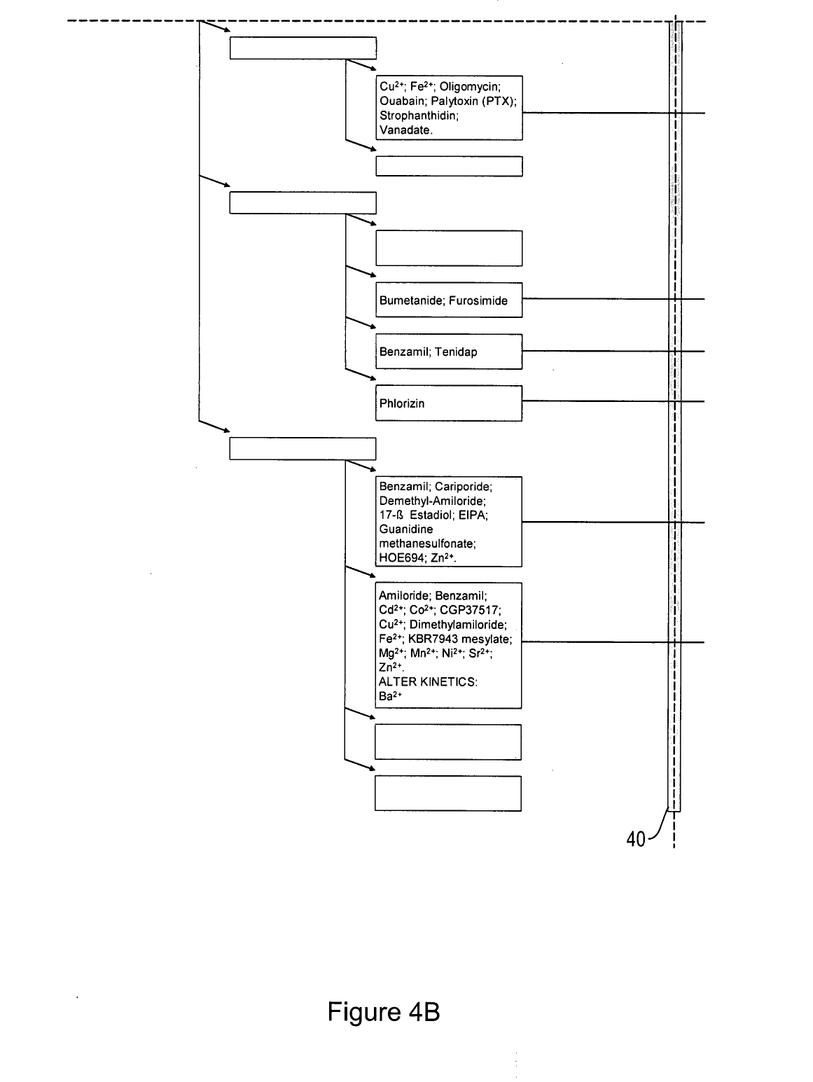

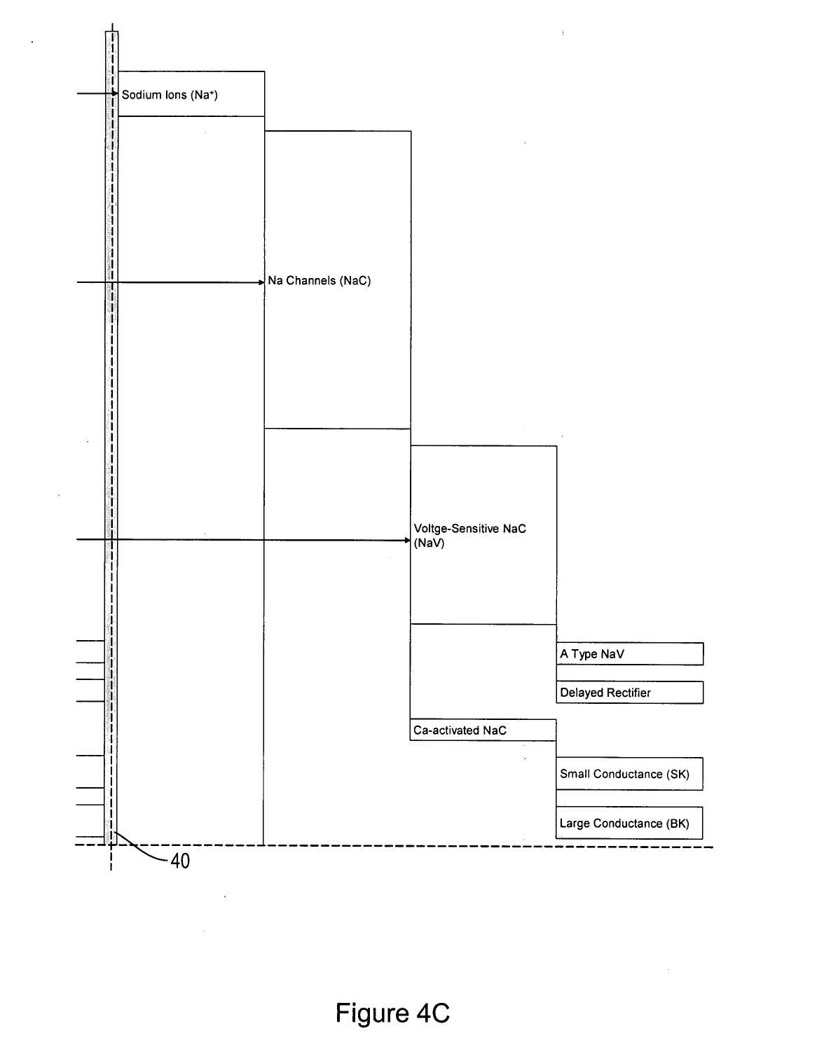

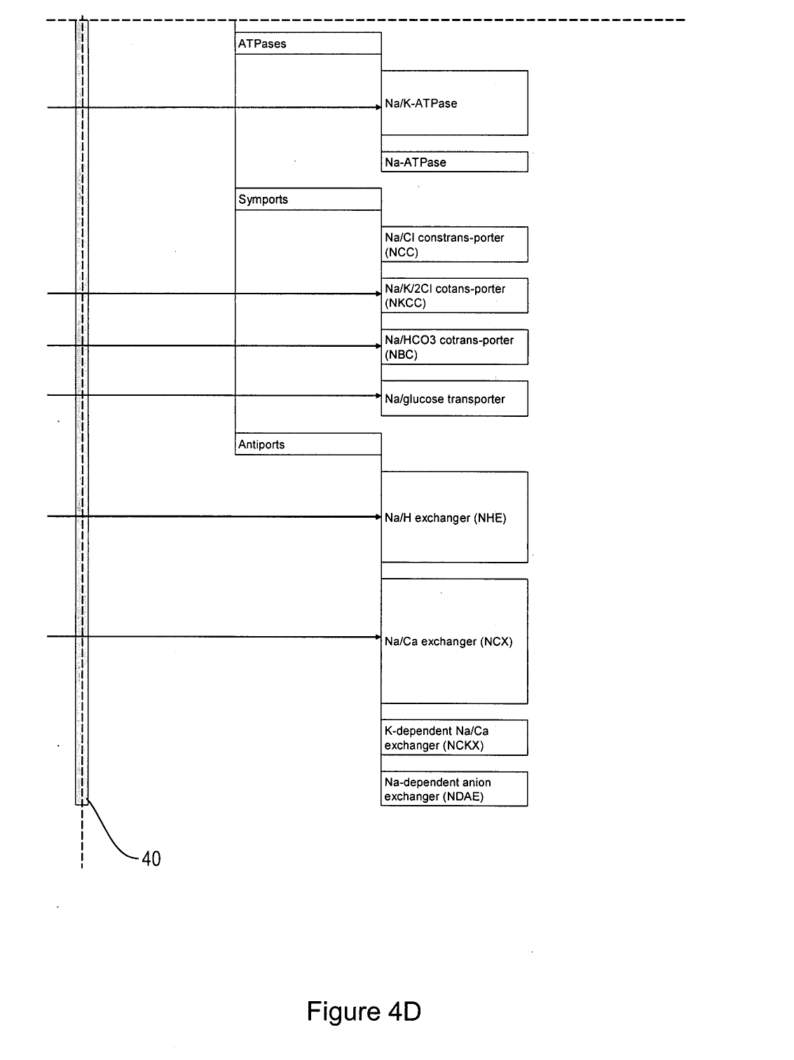

FIG. 4 provides an example of how this methodology can be used to systematically evaluate a role for sodium flux in a particular biological process and/or system. FIG. 4 summarizes a screening assay that may include up to four steps. Note that one can stop screening at any stage and begin analyzing the particular class of ion transporter proteins so identified. Furthermore, one can conduct additional round of screening. At each step, one can select a compound. Exemplary compounds that modulate the activity of a particular class of ion transporter proteins are depicted in the boxes shown to the left of the divider 40. The compounds have increasing specificity for a class of ion transporter proteins as the method progresses through steps 1, 2, 3, and 4. Thus, a step 3 compound modulates a more narrowly defined class of ion transporter proteins then a class 2 compound, and a class 4 compound modulates a more narrowly defined class of ion transporter proteins than a class 3 compound.

To illustrate, one selects from amongst the compounds depicted in the box under step 1. If one of these compounds produces a phenotype in your particular system, sodium ion flux is identified as involved in your particular system. Thus, in step 1, one has identified an involvement for a broad class of ion transporter proteins that mediate sodium ion flux.

In step 2, one selects from amongst the compounds depicted in either of the boxes under step 2. If, for example, amiloride hcl, benzamil, lidocaine, or a functionally similar listed or unlisted compound produces a phenotype in your system, the broad class of ion transporter proteins that mediate sodium flux in your system has been narrowed to the sodium channel class of transporter proteins.

In each subsequent step, increasingly specific compounds can be used to identify an increasingly more define class of ion transporter proteins that mediate sodium flux, thereby producing a phenotype in your particular biological system.

We note that, in FIG. 4, the compounds listed to the left of divider 40 are ion transporter inhibitors unless otherwise indicated. Compounds are shown to the left of divider 40 and targets (e.g., class of ion transporter proteins) are represented to the right of divider 40. We also note that the listed compounds are exemplary. Additionally, we note that an investigator may chose to conduct each step of the screen in parallel and test a different compound that functions to inhibit the same class of compounds in parallel populations of cells. Observing the same or a similar phenotype using multiple compounds that modulate the same class of ion transporter proteins can be used to confirm the result and helps control for non-specific effects of the compounds.

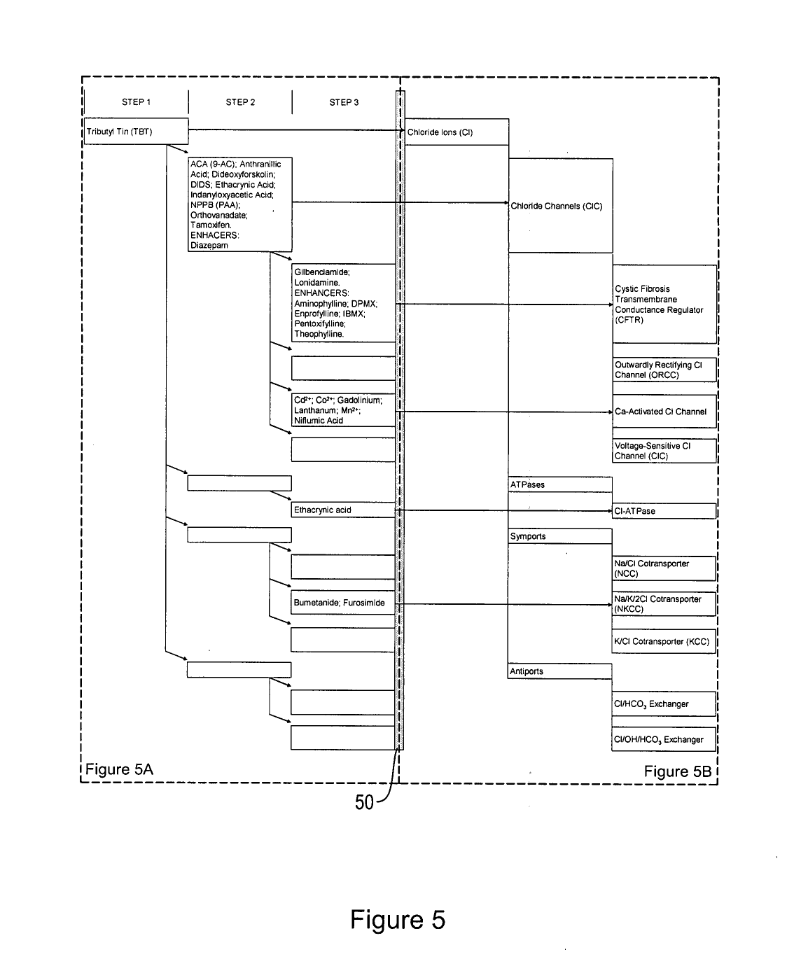

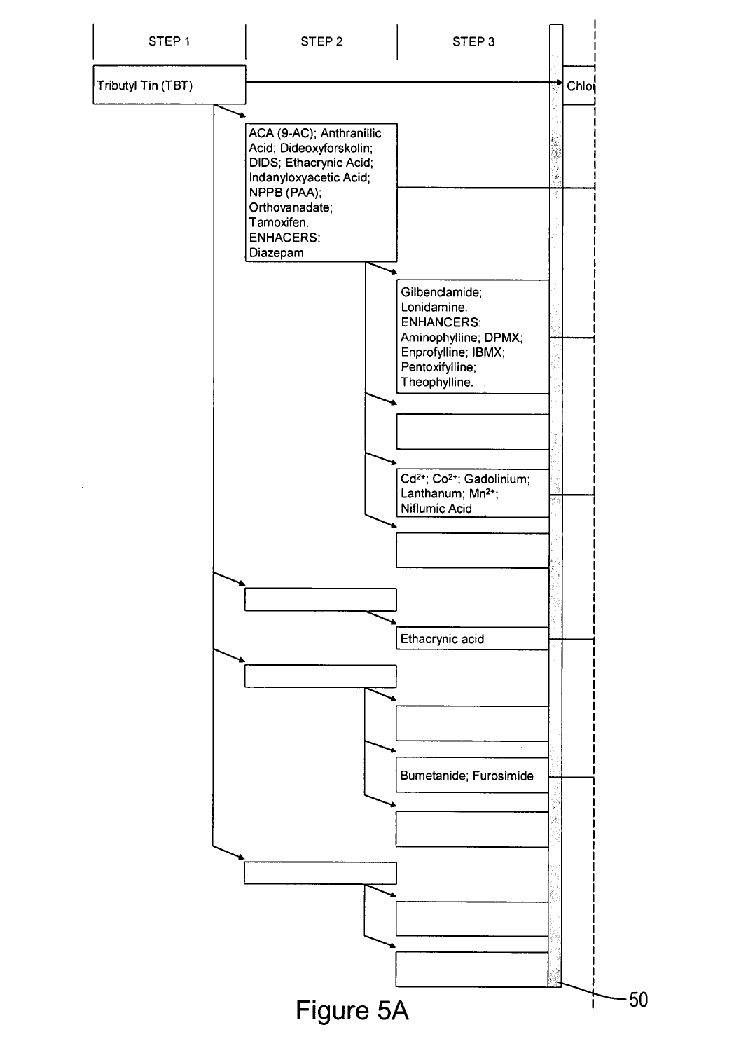

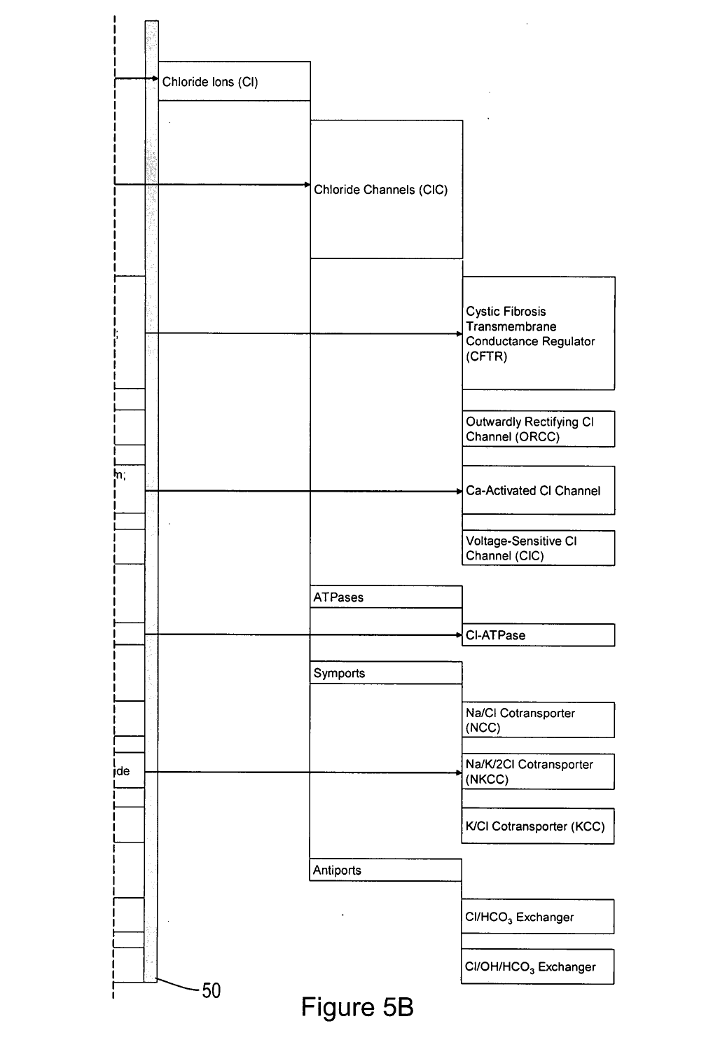

FIG. 5 provides an example of how this methodology can be used to systematically evaluate a role for chloride flux in a particular biological process and/or system. FIG. 5 summarizes a screening assay that may include up to three steps. Note that one can stop screening at any stage and begin analyzing the particular class of ion transporter proteins so identified. Furthermore, one can conduct additional round of screening. At each step, one can select a compound. Exemplary compounds that modulate the activity of a particular class of ion transporter proteins are depicted in the boxes shown to the left of the divider 50. The compounds have increasing specificity for a class of ion transporter proteins as the method progresses through steps 1, 2, and 3.

To illustrate, one selects from amongst the compounds depicted in the box under step 1. If one of these compounds produces a phenotype in your particular system, chloride ion flux is identified as involved in your particular system. Thus, in step 1, one has identified a role for a broad class of ion transporter proteins that mediate chloride ion flux.

In step 2, one selects from amongst the compounds depicted in either of the boxes under step 2. If, for example, 9-AC, anthranillic acid, tamoxifen or a functionally similar listed or unlisted compound produces a phenotype in your system, the broad class of ion transporter proteins that mediate chloride flux in your system has been narrowed to the chloride channel class of transporter proteins.

In each subsequent step, increasingly specific compounds can be used to identify an increasingly more define class of ion transporter proteins that mediate chloride flux, thereby producing a phenotype in your particular biological system.

We note that, in FIG. 5, the compounds listed to the left of divider 50 are ion transporter inhibitors unless otherwise indicated. Compounds are shown to the left of divider 50 and targets (e.g., class of ion transporter proteins) are represented to the right of divider 50. We also note that the listed compounds are exemplary. Additionally, we note that an investigator may chose to conduct each step of the screen in parallel and test a different compound that functions to inhibit the same class of compounds in parallel populations of cells. Observing the same or a similar phenotype using multiple compounds that modulate the same class of ion transporter proteins can be used to confirm the result and helps control for non-specific effects of the compounds.

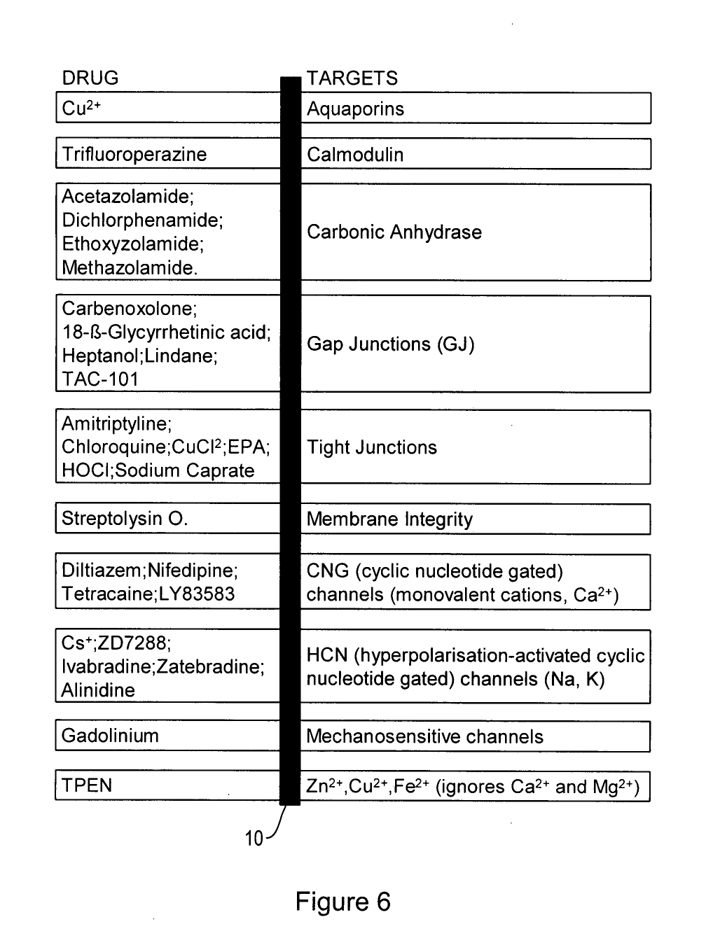

FIG. 6 provides a list of other exemplary compounds that can be used to evaluate whether particular classes of ion transporter proteins are involved in a particular biological process. Any of these drugs can be incorporated into a multi-step screen, or used as part of a single round screen to evaluate the role of a particular class of ion transporter proteins.

To illustrate, if contacting a population of cells with Cu produces a change in a particular biological process (e.g., a detectable phenotype), aquaporins are identified as having a role in modulating the biological process. If contacting a population of cells with TPEN produces a change in a particular biological process (e.g., a detectable phenotype), Zn, Cu, or Fe ion flux are identified as candidate class of ion transporter proteins.

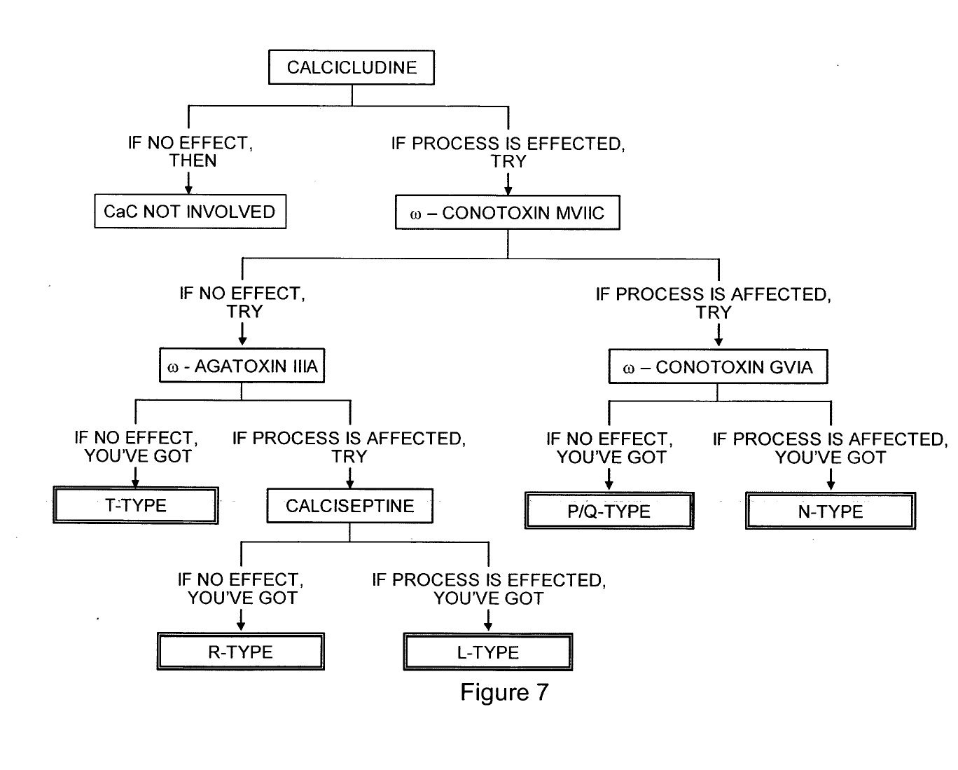

FIG. 7 depicts a detailed example of an exemplary multi-step screen for identifying a role for calcium ion flux in a particular biological process, and for further identifying a class of ion transporter proteins that mediate ion flux during the particular biological process. In a first step, a population of cells is contacted with calcicludine. If no effect is observed in the population of cells, the calcium channel class of ion transporter proteins does not modulate ion flux to mediate the particular biological process in this system. If a phenotype is observed, a second round of screening is conducted.

In the second round of screening, a second population of equivalent cells is contacted with conotoxin MVIIC. Depending on whether or not a phenotype is observed, the class of ion transporter protein involved in the biological process, can be further defined in a third, fourth, and fifth round of screening. Ultimately, the results of these rounds of screening can be used to distinguish which of the following class of calcium ion transporter proteins (e.g., T-type, P/Q-type, N-type, R-type, or L-type) mediate ion flux in a particular biological system.

Table 1 provides a list of exemplary compounds that modulate the activity of one or more classes of ion transporter proteins. Table 1 includes compounds that inhibit the activity of the class of ion transporter proteins, as well as compounds that promote the activity of the class of ion transporter proteins. Compounds that either inhibit or promote the activity of a class of ion transporter proteins can be used to disrupt the endogenous ion flux mediated by a particular class of ion transporter proteins. The existence of numerous compounds that modulate the activity of classes of ion transporters facilitates the screening hierarchy described in the present application. Skilled practitioners can select from amongst available compounds to manipulate ion transporter activity in the biological system being studied.

The above screening methodology allows an investigator to determine whether ion flux play a role in a particular biological process in a given model system. If so, the method can be used to define a class of ion transporter proteins that mediates ion flux, thereby modulating the biological process. By efficiently focusing the inquiry from amongst the thousands of possible ion transporter proteins to a relatively narrow subset of ion transporter proteins, the screening method allows investigators to define a tractable list of candidate ion transporter proteins for further study.

Once a class of ion transporter proteins is identified, the resources and techniques of molecular biology can be brought to bear to help identify the particular ion transporter protein involved in the particular biological process and system. For example, the mRNA and protein expression of individual ion transporter proteins within the identified class can be examined (e.g., so called expression analysis). Expression analysis can help determine which members of the identified class of ion transporter proteins are expressed in a spatio-temporal pattern consistent with a role in the particular biological process.

Alternatively or additionally, functional studies can be conducted in cells or whole organism. Expression or activity of individual ion transporter proteins can be modulated (e.g., inhibited or promoted) to assess whether alteration in the expression or activity of an individual ion transporter protein is sufficient to produce a phenotype. Expression or activity of individual ion transporter proteins can be modulated by administering a compound that specifically alters the expression or activity of an individual ion transporter protein. In certain embodiments, a compound that specifically alters the expression or activity of an ion transporter protein specifically inhibits the expression or activity. In certain other embodiments, a compound that specifically alters the expression or activity of an ion transporter protein specifically promotes the expression or activity. In certain embodiments, a compound that specifically alters the expression or activity of an ion transporter protein does not substantially alter the expression or activity of other ion transporter proteins in the identified class of ion transporter proteins. In certain other embodiments, a compound that specifically alters the expression or activity of an ion transporter protein does not substantially alter the expression or activity of other classes of ion transporter proteins.

By way of example, compounds that specifically inhibit or promote the expression or activity of an ion transporter protein include small organic or inorganic molecules. By way of further example, compounds that specifically inhibit the expression or activity of an ion transporter protein include nucleic acids (e.g., sense or antisense oligonucleotide, RNAi constructs, etc). By way of further example, compounds that specifically inhibit the expression or activity of an ion transporter protein include proteins (e.g., antibodies, polypeptides). By way of further example, compounds that specifically promote the expression or activity of an ion transporter protein include nucleic acids or proteins. By way of further example, compounds that specifically promote the expression or activity of an ion transporter protein include nucleic acid expression constructs of the ion transporter. Further embodiments are described in section ii.

Functional studies of individual ion transporter proteins can also be facilitated by expressing candidate ion transporter proteins as fusions proteins with a fluorescent protein such as GFP. This allows observation of the trafficking and subcellular localization of individual ion transporter proteins.

Additionally, individual ion transporter proteins or classes of ion transporter proteins can also be assessed to determine whether they modulate membrane potential and/or pH in the particular biological process. Furthermore, ion flux can be directly examined. The invention contemplates that direct detection of ion flux, intracellular pH, and/or membrane potential can be used at any stage of the screening and characterization process. In other words, such direct measurements of biophysical phenomenon can be conducted early in the screening process during evaluation of larger candidate classes of ion transporter proteins. Alternatively, direct measurements can be conducted when characterizing individual ion transporter proteins. In other embodiments, direct measurements of biophysical characteristics are not performed. Further embodiments are described in section v.

The foregoing methods can be conducted in cells in culture, in tissue samples maintained ex vivo, or in animals. When the method is conducted using cells in culture, the invention contemplates using cells derived from any organism, tissue, or stage of development. Furthermore, the invention contemplates that the cells may be primary cultures of cells, or transformed cell lines, and that the cells can either be wild type cells or cells containing one or more mutations. Mutant cells or cell lines may be models of a particular disease or injury, or may be derived from animals having a specific disease or injury (e.g. cancer cells harvested from an animal).

Cells may be derived from (e.g., derived from and cultured in vitro as populations of cells or tissues) or reside in (cells resident in a whole animal or portion of a whole animal) any of a number of animal species. Exemplary animals include, but are not limited to, flatworms, amphibians, fish, reptiles, birds, or mammals. Suitable flatworms include planarian. Suitable amphibians include Xenopus laevis, Xenopus tropicalis, and other species of frog. Suitable birds include chickens, as well as other birds commonly used or maintained in a laboratory setting. Suitable mammals include mice, rats, hamsters, goats, sheep, pigs, cows, dogs, cats, rabbits, non-human primates, and humans.

Regardless of the species of cells or animal selected, the invention contemplates that cells may be derived from or reside in an animal of virtually any stage of development. For example, the cells may be derived from or reside in an embryonic, larval, fetal, juvenile, or adult organism. The decision of whether to conduct a particular screen in cells, tissues, or animals, as well as the species and stage of development of the selected cells or animals can be readily made by one of skill in the art. The skilled artisan can select the approach, conditions, and system based on their expertise, resources, and the particular biological process they are investigating. Further embodiments are described in section iii.

In addition, the foregoing methods can be used to evaluate a role for ion flux mediated by a class of ion transporter proteins in virtually any biological process or system. By way of example, the methods can be used to evaluate a role for ion flux in cell proliferation, cell differentiation, cell survival, cell apoptosis, cell dedifferentiation, cell regeneration, or cell migration. These and other biological processes can be observed/assayed using morphological criteria (e.g., visual inspection) or the biological processes can be observed using cell biological or molecular tools and reagents. For example, the biological process can be assessed by detecting a change in gene expression (e.g., transcription or translation) using one or more molecular markers, a change in rate of cell proliferation (e.g., BrdU incorporation), a change in apoptosis (TUNEL analysis), and the like.

The foregoing represents a powerful strategy for using known pharmacological compounds to rapidly and inexpensively implicate and study specific candidates proteins for roles in any biological process (Adams and Levin, Chapter 9, Sater and Whitman, 2006). Although individual pharmacological drugs have been used previously in studies of toxicology, neuropharmacology, and teratology, these studies have failed to provide a hierarchical process whereby a role for particular proteins and signaling pathways can be efficiently evaluated.

Previous drug-based screens have generally fallen into two categories: exhaustive screening of large numbers of drugs (so-called “Sigma screens”) or specific testing of single compounds or small numbers of compounds to confirm the role of a target that has already been identified as a candidate via some other means. These approaches are fundamentally different from the screening system provided in the present application. Furthermore, approaches previously used in the art have serious limitations which are overcome by the system proposed in the present application.

The term “Sigma screens” is often used to refer to a large scale, exhaustive screening of compounds. The term is derived from the practice of screening through compounds available commercially through a major supplier such as Sigma. Usually, large numbers of compounds are tested. Although there may be an assay in which the function or efficacy of the compounds are evaluated, these screens did not typically provide any mechanism for relating effects (positive or negative) obtained using one compound with those obtained using another compound. In other words, these screens have not been performed in a hierarchical fashion which either relates effects across compounds or helps the scientist make decisions about what other compounds should be tested. In sharp contrast, the present invention provides a hierarchical approach that both relates information and helps guide the practitioner in selecting other compounds for testing.

Candidate screening approaches can be very useful to study a protein, pathway, or process that is already understood or for which considerable data already exists. However, this approach is laborious and time consuming for studying processes, proteins, or pathways for which considerable information is not already available. Furthermore, candidate screening approaches do not provide any way to interrelate the data obtained when testing individual candidate.

The approach of this application systematizes the screening process and provides a hierarchical approach to uncovering the role of molecular players (proteins and pathways) in interesting developmental events and biological processes. This approach described above, provides a highly efficient way to systematically identify and study the roles for proteins and pathways in a wide range of biological processes and systems.

(c) Methods of Identifying and/or Purifying Progenitor Cells

The present invention further provides methods for identifying and/or purifying progenitor cells from amongst heterogeneous populations of cells or from organisms. This aspect of the invention is based on the recognized correlation between the level of cell differentiation (e.g., how committed along a particular cell lineage is a particular cell?) and the membrane potential of that cell. This correlation can be used to identify cells among a population of cells whose membrane voltage characteristics are most consistent with a progenitor cell-like state. Identified cells can then be further cultured and/or studied to (i) confirm that the identified cells possess other characteristics of progenitor cells, (ii) purify the progenitor cells, and/or (iii) expand the progenitor cells.

Progenitor cells exist during embryonic, fetal, and adult development. There study has generated enormous scientific interest because progenitor cells are believed to provide promising potential treatments for a range of degenerative diseases and injuries. Furthermore, the study of progenitor cells will likely increase our understanding of normal development and regenerative processes.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from tissues other than the embryonic blastocyst. Adult stem cells include cells derived from non-blastocyst tissue, including tadpole, fetal, juvenile, and adult tissue. Stem cells have been isolated from a wide variety of adult tissues (e.g., non-blastocyst) including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the invention contemplates the identification of progenitor cells resident in any tissue, in any organism, during any stage of development.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the term “substantially pure” refers to a population of cells that contain fewer than about 20%, more preferably fewer than about 10%, most preferably fewer than about 5%, of lineage committed cells.

Research using progenitor cells has been hampered by a number of issues. Amongst the technical issues are the difficulty in readily identifying and isolating progenitor cells from amongst heterogeneous cell populations in vivo or in vitro. Progenitor cells are generally believed to constitute a very small percentage of the total number of cells in a given tissue, and even many available culture techniques still produce heterogeneous populations of cells containing both progenitor and non-progenitor cells. Difficulty in identify progenitor cells and difficulty in obtaining purified cultures of progenitor cell populations hampers further study, and may ultimately hamper the development of effective therapies based on progenitor cells.

The present invention provides methods of identifying progenitor cells from amongst heterogeneous populations of cells. Identified progenitor cells can then be separated or otherwise removed from amongst the heterogeneous population of cells. The separated cells can then be cultured to generated substantially purified populations of progenitor cells.

The methods of the invention are amenable to use with cells in vitro (e.g., to identify progenitor cells from amongst heterogeneous populations of cells in culture). The methods of the invention are amenable to use with cells in vivo (e.g., to identify progenitor cells resident in the tissues of animals or animal fragments). The methods of the invention can be used to identify progenitor cells present during any stage of development in any tissue type. As such, the present methods provide an approach of broad applicability to research across the stem cells field.

The identification and purification methods of the invention are based on the electrogenic properties of cells. Terminally differentiated cells and other non-proliferating cells generally have membrane potentials of less than −50 mV. For example, differentiated neurons have a membrane potential of approximately −90 mV, differentiated skeletal muscle has a membrane potential of approximately −75 mV, fat cells have a membrane potential of approximately −70 mV, kidney tubules have a membrane potential of approximately −65 mV, and smooth muscle cells have a membrane potential of approximately −60 mV. In contrast, proliferating cells including tumor cells and blastomeres of early cleavage stage embryos are depolarized. By way of example, blastomeres of a 16 cell embryo have a membrane potential of approximately −25 mV and the fertilized egg has a membrane potential of approximately −10 mV.

In addition to observations regarding the relative membrane potential of differentiated cells versus proliferating uncommitted cells, a correlation between intracellular pH and cell commitment also exists. Differentiated cells appear to have a higher intracellular pH in comparison to proliferating cells such as cancer cells. For example, a survey of several differentiated cell types recorded intracellular pH levels of between about 6.9-7.4. In contrast, intracellular pH levels of less than or equal to about 6.8 are consistently observed in various tumor cells in culture.

Based on the above correlations, as well as our observation of depolarized membrane potential during regeneration in model systems including planaria and frog, the present invention provides a generalized method for identifying progenitor cells from amongst heterogeneous populations of cells. The present invention provides methods for identifying progenitor cells. The method for identifying progenitor cells comprises contacting a population of cells with a voltage sensitive agent. The voltage sensitive agent produces a detectable signal in the presence of cells having a depolarized cell membrane. In this way, the voltage sensitive agent identifies the cells, if any, with a depolarized cell membrane. Cells having a depolarized cell membrane are identified, and these cells can be separated from the heterogeneous population of cells. The separated cells can be further studied and cultured. For example, separated cells can be analyzed with molecular markers indicative of particular progenitor cell populations to confirm that the cells are progenitor cells.

Based on previously observed correlations between membrane potential, one of skill in the art can select the appropriate membrane potential for the selection and separation of cells for further analysis. In one embodiment, cells having a depolarized cell membrane with a membrane potential of greater than or equal to −20 mV are identified. Such cells are candidate progenitor cells that are separated from the heterogeneous population of cells and further analyzed and cultured. In other embodiments, cells having a depolarized cell membrane with a membrane potential of greater than or equal to −15 mV, −10 mV, −5 mV, −3 mV, 0 mV, 5 mV, 10 mV, 15 mV, or mV are identified. Such cells are candidate progenitor cells that are separated from the heterogeneous population of cells and further analyzed and cultured.

The invention contemplates that the optimal membrane potential cut-off for effectively identifying progenitor cells without also including substantial numbers of non-progenitor cells may vary across tissues and organisms. For example, the optimal condition for identifying substantial numbers of progenitor cells while including a limited percentage of non-progenitor cells may differ across tissues and organisms. However, one of skill in the art can readily select the appropriate cut-off. The important point is that the first step of identifying progenitor cells allow one to separate a group of cells from a heterogeneous population of cells and substantially enrich for progenitor cells. Although the first step can produce a substantially purified population of progenitor cells, it need not do so. Even when the first step provides an initial identification of cells that include both progenitor cells and some non-progenitor cells, the method is still useful for producing populations of cells that are substantially enriched for progenitor cells. Additional rounds of screening and/or examination of known progenitor cell markers can be used, as necessary, to further enrich for progenitor cells or to confirm that a population of cells comprises progenitor cells.

As outlined above, intracellular pH is also correlated with the differentiation state and regenerative capacity of a cell. Accordingly, the foregoing methods can use, alternatively or in addition to, detection of intracellular pH to identify progenitor cells amongst a heterogeneous population of cells. In one embodiment, cells having an intracellular pH of cells than or equal to about 6.7 are identified for further study (e.g., separation, culture, further purification). In another embodiment, cells having an intracellular pH of less than or equal to about 6.6, 6.5, 6.4, 6.3, 6.2, 6.1, or 6.0 are identified for further study.

Detection of membrane voltage or intracellular pH can be used alone or in combination in the subject methods of identifying progenitor cells.

Methods and agents for detecting membrane voltage and pH are described in detail above. Numerous agents and methods exist and can be used in methods for identifying progenitor cells. By way of non-limited example, voltage sensitive agents include fluorescent dyes. Exemplary dyes include, but are not limited to, bis-(1,3-dibutylbarbituric acid)pentamethine oxonol (DiBAC₄(5)); bis-(1,3-dibutylbarbituric acid)trimethine oxonol (DiBAC₄(3)); bis-(1,3-diethylthiobarbituric acid)trimethine oxonol (DiSBAC₂(3)); 3,3′-diethyloxacarbocyanine iodide (DiOC₂(3)); 3,3′-diheptyloxacarbocyanine iodide (DiOC₇(3)); 3,3′-dihexyloxacarbocyanine iodide (DiOC₆(3)); 3,3′-dipentyloxacarbocyanine iodide (DiOC₅(3)); 1,1′,3,3,3′,3′-hexamethylindodicarbocyanine iodide (DiIC₁(5)); a structural variant thereof; or a functional variant thereof. Exemplary pH sensitive agents include fluorescent dyes. Such dyes include, but are not limited to, 2′,7′-bis-(2-carboxyethyl)-5-(and-6)-carboxyfluorescein (BCECF); 5-(and-6)-carboxy SNARF®-1; LysoTracker® Blue DND-22; LysoTracker® Green DND-26; LysoTracker® Red DND-99; LysoTracker® Yellow HCK-123; a structural variant thereof; or a functional variant thereof.

These and other methods and agents for detecting membrane voltage and pH are available and can be adapted for use in identifying progenitor cells in cells, tissues, or organisms. The appropriate methods and agents can be selected based on the organism and stage of development being examined. In addition to fluorescent agents such as pH or voltage sensitive dyes, the invention contemplates that other methods for identifying cells having a particular membrane potential or pH can be used to identify candidate progenitor cells. For example, patch-clamp or vibrating probes methods can be used.

In certain embodiments, cells identified as having a particular membrane potential and/or pH are separated for further culture and analysis. Separating the identified cells can involve dissecting the cells away from the microarchitecture in which they reside. For example, when the method is conducted in an organism, identified cells can be dissected out of the three-dimensional structures of the whole organism or tissue. Specific methods of microdissection can be selected based on the particular organism used, as well as the number of cells to be removed. Laser-based methods, as well as manual methods employing scalpel, tungsten needles, and other dissection tools can be readily employed.

When the methods of identification are conducted using cells cultured in vitro, separating identified cells may employ any of a number of methods. Identified cells can be dissected from the culture. Alternatively, the culture of cells can be dissociated, and the dissociated cells can be separated based on, for example, the detectable agent used to mark one or more cells as candidate progenitor cells. In certain embodiments, the culture of cells is dissociated, and automated sorting methods are used to separate the cells based on the detectable agent. Exemplary automated methods include FACS-scan analysis.

Once progenitor cells are identified, they can be cultured under conditions appropriate for maintaining progenitor cells derived from the particular organism and tissue. For example, media and culture conditions for maintaining mammalian mesenchymal stems cells in an undifferentiated state are known. Similarly, appropriate conditions and reagents for maintaining proliferating cultures of various progenitor cell populations are known. Such methods can be readily employed and adapted during the further study and analysis of progenitor cells identified by the present methods.

### (ii) Compounds

The present invention provides screening methods for evaluating the role of ion flux during a biological process. The present invention further provides methods for identifying and/or purifying progenitor cells. The present invention also provides methods for promoting or inhibiting dedifferentiation and/or regeneration in cells derived from or resident in an organism, as well as methods for screening to identify compounds that promote or inhibit dedifferentiation and/or regeneration, and pharmaceutical preparations comprising the identified compounds. Many of the methods and compositions of the present invention involve contacting populations of cells with compounds.

The invention contemplates the use of any of a wide range of compounds in the methods and screening assays of the invention. Exemplary classes of compounds include, but are not limited to, nucleic acids, peptides, polypeptides, small organic molecules, small inorganic molecules, peptidomimetics, antisense oligonucleotides, RNAi constructs, ribozymes, and antibodies. Compounds can be screened as single agents, multiple candidate agents, or libraries of agents. Exemplary classes of compounds are described in detail below. Tables 1 and 2 provide numerous available compounds that modulate the activity of particular ion transporters or classes of ion transporters.

Exemplary compounds include compounds that modulate ion flux by inhibiting the activity of an ion transporter protein or class of ion transporter proteins. Exemplary compounds also include compounds that modulate ion flux by promoting the activity of an ion transporter protein or class of ion transporter proteins. In certain embodiments, compounds that modulate ion flux by either promoting or inhibiting the activity of an ion transporter protein or class of ion transporter proteins also modulate membrane potential. In certain embodiments, compounds that modulate ion flux by either promoting or inhibiting the activity of an ion transporter protein or class of ion transporter proteins can be used to modulate dedifferentiation and/or regeneration.

Any of the classes of compounds can be formulated and administered as a composition or as a pharmaceutical composition.

Polypeptides and peptide fragments: In certain embodiments, the compounds are polypeptides or peptide fragments. Exemplary polypeptides or peptide fragments include wildtype, as well as variant sequences. Variant polypeptides include amino acid sequences at least 60%, 70%, 75%, 80%, 85%, 90%, 95%, 98% or 99% identical to a particular wild type polypeptide.

In addition to polypeptides and peptide fragments, the present invention also contemplates isolated nucleic acids comprising nucleotide sequences that encode said polypeptides and fragments. The term nucleic acid as used herein is intended to include fragments as equivalents, wherein such fragments have substantially the same function as the full length nucleic acid sequence from which it is derived. Equivalent nucleotide sequences will include sequences that differ by one or more nucleotide substitutions, additions or deletions, such as allelic variants; and will, therefore, include sequences that differ from the nucleotide sequence of, for example, the native nucleotide sequence. Equivalent sequences include those that vary from a known wildtype or variant sequence due to the degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to the native nucleotide sequence. Further examples of stringent hybridization conditions include a wash step of 0.2×SSC at 65° C. Equivalent nucleotide sequences will be understood to encode polypeptides which retain the activity of the polypeptide encoded by the native nucleotide sequence.

Equivalent nucleotide sequences for use in the methods described herein also include sequences which are at least 60% identical to a given nucleotide sequence. In another embodiment, the nucleotide sequence is at least 70%, 75%, 80%, 85%, 90%, 95%, 98%, 99%, or 100% identical to the nucleotide sequence of a native sequence.

Nucleic acids having a sequence that differs from nucleotide sequences which encode a particular polypeptide due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides may exist among individuals of a given species due to natural allelic variation.

In certain embodiments, the compound is a nucleic acid sequence that encodes an ion transporter protein. In certain other embodiments, the compound is a polypeptide corresponding to an ion transporter protein. In certain other embodiments, the compound is a nucleic acid sequence that encodes a portion of an ion transporter protein, and the portion of the ion transporter protein acts as a dominant negative construct that inhibits the expression or activity of an endogenous protein. In certain other embodiments, the compound is a polypeptide corresponding to a portion of an ion transporter protein, and the portion of the ion transporter protein acts as a dominant negative construct that inhibits the expression or activity of an endogenous protein. In certain embodiments, the compound is a nucleic acid sequence that encodes a gain or loss of function mutant ion transporter protein. In certain other embodiments, the compound is a polypeptide corresponding to a gain or loss of function mutant ion transporter protein. In certain embodiments, the compound is a nucleic acid sequence that encodes the V-ATPase H⁺ ion transporter protein. In certain other embodiments, the compound is a polypeptide corresponding to the V-ATPase H⁺ ion transporter protein. In certain embodiments, the compound is a nucleic acid sequence that encodes the P-type H⁺ ATPase. In certain other embodiments, the compound is a polypeptide corresponding to the P-type H⁺ ATPase. Ion transporter proteins or nucleic acid sequences encoding them may be of any species or genotype.

Antibodies: Exemplary compounds also include antibodies. Antibodies can have extraordinary affinity and specificity for particular epitopes. Without being bound by theory, antibodies can inhibit or potentiate the activity of proteins and signaling pathways in cells, thereby exerting or inducing a particular affect on cells, tissues, or organisms.

Monoclonal or polyclonal antibodies can be made using standard protocols (See, for example, Antibodies: A laboratory manual ed. by Harlow and Lane (Cold Spring Harbor Press: 1988)). A mammal, such as a mouse, a hamster or rabbit can be immunized with an immunogenic form of a peptide. Techniques for conferring immunogenicity on a protein or peptide include conjugation to carriers or other techniques well known in the art. An immunogenic portion of a protein can be administered in the presence of adjuvant. The progress of immunization can be monitored by detection of antibody titers in plasma or serum. Standard ELISA or other immunoassays can be used with the immunogen as antigen to assess the levels of antibodies. We note that antibodies may be immunospecific for a particular protein, may be immunospecific for a particular family of proteins, or may by less immunospecific and cross-react with multiple protein from related families of proteins. Antibodies which are immunospecific do not substantially cross-react with non-homologous protein. By not substantially cross react is meant that the antibody has a binding affinity for a non-homologous proteins which is at least one order of magnitude, more preferably at least 2 orders of magnitude, and even more preferably at least 3 orders of magnitude less than the binding affinity of the antibody for the protein or proteins for which the antibody is immunospecific.

The term antibody as used herein is intended to include fragments thereof which are also specifically reactive with a polypeptide or family of polypeptides. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibodies of the present invention are further intended to include bispecific and chimeric molecules having affinity for a protein conferred by at least one CDR region of the antibody.

In one variation, antibodies of the invention can be single chain antibodies (scFv), comprising variable antigen binding domains linked by a polypeptide linker. Single chain antibodies are expressed as a single polypeptide chain and can be expressed in bacteria and as part of a phage display library. The nucleic acid encoding the single chain antibody can then be recovered from the phage and used to produce large quantities of the scFv. Construction and screening of scFv libraries is extensively described in various publications (U.S. Pat. Nos. 5,258,498; 5,482,858; 5,091,513; 4,946,778; 5,969,108; 5,871,907; 5,223,409; 5,225,539).

The technology for producing monoclonal antibodies is well known. The preferred antibody homologs contemplated herein can be expressed from intact or truncated genomic or cDNA or from synthetic DNAs in prokaryotic or eukaryotic host cells. The dimeric proteins can be isolated from the culture media and/or refolded and dimerized in vitro to form biologically active compositions. Heterodimers can be formed in vitro by combining separate, distinct polypeptide chains. Alternatively, heterodimers can be formed in a single cell by coexpressing nucleic acids encoding separate, distinct polypeptide chains. See, for example, WO93/09229, or U.S. Pat. No. 5,411,941, for several exemplary recombinant heterodimer protein production protocols. Currently preferred host cells include, without limitation, prokaryotes including E. coli, or eukaryotes including yeast, Saccharomyces, insect cells, or mammalian cells, such as CHO, COS or BSC cells. One of ordinary skill in the art will appreciate that other host cells can be used to advantage.

Typically, the immortal cell line (e.g., a myeloma cell line) is derived from the same mammalian species as the lymphocytes. Preferred immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion are then selected using HAT medium, which kills unfused and unproductively fused myeloma cells (unfused splenocytes die after several days because they are not transformed). Hybridomas producing a desired antibody are detected by screening the hybridoma culture supernatants.

To produce antibody homologs that are intact immunoglobulins, hybridoma cells that tested positive in such screening assays were cultured in a nutrient medium under conditions and for a time sufficient to allow the hybridoma cells to secrete the monoclonal antibodies into the culture medium. Tissue culture techniques and culture media suitable for hybridoma cells are well known.

Alternatively, the desired antibody may be produced by injecting the hybridoma cells into the peritoneal cavity of an unimmunized mouse. The hybridoma cells proliferate in the peritoneal cavity, secreting the antibody which accumulates as ascites fluid. The antibody may be harvested by withdrawing the ascites fluid from the peritoneal cavity with a syringe.

Fully human monoclonal antibody homologs are another compound that can be used. In their intact form these may be prepared using in vitro-primed human splenocytes, as described by Boerner et al., 1991, J. Immunol., 147, 86-95. Alternatively, they may be prepared by repertoire cloning as described by Persson et al., 1991, Proc. Nat. Acad. Sci. USA, 88: 2432-2436 or by Huang and Stollar, 1991, J. Immunol. Methods 141, 227-236. U.S. Pat. No. 5,798,230 describes preparation of human monoclonal antibodies from human B cells.

In yet another method for producing fully human antibodies, U.S. Pat. No. 5,789,650 describes transgenic non-human animals capable of producing heterologous antibodies and transgenic non-human animals having inactivated endogenous immunoglobulin genes.

Large nonimmunized human phage display libraries may also be used to isolate high affinity antibodies that can be developed as human therapeutics using standard phage technology (Vaughan et al, 1996).

Yet another preferred binding agent is a humanized recombinant antibody homolog. Following the early methods for the preparation of true “chimeric antibodies” (where the entire constant and entire variable regions are derived from different sources), a new approach was described in EP 0239400 (Winter et al.) whereby antibodies are altered by substitution (within a given variable region) of their complementarity determining regions (CDRs) for one species with those from another. The process for humanizing monoclonal antibodies via CDR “grafting” has been termed “reshaping”. (Riechmann et al., 1988, Nature 332, 323-327; Verhoeyen et al., 1988, Science 239, 1534-1536).

Antisense, ribozyme and triplex techniques: Nucleic acid-based compounds include, but are not limited to, antisense oligonucleotides and ribozymes. Antisense oligonucleotides and ribozymes inhibit the expression of a protein, e.g., by inhibiting transcription and/or translation.

Binding of the oligonucleotide or ribozyme to the nucleic acid encoding the particular protein may be by conventional base pair complementarity, or, for example, in the case of binding to DNA duplexes, through specific interactions in the major groove of the double helix. In general, “antisense” therapy refers to the range of techniques generally employed in the art, and includes any therapy that relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a particular protein. Alternatively, the antisense construct is an oligonucleotide probe that is generated ex vivo and which, when introduced into the cell causes inhibition of expression by hybridizing with the mRNA and/or genomic sequences encoding a particular protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA encoding a particular protein. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451).

While antisense nucleotides complementary to the coding region of an mRNA sequence can be used, those complementary to the transcribed untranslated region and to the region comprising the initiating methionine are most preferred.

The antisense molecules can be delivered to cells or animals in vitro or in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. We note that these and other methods are have been used to deliver single antisense oligonucleotides, as well as libraries of oligonucleotides.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts and thereby prevent translation. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered in vivo or in vitro. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes, unlike antisense molecules, are catalytic, a lower intracellular concentration is required for efficiency.

Alternatively, endogenous gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

RNAi: In other embodiments, the compound is an RNAi construct. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

Peptidomimetics: In other embodiments, the invention contemplates that the agent is a peptidomimetic. Peptidomimetics are compounds based on, or derived from, peptides and proteins. Peptidomimetics can be obtained by structural modification of the amino acid sequence of a known protein using unnatural amino acids, conformational restraints, isosteric replacement, and the like. The subject peptidomimetics constitute the continuum of structural space between peptides and non-peptide synthetic structures.

Exemplary peptidomimetics can have such attributes as being non-hydrolyzable (e.g., increased stability against proteases or other physiological conditions which degrade the corresponding peptide), having increased specificity and/or potency, and having increased cell permeability for intracellular localization. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides. Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p 134). Also, see generally, Session III: Analytic and synthetic methods, in Peptides: Chemistry and Biology, G. R. Marshall ed., ESCOM Publisher: Leiden, Netherlands, 1988)

In addition to a variety of sidechain replacements which can be carried out to generate the subject peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Additionally, peptidomimetics based on more substantial modifications of the backbone of a peptide can be used. Peptidomimetics which fall in this category include (i) retro-inverso analogs, and (ii) N-alkyl glycine analogs (so-called peptoids).

Furthermore, the methods of combinatorial chemistry are being brought to bear, e.g., PCT publication WO 99/48897, on the development of new peptidomimetics. For example, one embodiment of a so-called “peptide morphing” strategy focuses on the random generation of a library of peptide analogs that comprise a wide range of peptide bond substitutes.

In an exemplary embodiment, the peptidomimetic can be derived as a retro-inverso analog of the peptide. Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enatio analog of a peptide. Retro-enantio analogs such as this can be synthesized using commercially available D-amino acids (or analogs thereof) and standard solid- or solution-phase peptide-synthesis techniques. For example, in a preferred solid-phase synthesis method, a suitably amino-protected (t-butyloxycarbonyl, Boc) residue (or analog thereof) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid is introduced by coupling with diisopropylcarbodiimide. The resin is again washed, and the cycle repeated for each of the remaining amino acids in turn. When synthesis of the protected retro-enantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

In still another illustrative embodiment, trans-olefin derivatives can be made for any of the subject polypeptides. A trans olefin analog can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible to couple the pseudodipeptides synthesized by the above method to other pseudodipeptides, to make peptide analogs with several olefinic functionalities in place of amide functionalities.

Still another class of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in designing peptidomimetics. To illustrate, the peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic, heteroaromatic, or biheteroaromatic nucleus.

Small organic or inorganic molecules: In certain embodiments, the compound is a small organic or inorganic molecule. Small organic or inorganic molecules can agonize or antagonize the function of a particular protein or class of proteins. By small organic or inorganic molecule is meant a carbon contain molecule having a molecular weight less than 5000 amu, preferably less than 2500 amu, more preferably less than 1500 amu, and even more preferably less than 750, 500, or 250 amu.

Small organic or inorganic molecules can be readily identified by screening libraries of organic molecules and/or chemical compounds to identify those compounds that have a desired function. Alternatively, single compounds or small numbers of candidate compounds can be screened individual or in combination. In certain embodiments, the small molecule (e.g., an inorganic or organic molecule) is a non-peptidyl compound containing two or fewer, one or fewer, or no peptide and/or saccharide linkages.

The foregoing are illustrative examples of classes of compounds that can be used in the various methods and screening assays of the present invention. One of skill in the art can select amongst available delivery methods to deliver the compound to the particular cells in vitro or in vivo. By way of example, many compounds readily transit epidermal barriers and other biological membranes. To administer such compounds to cells or to an animal, the compound can simply be dissolved and added to the fluid in which the cells or animal is cultured. Alternatively, the compound can be dissolved and added to the animals food or drinking water. In another alternative, the compound can be administered to the animal via local or systemic injection.

Certain compounds do not as readily transit epidermal barriers and biological membranes, and thus additional techniques have been adapted to administer such compounds to cells, tissues, and organisms. For example, RNAi constructs are often administered to animals by addition to their food or drinking water. Numerous types of nucleic acids are delivered via viral or plasmid-based expression vectors. Polypeptide-based compounds that do not readily transit membrane or that are not actively transported into cells via receptor-mediated mechanisms can be administered along with carriers that facilitate transit into cells and tissues. The foregoing exemplary administration methods are well known in the art and can be selected based on the compounds and organisms being employed in the particular screening assays or methods of use.

Whether compounds are being administered as part of a screening assay or as part of a method for modulating cells behavior, compounds can be administered alone or as pharmaceutical formulations. Exemplary pharmaceutical compositions are formulated for administration to cells or animals. In certain embodiments, the compound included in the pharmaceutical preparation may be active itself, or may be a prodrug, e.g., capable of being converted to an active compound in a physiological setting. In certain embodiments the subject compounds may be simply dissolved or suspended water, for example, in sterile water. In certain embodiments, the pharmaceutical preparation is non-pyrogenic, i.e., does not elevate the body temperature of an animal.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a subject compound. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, in certain embodiments the agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain aspects and embodiments, the present invention provides screening assays. In certain embodiments, the assays are screening assays which utilize compounds as a tool to identify and/or characterize a role for an ion transporter protein or a class of ion transporter protein during a particular biological process. Once identified, the compounds, candidate ion transporter proteins, or candidate class of ion transporter proteins can be further studied in other animals or in cell-based or cell-free assays in vitro.

In certain aspects and embodiments, the present invention provides screening assays to identify and/or characterize compounds (either known or novel) that modulate ion flux and/or membrane potential in a population of cells. Such compounds can be used to modulate cell behavior, for example to modulate cell dedifferentiation and/or regeneration, in vitro or in vivo. Identified compounds may be further characterized. Identified compounds may be useful for research directed to the further study of a particular behavioral, anatomical, or morphological process. Identified compounds may be useful for research directed to the further study of a particular ion transporter protein or class of ion transporter proteins. Furthermore, identified compounds may be useful in the development of a pharmaceutical, or even as a pharmaceutical product. Accordingly, the present invention provides for compounds and pharmaceutical compounds identified and/or characterized by any of the methods of the invention.

In certain embodiments, the identified compounds may be useful in the development of a pharmaceutical product. Further testing of a possible pharmaceutical product may involve administration to animals and may additionally involve study of the preferable route of administration. Thus, another aspect of the present invention provides pharmaceutically acceptable compositions comprising an effective amount of one or more of the compounds described above, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) opthalamic administration, for example, for administration following injury or damage to the retina. However, in certain embodiments the subject compounds may be simply dissolved or suspended in sterile water. In certain embodiments, the pharmaceutical preparation is non-pyrogenic, i.e., does not elevate the body temperature of a human or animal patient.

### (iii) Cells and Animals

As outlined throughout in reference to particular methods of the invention, the subject methods and screening assays can be conducted in vivo or in vitro in cells derived from or resident in virtually any organism. Exemplary organisms include, but are not limited to, plants, prokaryotes (e.g., bacteria), and fungi. Further exemplary organisms include, but are not limited to, chordates, hemichordates, protochordates, and invertebrates.

The foregoing methods can be conducted in cells in culture, in tissue samples maintained ex vivo, or in animals. When the method is conducted using cells in culture, the invention contemplates using cells derived from any organism, tissue, or stage of development. Furthermore, the invention contemplates that the cells may be primary cultures of cells, or transformed cell lines, and that the cells can either be wild type cells or cells containing one or more mutations. Mutant cells or cell lines may be models of a particular disease or injury, or may be derived from animals having a specific disease or injury (e.g. cancer cells harvested from an animal).

Cells may be derived from (e.g., derived from and cultured in vitro as populations of cells or tissues) or reside in (cells resident in a whole animal or portion of a whole animal) any of a number of animal species. Exemplary animals include, but are not limited to, flatworms, amphibians, fish, reptiles, birds, or mammals. Suitable flatworms include planarian. Suitable amphibians include Xenopus laevis, Xenopus tropicalis, and other species of frog. Suitable birds include chickens, as well as other birds commonly used or maintained in a laboratory setting. Suitable mammals include mice, rats, hamsters, goats, sheep, pigs, cows, dogs, cats, rabbits, non-human primates, and humans.

Regardless of the species of cells or animal selected, the invention contemplates that cells may be derived from or reside in an animal of virtually any stage of development. For example, the cells may be derived from or reside in an embryonic, larval, fetal, juvenile, or adult organism. The decision of whether to conduct a particular screen in cells, tissues, or animals, as well as the species and stage of development of the selected cells or animals can be readily made by one of skill in the art. The skilled artisan can select the approach, conditions, and system based on their expertise, resources, and the particular biological process they are investigating.

To further illustrate, in one embodiment, the foregoing methods are conducted using cells derived from or resident in a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes. C. elegans is perhaps the most well known and thoroughly studied nematode, and the invention contemplates using C. elegans or other nematodes.

In another embodiment, the foregoing methods are conducted in cells derived from or resident in a fish or amphibian species. Zebrafish (e.g., adult zebrafish and developing, e.g., embryonic fish) are a particular example of a fish well suited for study. Zebrafish are an extensively used developmental system, and genetic, cell biological, and molecular biological reagents and methods are well known and available. Additionally, numerous chemical and radiation-based screens have produced large numbers of mutant zebrafish that can also be used for study.

Xenopus laevis and Xenopus tropicalis (e.g., adult, embryonic, tadpole, etc. stage animals) are particular examples of amphibians well suited for study. Both species are used extensively, and well developed reagents exist. For example, the availability of these molecular reagents facilitates screening assays based on changes in gene or protein expression, either instead of or in addition to screening assays based on morphological criteria. Additionally, Xenopus tropicalis is a genetically tractable model organism, and mutants have been and continue to be generated and characterized. Xenopus cells and whole organisms are excellent systems for screening assays. The cells of early Xenopus embryos are relatively large, and thus easily manipulated, injected, and used for electrophysiological recording. Eggs and embryos can be collected in very large numbers. This allows high-throughput screening, and facilitates biochemical, pharmacological, and statistical analyses.

In another embodiment, the foregoing methods are conducted in cells derived from or resident in a flatworm. Exemplary flatworms are the free-living (e.g., non-parasitic) flatworm planaria. Planaria are in the phylum Platylhelmenthes and the class Turbellaria. There are numerous species of planaria, any of which can be readily used. Planaria exhibit much of the complexity of vertebrate systems: a well-differentiated nervous system, intestine, eyes, brain, three tissue layers, and bilateral symmetry. Planaria represent a critical breakthrough in the evolution of the animal body plan and are thought to very closely resemble the proto-bilaterian ancestor. It is the first organism to have both bilateral symmetry and encephalization, making it capable of detecting environmental stimuli quicker and more efficiently than the lower metazoans. Despite a simplistic appearance and evolutionary position, planaria possess a well-developed nervous system with true synaptic transmission and have what can be considered the first animal “brain” (Samat and Netsky (1985) Can J Neurol Sci. 12(4): 296-302). They have also developed sensory capabilities for the detection of light (Brown and Park (1975) Int J Chronobiol. 3(1):57-62; Brown et al., 1968), chemical gradients (Mason (1975) Anim Behav. May; 23(2): 460-9; Miyamoto and Shimozawa (1985) Zoological Science (Tokyo) 2: 389-396), vibration (Fulgheri and Messeri (1973) Boll Soc Ital Biol Sper. 49(20): 1141-5), electric fields (Brown and Ogden (1968) J Gen Physiol. 51(2)255-60), magnetic fields (Brown and Chow (1975) Physiological Zoology 48: 168-176; Brown (1966) Nature 209: 533-5), and weak γ-radiation (Brown and Park (1964) Nature 202: 469-471).

Like many of the other organisms described above, planaria are well suited for screening because of their small size. Furthermore, they are easy to raise and to subject to a multitude of reagents and manipulations. Their consistent, flat shape and active behaviors make it simple to observe the results of any behavioral or morphological perturbation. Moreover, evolutionarily, they are very similar to the ancestor of the bilateria clade, and have high relevance to human medicine and physiology both structurally and physiologically (Best and Morita (1982) Teratog Carcinog Mutagen. 2(3-4): 277-91; Samat and Netsky (1985) Can J Neurol Sci. 12(4): 296-302). Planaria offer an excellent combination of experimental tractability and sufficient complexity for asking a number of fascinating questions about basic functions of living systems (Eisenstein (1997) Behavioral Brain Research 82: 121-132). Crucially, as a model system, planaria are quickly acquiring a powerful set of molecular biological reagents and techniques, enabling genetic and cell-biological investigations into its structure and function (Agata et al., 2003; Alvarado et al., 2002; Cebria et al. (2002) Nature 419: 620-4).

Planaria have exceptional regenerative capacity. A bisected flatworm readily regenerates. Thus planaria, either whole animals or fragments, serve as an excellent model system in which to study the implications of ion flux on cell dedifferentiation and regeneration, as well as on progenitor cell identification and characterization. In addition to planaria, other model systems have enhanced regenerative capacity, and these systems are especially well suited for studies of ion flux on cell dedifferentiation and regeneration. By way of example, fish and amphibian species may be especially useful as model systems in such studies.

The invention contemplates the use of any of the foregoing animals, as well as plants, prokaryotes, and fungi. Each of these has numerous characteristics that make them suitable for particular screening assays. The appropriate model organism can be readily selected based on the particular assays being conducted, as well as space and resource constraints. An investigator can readily determine whether to conduct the assays in cells cultured in vitro, tissue explants cultured in vitro, or in whole animals or animal fragments. Furthermore, the appropriate developmental stage can be readily selected. Exemplary developmental stages include, but are not limited to, embryonic stages, tadpole stages, larval stages, juvenile stages, and adult stages. Furthermore, the invention contemplates studying whole animals, as well as animal fragments. An exemplary animal fragment is a bisected or trisected organism. In one embodiment, the animal fragment is a bisected or trisected planarian. In another embodiment, the animal fragment is formed by fission of a whole animal. Additionally, the invention contemplates the use of wild type or mutant animals. In one embodiment, the animal is a wild type embryonic, tadpole, larval, fetal, juvenile, or adult stage animal. In another embodiment, the animal is a mutant embryonic, tadpole, larval, fetal, juvenile, or adult stage animal. In still another embodiment, the animal includes an injury. In yet another embodiment, the animal is a model of regeneration (e.g., an animal or tissue in an animal that endogenously possesses robust regenerative capacity).

It is often informative to use more than one species, both to overcome difficulties inherent in one type of model system, as well as to gain insight into the evolutionary biology of ion flux related phenomena.

Many of the foregoing animals are particularly attractive due to the availability of additional experimental tools and reagents to facilitate assay methods, as well as further study. For example, cell biological, genetic, and/or molecular reagents exist for many of these model organisms. To illustrate, the availability of fluorescent reagents to label living or non-living animals and cells may facilitate further study and analysis. Additional cell and molecular tools including reagents for RNA analysis (e.g., Northern blot hybridization, RT-PCR, RNase protection, in situ hybridization, GeneChip analysis) and/or protein analysis (e.g., immunohistochemistry, Western blot analysis) may be useful.

Regardless of the particular model organism selected, and regardless of whether the subject methods are conducted in vitro or in vivo, cells or animals may be of any developmental stage including, but not limited to, embryonic, fetal, larval, tadpole, juvenile, and adult stage cells or organisms. One of skill in the art can select the proper animal and developmental stage depending on the particular assay being conducted, the particular compounds being assessed, and the particular developmental or behavioral process being investigated. Furthermore, one of skill in the art can select the appropriate animal and developmental stage based on the research interests of the investigator, time, and cost considerations, as well as the availability of other complementary research reagents. Additionally, even when whole organisms or large fragments of whole organisms are used, one of skill in the art may choose to examine a particular biological process in only a portion of the whole organism or fragment.

In one embodiment, the animal or tissue (including whole animals, injured animal, fragments, or cell derived therefrom) is selected based on its robust regenerative ability. Cells, tissues, or animals with an enhanced regenerative ability may be useful in methods for identifying and characterizing a role for ion transporter proteins, ion flux, membrane potential, and/or pH in dedifferentiation and regeneration. Exemplary animals and systems with enhanced regenerative capacity include, but are not limited to, planaria, the zebrafish tail, the amphibian (e.g., Xenopus) tail, and the amphibian limb. An understanding of how regeneration is modulated in any of these systems can be used to increase/stimulate regenerative capacity in organisms and systems whose endogenous regenerative capacity is less robust. In another embodiment, the animal or tissue is selected for screening and study specifically because its endogenous regenerative capacity is not robust. Such systems include any cells or tissues derived from organisms, such as mammals, whose endogenous regenerative capacity is not robust. Such systems also include endogenously non-regenerating cells or tissues derived from particular regions of otherwise robustly regenerative organisms.

In one embodiment, the animal/organism is a protochordate. Protochordates possess a hollow dorsal nerve cord, gill slits, and a notochord. Exemplary protochordates include tunicata (e.g., sea squirts, etc.) and cephalochordate (e.g., amphioxus). Exemplary amphioxus include, but are not limited to Ciona intestinalis and Branchiostoma floridae (Holland and Gibson-Brown (2003) BioEssays 25: 528-532; Gostling and Shimeld (2003) Evolution and Development 5: 136; Dehal et al. (2002) Science 298: 2157-2167; Nishiyama et al. (1972) Tohoku J Exp Med 107: 95-96; Ogasawara et al. (2002) Develop Genes Evol 212: 173-185; Pope and Rowley (2002) J Exp Biology 205: 1577-1583).

In another embodiment, the animal/organism is a hemichordate. Exemplary hemichordates include acorn worms (Tagawa et al. (2001) Evol and Develop 3: 443).

In another embodiment, the animal/organism is a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes.

C. elegans is perhaps the most well known and thoroughly studied nematode, and the invention contemplates using C. elegans or other nematodes. Although C. elegans is considered a soil nematode, methods for culturing C. elegans in various quantities of liquid media (e.g., in a fluid) are well developed. See, http://elegans.swmed.edu/. Accordingly, the methods and apparatuses of the invention for conducting assays in aquatic animals can be readily used to conduct assays in C. elegans.

In another embodiment, the animal is a fish or amphibian. Exemplary amphibians include frog (e.g., species of Xenopus) and salamanders (e.g., species of Axolotls).

In another embodiment, the animal is a flatworm. Exemplary flatworms are the free-living (e.g., non-parasitic) flatworm planaria. Planaria are in the phylum Platylhelmenthes and the class Turbellaria. There are numerous species of planaria, any of which can be readily used.

In another embodiment, the organism is a mammal such as a mouse, rat, rabbit, pig, cow, dog, cat, non-human primate, or human.

The invention contemplates the use of any of the foregoing animals. Each of these has numerous characteristics that make them suitable for particular screening assays or for particular methods of promoting/inhibiting dedifferentiation and/or regeneration. The appropriate model organism can be readily selected based on the particular assays being conducted, as well as space and resource constraints. Furthermore, the appropriate developmental stage can be readily selected. Exemplary developmental stages include, but are not limited to, embryonic stages, fetal stages, tadpole stages, larval stages, juvenile stages, and adult stages. In certain embodiments, the animal is chosen due to its optical accessibility. Furthermore, the invention contemplates studying whole animals, animal fragments, or animals inflicted with an injury. An exemplary animal fragment is a bisected or trisected organism. In one embodiment, the animal fragment is a bisected or trisected planarian. In another embodiment, the animal fragment is formed by fission of a whole animal. Additionally, the invention contemplates the use of wild type or mutant animals. In one embodiment, the animal is a wild type embryonic, tadpole, larval, fetal, juvenile, or adult stage animal. In another embodiment, the animal is a mutant embryonic, tadpole, larval, fetal, juvenile, or adult stage animal.

In certain embodiments, it may be desirable to conduct an assay, for example an assay to identify and/or characterize a compound that modulates a particular developmental process, in a relatively simple system. Identified compounds or candidate ion transporter proteins can later be analyzed in higher organisms including mice, rats, non-human primates, and humans.

In certain other embodiments, it may be desirable to conduct an assay in parallel using different populations of cells. For example, screening assays can be conducted in parallel using cells derived from or resident in different organisms. Alternatively, screening assays can be conducted in parallel using cells of varying developmental stages derived from or resident in the same organism. In still another embodiment, screening assays can be conducted in parallel using cells of different developmental lineages (e.g., different cell or tissue types) derived from or resident in the same model organism. In this embodiment, the cells of differing developmental lineages can be of the same or varying developmental stages.

Depending on the particular model system and biological process chosen (e.g., organism, cell type, developmental stage, etc) for study or manipulation, one of skill in the art can select the appropriate culture conditions and methods for monitoring changes in the model system. For example, certain phenotype changes can be observed and monitored based on visual inspection with either the aided or unaided eye. Other phenotypic changes can be observed using molecular, cell biological, or biophysical reagents available in the art. For example, changes in the expression of one or more molecular markers can be assessed using known techniques including, but not limited to, RT-PCR, in situ hybridization, Northern blot analysis, Western blot analysis, immunocytochemistry, immunohistochemistry, and GeneChip analysis. Further tools including, but not limited to, method of detecting changes in cell proliferation, cell death, cell survival, membrane potential, intracellular pH, ion flux and the like can also be used to detect and assess phenotypic changes in cells or organisms.

### (iv) Exemplary Diseases and Injuries

As outlined above, the present invention provides methods for identifying ion transporter proteins and classes of ion transporter proteins that mediate ion flux and/or membrane potential. Identified transporters, as well as compounds that modulate (e.g., inhibit or promote) the activity of those transporters may be useful for modulating a biological process in vitro or in vivo. In certain embodiments, one or more ion transporter proteins or compounds that modulate the expression and/or activity of ion transporter proteins may be useful in modulating dedifferentiation and/or regeneration. The invention contemplates the use of a single compound to modulate a single ion transporter protein or class of ion transporter proteins. The invention further contemplates the use of multiple compounds (2, 3, 4, etc) to modulate a single ion transporter protein or a class of ion transporter proteins. The invention further contemplates the use of multiple compounds (2, 3, 4, etc) to modulate a multiple ion transporter proteins or multiple classes of ion transporter proteins.

Compounds, and pharmaceutical preparations thereof, that modulate dedifferentiation and/or regeneration may be useful in the treatment of injury or degenerative disease. Such compounds can be administered to a human or non-human patient in need of augmenting a regenerative response to disease or injury. Briefly, compounds that promote regeneration may be administered to promote the combination of proliferation, differentiation, and/or dedifferentiation needed to regenerate damaged, diseased, or injured tissue.

The invention contemplates the use of compounds individually or in combination. Suitable combinations include combinations of multiple compounds identified as promoting dedifferentiation and/or regeneration by modulating ion flux and/or membrane potential. Suitable combinations also include a compound that promotes dedifferentiation and/or regeneration by modulating ion flux and/or membrane potential along with one or more agents conventionally used in the treatment of the particular injury or degenerative disease.

Multiple agents may act additively or synergistically, and include combinations of agents that may show little or no effect when administered alone. Furthermore, the invention contemplates the use of agents in combination with known factors that influence proliferation, differentiation, or survival of a particular cell type. Still further, the invention contemplates the use of agents as part of a therapeutic regimen along with other surgical, radiological, chemical, homeopathic, or pharmacologic intervention appropriate for the particular cell type, disease or condition.

Agents which possess one of more of these characteristics may be useful in a therapeutic context. For example, injuries and diseases of the central and peripheral nervous system effect a tremendous number of people and exact a large financial and person toll. Injuries include traumatic injuries (i.e., breaks, blunt injury, burns, lacerations) to the brain or spinal cord, as well as other injuries to any region of the CNS or PNS including, but not limited to, injuries caused by bacterial infection, viral infection, cell damage following surgery, exposure to a toxic agent, cellular damage caused by cancer or other proliferative disorder, ischemia, hypoxia, and the like. Currently, effective treatments for injuries of the CNS and PNS are limited, and individuals often experience long-term deficits consistent with the extent of injury, the location of the injury, and the types of cell that are effected.

In addition to injuries of the CNS and PNS, there are a wide variety of neurodegenerative diseases that effect particular regions and/or cell types of the CNS or PNS. These diseases are often progressive in nature, and individuals afflicted with many of these diseases have few treatment options at there disposal. Exemplary neurodegenerative diseases include, but are not limited to, Parkinson's disease, Huntington's disease, Alzheimer's disease, ALS, multiple sclerosis, stroke, macular degeneration, peripheral neuropathy, and diabetic neuropathy.

In certain embodiments, compounds can be administered to promote regeneration of mesodermal or endodermal cell and tissue types. Injuries and diseases of tissues derived from the mesoderm or endoderm include, but are not limited to, myocardial infarction, osteoarthritis, rheumatoid arthritis, diabetes, cirrhosis, polycystic kidney disease, inflammatory bowel disease, pancreatitis, Crohn's disease, cancer of any mesodermal or endodermal tissue (e.g, pancreatic cancer, Wilms tumor, soft cell carcinoma, bone cancer, breast cancer, prostate cancer, ovarian cancer, uterine cancer, liver cancer, colon cancer, etc), and injuries to any mesodermal or endodermal tissue including breaks, tears, bruises, lacerations, burns, toxicity, bacterial infection, and viral infection.

Furthermore, agents identified by the methods of the present invention may be used to modulate cells of the blood and blood vessels. Exemplary agents can be used to modulate (promote or inhibit) angiogenesis. Inhibition of angiogenesis is of particular use in the treatment of many forms of cancers, as well as in conditions aggravated by excess angiogenesis such as macular degeneration. Promotion of angiogenesis is of particular use in the treatment of conditions caused or aggravated by decreased blood flow. Exemplary conditions include, but are not limited to, myocardial infarction, stroke, and ischemia. Additionally, agents identified by the methods of the present invention can be used to promote proliferation and differentiation of various cell types of the blood and can be used in the treatment of anemia, leukemia, and various immunodeficiencies.

For any of the foregoing, the application contemplates that agents may be administered alone, or may be administered in combination with other agents. Further, the application contemplates that agents identified according to the subject methods can be administered as part of a therapeutic regimen along with other treatments appropriate for the particular injury or disease being treated. For example, in the case of Parkinson's disease, a subject agent may be administered in combination with L-dopa or other Parkinson's disease medications, or in combination with a cell based neuronal transplantation therapy for Parkinson's disease. In the case of an injury to the brain or spinal cord, a subject agent may be administered in combination with physical therapy, hydrotherapy, massage therapy, and the like. In the case of peripheral neuropathy, as for example diabetic neuropathy, a subject agent may be administered in combination with insulin. In the case of myocardial infarction, the subject agent may be administered along with angioplasty, surgery, blood pressure medication, and/or as part of an exercise and diet regimen.

Physical injuries may result in cellular damage that ultimately limits the function of a particular cell or tissue. For example, physical injuries to cells in the CNS may limit the function of cells in the brain, spinal cord, or eye. Examples of physical injuries include, but are not limited to, crushing or severing of neuronal tissue, such as may occur following a fall, car accident, gun shot or stabbing wound, etc. Further examples of physical injuries include those caused by extremes in temperature such as burning, freezing, or exposure to rapid and large temperature shifts.

Physical injuries to mesodermal cell types include injuries to skeletal muscle, cardiac muscle, tendon, ligament, cartilage, bone, and the like. Examples of physical injuries include, but are not limited to, crushing, severing, breaking, bruising, and tearing of muscle tissue, bone or cartilage such as may occur following a fall, car accident, gun shot or stabbing wound, etc. Further examples of physical injuries include breaking, tearing, or bruising of muscle tissue, bone, cartilage, ligament, or tendon as may occur following a sports injury or due to aging. Further examples of physical injuries include those caused by extremes in temperature such as burning, freezing, or exposure to rapid and large temperature shifts.

Physical injuries to endodermal cell types include injuries to hepatocytes and pancreatic cell types. Examples of physical injuries include, but are not limited to, crushing, severing, and bruising, such as may occur following a fall, car accident, gun shot or stabbing wound, etc. Further examples of physical injuries include those caused by extremes in temperature such as burning, freezing, or exposure to rapid and large temperature shifts.

Further examples of an injury to any of the aforementioned cell types include those caused by infection such as by a bacterial or viral infection. Examples of bacterial or viral infections include, but are not limited to, meningitis, staph, HIV, hepatitis A, hepatitis B, hepatitis C, syphilis, human pappiloma virus, strep, etc. However, one of skill in the art will recognize that many different types of bacteria or viruses may infect cells and cause injury.

Additionally, injury to a particular cell type may occur as a consequence or side effect of other treatments being used to relieve some condition in an individual. For example, cancer treatments (chemotherapy, radiation therapy, surgery) may cause significant damage to both cancerous and healthy cells. Surgery; implantation of intraluminal devices; the placement of implants, pacemakers, shunts; and the like can all result in cellular damage.

A wide range of neurodegenerative diseases cause extensive cell damage (i.e., injury) to cells of the CNS and PNS. Accordingly, neurodegenerative diseases are candidates for treatment using the described agents. Administration of a subject agent can promote neuronal regeneration in the CNS or PNS of a patient with a neurodegenerative disease, and the promotion of neuronal regeneration can ameliorate, at least in part, symptoms of the disease. Agents may be administered individually, in combination with other agents of the invention, or as part of a treatment regimen appropriate for the specific condition being treated. The following are illustrative examples of neurodegenerative conditions which can be treated using the subject agents.

Parkinson's disease is the result of the destruction of dopamine-producing neurons of the substantia nigra, and results in the degeneration of axons in the caudate nucleus and the putamen degenerate. Although therapies such as L-dopa exist to try to ameliorate the symptoms of Parkinson's disease, to date we are unaware of treatments which either prevent the degeneration of axons and/or increase neuronal regeneration. Administration of agents with promote neuronal regeneration can help to ameliorate at least certain symptoms of Parkinson's disease including rigidity, tremor, bradykinesia, poor balance and walking problems.

Alzheimer's disease, a debilitating disease characterized by amyloid plaques and neurofibrillary tangles, results in a loss of nerve cells in areas of the brain that are vital to memory and other mental abilities. There also are lower levels of chemicals in the brain that carry complex messages back and forth between nerve cells. Alzheimer's disease disrupts normal thinking and memory. The incidence of Alzheimer's disease will only increase as the average life expectancy continues to rise around the world. One of the most notable features of Alzheimer's disease is that affected individuals can live for extended periods of time (ten or more years) while being in an extremely debilitated state often requiring round the clock care. Accordingly, the disease takes not only an enormous emotional toll, but also exacts a tremendous financial toll on affected individuals and their families. Therapies which improve neuronal function have substantial utility in improving the quality of life of Alzheimer's sufferers.

Huntington's disease is a degenerative disease whose symptoms are caused by the loss of cells in a part of the brain called the basal ganglia. This cell damage affects cognitive ability (thinking, judgment, memory), movement, and emotional control. Symptoms appear gradually, usually in midlife, between the ages of 30 and 50. However, the disease can also strike young children and the elderly. Huntington's disease is a genetic disorder. Although people diagnosed with the disease can often maintain their independence for several years following diagnosis, the disease is degenerative and eventually fatal. Currently, there are no treatments available to either cure or to ameliorate the symptoms of this disease. Furthermore, the onset of Huntington's disease is typically in middle-age (approx age 40), at a time when many people have already had children. Thus, people have usually passed this fatal genetic disorder to their off-spring before they realize that they are ill.

Amyotrophic lateral sclerosis (ALS), often referred to as “Lou Gehrig's disease,” is a progressive neurodegenerative disease that attacks motor nerve cells in the brain and the spinal cord. Degeneration of motor neurons affect the ability of the brain to initiate and control muscle movement. With all voluntary muscle action affected, patients in the later stages of the disease become totally paralyzed, and eventually die.

Multiple sclerosis (MS) is an illness diagnosed in over 350,000 persons in the United States today. MS is characterized by the appearance of more than one (multiple) areas of inflammation and scarring of the myelin in the brain and spinal cord. Thus, a person with MS experiences varying degrees of neurological impairment depending on the location and extent of the scarring. The most common characteristics of MS include fatigue, weakness, spasticity, balance problems, bladder and bowel problems, numbness, vision loss, tremor and vertigo. The specific symptoms, as well as the severity of these symptoms, varies from patient to patient and is largely determined by the particular location within the brain of the lesions.

MS is considered an autoimmune disease. Recent data suggest that common viruses may play a role in the onset of MS. If so, MS may be caused by a persistent viral infection or alternatively, by an immune process initiated by a transient viral infection in the central nervous system or elsewhere in the body. Epidemiological studies indicating the distribution of MS patients suggest that there is a triggering factor responsible for initiating onset of the disease. Without being bound by theory, it appears that some environmental factor, most likely infectious, must be encountered.

The incidence of MS is higher in North America and Europe and this geographic distribution is further suggestive of an environmental influence(s) underlying onset of MS. Additionally, MS is more prevalent in women than in men, and is more common amongst Caucasians than within either Hispanic or African-American populations. Interestingly, MS is extremely rare within Asian populations.

Macular degeneration is a catch-all term for a number of different disorders that have a common end result: the light-sensing cells of the central region of the retina—the macula—malfunction and eventually die, with gradual decline and loss of central vision, while peripheral vision is retained. Most cases of macular degeneration are isolated, individual, occurrences, mostly in people over age 60. These types are called Age Related Macular Degeneration (AMD). More rarely however, younger people, including infants and young children, develop macular degeneration, and they do so in clusters within families. These types of macular degeneration are collectively called Juvenile Macular Degeneration and include Stargardt's disease, Best's vitelliform macular dystrophy, Doyne's honeycomb retinal dystrophy, Sorsby's fundus dystrophy, Malattia levintinese, Fundus flavimaculatus, and Autosomal dominant hemorrhagic macular dystrophy.

The present invention makes available effective therapeutic agents for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a Taxation of a joint by a torn ligament, misalignment of joints, bone fracture, or by hereditary disease. The present reparative method is also useful for remodeling cartilage matrix, such as in plastic or reconstructive surgery, as well as periodontal surgery. The present method may also be applied to improving a previous reparative procedure, for example, following surgical repair of a meniscus, ligament, or cartilage. Furthermore, it may prevent the onset or exacerbation of degenerative disease if applied early enough after trauma.

Such connective tissues as articular cartilage, interarticular cartilage (menisci), costal cartilage (connecting the true ribs and the sternum), ligaments, and tendons are particularly amenable to treatment. As used herein, regenerative therapies include treatment of degenerative states which have progressed to the point of which impairment of the tissue is obviously manifest, as well as preventive treatments of tissue where degeneration is in its earliest stages or imminent. The subject method can further be used to prevent the spread of mineralisation into fibrotic tissue by maintaining a constant production of new cartilage.

In an illustrative embodiment, the subject method can be used to treat cartilage of a diarthroidal joint, such as a knee, an ankle, an elbow, a hip, a wrist, a knuckle of either a finger or toe, or a temperomandibular joint. The treatment can be directed to the meniscus of the joint, to the articular cartilage of the joint, or both. To further illustrate, the subject method can be used to treat a degenerative disorder of a knee, such as which might be the result of traumatic injury (e.g., a sports injury or excessive wear) or osteoarthritis.

In still further embodiments, agents of the present invention can be employed for the generation of bone (osteogenesis) at a site in the animal where such skeletal tissue is deficient. For instance, administration of an agent that promotes the differentiation of stem cells to bone can be employed as part of a method for treating bone loss in a subject, e.g. to prevent and/or reverse osteoporosis and other osteopenic disorders, as well as to regulate bone growth and maturation. For example, preparations comprising the identified agents can be employed, for example, to induce endochondral ossification. Therapeutic compositions can be supplemented, if required, with other osteoinductive factors, such as bone growth factors (e.g. TGF-β factors, such as the bone morphogenetic factors BMP-2 and BMP-4, as well as activin), and may also include, or be administered in combination with, an inhibitor of bone resorption such as estrogen, bisphosphonate, sodium fluoride, calcitonin, or tamoxifen, or related compounds.

The present invention provides methods and compounds that can be used to promote regeneration, for example, regeneration of endodermally derived cells, tissues, and organs. Such methods and compositions can be used to treat conditions associated, in whole or in part, by loss of, injury to, or decrease in functional performance of endodermal cell types. By way of example, definitive endodermal cell type include, but are not limited to, hepatocytes of the liver, pancreatic cell types such as β-islet cells, cells of the lung, and cells of the gastrointestinal tract. The following are illustrative of disease states that can be treated using agents that promote regeneration of specific endodermal cell types.

### Pancreatic Diseases

### 1. Diabetes Mellitus

Diabetes mellitus is the name given to a group of conditions affecting about 17 million people in the United States. The conditions are linked by their inability to create and/or utilize insulin. Insulin is a hormone produced by the beta cells in the pancreas. It regulates the transportation of glucose into most of the body's cells, and works with glucagon, another pancreatic hormone, to maintain blood glucose levels within a narrow range. Most tissues in the body rely on glucose for energy production.

Diabetes disrupts the normal balance between insulin and glucose. Usually after a meal, carbohydrates are broken down into glucose and other simple sugars. This causes blood glucose levels to rise and stimulates the pancreas to release insulin into the bloodstream. Insulin allows glucose into the cells and directs excess glucose into storage, either as glycogen in the liver or as triglycerides in adipose (fat) cells. If there is insufficient or ineffective insulin, glucose levels remain high in the bloodstream. This can cause both acute and chronic problems depending on the severity of the insulin deficiency. Acutely, it can upset the body's electrolyte balance, cause dehydration as glucose is flushed out of the body with excess urination and, if unchecked, eventually lead to renal failure, loss of consciousness, and death. Over time, chronically high glucose levels can damage blood vessels, nerves, and organs throughout the body. This can lead to other serious conditions including hypertension, cardiovascular disease, circulatory problems, and neuropathy.

### 2. Pancreatitis

Pancreatitis can be an acute or chronic inflammation of the pancreas. Acute attacks often are characterized by severe abdominal pain that radiates from the upper stomach through to the back and can cause effects ranging from mild pancreas swelling to life-threatening organ failure. Chronic pancreatitis is a progressive condition that may involve a series of acute attacks, causing intermittent or constant pain as it permanently damages the pancreas.

Normally, the pancreatic digestive enzymes are created and carried into the duodenum (first part of the small intestine) in an inactive form. It is thought that during pancreatitis attacks, these enzymes are prevented or inhibited from reaching the duodenum, become activated while still in the pancreas, and begin to autodigest and destroy the pancreas. While the exact mechanisms of pancreatitis are not well understood, it is more frequent in men than in women and is known to be linked to and aggravated by alcoholism and gall bladder disease (gallstones that block the bile duct where it runs through the head of the pancreas and meets the pancreatic duct, just as it joins the duodenum). These two conditions are responsible for about 80% of acute pancreatitis attacks and figure prominently in chronic pancreatitis. Approximately 10% of cases of acute pancreatitis are due to idiopathic (unknown) causes. The remaining 10% of cases are due to any of the following: drugs such as valproic acid and estrogen; viral infections such as mumps, Epstein-Barr, and hepatitis A or B; hypertriglyceridemia, hyperparathyroidism, or hypercalcemia; cystic fibrosis or Reye's syndrome; pancreatic cancer; surgery in the pancreas area (such as bile duct surgery); or trauma.

### Acute Pancreatitis

About 75% of acute pancreatitis attacks are considered mild, although they may cause the patient severe abdominal pain, nausea, vomiting, weakness, and jaundice. These attacks cause local inflammation, swelling, and hemorrhage that usually resolves itself with appropriate treatment and does little or no permanent damage. About 25% of the time, complications develop, such as tissue necrosis, infection, hypotension (low blood pressure), difficulty breathing, shock, and kidney or liver failure.

### Chronic Pancreatitis

Patients with chronic pancreatitis may have recurring attacks with symptoms similar to those of acute pancreatitis. The attacks increase in frequency as the condition progresses. Over time, the pancreas tissue becomes increasingly scarred and the cells that produce digestive enzymes are destroyed, causing pancreatic insufficiency (inability to produce enzymes and digest fats and proteins), weight loss, malnutrition, ascities, pancreatic pseudocysts (fluid pools and destroyed tissue that can become infected), and fatty stools. As the cells that produce insulin and glucagons are destroyed, the patient may become permanently diabetic.

### 3. Pancreatic Insufficiency

Pancreatic insufficiency is the inability of the pancreas to produce and/or transport enough digestive enzymes to break down food in the intestine and allow its absorption. It typically occurs as a result of chronic pancreatic damage caused by any of a number of conditions. It is most frequently associated with cystic fibrosis in children and with chronic pancreatitis in adults; it is less frequently but sometimes associated with pancreatic cancer.

Pancreatic insufficiency usually presents with symptoms of malabsorption, malnutrition, vitamin deficiencies, and weight loss (or inability to gain weight in children) and is often associated with steatorrhea (loose, fatty, foul-smelling stools). Diabetes also may be present in adults with pancreatic insufficiency.

### Liver Diseases

### 1. Hepatitis

There are two major forms of hepatitis: one in which the liver is damaged quickly (called acute hepatitis) and one in which the liver is damaged slowly, over a long time (called chronic hepatitis). Hepatitis can be caused by chemicals, however, it is most commonly due to infection by one of several viruses that mainly damage the liver, termed hepatitis viruses. These viruses have been named in the order of their discovery as hepatitis A, B, C, D, and E. Hepatitis A is spread through infected water and food and is especially common in children. Most infected people don't even know they have been exposed to the virus. Hepatitis B is fairly common, especially in Asia and Africa. Although hepatitis B is less common in other parts of the world, it is still the most common cause of acute viral hepatitis in North America and Europe. Hepatitis B can be spread by exposure to blood, through sexual relations, and during pregnancy and childbirth. Symptoms of hepatitis B may be absent, mild and flu-like, or acute. Approximately 1-3% of patients become chronically infected, able to continue to infect others, and often have chronic damage to the liver. Those with weakened or compromised immune systems are at an increased risk to become carriers (about 10%). Newborns are especially vulnerable, with over 90% becoming carriers. Hepatitis C is passed the same way as hepatitis B. Hepatitis C is less common than B as a cause of acute hepatitis, but the majority of the people who contract it become chronically infected, able to spread the infection to others, and usually have chronic damage to the liver. Hepatitis D and E are rare in the United States, however, they are responsible for liver damage elsewhere in the world.

### 2. Cirrhosis

Anything that causes severe ongoing injury to the liver can lead to cirrhosis. It is marked by cell death and scar formation and is a progressive disease that creates irreversible damage. Cirrhosis has no signs or symptoms in its early stages, but as it progresses, it can cause fluid build-up in the abdomen (called ascites), muscle wasting, bleeding from the intestines, easy bruising, enlargement of the breasts in men (called gynecomastia), and a number of other problems.

### 3. Obstruction

Gallstones, tumors, trauma, and inflammation can cause blockage or obstructions in the ducts draining the liver (bile ducts). When an obstruction occurs, bile and its associated wastes accumulate in the liver and the patient's skin and eyes often turn yellow (jaundice). Bilirubin accumulating in the urine turns it a dark brown color, while lack of bilirubin in the intestines causes the stool to become very pale colored.

Obstruction of the hepatic vein, the vein from the liver, may also occur, reducing blood flow out of the liver. This obstruction may be due to tumors pushing against the vein or from blood clot formation within the vein. Obstructions may be chronic and cause few symptoms, but they can also be acute and life threatening. Some can be treated with medications; others require surgery.

### 4. Fatty Liver

Fatty liver causes liver enlargement, tenderness, and abnormal liver function. The most common cause is excessive alcohol consumption. Another cause of fatty liver is NASH (nonalcoholic steatohepatitis). While symptom of fatty liver are often fairly mild, the condition can lead to chronic hepatitis and cirrhosis.

### 5. Genetic Liver Disorders

Hemochromatosis is the most common genetic liver disorder. It involves excess iron storage and is usually diagnosed in adults. There are numerous genetic liver diseases that affect children. Most of the diseases involve a defective element that results in liver injury (such as biliary atresia, where the bile ducts are absent or too small) or a missing enzyme or protein that leads to damaging deposits in the liver (such as galactosemia, the absence of a milk sugar enzyme, which leads to milk sugar accumulation; and Wilson's disease, where copper builds up in the liver).

Liver disease is often discovered during routine testing. It may not cause any symptoms at first or the symptoms may be vague, like weakness and loss of energy. In acute liver disease, symptoms related to problems handling bilirubin, including jaundice (yellowing of the skin and eyes), dark urine, and light stools, along with loss of appetite, nausea, vomiting, and diarrhea are the most common. Chronic liver disease symptoms include jaundice, dark urine, abdominal swelling (due to ascites), pruritus (itching), unexplained weight loss or gain, and abdominal pain.

### (v) Detection Methods

Direct biophysical measurements can be obtained in any of a number of ways. Electrophysiological techniques are the classic approach used to measure bioelectrical phenomena, often using KCl-filled microelectrodes connected via Ag—AgCl junctions to a very high input impedance preamplifier (to avoid draining current from the system) to measure voltage levels within cells or beneath epithelia. Intracellular recording and voltage clamping are used to measure membrane voltage and whole cell currents. Patch recording and patch clamping can be used to measure ion flux through a limited number of channels in a small area of membrane. These techniques are accepted and powerful, and have been reviewed extensively (Jurkat-Rott and Lehmann-Horn, 2004, Curr Pharm Biotechnol 5: 387-395; Park et al., 2002, Pflugers Arch 444: 305-316). A newer tool, the self-referencing “vibrating” ion-selective probe (SERIS) is a non-invasive technique for detecting and measuring ion gradients at the surface of cells (Hotary et al., 1992, Development 114: 985-996; Nuccitelli, 1980, Federation Proceedings 39: 2129; Nuccitelli, 1987, Biophysical Journal 51: A447; Smith et al., 1999, Microscopy Res & Techniques 46: 398-417; Smith and Trimarchi, 2001, Am J of Physiology-Cell Physiology 280: C1-C11). The technique utilizes ion-specific ionophore-filled microelectrodes. The tip of the electrode is vibrated at about 300 Hz between two points about 10 μm apart, one closer one farther away from a cell's plasma membrane. A difference in concentration at the two points indicates a gradient, which is measured quantitatively. An ion gradient near the outer surface of a cell implies transport of that ion across that membrane (without transport the gradient would quickly dissipate). This technique is powerful and allows the characterization of ion flux that can be used to infer the physiological state of the cell, as well as reveal the bioelectrical signals that the given cell or tissue is sending to its neighbors.

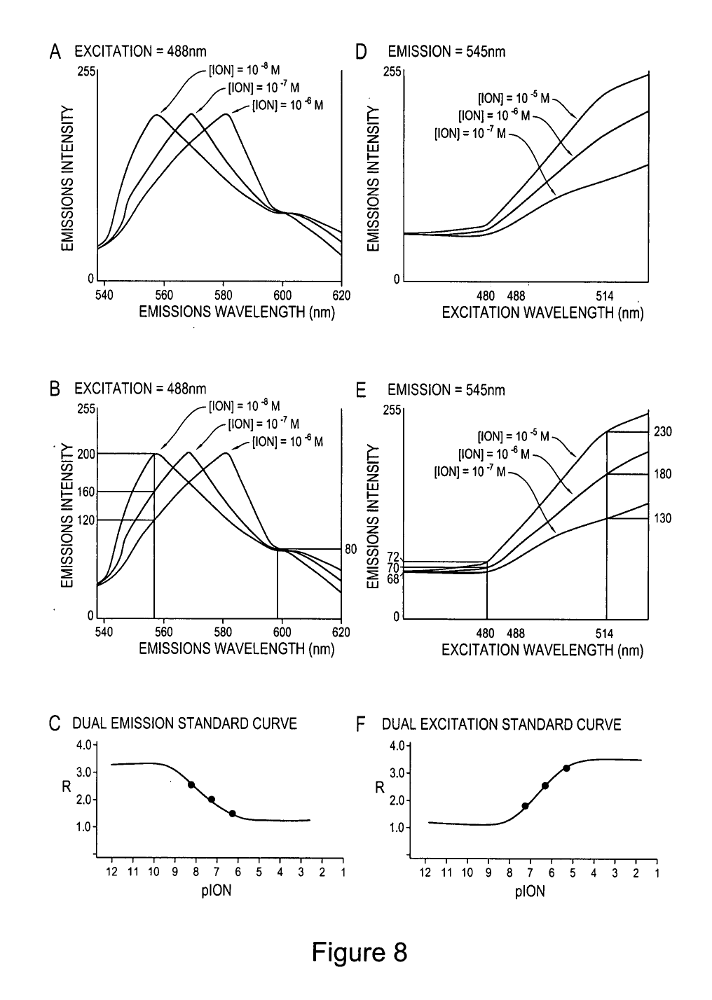

Fluorescent and other detectable ion-reporting can also be used (Amirand et al., 2000, Biol Cell 92: 409-419; Bassnett, 1990, Am J Physiol 258: C171-178, Bassnett, 1990 J Physiol 431: 445-464; Dascalu et al., 1993, J Physiol 461: 583-599; De Clerck et al., 1994, J Immunol Methods 172: 115-124; Edwards et al., 1998, Hum Reprod 13: 3441-3448; Epps et al., 1994, Chem Phys Lipids 69: 137-150; Franck et al., 1996, J Biotechnol 46: 187-195; Gasalla-Herraiz et al., 1995, Biochem Biophy Res Comm 214: 373-388; Krotz et al., 2004, Arterioscler Thromb Vasc Biol 24: 595-600; Sater et al., 1994, Development 120: 433-442). Such dyes are also referred to throughout the present application as ‘voltage sensitive agents’, ‘voltage sensitive agents that produce a detectable signal’, and ‘pH sensitive agents.’ Ion-reporting agents are readily available. They can be used to monitor (i) ion flux of virtually any ion species, (ii) membrane voltage, and (iii) intracellular pH. The ion-sensing ability of these dyes are based on the principle that binding of an ion changes the conformation of the molecule sufficiently to alter its fluorescence spectrum, a phenomenon referred to as a “spectral shift” or a “spectral response”. This is illustrated in FIG. 8. Each of the curves illustrated in FIGS. 8A-8F is a spectrum of the same (imaginary) dye. The curve is made by measuring the intensity of the light emitted by the dye at two different wavelengths. As the concentration of the sensed ion (“[Ion]”) changes, the curve moves, i.e. the spectrum changes. This change in the spectrum is known as the spectral shift. A graph of the spectral shift can be used as a standard curve.

Voltage sensitive agents can be loaded into cells by injection, electroporation, or, most commonly, by soaking the cells in membrane permeant (acetoxymethyl—AM) forms of the agent. When acetoxymethyl forms of voltage sensitive agents are internalized by cells, esterases present intracellularly cleave off the AM moiety, thus trapping the active form of the dye inside cells.

A voltage sensitive agent dye can be used for ion sensing if the bound and unbound states fluoresce differently. Referring to FIG. 8A, note that when excited by 488 nm light, this imaginary dye fluoresces most intensely at about 555 nm in the presence of 10⁻⁸ M ion (when fewer dye molecules are bound), whereas it fluoresces most intensely at about 585 nm in the presence of 10⁻⁶ M ion (when more dye molecules are bound). The units of emission intensity are not important. The scale can be calibrated depending on whether the fluorescence is evaluated using a confocal microscope, a dissecting microscope fitted with fluorescent filters, a fluorimeter, or a cytometer. Regardless of the particular scale or method used to evaluate the fluorescence, the important point is there is a measurable difference between the curves.

There are two ways that a fluorophore can “fluoresce differently”: it's emission, when excited at a single wavelength, can be ion-concentration sensitive (FIG. 8A) or its wavelength of maximal excitation, when its emission is monitored at a single wavelength, can be ion-concentration sensitive (FIG. 8D). cSNARF-1, an H⁺-reporting dye used for monitoring pH and/or H⁺ flux, is a so-called “dual emission” dye. Fluo-3, a widely used Ca²⁺-reporting dye, is a “dual excitation” dye. The particular agent selected depends on the model system, as well as the equipment available to analyze the your results.

In certain embodiments, the ion-reporting dyes are ratiometric. The ratiometric dyes allow the user to correct for artifacts that may affect the spectral shift, such as bleaching, differences in dye concentration or cell thickness, and spatial variation in instrument sensitivity. The easiest way to correct for local conditions is to measure them, and divide out their influence. That is, take a ratio: the intensity of the wavelength of interest (called λ₁) over the intensity of the local-condition light. To get the latter, you measure the same spot (the same dye molecules, in fact) at a second wavelength (called λ₂). If one is using a dual emission dye, emitted light of a different wavelength is collected. If one is using a dual excitation dye, light emitted at the same wavelength, but excited by a different wavelength is collected.

For example, using the imaginary dual-emission dye of FIG. 8A, one could illuminate with 488 nm light, then collect 555 nm light and 598 nm light (FIG. 8B). By way of further example using the imaginary dual-excitation dye of FIG. 8D, one would excite with 480 nm light and collect 545 nm light, then excite with 514 nm light and collect 545 nm light (FIG. 8E). Using either approach, producing two values of intensity: one for the wavelength that varies as a function of ion concentration (λ₁), and another that represents local conditions only (λ₂). In FIG. 8C, the intensity of the light emitted at ≈555 nm (λ₁) is very sensitive to ion concentration, while the light emitted at ≈590 (λ₂) is actually pH insensitive (this is called the isobestic point of the dye). In FIG. 8E, the intensity of the 545 nm light emitted when the dye is excited at 514 nm is very sensitive to ion concentration (λ₁), while at 480 nm, it is not (λ₂). The great advantage of using the ratio λ₁/λ₂ (often referred to as R) is that the two intensities will be similarly affected by local conditions, meaning their ratio will only be sensitive to ion concentration.

FIG. 8C represents the calibration (or standard curve) for the imaginary dual emission dye. The three points represent R from each of the three curves shown in 8B: 120/80=1.5, 160/80=2.0, 200/80=2.5. They are graphed as a function of the negative log of the concentration (pIon; if the ion were H⁺, this would be the pH scale). FIG. 8F is the calibration resulting from taking the ratios for the dual excitation dye, i.e. the data from FIG. 8E. The S-curves drawn through those points illustrate a critical point, which is that any given dye is only ion-concentration sensitive in a defined range of concentrations; above that concentration the dye is all bound, so the spectrum can not shift anymore, while below that concentration, the dye is all unbound, with the same result. The pK_{d} of the dye is the value of ion concentration that gives a ratio exactly half way between the completely bound and completely unbound states: for the imaginary dual-emission dye, pK_{d} is approximately 7.2; for the dual-excitation dye, pK_{d} is approximately 6.2. This value will be available from the manufacturer. The concentrations one wishes to measure must be near the pK_{d} of the dye so that the dye is ion-concentration sensitive.

Probably the most familiar of the ratiometric ion-sensing fluorescent probes are the calcium indicators, including the Fluo, Fura and INDO dyes. pH sensitive dyes, such as BCECF and the SNARF dyes, are also available. There are also probes for many other ions including Mg²⁺, Na⁺, K⁺, and Cl⁻. The Molecular Probes catalog is an invaluable resource exemplary reagents. The invention contemplates the use of agents including, but not limited to, ion sensing dyes that can be used to indicate ion flux of a particular species of ion. The invention further contemplates the use of voltage sensitive agents (e.g., voltage sensitive reporting dyes) and pH sensitive agents (e.g., pH sensitive dyes).

In addition to ion sensing dyes, there are also Vₘ reporting dyes. These fluorophores undergo a spectral shift in response to Vₘ. There are two categories of Vₘ dyes, those that report fast changes, such as action potentials, and those that react more slowly, reporting Vₘ averages over longer periods of time. The fast-response dyes, such as Di-8-ANEPPS, localize to the membrane, and undergo a spectral shift due to a redistribution of intramolecular charge caused by a change in Vₘ. Thus, their spectral shift occurs quickly and can be used as a measure of fast changes in Vₘ. The slow-response dyes, in contrast, are anionic or cationic molecules that accumulate inside the cell due, it is thought, to an electrophoretic mechanism driven by the voltage across the membrane. The spectrum of these dyes is affected by both environment (intra- vs. extracellular) and concentration. For a cationic dye, such as TMRE, persistent hyperpolarization will cause the accumulation of more dye molecules, and the intensity of the shifted light will increase. For an anionic dye, such as DiBAC₄(3), persistent depolarization will have the same effect. Therefore, one consideration when choosing a dye is the expected voltage, and the expected direction of it's change. In certain embodiment, experiments can be conducted in parallel using both an anionic and a cationic dye. In such embodiments, detecting opposite changes in the respective intensities of the two dyes can serve as a good control. Because the spectral shift of the slow-response probes depends on the movement of molecules, these dyes are appropriate for measuring longer-term phenomena, such as changes in the resting potential.

In one embodiment of any of the foregoing, the voltage sensitive agent produces a detectable fluorescent signal. Exemplary voltage sensitive agents include, but are not limited to, bis-(1,3-dibutylbarbituric acid)pentamethine oxonol (DiBAC₄(5)); bis-(1,3-dibutylbarbituric acid)trimethine oxonol (DiBAC₄(3)); bis-(1,3-diethylthiobarbituric acid)trimethine oxonol (DiSBAC₂(3)); 3,3′-diethyloxacarbocyanine iodide (DiOC₂(3)); 3,3′-diheptyloxacarbocyanine iodide (DiOC₇(3)); 3,3′-dihexyloxacarbocyanine iodide (DiOC₆(3)); 3,3′-dipentyloxacarbocyanine iodide (DiOC₅(3)); 1,1′,3,3,3′,3′-hexamethylindodicarbocyanine iodide (DiIC₁(5)); a structural variant thereof; or a functional variant thereof.

In another embodiment, the pH sensitive agent produces a detectable fluorescent signal. Exemplary pH sensitive agents include, but are not limited to, 2′,7′-bis-(2-carboxyethyl)-5-(and-6)-carboxyfluorescein (BCECF); 5-(and-6)-carboxy SNARF®-1; LysoTracker® Blue DND-22; LysoTracker® Green DND-26; LysoTracker® Red DND-99; LysoTracker® Yellow HCK-123; a structural variant thereof; or a functional variant thereof.

In another embodiment, the agent is an ion sensing dye. Ion sensing dyes capable of detecting ion flux of species including, but not limited to, Ca2⁺, H⁺, Cl—, K⁺, Na⁺, and Mg2+ can be used.

In addition to the foregoing methods, some particular reagents exist for further analyzing the role of gap junctions in a particular biological process. Junctional paths can be traced by microinjection of fluorescent small molecule dyes in large cells (Guthrie et al., 1988, Development 103: 769-783) or scrape-loaded into smaller cells. Various fluorescent techniques such as FRAP and photo-bleaching/uncaging of permeable molecules can be used to study GJC in vivo (Bedner et al., 2003, Exp Cell Res 291: 25-35; Braet et al., 2003, Cell Calcium 33: 37-48; Lee et al., 1995, Glia 15: 195-202; Pappas et al., 1996, Glia 16: 7-15; Suadicani et al., 2004, Glia 48: 217-229). A system of two differentially fluorescent molecules can be used to evaluate whether gap junctional communication between two cells exists. For example, two different dyes—a larger MW dye which does not pass through gap junctions (such as Molecular Probes' “Rhodamine-linked 10 kDa Dextran”) together with a small (<1 kD) tracer—can be used. The inclusion of the larger dye controls for artifacts since only cell pairs which show transfer of the small dye but not the large dye represent a true instance of GJC. The permeability of the specific gap junctions involved in your system can be tested using a panel of fluorescent small molecule probes with different shape/charge/size characteristics. The following exemplary agents can be used: Lucifer yellow (MW=443, charge of −2); 2′,7′-dichlorofluorescein (MW=401, charge of −1); neurobiotin (MW=287, charge of +1); 6-carboxyfluorescein (MW=376, charge of −2); DAPI (MW=350, charge of +1); ethidium bromide (MW=314, charge of +1); propidium iodide (MW=414, charge of +2); biocytin (MW=373, charge of 0); biotin-X cadaverin (MW=442, charge of +1); alexa 350 hydrazide (MW=349, charge of −1); alexa 488 hydrazide (MW=570, charge of −1). These and other agents are described in Meda, 2000, Methods 20: 232-244.

### EXEMPLIFICATION

The invention now being generally described, it will be more readily understood by reference to the following examples, which are included merely for purposes of illustration of certain aspects and embodiments of the present invention and are not intended to be limiting in any way.

### Example 1

### Identification of a Role for Ion Flux in a Biological Process

### Left-Right Asymmetry

We performed a compound screen to identify an ion transporter protein or a class of ion transporter proteins involved in left-right asymmetry. We employed a candidate approach to identify a manageable number of promising candidates for further molecular analysis. Briefly, groups of Xenopus embryos were treated from fertilization to stage 7 with inhibitors of various pumps, channels, and other ion transporters. Embryos cultured in the presence of compound were assayed to assess whether the compound had an effect on left-right asymmetry. Specifically, reversals of the heart, gut, or gall-bladder were assessed by morphological inspection at stage 45.

All of the reagents used in this screen were selected on the basis of high specificity for known electrogenic targets, and were titered to ensure that the DAI (dorsoanterior index) of the treated embryos was normal, thus avoiding confounding randomization caused by midline defects. Of the compounds evaluated, various inhibitors of the H⁺-V-ATPase all induced strong independent randomization of the sidedness of the 3 organs assayed (e.g., compounds that specifically inhibited the activity of this ion transporter protein induced heterotaxia).

For example, the potent and highly-specific V-ATPase blockers concanamycin and bafilomycin induced heterotaxia in 35% (n=100) and 36% (n=100) of the embryos, respectively. In all positive cases, embryos were free of generalized toxicity or gastrulation defects. Importantly, inhibition of the other two major classes of H⁺ transporters, carbonic anhydrase and the sodium-hydrogen exchanger (NHE), did not affect laterality. Taken together, specific randomization of asymmetry, in the absence of disturbances in anterior-posterior or dorsal-ventral patterning, was induced by inhibitory compounds targeting the H⁺-V-ATPase, but not by compounds that modulated the activity of other H⁺ pumps or of a wide range of other ion transporters. These data implicate endogenous H⁺-V-ATPase in this particular biological process in Xenopus embryos.

We confirmed the screen results by characterizing the function of the H⁺-V-ATPase using molecular reagents. To unequivocally test the requirement for H⁺-V-ATPase activity, we analyzed left-right asymmetry in embryos injected with YCHE78 mRNA (e.g., we overexpressed this nucleic acid into embryos). This nucleic acid encodes a well-characterized dominant negative H⁺-V-ATPase subunit E (Lu et al., 2002, J Biol Chem 277: 38409-38415). Misexpression of this dominant negative construct specifically induced 20% heterotaxia ([n=191], controls=4% [n=422], χ²=40.6, p<<0.001). In contrast, injection of mRNAs encoding unrelated ion transporter proteins—the Xenopus H,K-ATPase α subunit or the dominant negative Kir2.2 subunit—did not cause heterotaxia (1% heterotaxia each, n=85 and 93, respectively). These data are consistent with the screen results, and support a role for ion flux mediated by a specific ion transporter protein —H⁺-V-ATPase—in LR patterning.

As outlined above, effects on asymmetry were initially evaluated by inspection of embryo morphology. To extent these results, we examined expression of transcripts known to be involved in establishing the embryonic left-right axis during development. Embryos were treated with the H⁺-V-ATPase inhibitory compound concanamycin. In addition to morphologically observable effects on asymmetry, perturbation of H⁺ flux by inhibiting the function of H⁺-V-ATPase perturbed the normally left-sided gene expression of the molecular marker XNr-1.

To further characterize a role for this specific ion transporter protein (H⁺-V-ATPase), we examined its expression. We performed immunohistochemical analysis of early embryo sections to determine whether H⁺-V-ATPase localization is consistent with a role in asymmetry. We first characterized an antibody successfully used to specifically detect subunit A of the H⁺-V-ATPase in mammalian tissues. Western blot analysis on Xenopus early embryo extracts revealed a single clean band of the predicted size, and negative controls exhibited no visible signal. Immunhistochemisty with this antibody revealed that at the 2-cell stage, H⁺-V-ATPase subunits exhibited “fingers” of localization extending up from a pool in the vegetal cytoplasm into the animal half. At the four cell stage, staining is still asymmetrical, and is observed on the right side. The spatio-temporal patter of expression of H⁺-V-ATPase subunits during early development is consistent with a role for this ion transporter protein in regulating early left-right asymmetry.

We additionally characterized the expression pattern of H⁺-V-ATPase subunits in chick and zebrafish embryos. In situ hybridization analysis of subunits A, C, and F, and immunocytochemistry for subunits F and C indicated that the H⁺-V-ATPase is expressed in the primitive streak of chick embryos at stages 2, 3 and 4, and in the node at stage 4. This spatio-temporal pattern of expression correlates with the time-period in which chick LR sidedness is determined (Levin et al., 2002, Cell 111: 77-89).

In zebrafish embryos, immunohistochemical analysis using antibodies to subunit C reveals expression in two- and four-cell stage embryos. Expression was also evaluated and detected later in development (e.g., the eight-cell stage, the thirty-two-cell stage, epiboly). Thus, H⁺-V-ATPase subunits are expressed at stages relevant to LR patterning in zebrafish.

### Example 2

### H⁺-V-ATPase-Dependent, Asymmetric H⁺ Flux

We examined ion flux using a self-referencing ion probe to measure H⁺ flux from living early blastomeres. The H⁺-V-ATPase endogenously acts to pump protons out of the cell (e.g., it mediates proton efflux). We note, however, that other ion transporters modulate influx of ions. The term ion flux refers to movement of ions across membranes, regardless of the direction. The techniques provided herein can be used to evaluate changes in either ion efflux or influx, and thus can generally be used to evaluate ion flux and changes in ion flux following manipulation of an ion transporter protein or a class of ion transporter proteins.

We detected a large net efflux of protons from the cleavage furrow of the two-cell stage (in Xenopus embryos). This efflux averaged 12.7±22 pmole cm⁻² s⁻¹ (n=5) at about the midpoint of cleavage. Importantly, we also found evidence for asymmetry of H⁺-flux. As early as the four-cell stage, a distinct difference in proton efflux was detected across the ventral midline, with larger efflux occurring on the right side, consistent with the immunological localization of H⁺-V-ATPase subunits.

In measurements on 15 embryos made between the four-cell stage and stage 6, the average proton efflux from the middle of the right ventral quadrant was 4.1±0.48 pmole cm⁻²s⁻¹ and from the left ventral quadrant was 1.9±0.29 pmole cm⁻²s⁻¹. The average ratio of the efflux from the right side to efflux from the left side was 2.3±0.3.

To confirm that the asymmetric efflux was due to asymmetric H⁺-V-ATPase function, we compared H⁺ efflux in control embryos to efflux from embryos treated with a variety of ion flux inhibitors. Neither the absolute net proton fluxes nor their right/left ratios were affected by the application of Omeprazole (0.27 μM), an inhibitor of H⁺/K⁺-ATPase (e.g., a different ion transporter protein). However, the highly specific H⁺-V-ATPase inhibitor concanamycin reduced the proton efflux on the right side to about half of its original value and eliminated the left/right asymmetry in the proton fluxes. Taken together, these data reveal the existence of consistent physiological asymmetry (H⁺ flux) in the four-cell embryo, and confirm that the asymmetry in the flow of H⁺ ions out of the early blastomeres is due specifically to differential H⁺-V-ATPase activity.

Because in many systems H⁺-V-ATPase activity contributes significantly to the membrane voltage gradient Vₘₑₘ, we also used the reporting dye DiBAC₄(3) to determine whether there is a consistent asymmetry to Vₘₑₘ. Embryos marked with Alexa 647 dextran in the right ventral cell (RV) at the four cell stage (to allow orientation with respect to both axes) were monitored using DiBAC₄(3) at the 4, 8, 16, and 32 cell stages. At 16 cells, we observed hyperpolarization of the RV quadrant, relative to the LV quadrant. DiBAC₄(3) intensity on the left was greater than on the right. This pattern and its embryological timing are consistent with the self-referencing probe data, and with the hypothesis that higher net H⁺ efflux from the right side results in a difference in membrane potential across the ventral midline.

### Example 3

### Misexpression of an Ion Transporter Protein

To complement the experiments conducted using inhibitory compounds, we examined the effects of a gain-of-function treatment that would produce an excess, equal H⁺-flux across the plasma membrane on both sides of the midline. Because the H⁺-V-ATPase is a multi-subunit complex and may be difficult to re-constitute, we induced an ectopic H⁺ flux by expressing a well-characterized single-subunit plasma-membrane H⁺-pump, PMA1.2 (Masuda and Montero-Lomeli, 2000, Biochemistry and Cell Biol 78: 51-58). Use of this construct also allowed us to address whether it is the balance of H⁺ flux at the cell membrane that is important for LR asymmetry, since the H⁺-V-ATPase is also known to occur in vacuoles. In contrast, the PMA1.2 pump functions only in the cell membrane.

Misexpression of a H⁺ pump at cell surfaces throughout the embryo by microinjection of mRNA at the one-cell stage caused significant heterotaxia of embryos (PMA1.2=21% heterotaxic [n=135, unscored=22%], untreated=2% [n=187, unscored=3%], χ²=28.3, p<<0.001). These data are consistent with the importance of differential H⁺ flux in modulating left-right asymmetry.

### Example 4

### Manipulation of pH or Membrane Voltage Effects Asymmetry

We separately tested its two physiological roles—regulation of pH and membrane voltage gradients—by experimentally altering these two parameters independently of direct manipulations of the H⁺-V-ATPase. We first examined the effect of changing the pH of the embryo's external environment by raising or lowering the pH of the 0.1×MMR culture medium in which embryos were maintained. While neutral to high pH (7 to 11) had no effect on LR patterning, pH 5 to 6 caused a low level of LR patterning defects (6%, p=0.003), and pH 4 caused a significant level of heterotaxia (19%, p<<0.001). These data are consistent with the idea that a high external proton concentration will inhibit the activity of pumps, including H⁺-V-ATPase, that normally extrude H⁺ from the cytoplasm.

To alter embryonic pH without changing Vₘₑₘ, we cultured embryos in the electroneutral OH⁻/C⁻ exchanger tributyltin chloride (TBT) to raise internal pH by increasing OH⁻ influx. Treatment with 0.02 μM TBT from the one-cell stage to stage 13 caused heterotaxia in 17% of embryos ([n=151]; untreated 1% [n=361]; χ²=44.7, p<<0.001).

To study the effect of isolated changes in pH using greater molecular specificity, we overexpressed the electroneutral, plasma-membrane Na⁺/H⁺-exchanger NHE3 (Praetorius et al., 2000, American Journal of Physiology 278: G197-G206; Sabirov et al., 1999, J of Membrane Biol 172: 67-76). Injection of this antiporter mRNA within one hour of fertilization caused heterotaxia (NHE3=16% [n=77, unscored=32%], untreated=3% [n=114, unscored=5%], χ²=8.9, p=0.003) in the absence of non-specific toxicity. These findings confirm the importance of pH for LR asymmetry, and, similar to the PMA1.2 data, indicate that the relevant H⁺ flux occurs at the cell membrane, not in vesicles.

The H⁺-V-ATPase is electrogenic and can therefore significantly contribute to the steady-state Vₘₑₘ in addition to affecting pH. We confirmed this in Xenopus blastomeres by imaging Vₘₑₘ in vivo in control and concanamycin-treated embryos. Predicting that the membrane would depolarize if H⁺-V-ATPase-based proton efflux was inhibited, we monitored Vₘₑₘ using the voltage-reporting dye DiBAC₄(3). As predicted, inhibition of H⁺-V-ATPase depolarizes Vₘₑₘ.

To address the role of Vₘₑₘ in the absence of pH changes, we altered Vₘₑₘ directly by incubating embryos in the Na⁺/K⁺ inhibitor palytoxin (PTX), which converts the Na⁺/K⁺-ATPase into a non-specific ion channel thus dissipating the voltage gradient and depolarizing cells (Hilgemann, 2003, PNAS 100: 386-388). PTX treatment (2 nM) of embryos, from the one-cell stage to stage 6, caused abnormal LR patterning in 20% of treated embryos ([n=60, unscored=84%], untreated=2% [n=58, unscored=3%], χ²=8.3, p=0.004). We conclude that normal Vₘₑₘ, or patterned differences in Vₘₑₘ, are necessary for proper LR patterning.

### Example 5

### Examination of H⁺-V-ATPase Function in Other Model Systems

To determine whether chick H⁺-V-ATPase plays a role in LR asymmetry, we first examined the pattern of cellular pH in the early chick blastoderm. We then assessed the effect of inhibiting H⁺-V-ATPase activity using concanamycin. Using the pH reporting dye SNARF-1-AM (Buckler and Vaughan-Jones, 1990, Pfugers Archiv—European J of Physiology 417: 234-239; Morley et al., 1996, Biophysical Journal 70: 1294-1302), we found that area pellucida cells maintained higher pH than primitive streak cells in control embryos. As predicted by the expression of the H⁺-V-ATPase in the primitive streak, treatment with the H⁺-V-ATPase inhibitor concanamycin lowered the pH of cells in the streak.

We examined the effect of inhibiting H⁺-V-ATPase activity on the expression of the early left-sided marker Sonic hedgehog (Shh). Inhibition of H⁺-V-ATPase activity following treatment with concanamycin or DCCD specifically randomized the expression of Shh, as well as that of and the downstream marker Nodal. Compared to controls, in which these two markers are always left-sided, concanamycin induced aberrant sidedness of Shh expression in 67% and Nodal in 24% of embryos. Thus, as in Xenopus, H⁺-V-ATPase is upstream of the known asymmetric gene cascade, and is required for normal LR patterning in chicks. Perturbation of this specific ion transporter protein using a compound that specifically inhibited ion flux mediated by this protein modulated left-right asymmetry.

We also examined the consequences of disrupting endogenous cell-membrane pH gradients in Danio rerio embryos. As reported above for Xenopus, microinjection into fertilized zebrafish eggs of mRNAs encoding YCHE78 (thus equalizing patterned H⁺ fluxes by loss-of-function mediated by the dominant negative H⁺-V-ATPase subunit E), the plasma membrane H⁺-pump PMA1.2, and the electroneutral Na⁺/H⁺ antiporter NHE3 (the latter constructs equalizing patterned H⁺ fluxes by ubiquitous over-expression of exogenous plasma membrane H⁺ pumps), all specifically randomized LR patterning of the visceral organs. Down-regulating H⁺-V-ATPase activity by injection of a dominant negative subunit E mRNA caused a high level of heterotaxic organ situs (YCHE78=36% [N=42, unscored=0%], untreated=5% [N=96, unscored=4%], χ²=19.5, p<<0.001). Injections of mRNA for NHE3, to alter cytoplasmic pH only, caused 34% heterotaxia, ([N=99, unscored=6%], untreated=5% [N=94, unscored=1%], χ²=23.4, p<<0.001). Expression of PMA1.2, to exogenously increase H⁺ flux, caused 29% heterotaxia, ([N=52, unscored=9%], untreated=5% [N=96, unscored=4%], χ²=14.2, p<<0.001).

Taken together, these data strongly support the hypothesis that specific levels and/or distributions of H⁺-V-ATPase activity are necessary for correct LR asymmetry in zebrafish embryos. Compounds that specifically modulated the activity of this ion transporter protein modulated left-right asymmetry during embryonic development.

The results summarized in Examples 1-5 indicate a role for ion flux in a particular biological process. Furthermore, these results indicate that one can use the methods of the invention to identify a particular ion transporter protein or class of ion transporter proteins that mediate ion flux and thereby modulate a particular biological process. Once a particular ion transporter protein or class of ion transporter proteins are identified, expression analysis and functional analysis can be used to further analyze the role of the ion transporter protein during the biological process. Expression and functional studies can be used to extend the initial findings into other model systems or stages of development.

In Examples 1-5, the biological process was left-right asymmetry during embryonic development. One of the implicated targets, the H⁺/K⁺-ATPase, has been previously characterized in Xenopus and chick embryos (Levin et al., 2002, Cell 111: 77-89). We now present evidence for a role for H⁺ flux generally, and the H⁺-V-ATPase specifically, during the establishment of left-right asymmetry during embryonic development.

The H⁺-V-ATPase complex, more commonly referred to as the V-ATPase, is found in the membranes of vacuoles and other intracellular vesicles where it acidifies the intravesicular environment, thus activating enzymatic or other vesicle-dependent processes (Inoue et al., 2003, J Bioenerg Biomembr 35: 291-299; Kawasaki-Nishi et al., 2003, FEBS Letter 545: 76-85; Nishi, 2002, Nature Reviews Molecular Cell Biology 3: 94-103). In many cell types, including osteoclasts, kidney collecting duct, and chick chorioallantoic membrane, the H⁺-V-ATPase is also present in the plasma membrane (Baron et al., 1985, J Cell Biol 101: 2210-2222; Brown et al., 1987, J Cell Biol 105: 1637-1648; Klein et al., 1997, J Membrane Biology 157: 117-126; Narbaitz et al., 1995, Journal of Anatomy 186: 245-252; Nishi, 2002, Nature Reviews Molecular Cell Biology 3: 94-103; Schweikl et al., 1989, Journal of Biol Chem 264: 11136-11142), where, by pumping protons out of the cell, it affects cytoplasmic pH and the pH of the immediate extracellular environment (Brown and Breton, 2000, J Exp Biol 203: 137-145; Kawasaki-Nishi et al., 2003, FEBS Letter 545: 76-85; Morsomme and Boutry, 2000, Biochimica et Biophysica Acta 1465: 1-16; Nishi, 2002, Nature Reviews Molecular Cell Biology 3: 94-103; Scarborough, 2000, Cell Mol Life Sci 57: 871-883). The H⁺-V-ATPase is also electrogenic, and can contribute to the potential of the cell membrane in which it resides (Harvey, 1992, Physiology of V-ATPases. In V-ATPases, vol. 172, ed. W. Harvey and N. Nelson; Slack and Warner, 1975, Journal of Physiology 248: 97-120; Wieczorek, 1999, Bioessays 21: 637-648).

The H⁺-V-ATPase comprises two domains, the V0 and the V1, which are analogous, and largely homologous, to the F0 and F1 domains of mitochondrial ATPases. For every ATP molecule that is hydrolyzed by the V1, two protons are pumped across the membrane through proteins of the V0 domain (Nishi, 2002, Nature Reviews Molecular Cell Biology 3: 94-103).

METHODS: The following methods were used in the experiments summarized in Examples 1-5

For experiments using Xenopus laevis, embryos were collected according to standard protocols in 0.1× Modified Marc's Ringers (MMR) pH 7.8 supplemented with 0.1% Gentamicin. Embryo developmental stage was determined according to standard criteria (Niewkoop and Faber). For experiments using chicken embryos, standard pathogen-free white leghorn chick embryos from Charles River Laboratories (SPAFAS) were used. The embryos were maintained at 38° C., and developmental stage was determined according to standard criteria (Hamburger and Hamilton). For experiments using zebrafish embryos, Danio rerio embryos were obtained using standard methods and maintained at 28.5° C. in fish-system water, containing 1 drop per gallon Methylblue. Organ situs in fish embryos was evaluated at 5-7 days post-fertilization (dpf).

### Assaying Organ Situs

Xenopus embryos at stage 45 were analyzed for position (situs) of three organs: the heart, stomach, and gallbladder. Only embryos with normal dorsoanterior development (DAI=5) and clear left- or right-sided organs were scored. Embryos with ambiguous (unscoreable) situs were also counted, and while not included in statistics, were used to gauge embryo quality and treatment toxicity. An embryo was considered heterotaxic if one, two, or all three organs were abnormally positioned. Experimental organ situs percentages were compared to that of untreated controls using a χ² test with Pearson correction (assuring increased stringency for significance). The autofluorescence of gallbladder and pancreas was used to score visceral situs in zebrafish embryos. Tricaine-anaesthetized 5-6 day larvae were examined on a Zeiss StemiSV11 dissecting microscope under 488/40 nm illumination, using a 510 nm barrier filter. An embryo was considered heterotaxic if either or both organs were on the side opposite normal.

### Pharmacological Treatments

Xenopus control embryos were incubated from 60 minutes post-fertilization to stage 6-7 in 8 to 10 ml of 0.1×MMR/pH 7.8. For the screen, experimental embryos were incubated in 0.1×MMR containing compound. At stage 6-7 embryos were transferred to 0.1×MMR and maintained as described until scoring at stage 45. For all data shown, normal midline development and DAI were observed.

To perform a pharmacological screen on chick embryos with minimal disturbance to normal morphogenesis, we optimized a chicken in ovo culture system. A small hole was made on the top of each egg (prior to incubation), and 5 ml of light albumin was removed. The experimental solution, consisting of pharmacological compound in chicken light albumin and Pannett-Compton (PC) solution at a ratio of 5:1, was placed into the egg. Eggs were securely wrapped with Scotch tape, incubated at 37.5° C. to the desired stages, then fixed for analysis of laterality markers by in situ hybridization.

Danio rerio embryos were incubated at 28.5° C. in 25 ml of unadulterated or lobatomide A16- or concanamycin-containing fish water, from the one- to two-cell stage to 50% epiboly. Lobatomide A16 was used at 1 μM; concanamycin (Sigma) was used at 250 nM.

### Self-Referencing Ion-Selective (Seris) Probe

The self-referencing ion-selective (Seris) probe consists of an ion selective microelectrode that is translated between two points in the extracellular space near the plasma membrane of a cell. If a net flux of ion into or out of the cell is detected, the resultant concentration gradient will be detected as a voltage difference from which the ion flux through the membrane can be calculated. In these studies, micropipettes with tip diameters of 3 μm were backfilled with a short (1-2 mm) column of 100 mM NaCl buffered with 10 mM HEPES at pH 7.0. Pressure from a syringe was applied to force the saline to the tip of the silanized pipette, the tip was inserted into a proton ionophore cocktail (Fluka Hydrogen Ionophore I-Cocktail B), and a short (15-20 μm) column of cocktail allowed to flow into the tip, displacing the saline. The electrodes were calibrated by moving between solutions of embryo culture medium (see below) of pH 6.5 and 7.5. Potassium selective electrodes were made using a valinomycin-based cocktail (Fluka Potassium Ionophore 1-Cocktail B), using a 100 μm column; the filling solution was 100 mM KCl. Calibration was performed in culture medium with K⁺ concentrations between 0.1 mM and 1.0 mM. Electrodes always gave at least a 58 mV change for a 10× change in solution K⁺.

Seris Measurements

For Seris measurements of proton flux, healthy Xenopus embryos were cultured in 0.1×MMR at pH 7.0. Embryos were illuminated from below with dark-field optics and from above with transmitted light, and viewed on a video monitor located outside the Faraday cage enclosing the electrophysiological apparatus. H⁺ flux measurements were made to the left and right, equidistant from the ventral midline, near the animal-vegetal (AV) midline, slightly on the animal side, and approximately in the middle of the left-ventral quadrant and the right-ventral quadrant of the embryo. The displacement of the electrode was along a line that lay 45° from the normal to the embryo's surface. This was true for both the left-ventral and right-ventral measuring position. Using the analysis of Arif et al. (Arif et al., 1995, Plant Cell Environment 18: 1319-1324), we calculate a correction factor of 105 for the presence of 0.5 mM HEPES buffer and have applied that factor to generate the absolute flux values provided. The ratio of the fluxes across the ventral midline is unaffected by this correction.

### Membrane Voltage Sensitive Dye DiBAC₄(3)

Bis-(1,3-dibarbituric acid)-trimethine oxanol (DiBAC₄(3), Molecular Probes, Eugene, Oreg.) is a membrane-permeant, fluorescent molecule that accumulates inside a cell in proportion to the membrane voltage. Because it is anionic, the more depolarized a cell, the greater the accumulation of DiBAC₄(3) and the greater the intensity of intracellular, relative to extracellular, fluorescence. Stock DiBAC₄(3) (1 mg/ml in DMSO) was diluted 1:10 in distilled water, then 1:100 in 0.1×MMR, for a final concentration of 1.9 μM. Xenopus embryos were soaked in dye solution for at least 30 minutes, then imaged submerged in dye, using a Leica TCS SP2 Spectral Confocal Imaging System mounted on a Leica upright DM RXE microscope. The dye was excited with 488 nm light from an argon laser and a 20 nm band of emission wavelengths centered at 515 was collected.

### In Situ Hybridization

Whole mount in situ hybridization (WISH) on Xenopus was performed according to standard methods. DNA used to transcribe in situ hybridization probes were XNr-1 (Lohr et al., 1998, Developmental Genetics 23: 194-202; Lowe et al., 1996, Nature 381: 158-161); cShh (Levin et al., 1995, Cell 82: 803-814); cNodal (Levin et al., 1995, Cell 82: 803-814).

WISH analysis of zebrafish embryos was performed according to standard methods. Briefly, embryos were collected at the 20-22 somite stage, fixed in 4% PFA in phosphate buffered solution containing 0.1% Tween 20, and dehydrated in methanol. Embryos were rehydrated, digested with 10 μg/ml Proteinase K, and refixed in 4% PFA. Prehybridization was performed at 65° C. for 3 hours in 50% formamide, 5×SSC, 0.1% Tween-20, 4.6 mM citric acid, 50 μg/ml Heparin, and 500 μg/ml tRNA. Digoxigenin-labeled anti-sense riboprobes (Roche Applied Science, Penzberg, Germany) were added directly to pre-hybridization mix and allowed to incubate overnight at 65° C. Riboprobe was synthesized from linearized plasmid containing spaw cDNA. Washes were performed the following day at 65° C. in graded solutions from 100% hybridization mix to 100% 2×SSC, and then in 0.2×SSC. Embryos and α-dig antibody were pre-blocked at room temperature for at least 3 hours in a solution containing 1 part 10% Boehringer blocking reagent dissolved in 1M Maleic Acid, 1 part lamb serum, and 3 parts filtered Maleic Acid Buffer (MAB: 100 mM Maleic Acid, 150 mM NaCl, 0.1% Tween 20, 7.9 g/L NaOH, pH to 7.5). After pre-blocking, embryos were transferred to the α-dig antibody solution and blocked overnight at 4° C. The following morning embryos were washed first in MAB, and then in AP buffer solution (60 mM Tris HCL pH to 9.5, 60 mM NaCl, 30 mM MgCl2, and 0.1% Tween-20). Staining was achieved with NBT and BCIP in AP buffer. After staining, embryos were refixed in 4% PFA, dehydrated in 100% methanol overnight to remove background staining, and stored in 80% Glycerol.

### Immunocytochemistry

Immunocytochemistry was performed as described according to standard methods. Briefly, Xenopus embryos were fixed overnight at 4° C. in MEMFA; chick embryos were fixed overnight in 4% PFA. Some embryos were embedded for sectioning at 40 μm on a Leica Vibratome and others were left intact for whole mount analysis. After washing 3× in 1×PBS+0.1% Triton X-100 (PBST), samples were blocked with 20% Goat serum+0.2% BSA and incubated overnight at 4° C. with primary antibody at the following dilutions: anti-subunit A, 1:500; anti-myosin V, 1:500 (Chemicon #AB5887), anti-RFX3, 1:500; anti-subunit F, 1:500; anti-subunit c, 1:500 (antibody against peptide DAGVRGTAQ). After washing 6× with PBST, samples were incubated overnight at 4° C. with alkaline phosphatase conjugated secondary antibodies. After 6 more washings in PBST, detection was carried out using NBT and BCIP; the chromogenic reaction was stopped when the signal to noise ratio appeared optimal. Patterns reported for localization of V-ATPase subunits represent a consensus of data obtained from at least 15 embryos.

Microinjection of mRNA

Xenopus embryo mRNA injections were performed with capped, synthetic mRNA, according to standard protocols. Zebrafish embryo microinjection was performed using standard protocols. Three different constructs were used: YCHE78, PMA1.2, and NHE3. YCHE78 encodes a partial H⁺-V-ATPase subunit E (Lu et al., 2002, Journal of Biol Chem 277: 38409-38415) that acts as a dominant negative. PMA1.2 encodes a P-type H⁺-ATPase from Saccharomyces cerevisiae (Masuda and Montero-Lomeli, 2000, Biochemistry and Cell Biology 78: 51-58) which mimics the activity of H⁺-V-ATPase. NHE3 is an electroneutral proton pump (Soleimani et al., 1994, Biochim Biophys Acta 1195: 89-95; Counillon and Pouyssegur, 2000, J Biol Chem 275: 1-4). Results of injections are reported as: % of otherwise normal embryos that were heterotaxic; the sample size (n); when possible, the percent of injected embryos that died or were abnormal after gastrulation and thus not scored; the value of χ² and the p value comparing treated to controls.

### Example 6

### A Role for Ion Transporter Proteins in Dedifferentiation and Regeneration

The Xenopus embryo, a highly tractable vertebrate model system, regenerates its tail. The tail is thus an ideal model system for evaluating the effect of ion flux and membrane potential on regeneration. Within hours of amputation, the tail forms a blastema that then rapidly regenerates a perfect duplicate of the original tail. We used this system to evaluate the role of ion transporter proteins in regeneration. Furthermore, studies performed in the Xenopus tail are broadly applicable to studies of regeneration in multiple cell types because the tail is a complex appendage including somites (mesodermal structures), blood vessels, and neural tube. Other Xenopus structures that have an enhanced regenerative capacity include the limbs.

Having identified the Xenopus tail as an experimentally-tractable model of regeneration, we carried out several experiments to test whether bioelectric phenomena were relevant to this process. As previously reported by others, in the absence of any compounds or other manipulations, tails amputated at the midpoint between the posterior end of the hindgut and the caudal tip of the tail at approximately stage 40 regenerate with normal morphology. The process is robust (occurs normally in >90% of the embryos) and rapid. By stage 46, the tails of cut embryos are the same length (and shape) as uncut sibling controls (4 days at 22° C.).

To test whether ion flux was relevant to tail regeneration, we conducted a pharmacological screen using compounds that inhibit the activity of different types of ion channels, pumps, and co-transporters. We quantified the effects using a “regeneration index” which scores several specific aspects of regeneration and thus provides a continuous measure of regeneration.

In a first round of screening, we cultured embryos in medium containing a compound beginning immediately after tail amputation (stage 40-41). The following compounds had no effect on the rate or extent of tail regeneration: SCH28080 (inhibitor of H⁺/K⁺-ATPase); 9-anthracene-carboxylic acid (inhibitor of Cl— channels); heptanol (clocker of gap junctional communications); diazoxide (opener of Katp channels); glibenclamide (inhibitor of Katp channels); ouabain (inhibitor of Na⁺/K⁺-ATPase); EIPA (inhibitor of Na⁺/H⁺ antiporter); THB (inhibitor of voltage-sensitive K⁺ channels); WAY-123 (blocker of ERG-K channels); amiloride (inhibitor of Na/H antiporter); 18-β-glycyrrhetinic acid (blocker of gap junctional communication); EM12 (inducer of gap junctional communication). Additional screening identified further compounds that had no effect on the rate of tail regeneration (Table 2). Given that compounds that modulate ion flux mediated by the foregoing classes of ion transporters had no effect on the rate or extent of regeneration, we are not pursuing these classes of transporters in these biological processes.

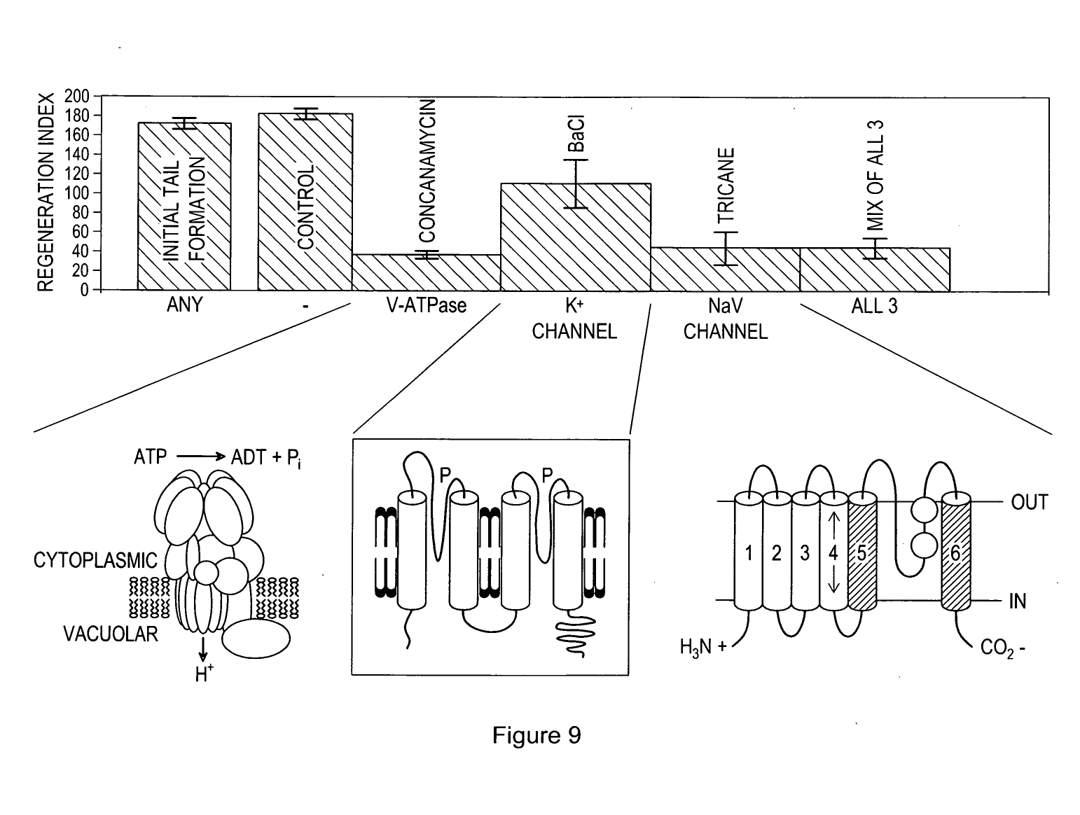

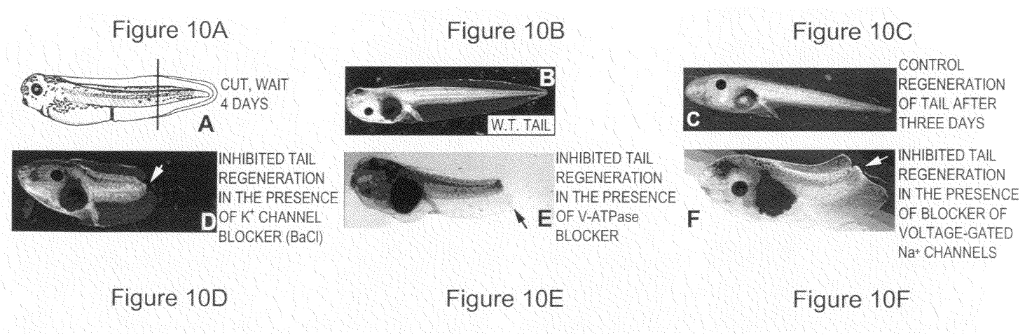

FIG. 9 summarizes experiments performed using compounds that altered the regeneration index, and thus implicated a role for three classes of ion transporter proteins: K⁺ channels, the V-ATPase, and voltage-gated sodium channels. In contrast to amputated embryos exposed to vehicle, which regenerated normally, embryos exposed to blockers of the V-ATPase (e.g., concanamycin), K⁺ channels (BaCl), or voltage-gated Na⁺ (NaV) channels (tricaine) continued normal development in the absence of regeneration (FIG. 10).

These effects were specific to regeneration because (a) exposed embryos were alive and exhibited fairly typical developmental morphology, even in the absence of normal tail regeneration; and (b) despite the lack of tail regeneration, the wound created when the tail was amputated healed successfully.

These results demonstrated that tail regeneration depends on the activity of the three implicated target classes. The effect of BaCl strongly suggests a K⁺ channel. However, the lack of effect on regeneration observed in the presence of THB and glibenclamide suggests that the subject K⁺ channel is not a member of either the class of 6 transmembrane voltage-gated potassium channels or the class of inwardly-rectifying channels. The profile obtained from the compound screen suggests that K⁺ flux during tail regeneration is mediated by the class of two-pore channels (4 or 8 TM domains) of the KCNK family (Goldstein et al., 2001).

These results indicated that members of all three classes of ion transporter proteins are important during tail regeneration. Inhibition of any one of the three classes alone abolishes regeneration. However, the effect was not additive.

### Example 7

### A Role for Ion Transporter Proteins in Dedifferentiation and Regeneration

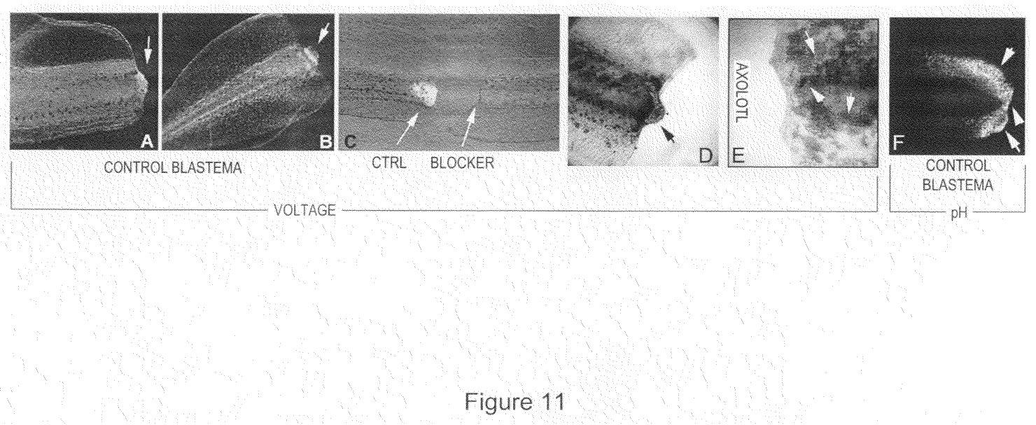

We next examined membrane voltage and pH during tail regeneration. We used the voltage-sensitive fluorescent dye DIBAC₄(3), and examined cultured embryos using confocal microscopy (FIG. 11). Within 12 hours of amputation, the blastema cells exhibit a clear depolarization. Moreover, while the depolarization could be easily observed in blastema cells in embryos cultured without inhibitory compounds (FIG. 11C left), embryos in which regeneration was blocked by an inhibitory compound did not exhibit the same region of depolarization (FIG. 11C right). These observations support a model whereby amputation triggers the formation of a zone of depolarization which is necessary for subsequent regeneration steps.

We also detected a pH gradient at the Xenopus blastema. Based on our inhibitor studies, this pH gradient is likely driven by the V-ATPase (FIG. 11F).

### Example 8

### Expression Analysis of Ion Transporter Proteins During Tail Regeneration

We conducted expression analysis to evaluate the spatio-temporal expression of targets implicated in regeneration based on the compound screen. We performed immunohistochemical analysis using the following antibodies: an antibody raised against the V-ATPase subunit c′; a commercial NaV antibody (Sigma) and a KCNK1 antibody. We detected protein expression using each of these antibodies in Xenopus tail regenerates. Additionally, we evaluated proliferation, and observed a zone of proliferating cells in the blastema.

These results implicate three classes of ion transporter proteins in Xenopus tail regeneration: the V-ATPase H⁺ pump, a K⁺ channel, and a voltage-gated Na⁺ channel. When these targets are specifically inhibited after amputation, wound healing and normal embryonic development continues, while tail regeneration is absent. Moreover, using a voltage-sensitive fluorescent dye, we directly observed the depolarization appearing in the blastema of control embryos, and its alteration in embryos which do not regenerate due to inhibition of the three targets.

### Example 9

### Further Characterization of a Role for Ion Flux During Tail Regeneration

To further analyze the role for ion flux during tail regeneration, the following experiments are performed. Compounds that selectively inhibited candidate ion transporter proteins are used to evaluate the effect on regeneration, ion flux, pH, and/or membrane potential. Exemplary compounds that can be used include small organic molecules, small inorganic molecules, antisense oligonucleotides (e.g., morpholinos, etc), RNAi constructs, blocking antibodies, and dominant negative constructs (e.g., nucleic acids encoding dominant negative proteins).

To further analyze the role for ion flux during tail regeneration, expression of known regeneration marker genes will be analyzed. Expression of particular genes uniquely expressed in regenerating tissue (such as pentraxin I, ld1, ld2, hes1, Wnt pathway genes, Notch pathway genes, etc.), as well as markers of undifferentiated cells, proliferating cells, neuronal precursors, and apoptotic cells can be examined. Expression is examined during normal regeneration, as well as in the presence of compounds that specifically perturb (e.g., inhibit or promote) the activity of one or more of the implicated classes of ion transporter proteins. Additionally, continued examination of ion flux, pH, and membrane potential can be examined.

METHODS: The following methods were used in the experiments performed and discussed in Examples 6-9.

### Amputation Protocol:

Tail amputation technique was performed on wild-type embryos at stage. 40, using a single cut with a fresh scalpel blade under a dissecting microscope.

### Analysis of Morphology:

Control and experimental instances of regeneration can be visually evaluate. In some instances, time-lapsed image analysis using a digital camera over several days can be used to provide MPEG movies of normal and altered regeneration. Regardless of whether effects on regeneration are evaluated with the aid of digital photography, statistical analysis of the effects of various treatments on regeneration was and will be performed using two methods. The most basic is a regeneration index: using a constant magnification on a dissecting microscope, embryos undergoing control and experimental regeneration can be photographed, and the analysis tools of the OpenLab software can be used to measure the length of the tails from the end of the hindgut to the caudal tip, along the midline. The degree of regeneration can be quantified as the percent length of the regenerate relative to control. Such indexes can easily be averaged or subjected to ANOVA or other analyses.

### Confocal Microscopy of Ion Flux:

As outlined above, imaging using any of a number of fluorescent dyes including pH- and voltage-sensitive dyes such as SNARF, BCECF, and DIBAC₄(3) can be performed. For specific details regarding use of DiBAC and SERIS, see above methods.

### Example 10

### Regeneration Studies in Planaria

### A Role for Gap Junctional Communication in Anterior Character of Regenerating Planaria

The restoration of body structures following injury requires an initiation of growth and an imposition of correct morphology upon the regenerating tissue. Understanding this process is crucial both for the basic biology of pattern formation as well as for developing novel biomedical approaches. As used throughout, the term regeneration reflects an appreciation that methods of inducing regeneration (e.g., regeneration of cells or of tissues) require an interplay of proliferative and differentiation events, and may in some tissues also involve dedifferentiation.

Planaria possess remarkable powers of regeneration. Regeneration is fairly rapid (complete after, 7 days) and is dependent upon a population of stem cells (neoblasts). After bisection across the main body axis, the anterior blastema will regenerate a head while the posterior blastema will regenerate a tail.

Genes from the family now known as Innexins (formerly called OPUS) comprise a set of important developmental proteins that show no sequence homology to connexins but have the same topology, including four transmembrane domains. The ability of innexins to form functional gap junction channels has been demonstrated directly for a number of innexins (Landesman et al., 1999, J Cell Sci 112: 2391-2396; Phelan et al., 1998b, Nature 391: 181-184; Stebbings et al., 2000, Mol Biol Cell 11: 2459-2470). Developmental roles of this gene family have been investigated in Drosophila and C. elegans, where analysis of genetic mutants implicated innexins in the development of muscle and neuronal cell types. However, a role for GJC mediated by innexins during regeneration had not previously been investigated or identified.

To facilitate analysis of GJC mediated by innexins, we cloned the members of the Innexin family in the planarian, Dugesia japonica, and characterized their expression in intact worms and during stages of regeneration. We then performed loss-of-function experiments using compounds that inhibit the expression or activity of multiple innexin family members to test the role of gap junctions in planarian regeneration. The induction of bipolar 2-headed animals following exposure to a GJC blocker that inhibits expression of multiple innexin family members demonstrated a role for GJC during regeneration in planaria.

Current efforts by the community of planaria researchers have failed to identify connexin genes in the planaria genome. Therefore, we focused our study of a potential role for gap junctional communication during planaria regeneration on another class of gap junctions: the innexins. Innexin genes are known to underlie gap junctional communication in invertebrates many invertebrate species (Dykes et al., 2004, J Neurosci 24: 886-894; Landesman et al., 1999, J Cell Sci 112: 2391-2396; Phelan et al., 1998, Trends in Genetics 14: 348-349; Phelan and Starich, 2001, Bioessays 23: 388-396).

To isolate planarian innexin genes, we pursued degenerate PCR amplification of innexin gene fragments, using the planarian cDNAs as the templates. We isolated 6 fragments of innexin-like clones, inx1 to 6. We screened a cDNA library to isolate full-length clones. We isolated and sequenced full-length clones for inx1-5. However, inx6 was not present in the cDNA library. We then searched the planarian D. japonica EST database and found an additional 7 putative innexins which were present as incomplete fragments. Based upon these, we screened a cDNA library, and isolated full-length cDNA clones, inx7 to inx13.

All cDNA clones included the initiation codon and the 5′ and 3′ untranslated sequences. The completed sequences of cDNA clones (inx1-5 and inx7-13) and the sequence of PCR fragment of inx6 have been deposited in the DDBJ/EMBL/GeneBank Library database under accession numbers AB189262, AB189252, AB189253, AB189254, AB189255, AB196957, AB189256, AB189257, AB189258, AB189259, AB189260, AB178521 and AB189261. The foregoing accession numbers and accompanying information is hereby incorporated by reference in their entirety.

The conserved four transmembrane domains, cysteine residues in the extracellular loops and tetrapeptide sequence (YYQW, located near the end of the first extracellular loop next to the second transmembrane domain), which exist specifically in all innexin sequences reported so far were also present in the planarian innexin sequences, except for D. japonica inx1 (because it has a stop codon in the third transmembrane domain). These data indicated that these clones are members of the innexin gene family.

To gain insight into possible roles of GJC in regeneration, we characterized the expression of innexin genes in the planarian using whole-mount in situ hybridization. inx1- and inx7-positive cells were present throughout the anterior and two posterior branches of the intestine. Expression changed dynamically during regeneration. In head fragments at 2 days after cutting, inx1 and inx7 were expressed in the two small projections corresponding to the early regenerating posterior branches of the intestine. The regenerating branches expressing inx1 and inx7 extended posteriorly and the regenerating pharynx appeared in the anterior region between them at 5 days after cutting. In 1-2 day tail fragments, the intestine branches expressing inx1 and inx7, which had been originally the posterior branches in the intact worms, integrated at an anterior position. inx1 and inx7 were also expressed in one small projection that appeared at the anterior position of the integrated branches, corresponding to the early regenerating anterior branch.

inx2, inx3, inx4, and inx13 were expressed in the nervous system. Although they were expressed in both the brain and ventral nerve cord (VNC), the distribution of positive cells was different among these genes. inx2 was expressed weakly in the medial region of brain and the medial-distal region of brain branches. inx2 was expressed very weakly in the VNC. inx3 was expressed throughout the brain, and strongly in the medial and lateral regions of the brain and branches. inx3 expression extended to the distal region of the brain branches. inx3 was expressed in the VNC, though the intensity of expression was very low in intact worms, compared to the high expression observed during regeneration.

inx4 was expressed in the brain branches and the medial and lateral regions of the brain. In contrast to inx2 and inx3, inx4 was expressed in neuron-like cells throughout the peripheral region of the head, where sensory organs are aligned and project to the brain branches. inx4 was expressed in the posterior blastema at 5 days after cutting. inx4 was also expressed in the VNC, and this expression was up-regulated in the anterior region at 5 days after cutting.

Additionally, inx4 was expressed in a number of cells throughout the body; with especially strong expression detected in the photoreceptor cells that plug the eyecup of the pigment cells. During regeneration, the expression of inx4 in photoreceptor cells begins at 4 days after cutting and prior to the appearance of the maturely pigmented eyecup at 5 days after cutting.

inx13 was expressed in the lateral and medial region of the brain. In intact worms, the expression in the lateral region was much higher than in the medial region. It was expressed in the brain branches, though the expression was restricted to the stem region. Expression of inx13 in the VNC was very weak, but was up-regulated in the posterior region in the VNC during regeneration.

In addition to the expression observed in the nervous system, these genes were expressed in the pharynx. inx2, inx3, and inx13 were expressed in the posterior region of the pharynx, while inx4 was expressed in the anterior and posterior regions. Despite expression of these innexin family members in the pharynx, the intestine-type innexins (inx1, inx7) were not expressed in the pharynx.

The expression of inx2, inx3, inx4 and inx13 changed dynamically during brain regeneration. We categorized dynamic expression into two categories: early (initiating in the regenerating brain within 1 day after cutting) and late (initiating at 2 days after cutting). inx2 and inx4 were late genes. The expression of inx2 was initiated in the medial and lateral region of the regenerating brain at 2 days after cutting. At 3 days to 4 days after cutting, inx2 was expressed in the broad region regenerating the brain branches in the anterior blastema. The expression of inx4 was initiated at the anterior-medial region of the regenerating brain at 2 days after cutting. At 3 to 5 days after cutting, inx4 was expressed in the medial region of the regenerating brain. The expression of inx4 was up-regulated transiently in the medial region of the regenerating brain at 4 days after cutting.

In contrast, inx3 and inx13 were early genes. Expression was first detected at 18 hours and 1 day after cutting, respectively. The expression of inx3 initiated in the early regenerating brain in the anterior region of the blastema within 1 day. The earliest detectable signal was seen at 18 hours after cutting. At 2 days, inx3 was expressed in the medial and lateral region of the regenerating brain. At 4 to 5 days, the strong expression of inx3 delineated clearly the structure of brain branches. The expression of inx13 initiated in the early regenerating brain in the anterior region of the blastema at 1 day after cutting. At 2 days after cutting, inx13 was expressed in the medial and lateral region of the regenerating brain. At 3 days, inx3 was expressed in the stems of early regenerating brain branches. At 4 to 5 days after cutting, the expression of inx13 in the regenerating brain branches grew out peripherally, following the regeneration of the brain branches, but did not extend completely to the tip of the brain branches

In intact worms, inx5 was expressed at the edge of the head where sensory organs are aligning and in the scattered cells distributing throughout the dorsal side of the body. Inx5 expression exhibiting gradated distribution from the head to tail along the AP axis, and in a number of cells along the VNC, with a dense distribution along the VNC in the head region. During regeneration, inx5 was expressed in the blastema. At 2 days after cutting, inx5 was initially expressed at the edge of the anterior blastema and in some scattered blastema cells. Sectioning revealed that inx5 was expressed at the leading edge of head mesenchyme in the regenerating head. Following brain regeneration, the inx5-positive cells appeared at a high density along the VNC in the regenerating head region and in the regenerating tail region.

inx12 was expressed very weakly in the head and tail region in intact worms. During regeneration, inx12 was expressed in both of the anterior and posterior blastema and weakly in the midline in the posterior region of the body. Sectioning revealed that inx12 was expressed in the mesenchyme anterior to the regenerating intestine in the anterior blastema at 2 days after cutting. At 5 days after cutting, the expression level of inx12 was reduced in the blastema, and the expression was mostly restricted at the edge of the regenerating head. Following brain regeneration, inx12 was expressed in cells outlining the VNC in the regenerating head.

inx8 and inx9 were expressed in the mesenchyme throughout most of the body but not in the intestine. inx8 and inx9 were expressed in the mesenchyme between the epithelium/muscle, intestine and nervous system, though there were some differences: inx8 was strongly expressed in some mesenchyme cells around and between the small branches of intestine and between the intestine branch and pharynx, as well as in the pharynx; inx9 was more ubiquitously expressed in the mesenchyme, but not expressed between the intestine branch and pharynx. Both inx8 and inx9 were strongly expressed in the mesenchyme tissue around the pharynx and at the midline in the tail region. Although inx8 and inx9 were strongly expressed in the regenerating head and tail at a late stage of regeneration, inx9 was highly expressed in the anterior blastema. Sectioning revealed inx9 in the thin mesenchyme layer outlining the anterior part of the regenerating intestine in the anterior blastema at 2 days after cutting. inx11 was also expressed in the mesenchyme. Additionally, inx11 was strongly expressed in the dorsal midline of the body. In contrast to the expression pattern of inx8 and inx9, the expression of inx11 was restricted to the medial region in the head mesenchyme.

inx10 was expressed in a number of small thread-like structures mainly in the lateral-peripheral region in the intact worms. The threadlike structures were sparsely distributed in the mesenchyme tissue underneath the epithelium. This was similar to the known distribution of the protonephridia observed in electron microscopy studies reported previously. During regeneration, the shape of threadlike structures expressing inx10 changed dynamically in the blastemas. inx10 was expressed also in the anterior and posterior regions of the pharynx, similarly to the expression as inx4.

To test the hypothesis that gap junctional communication was required for correct patterning during regeneration, we sought an inhibitory compound that would broadly disrupt the function of this class of gap junctions. Currently-popular RNAi approaches are not well-suited for this purpose because they target individual innexin transcripts. Thus, we selected heptanol, a compound that specifically disrupts a broad classes of gap junctions. Heptanol and other long-chain n-alkanols are efficient and rapidly-reversible inhibitors of both electrical and chemical GJC in both connexin and innexin-based gap junctions. In the context of the planarian system, heptanol can be used to disrupt the activity of the broad class of innexin gap junctions.

Regenerating worms were contacted with the compound at an early stage of regeneration (2 days after cutting). 1-10 μM heptanol was dissolved in the medium. This concentration was not toxic to the worms, and additionally did not cause observable morphological defects in intact worms. At 7 days post-cutting, we assayed the worms for the morphology of blastemas. Trunk fragments of worms exposed to heptanol exhibited clear anteriorization of both blastemas in 43% of the cases (n=423). The range of anteriorized phenotypes included a loss of tail development, ectopic pharynx posterior to the primary pharynx, appearance of an ectopic eye in the posterior blastema, or a complete head at the posterior end (16% for complete bipolar heads); such bipolar anterior (janus) animals were fully viable. Thus, inhibition of this class of ion transporter proteins modulated regeneration in planaria.

In contrast, all worms regenerating in spring water exhibited normal regeneration (n=107). Exposure to hexanol, a reagent similar to heptanol but which is much less effective at blocking GJC, never induced strong anteriorization of the posterior blastema. However, this weaker compound did inhibit tail regeneration (the weakest class of anteriorization). This phenotype is consistent with a dependence of anteriorization upon the degree of GJC inhibition. Importantly, GJC inhibition induced the growth of anterior structures (in many cases, well-formed ectopic heads) and not simply a cessation of regeneration, ruling out toxicity as the mechanism and implicating GJC in events that determine the axial identity of the structure formed during regeneration.

We next sought to ascertain whether the anteriorizing effect was dependent on the AP (anterior-posterior) level from which the fragment originated. Worms were amputated at four levels to make five body fragments: head, pre-pharyngeal, trunk (including the pharynx), post-pharyngeal and tail fragments. To enable quantitative analysis of the effect on regeneration, we defined a simple continuous “anteriorizing index” on which each worm was scored as normal or exhibiting weak/strong/complete anteriorization. This allowed a direct comparison of the effects observed in each treated group. To briefly summarize, the strongest anteriorization due to GJC inhibition was observed in the pre-pharyngeal and trunk fragments (anteriorization indexes of 25.8 and 27.6 respectively). The head and post-pharyngeal fragments were less sensitive (anteriorization indexes of 5.6 and 6.2 respectively). No effect was observed on tail fragments. These data are consistent with a role for GJC in mediating the axial patterning along the anterior-posterior axis during regeneration in the planarian.

To analyze at a molecular level the patterning changes induced in regenerating worms following inhibition of innexin-mediated GJC, we performed whole-mount in situ hybridization analysis of marker genes in bipolar worms. The CNS marker DjPC2 (Agata, 1998, Zoological Science 15: 433-440) was expressed in the brain, VNC and posterior position of the pharynx in the control worms. In perturbed worms exhibiting the bipolar head phenotype, DjPC2 was expressed in the brains (two brains—one brain located at each end) and two pharynxes that lay asymmetrically as mirror images.

The brain marker DjotxB (Umesono et al., 1999, Dev Genes Evol 209: 31-39) was expressed in the brain and the cells outlining the posterior half of mouth in the control worms. In perturbed worms exhibiting the bipolar head phenotype, DjotxB was expressed in the brains (two brains—one brain located at each end) and in the mirror imaged-mouths.

The innexin gene, inx7, is a good marker of the intestine. Normally, the intestine has an asymmetric shape along the AP axis: it has one intestine branch anteriorly connected to the pharynx and two intestine branches located posteriorly. In perturbed worms exhibiting the bipolar head phenotype, inx7 expression indicated that the intestine were symmetrically aligned.

In control, untreated worms, the tail marker DjAbd-Ba (Nogi and Watanabe, 2001, Develop Growth Differentiation 43: 177-184) was expressed strongly in the tail region posteriorly to the pharynx. In perturbed worms exhibiting the bipolar head phenotype, DjAbd-Ba was expressed weakly and broadly in the domain laterally to the pharynxes in the trunk region, and was not expressed in the originally-posterior region in the body.

These results demonstrated a role for gap junctional communication mediated by the innexin class of ion transporter proteins in regeneration in planaria. Specifically, gap junctional communication modulates regeneration, as well as the anterior character of regenerating fragments.

### Worm Husbandry

The asexual clonal strain GI of the planarian, Dugesia japonica, was used in these study. In all experiments, the worms were starved for 1 week before use.

### PCR-Based Cloning of the Innexin Genes

cDNA from regenerating head and tail fragments of planarians (mixed stages at 1-6 days after cutting) were used as templates for PCR to amplify the planarian innexin genes from a library (5×10⁶ independent clones) using the forward primer 5′-CGCGGATCCWSNRRNCARTAYGTNGG-3′ and degenerate reverse primer 5′-CGGAATTCGGNACCCAYTGRTARTA-3′, corresponding to the highly conserved regions of innexin genes. The amino acid sequences of these highly conserved regions are (S/T)(K/G)QYVG and YYQWVP, respectively. The PCR amplification was carried out with one cycle at 94° C. for 1 min, followed by 40 cycles of 30 sec at 94° C., 30 sec at 45° C. and 30 sec at 72° C., and by a final extension at 72° C. for 5 min. The library was screened by the PCR-based stepwise dilution method (Watanabe et al., 1997, Anal Biochem 252: 213-214).

### Whole-Mount In Situ Hybridization

Whole-mount in situ hybridization was performed as described previously according to standard methods with the following modifications employed for greater sensitivity and lower background. Prior to prehybridization, the samples were incubated twice in 0.1 M triethanolamine, pH 7.6, for 15 min at room temperature, and were acetylated using an acetic anhydride series (0.25% and 0.5%) in 0.1 M triethanolamine, pH 7.6, for 15 min each at room temperature. Hybridization was carried out in hybridization solution (50% formamide, 5×SSC, 100 μg/ml yeast tRNA, 100 μg/ml heparin sodium salt, 0.1% Tween-20, 10 mM DTT, 5% dextran sulfate sodium salt) including about 40 ng/ml digoxygenin (DIG)-labeled antisense riboprobe that had been denatured at 70° C. for 10 mm.

### Drug Exposure for GJC Inhibition

Intact worms 1-1.5 cm long were put into heptanol (or hexanol) solution (0.0045-0.006% vigorously vortexed into spring water) immediately prior to amputation to equilibrate the worms with the drug solution. The worms were amputated at four levels to generate the head, pre-pharyngeal, trunk (or “pharyngeal”), post-pharyngeal, and tail fragments. Worm fragments were incubated at 22° C. for 2 days. The heptanol solution was exchanged for fresh solution every day. The worms were then washed with water twice and incubated in worm water for 14-20 days to monitor the phenotypes.

### Scoring System for Anterior-Posterior Phenotype of Regenerates

We developed a quantitative scheme allowing comparison of degree of anteriorization among groups of worms. Each worm was scored on the following scale by observing the posterior blastema: 0 points—normal (a normal worm with a fully-patterned tail), 1 point—weak anteriorization of posterior blastema (missing tail or bipolar pharynx), 2 points—stronger anteriorization of posterior blastema (incomplete ectopic head with eye structures), or 3 points—complete anteriorization of posterior blastema (bipolar head, where the ectopic head has complete development with 2 normal eyes). For each group of worms, we calculated an average score that is the sum of all scores for the worms divided by the total number of worms. For convenience, the index was scaled from 0 to 100 (final index=average score*100/3). On this scale, a group of worms that were all normal would score 0, while a group of worms all of which were fully double-head would score 100. This scheme was focused on ascertaining the extent of anteriorization as judged by external morphology.

### Example 11

### Characterization of the Role of V-ATPase H⁺ Pump in Regeneration

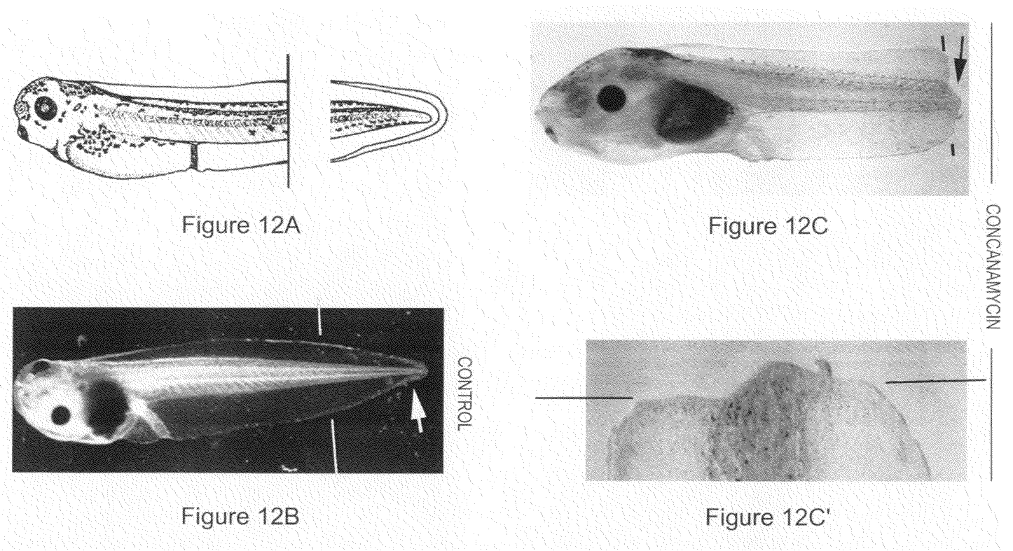

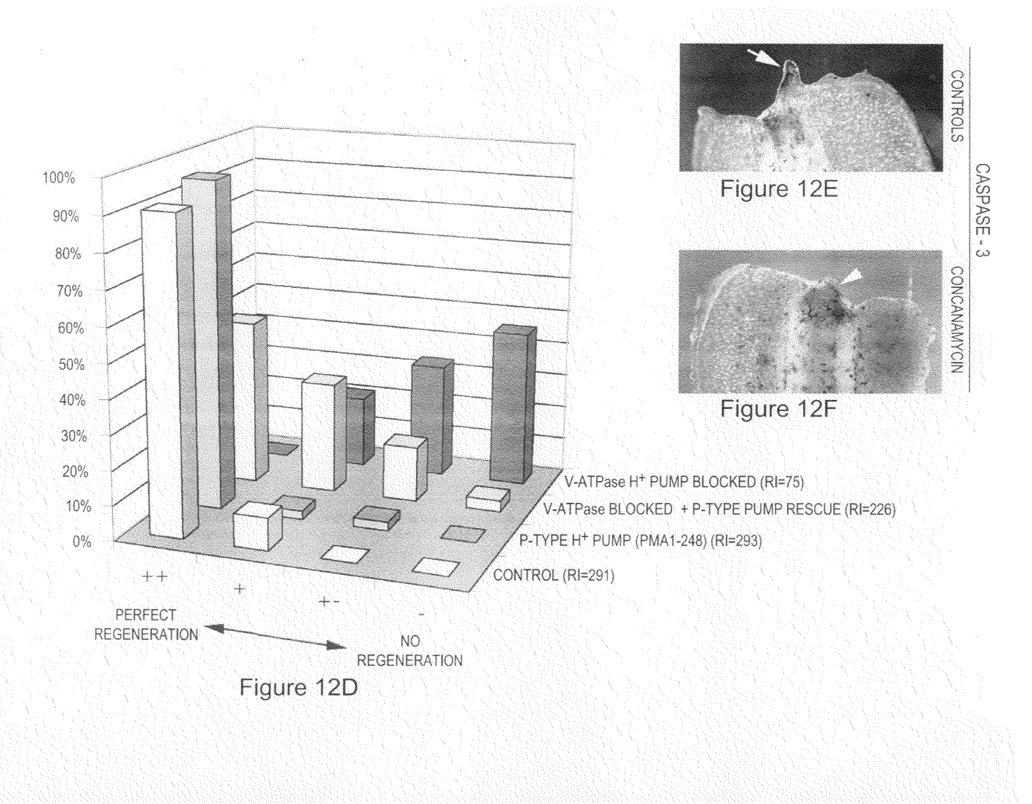

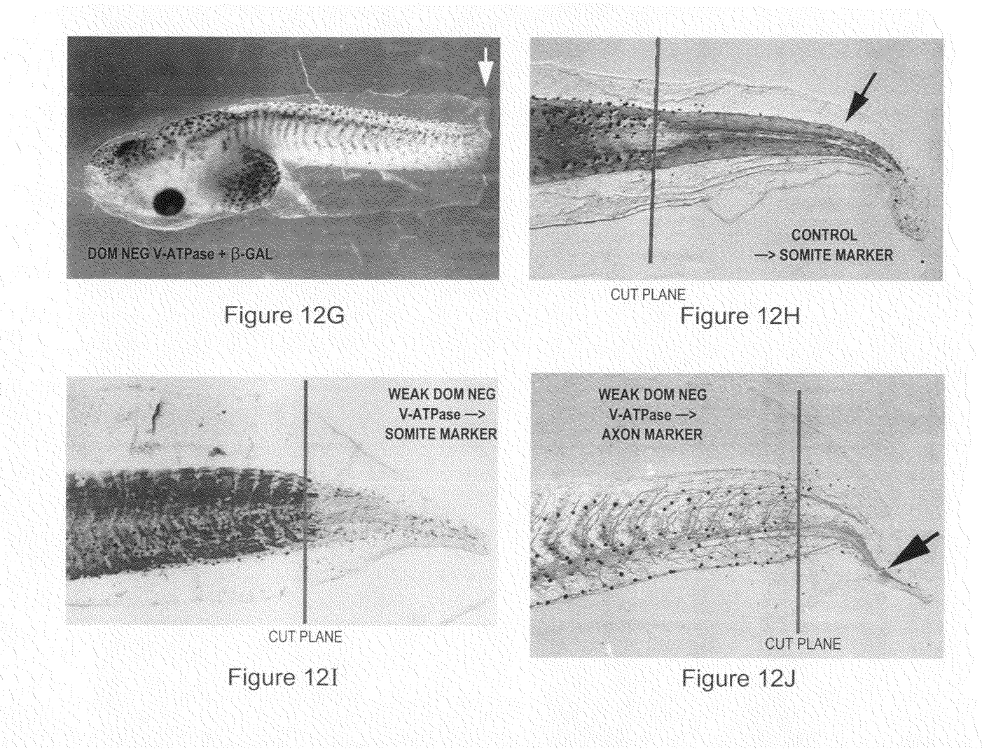

Further experiments were done in the Xenopus system based on the results of the pharmacological screen (see Example 6). In contrast to normal regeneration taking place when larvae are amputated at stage 41 (FIG. 12A,B), exposure to 150 nM concanamycin, a potent and highly specific inhibitor of the V-ATPase H⁺ pump, results in a strong inhibition of regeneration in the absence of general toxicity (N=226, U=11628, Z=11.357; FIG. 12C, Table 3, and see methods). The V-ATPase (Nishi, 2002, Nature Reviews Molecular Cell Biology 3: 94-103) generates strong pH and voltage gradients at the expense of ATP, when expressed in vesicular or cell plasma membranes. Analysis of the localization of activated caspase-3 in control and V-ATPase-inhibited larvae (FIG. 12E,F) revealed that regenerating tails normally possess a small apoptotic cell group, but no significant difference was observed in the degree of apoptosis under V-ATPase inhibition, suggesting that an increase of cell death does not account for this failure to regenerate.

The ability to target different embryonic regions with early injections of mRNA provided an opportunity to phenocopy the pharmacological phenotype with a molecular loss-of-function construct and also to test the spatial requirements for V-ATPase activity. Using a well-characterized dominant negative V-ATPase E subunit YCHE78 (Lu et al., 2002, Journal of Biological Chemistry 277: 38409-15), we confirmed the same phenotype obtained with concanamycin; YCHE78 misexpression at high levels in the tail (detected by GFP lineage label) prevented regeneration as compared with injected animals not exhibiting YCHE78 expression in the tail (N=66, H=100.232, Q=3.556, p<0.01). These data strongly support the necessity for endogenous V-ATPase function in the tail for regeneration.

The strong inhibition of regeneration (red bars) by pharmacological V-ATPase blockade was largely prevented (FIG. 12D; white bars=no treatment; green bars=PMA injection only) by misexpression of a concanamycin-insensitive yeast P-type H⁺ pump (PMA1.248, yellow bars) 28 in the tail (301% increase in R1 relative to concanamycin-exposed embryos; N=127, H=81.486, Q=4.672, p<0.01). This rescue experiment demonstrated that it is indeed H⁺ pumping provided by the V-ATPase during normal regeneration that is blocked by concanamycin exposure and normally ensures complete regeneration.

To test for cell-autonomy of the V-ATPase mechanism in regeneration, we isolated embryos in which the dominant negative V-ATPase construct YCHE78 was localized to somites (confirmed by β-gal lineage label, FIG. 12G). Control regenerates possess a significant muscle component by 96 hours post-fertilization (hpa) as detected by expression of the 12-101 skeletal muscle marker (FIG. 12H). In contrast, regenerating tissue in those few larvae exhibiting a weaker YCHE78 effect, and thus some degree of regeneration, contains little or no detectable muscle marker signal (FIG. 12I). Axon outgrowth patterns in YCHE78 injected animals were, however, completely normal in the partially regenerated tail (FIG. 12J). These data suggest that the V-ATPase activity in the somites is required for muscle regeneration but not for nerve regeneration, supporting a cell-autonomous role for H⁺ pumping in the somites.

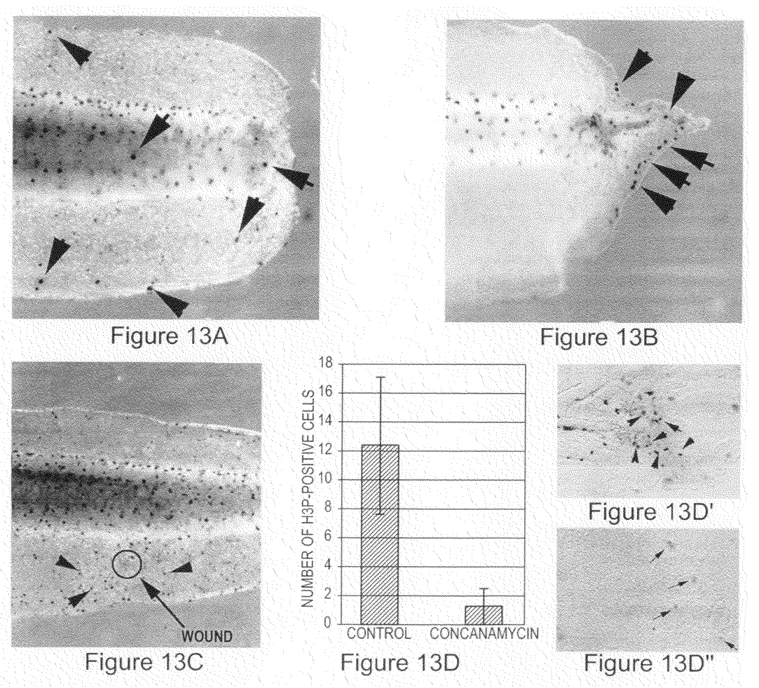

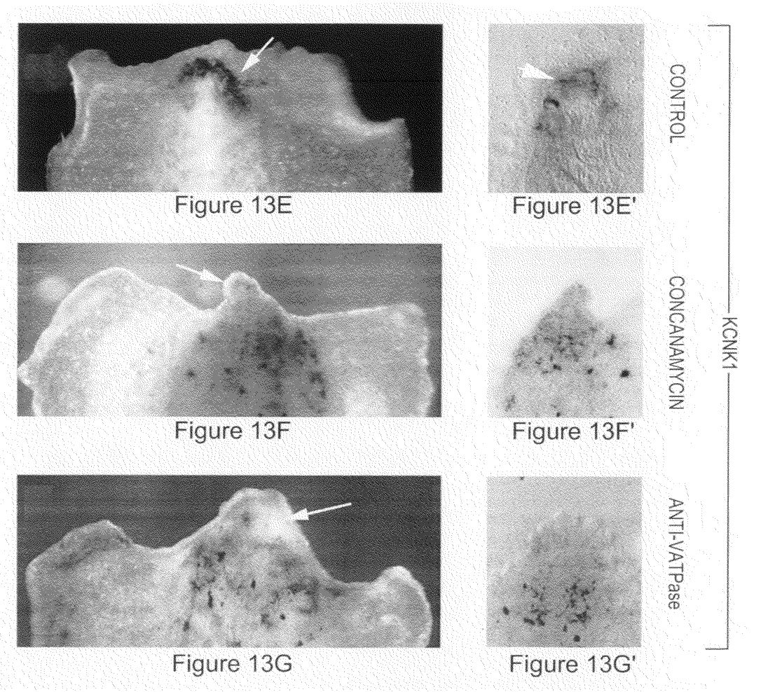

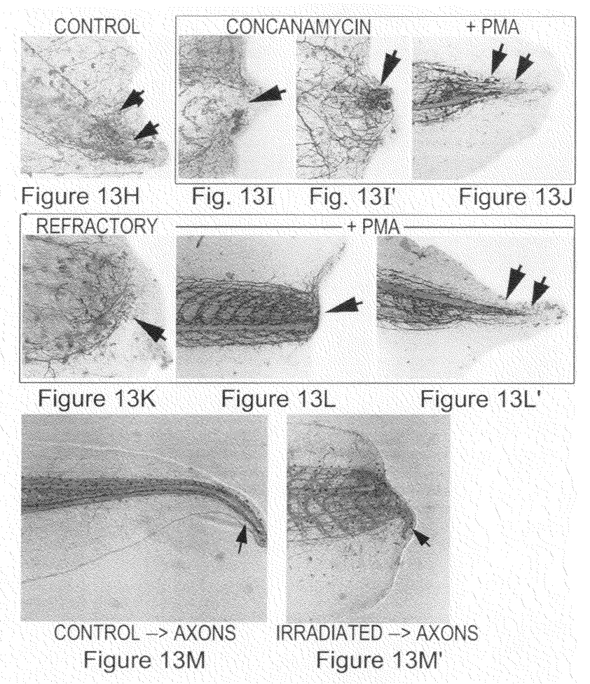

We next examined the endogenous expression of the V-ATPase in the regeneration bud compared to most targets (FIG. 13A,A′). Expression of the c subunit of the V-ATPase could be detected at the mRNA (FIG. 13B,B′) and protein (FIG. 13C,C′) levels specifically in the regeneration bud within 6 hours of amputation; other V-ATPase subunits were also up-regulated (data not shown). A low level of background expression elsewhere in the trunk was detected, due to the ubiquitous vacuolar form of the V-ATPase (data not shown). However, the strong plasma membrane expression was observed only in the regeneration bud. Thus, the pump is endogenously expressed in a spatio-temporal pattern consistent with an endogenous role in regeneration.

We also investigated V-ATPase expression in tails cut during a refractory period, during which Xenopus larvae cannot regenerate (Beck et al., 2003, Dev Cell 5: 429-39). Expression was normal in refractory tails (FIG. 13D), suggesting that their inability to regenerate was not due to the failure to turn on V-ATPase expression in the regeneration bud but rather to a post-translational step in V-ATPase function. To determine whether the V-ATPase is normally up-regulated in existing cells or produced by a new cell population generated in response to amputation we irradiated larvae—a procedure known to abolish cell proliferation (Li et al., 2001, Comp Biochem Physiol A Mol Integr Physiol 130: 133-40; Salo and Baguna, 1985, J Embryol Exp Morphol 89: 57-70). Irradiated larvae still up-regulated V-ATPase expression in the wound (FIG. 13E). We confirmed the loss of proliferative cells (FIG. 13F) after X-irradiation, and the resulting failure to regenerate (FIG. 13G). These data suggest that the V-ATPase up-regulation takes place in existing wound cells and does not require the production of a new cell type in the regeneration bud.

We next directly examined the physiology of the regeneration bud using the voltage reporter dye DiBAC₄(3). Consistent with IHC localization of V-ATPase in the bud (FIG. 13B-C′), we found that in a normal regenerating tail, the bud is depolarized relative to the rest of the tail (FIG. 13H). To determine relative depolarization of uncut versus treated tails, the gain of the photomultiplier tube was reset below that used to generate FIG. 13H. With these adjusted conditions, it is possible to see that control regeneration buds (FIG. 13J) are somewhat depolarized relative to uncut tails (FIG. 13I), while V-ATPase-inhibited (FIG. 13K) are more dramatically depolarized, as evidenced by the higher fluorescence intensity. Refractory tails also exhibited a strong depolarization (FIG. 13L) despite normal expression of the V-ATPase.

To ask whether the V-ATPase controls regeneration directly via its ion pumping activity, we depolarized tails with a method not relying on V-ATPase: 2 nM palytoxin (Castle and Strichartz, 1988, Toxicon 26: 941-51; Hilgemann, 2003, Proc Natl Acad Sci USA 100: 386-8). This resulted in a 33% reduction of regeneration index (N=81, U=990.5, Z=2.926, p=0.002), suggesting that it is indeed the membrane voltage level (and downstream effects on voltage-sensitive proteins) that is crucial for regeneration. We conclude that consistent with its expression, the ion pumping activity of the V-ATPase is an important determinant of the steady-state membrane polarization level in the regeneration bud cells. Moreover, these data demonstrate that refractory regeneration buds are unable to maintain normal polarization because of a process downstream of, or distinct from, V-ATPase expression. One possibility is the existence of an as-yet-unidentified depolarizing transporter functioning in refractory tails.

To determine whether induction of H⁺ flow is a promising strategy for inducing regeneration in a gain-of-function application, we tried to rescue the ability to regenerate during the refractory period by misexpression of the yeast PMA1.2H⁺ pump. Remarkably, expression of PMA1.2 led, in 21/55 refractory tadpoles, to significant regeneration (FIG. 13M,M′; ectopic up-regulation of outgrowth occurring in and at 90° to the neural tube (red arrows); RI increase of 287% over control refractory tails; N=103, U=1638.5, Z=13.005, p<<0.001). This is the first example of the induction of regeneration by molecular expression of an ion transporter and provides a novel entrypoint into this complex process that may be exploited by future clinical augmentation efforts. Consistent also with a general control of growth by H⁺ transport (Cone and Tongier, 1971, Oncology 25: 168-82; Gillies et al., 1992, Cell Physiology and Biochemistry 2: 159-179), misexpression of this ion pump can activate growth even in uncut tissue, and this effect is not restricted to the tail (FIG. 13M″).

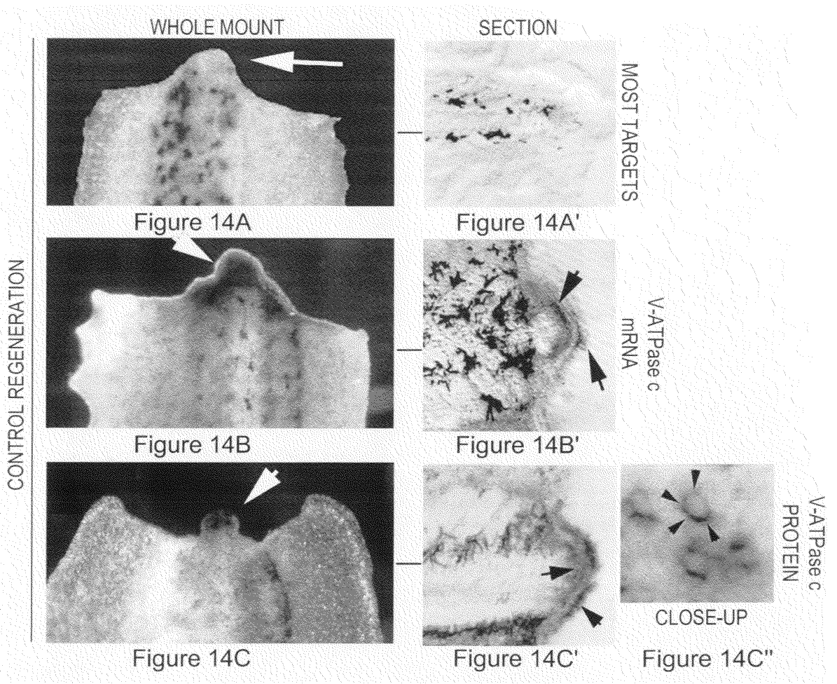

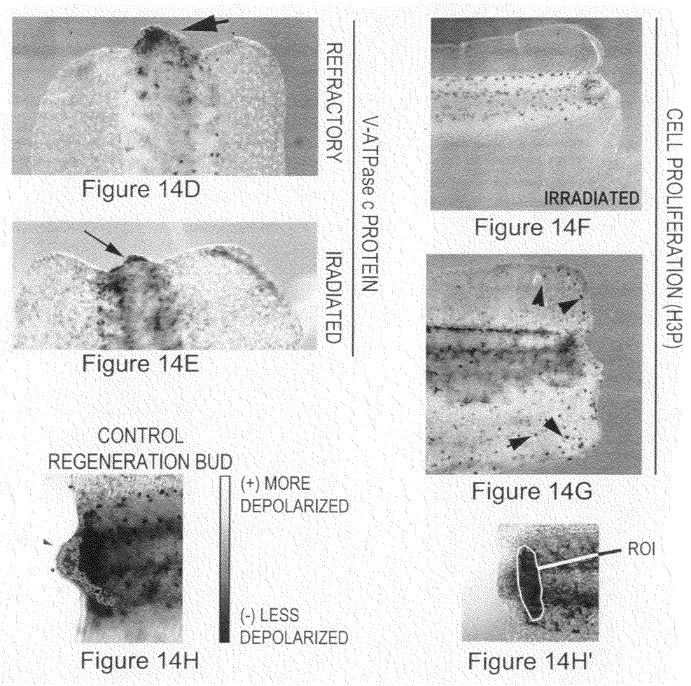

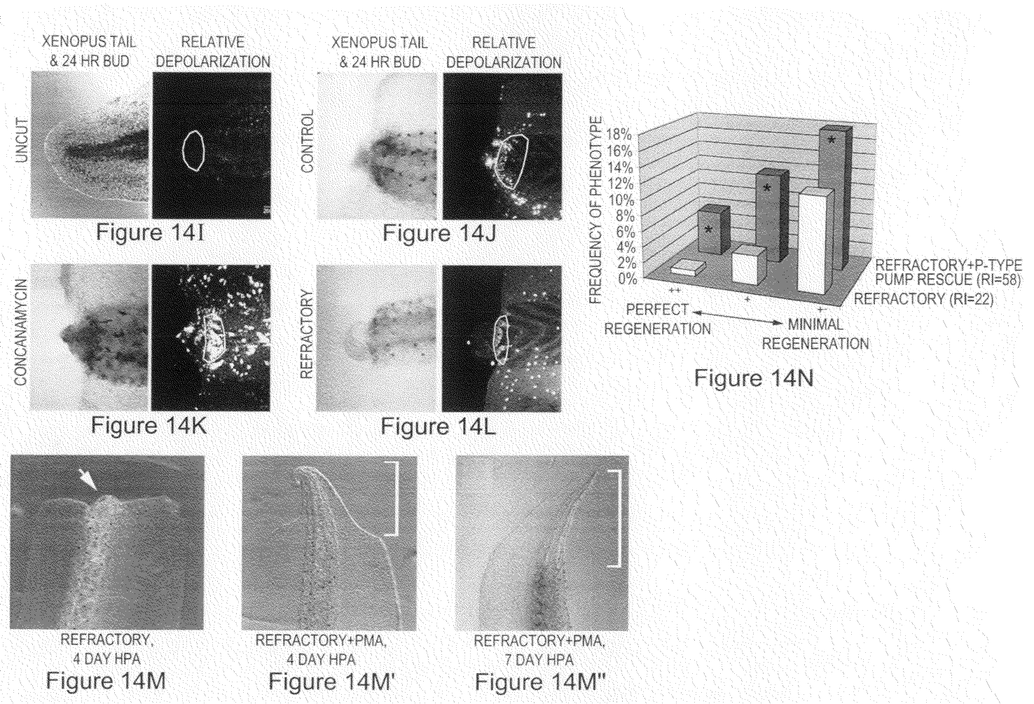

To gain insight into the cellular mechanisms by which the V-ATPase participates in regeneration, we characterized the pattern of proliferating cells during regeneration, using an antibody to the phosphorylated Histone 3B—a standard marker of cells in the G₂/M transition of the cell cycle, useful for identifying mitotic cells in regenerating systems including Xenopus (Saka and Smith, 2001, Dev Biol 229: 307-18; Sanchez Alvarado, 2003, Current Opinion in Genetics & Development 13: 438-444). At 24 hpa, this subset of proliferating cells is homogenously distributed throughout the growing tail (FIG. 14A). In contrast, by 48 hpa, these cells are (as expected) highly enriched in the regeneration bud, but, surprisingly, are largely absent from the region of the flank anterior to the amputation (FIG. 14B). Thus, normal regeneration includes two components: an increase in proliferation in the bud, and a >2.3-fold reduction of proliferation in the flank rostral to the amputation plane. The reduction of surrounding proliferating cells does not occur when a puncture wound is made (FIG. 14C; red arrows point to H3P-positive cells [see Methods]; green circle indicates the location of the puncture wound), providing a mechanistic readout of the difference between true regeneration and wound healing. Specific inhibition of the V-ATPase resulted in an approximately 6-fold decrease in the number of proliferating cells in the regeneration bud (FIG. 14D,D′,D″), and approximately a 2.5-fold decrease of proliferation in the flank (this reduction did not noticeably impair larval development or behavior). Moreover, V-ATPase-inhibition results in the complete failure of the normal strong reduction of proliferating cells in the flank at 48 hpa (F=11.02, p=0.0002, Table 4). These data suggest that the V-ATPase is required for the up-regulation of proliferation in the growth zone after amputation, as well as for the normal loss of proliferation in the flank. The purpose of this mid-flank reduction is unknown, but it demonstrates that regeneration is not a purely local phenomenon (since mid-flank tissues at a distance of up to 3 mm anterior to the regeneration bud must receive a regeneration-specific signal to stop proliferating) and that such long-range signaling is V-ATPase dependent. Gap junctions are another ion flow control mechanism that has recently been shown to carry long-range information in a complex regenerating system (Nogi and Levin, 2005, Dev Biol 287: 314-35).

We then sought to functionally link V-ATPase activity to gene expression in the regeneration bud. Existing markers include Notch pathway genes². However, these are only expressed in tissue that does not exist in V-ATPase-inhibited larvae, and thus cannot be examined in V-ATPase loss-of-function regeneration buds. Because of this, and because the V-ATPase is expressed so soon following amputation, we utilized an earlier marker that is normally expressed by 12 hpa: the K⁺ channel KCNK1 (FIG. 14E,E′). In larvae in which V-ATPase activity was abrogated by concanamycin or a function-blocking anti-V-ATPase antibody, KCNK1 expression was absent (N=13, FIG. 14F,F′-G,G′). Thus, V-ATPase is upstream of some gene expression in the regeneration bud, including other ion transporters also specifically expressed during early stages of regeneration.

Finally, to characterize downstream morphogenetic consequences of V-ATPase abrogation, we traced axonal paths during regeneration using immunohistochemistry with an acetylated α-tubulin antibody. In normally regenerating tails, axons appear increased in number (relative to the uncut portion of tail) and they extend into the bud in bundles parallel to the main anterior-posterior axis of the tail (FIG. 14H; green arrows indicate normal axon patterning, while black arrows indicate abnormal axon number and/or location). In contrast, the axons of V-ATPase-inhibited tails increase in density, but axon patterning is abnormal, with axons absent from the middle of the regeneration bud (FIG. 14I) or appearing tangled at the tail tip (FIG. 14I″). These data demonstrate that V-ATPase is required not only for expression of marker genes in the regeneration bud and the increase in proliferation in the growth zone, but also for the patterning of axons in the tail. The known dependence of axon orientation on electrical cues (McCaig et al., 2002, Trends in Neurosciences 25: 354-9) suggests a testable model whereby regeneration currents serve to orient the growth of axons during regeneration of the tail. Consistent with this, our data show that expression of the yeast proton pump, which was able to rescue V-ATPase-inhibited and refractory-inhibited regeneration, also restored normal axon patterning to concanamycin-treated tails (FIG. 14J). Moreover, in normal refractory tails, there is no apparent increase in the number of axons, and those that are present terminate well anterior of the tail tip (FIG. 14K). However, expression of the yeast proton pump PMA in refractory tails induced both the proliferation and axonal patterning normal to regeneration, in tails in which morphological regeneration was and was not induced (FIG. 14L,L′). In those larvae in which normal tail outgrowth was not rescued by PMA, the presence of axons at the very edge of the wound was induced in 25/30 animals (FIG. 14L), demonstrating that the neural patterning and outgrowth are distinct components of the regenerative response, both downstream of H⁺ flux.

We next sought to probe the relationship between the two major patterning mechanisms observed to depend on the V-ATPase. In order to determine whether the abnormal axonal patterning observed in V-ATPase-inhibited larvae is caused by the inhibition of cell proliferation, we γ-irradiated larvae to abolish proliferation (FIG. 13F). In such animals, despite a lack of regeneration (FIG. 13G), axonal patterning extends all the way to the tip in bundles parallel to the main axis of the bud (FIG. 14M,M′), unlike what we observed due to V-ATPase inhibition (FIG. 14I,I′). Thus, the patterning of axons depends upon V-ATPase activity in a pathway parallel to the induction of cell proliferation, and is not a secondary consequence of mitotic activity.

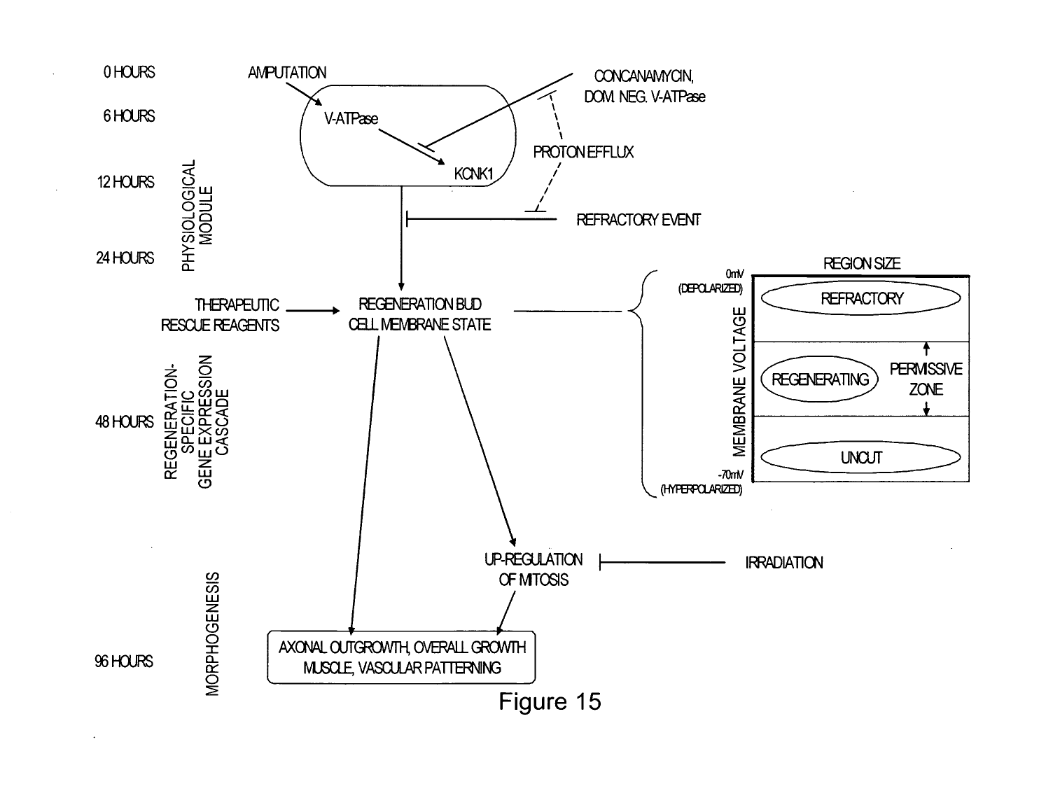

Our characterization and loss- and gain-of-function data demonstrate a consistent expression and function of the V-ATPase, and reveal it as a novel biophysical component required for early steps of regeneration of a complex vertebrate appendage. Based on these data, we suggest a model that integrates the known molecular genetic and physiological components (FIG. 15). Amputation triggers a cassette of ion transporter expression in existing cells, with V-ATPase functionally upstream of KCNK1. The activity of the V-ATPase and its downstream transporters results in a specific range of membrane polarization in the regeneration bud cells, leading to an up-regulation of mitosis and axonal outgrowth, ultimately resulting in the regeneration of the tail. Refractory stage larva cannot regenerate due to an extreme depolarization occurring despite normal V-ATPase expression. It is the crucial events downstream of the ion pumping activity of the V-ATPase (occurring as early as 6 hpa) that are disrupted by concanamycin or dominant negative V-ATPase mutant expression. The voltage gradient in the bud depends on the V-ATPase, but the precise physiological state is likely to be a complex function of a module including a number of other ion transporters. Ectopically-induced H⁺ flux can be used to rescue upstream steps and initiate the program of regeneration, representing a tractable “master control” point for therapeutic approaches.

Comparison of regenerating, refractory, and uncut tails' voltage maps (FIG. 13H-L) revealed that regenerating buds maintain a moderate level of membrane voltage depolarization, and that deviations from this permissive zone towards either strong depolarization (in refractory, V-ATPase-inhibited) or hyperpolarization (in uncut) is associated with a quiescent, non-growing condition. The permissive physiological state is established by 24 hours, and this level of membrane voltage in regeneration bud cells is not achieved by refractory tails (this physiological property is currently the earliest known mechanistic difference between permissive and refractory stages). Consistent with the known dependence of mitosis on membrane voltage and ion transport activity (Cone, 1974, Annals of the New York Academy of Sciences 238: 420-35; Cone and Cone, 1976, Science 192: 155-8; Olivotto et al., 1996, Bioessays 18: 495-504), this bioelectrical state leads to the required proliferation in the bud and, in parallel, to axonal outgrowth into bud tissues. Galvanotactic guidance is a likely mechanism (Gruler and Nuccitelli, 1991, Cell Motility and the Cytoskeleton 19: 121-133; McCaig et al., 2002, Trends in Neurosciences 25: 354-9) for the effect on neuronal patterning.

METHODS: The following methods were used in the experiments summarized in Example 11.

Amputation procedure and pharmacological screen Xenopus laevis larvae at stage 40-41 (Nieuwkoop and Faber, 1967) had their tails amputated under a dissecting microscope using a scalpel blade at the point where the tail begins to taper. Amputated larvae were cultured in 0.1×MMR/gentamycin drug). Larvae were kept at 22° C. for 7 days and scored for regeneration as below. Drug experiments were carried out at least in duplicate (see Table 2). This screen strategy relies on iterative use of blocker reagents, proceeding from substances of broad targeting to those with high specificity; this results in a binary search that rapidly and inexpensively probes an enormous family tree of all known transporters. Due to the high conservation of ion transporters among phyla, reagents developed in mammalian systems are often useful in invertebrate preparations (Alshuaib and Mathew, 2004, Int J Neurosci 114: 639-50; Carvelli et al., 2004, Proc Natl Acad Sci USA 101: 16046-51; Etter et al., 1999, J Neurochem 72: 318-26; Gasque et al., 2005, J Neurosci 25: 2348-58; Pyza et al., 2004, J Insect Physiol 50: 985-94), and a number of labs have utilized this technique to uncover novel transporters involved in morphogenetic events in both vertebrates and invertebrates (Etter et al., 1999, J Neurochem 72: 318-26; Gasque et al., 2005, J Neurosci 25: 2348-58; Hibino et al., 2006, Development, Genes, and Evolution in press: Pyza et al., 2004, J Insect Physiol 50: 985-94; Shimeld and Levin, 2006, Developmental Dynamics in press). However, because of possible structural divergences among species, the screen results do not conclusively (and we do not claim) that targets not implicated in the screen are not involved. Rather, the screen allowed us to efficiently implicate a small number of specific transporters for molecular validation and characterization.

Palytoxin (PTX) exposure: palytoxin is a protein from Palythoa tuberculosa that converts ubiquitous Na⁺/K⁺ transporters into a non-specific pore leading to rapid depolarization (Castle and Strichartz, 1988, Toxicon 26: 941-51; Hilgemann, 2003, Proc Natl Acad Sci USA 100: 386-8; Tosteson et al., 1997, Ann N Y Acad Sci 834: 424-5; Tosteson et al., 2003, J Membr Biol 192: 181-9). We determined that at 2 nM larvae were healthy and behaved normally, despite the inability to regenerate. The penetrance of the regeneration phenotype could be raised by increasing the dose of the PTX, but only at the cost of general toxicity. Thus, in the data described (2 nM), we demonstrate that the regeneration bud is more dependent on membrane voltage level than other cells in the embryo, and we identified this as a dose that could dissociate housekeeping levels from regeneration-specific physiological parameters.

### Scoring of Regeneration Efficiency

To quantify and compare regeneration efficiency of larvae treated with different reagents, we introduced the “Regeneration Index” (RI). Individual larvae within a Petri dish comprising a specific treatment were each scored as follows:
++: complete regeneration (regenerated tail, indistinguishable from uncut controls).
+: robust regeneration in presence of minor defects (missing fin, curved axis).
+/−: poor regeneration (hypomorphic/defective regenerates).
−: no regeneration.

The raw numbers of larvae belonging to each category were calculated; percentages were then multiplied by 3, 2, 1 or 0, for, respectively, ++, +, +/− and −. The RI for that dish, ranges 0-300, with the extreme values corresponding respectively to no regeneration and full regeneration in 100% of the larvae in the sample. The RI evaluates the efficiency of regeneration at the single dish level and allows ready comparison of the effect of treatments to controls.

### Statistical Analysis

To compare among three or more treatments, raw data from the above-described scoring were analyzed using a Kruskal-Wallis test for ordinal data, with H corrected for tied ranks. Post-hoc comparisons were made using Dunn's Q. To compare between two treatments, raw scoring data were analyzed using a Mann-Whitney U test for ordinal data with tied ranks, and using a normal approximation for large sample sizes. Flank cell data were analyzed using a two-factorial (age and treatment) ANOVA. In all analyses, differences were considered significant if p was less than 0.01.

### Proliferating Cell Quantification

H3P staining marks cells in the G₂/M transition of the cell cycle, and is commonly used for identifying mitotic cells in regenerating systems including Xenopus (Saka and Smith, 2001, Dev Biol 229: 307-18; Sanchez Alvarado, 2003, Current Opinion in Genetics & Development 13: 438-444). Fixed specimens at the stages indicated were processed for H3P staining as above using an alkaline-phosphatase secondary. Bleaching of the natural pigments in samples allowed easy counting of H3P-positive cells. The close-up panel to the left illustrates the distinction between purple alkaline-phosphatase signal (H3P-positive nuclei) and brown melanocytes. For quantification, cells were counted manually in the region past the amputation plane. Between 4 and 6 samples were counted for each stage and each condition. Similar numbers were obtained in immunohistochemistry performed on sections as in wholemounts and in fluorescent detection (i.e., reagent penetration and chromogenic staining are not confounding factors in H3P-positive cell detection).

### In Situ Hybridization

Larvae were fixed in MEMFA (Sive et al., 2000) and dehydrated in methanol. In situ hybridization was carried out according to standard protocols (Harland, 1991, Xenopus laevis: Practical uses in cell and molecular biology 36: 685-695). The ion transporter constructs used to generate probes for in situ hybridization (ISH) experiments were: Kcnk1 (TWIK-1, BC042262, Open Biosystems), V-ATPase 16 kDa subunit (BE025959, RZPD). Plasmids were used to generate anti-sense riboprobes by in vitro transcription. Experiments included sense probe controls, which exhibited no signal as expected (data not shown). Expression indicated represents consistent consensus patterns obtained from analysis of at least 15 larvae in all experiments.

### Immunohistochemistry

Xenopus larvae were fixed overnight in MEMFA, heated for 2 hrs at 65° C. in 50% formamide (to inactivate endogenous alkaline phosphatases; this procedure was not done when using fluorescent secondary antibodies), permeabilized in PBTr+0.1% Triton X100 for 30 min, and processed for immunohistochemistry using alkaline phosphatase secondary antibody (Levin, 2004, Journal of Biochemical and Biophysical Methods 58: 85-96) until signal was optimal and background minimal (usually 12 hrs). Anti-ductin (V-ATPase c′ subunit) antibody generated against peptide DAGVRGTAQQPR by Invitrogen reveals 1 single clear band of predicted size on Western blot and was used at 1:500. anti-Caspase-3 (Abcam #AB13847), anti-acetylated α-tubulin (Sigma #T6793), and anti-phospho-H3 (Upstate #05-598) were used at 1:1000. Anti-KCNK1 (a generous gift of Dr. S. A. Goldstein and D. Bockenhauer) was used at 1:500. The 12-101 muscle marker (Gurdon et al., 1985, Cell 41: 913-22; Kintner and Brockes, 1984, Nature 308: 67-9) was used at 1:1 dilution; this monoclonal antibody developed by Jeremy P. Brockes was obtained from the Developmental Studies Hybridoma Bank developed under the auspices of the NICHD and maintained by The University of Iowa, Department of Biological Sciences, Iowa City, Iowa 52242.

Axon and muscle marker detection was performed using fluorescent secondary antibodies. Alexafluor 555-conjugated goat anti-mouse secondary (Invitrogen) was used at 1:500 dilution. Fluorescence images were collected on a Leica TCS SPZ confocal imaging system; λₑₓ=543. Some larvae were embedded in JB4 (Polysciences) and sectioned at 30 μm. Control experiments using no primary antibody and no secondary antibody showed no signal (data not shown). Images in panels H-I of FIG. 12 and in panels H-M′ of FIG. 14 were processed using Adobe Photoshop as follows. Background autofluorescence was removed by segmentation of the original fluorescence images. The brightest pixels could readily be selected such that the selection best represented the pattern of axons. This selection was then pasted onto the transmitted light photographs of the same sample. The original un-manipulated images are available on request. Overall image brightness was adjusted for optimal clarity. Images and localization data presented in all figures represent consensus patterns obtained from analysis of at least 15 larvae in all experiments.

### Confocal Imaging of Membrane Voltage

Xenopus larvae were soaked in voltage-sensitive dye DiBAC₄(3), (Molecular Probes), at a final concentration of 10 ng/ml in 0.1×MMR in the dark for 30 minutes then imaged with a Leica TCS SP2 Confocal Imaging system, mounted on a Leica upright DM RXE microscope. Because DiBAC₄(3) is anionic, the more depolarized a cell, the greater the accumulation of the permeant dye, and the greater the intensity of intracellular, relative to extracellular, fluorescence. The dye was excited at 488 nm and a 20 nm band of emission wavelengths centered at 515 was collected. If images were to be compared, all images were collected on a single day and photomultiplier gain was kept constant. Fluorescence and transmitted light images were collected. Figures were created using Photoshop™. Transmitted light images may have been manipulated for clarity; fluorescence photos were not manipulated other than by creating overlays, rotating, or cropping.

### γ-Irradiation

Intact larvae were subjected to 10⁴ rads of gamma-irradiation in a Cs¹³⁷ irradiator. The group of larvae was then split in two subgroups, one of which underwent amputation 24 hrs after the irradiation procedure. Larvae were examined periodically and a few of them, for each condition, were fixed at different times after irradiation for immunohistochemistry.

### Ion Transporter Misexpression

Synthetic mRNA was transcribed by the SP6 polymerase from linearized pCS2+plasmids containing the individual cDNAs (YCHE78 and pMA1.2). About 5 ng of each construct mRNA was mixed with 50 ng of RLD and 250 pg of mRNA encoding β-galactosidase, RFP, or GFP (Zernicka-Goetz et al., 1996, Development 122: 3719-24) (as lineage labels) and injected into the 1-cell embryo within 1 hour of fertilization.

All publications, patents and patent applications are herein incorporated by reference in their entirety to the same extent as if each individual publication, patent or patent application was specifically and individually indicated to be incorporated by reference in its entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1

| | | Target ion | Inhibit/promote |
|---|---|---|---|
| | | transporter or class | activity of the |
| COMPOUND | Ion Species | of transporters | target |
| 1-EBIO | K | Ca-activated K | Promote |
| | | channels | |
| A23187 | Ca | Membrane | Inhibit |
| A23187 (4-Bromo- | Ca | Membrane | Inhibit |
| A23187) | | | |
| ACA (9-ACA) | Cl | Cl channels | Inhibit |
| Acetazolamide | H, HCO₃ | Carbonic Anhydrase | Inhibit |
| Aconitine | Na | Na channel kinetics | Promote |
| Aflatrem | K | Ca-activated K | Inhibit |
| | | channels, BK | |
| Agatoxin IIIA (w- | Ca | L-, N-, P/Q- and R- | Inhibit |
| Agatoxin IIIA) | | type Ca channels | |
| Agatoxin IVA (w- | Ca | P/Q-type Ca channels | Inhibit |
| Agatoxin IVA) | | | |
| Agitoxin-2 | K | Kv channels | Inhibit |
| Alinidine | K | K-ATP channels | Inhibit |
| Allethrin | Ca | Cav channels | Inhibit |
| | Na (to a lesser | | |
| | extent) | | |
| Allethrin | Na | Na channel kinetics | Promote |
| | Ca (to a lesser | | |
| | extent) | | |
| Allyl isothiocyanate | H, K | H/K-ATPase | Inhibit |
| AM 92016 HCl | K | K inward rectifier | Inhibit |
| Amantadine | H | M2 channels | Inhibit |
| Amiloride | Na, Ca | Na/Ca exchanger | Inhibit |
| | H (to a lesser | | |
| | extent) | | |
| Amiloride | H, Na | Na/H exchanger | Inhibit |
| | Ca (to a lesser | (NHE) | |
| | extent) | | |
| Amiloride HCl | Na | Na channels | Inhibit |
| | Ca and H (to a | | |
| | lesser extent) | | |
| Aminophylline | Cl | CFTR | Promote |
| Aminopyridine (4- | K | Kv channels | Inhibit |
| Aminopyridine; 4- | | | |
| AP) | | | |
| Amiodarone | Ca | Cav channels | Promote |
| Amitriptyline | | tight junctions | Inhibit |
| Amlodipine | Ca | Cav channels | Inhibit |
| Amlodipine | Ca | N- and P/Q-type Ca | Inhibit |
| | | channels | |
| Anthranilic acid | Cl | Cl channels | Inhibit |
| (AA) | | | |
| Apamin | K | Ca-activated K | Inhibit |
| | | channels, SK | |
| Aprikalim | K | K-ATP channels | Promote |
| Astemizole | K | KCNH2 (ERG) | Inhibit |
| Aurovertin B | H | ATP Synthase (F- | Inhibit |
| | | type H-ATPase) | |
| Azimilide | K | KCNQ1 (KvLQT1) | Inhibit |
| Ba²⁺ | K | K channels | Inhibit |
| | (Na and Ca to a | | |
| | lesser extent) | | |
| Ba²⁺ | Na, Ca | Na/Ca exchanger | Inhibit |
| | (K to a lesser | | |
| | extent) | | |
| Bafilomycin | H | H-ATPase (V-type H- | Inhibit |
| | | ATPase) | |
| BAPTA | Ca | Aqueous | Inhibit |
| | | compartments | |
| Barnidipine | Ca | N- and P/Q-type Ca | Inhibit |
| | | channels | |
| Batrachotoxin | Na | Na channel kinetics | Promote |
| Bay K 8644 [(R)- | Ca | Cav channels | Inhibit |
| (+) isomer] | | | |
| Bay K 8644 [(S)- | Ca | Cav channels | Promote |
| (+) isomer] | | | |
| Benidipine | Ca | Cav channels | Inhibit |
| Benidipine | Ca | N- and P/Q-type Ca | Inhibit |
| | | channels | |
| Benzamil | Na | Na channels | Inhibit |
| | (H and Ca to a | | |
| | lesser extent) | | |
| Benzamil | Na, Ca | Na/Ca exchanger | Inhibit |
| | (H to a lesser | | |
| | extent) | | |
| Benzamil | Na, H | Na/H exchanger | Inhibit |
| | (Ca and Na to a | (NHE) | |
| | lesser extent) | | |
| Benzamil | Na | Na/HCO3 | Inhibit |
| | | cotransporter | |
| Benzocaine | Na | NaV channels | Inhibit |
| Benzopyran | K | K-ATP channels | Promote |
| Benzothiazepines | Ca | Cav channels | Inhibit |
| BHQ | Ca | SERCA | Inhibit |
| Bifenthrin | Na | NaV channels | Inhibit |
| Bimakalim | K | K-ATP channels | Promote |
| BL-1743 | H | M2 channels | Inhibit |
| BMS-180448 | K | K-ATP channels | Promote |
| Bradykinin | Ca | Ca stores (internal) | Inhibit |
| | (K to a lesser | | |
| | extent) | | |
| Bradykinin | K | Ca-activated K | Promote |
| | (Ca to a lesser | channels | |
| | extent) | | |
| Brevetoxin | Na | NaV channels | Inhibit |
| BRL 55834 | K | K-ATP channels | Promote |
| Bumetanide | Na, K, Cl | Na/K/Cl cotransporter | Inhibit |
| Bupivacaine | K | K-ATP channels | Inhibit |
| Butanedione | Ca | SERCA | Promote |
| monoxime (2,3- | | | |
| Butanedione | | | |
| monoxime) | | | |
| Caffeine | Ca | SERCA | Inhibit |
| Calciseptine | Ca | L-type Ca channels | Inhibit |
| cAMP | Ca | SERCA | Promote |
| Carbenoxolone | | Gap Junctions | Inhibit |
| Carbocyanine | K | Ca-activated K | Inhibit |
| | | channels, IK | |
| Cariporide | Na, H | Na/H antiporter, | Inhibit |
| (HOE642) | | (NHE) | |
| Cassigarol A | H, K | H/K-ATPase | Inhibit |
| Catechins | H, K | H/K-ATPase | Inhibit |
| CCCP | H | Membrane | Inhibit |
| Cd²⁺ | Cl | Ca-activated Cl | Inhibit |
| | (C, K, Na to a | channels | |
| | lesser extent) | | |
| Cd²⁺ | K | KCNQ1 (KvLQT1) | Inhibit |
| | (Ca, Cl, Na to a | | |
| | lesser extent) | | |
| Cd²⁺ | Ca | L- and N-Type Ca | Inhibit |
| | (Cl, K, Na to a | channels | |
| | lesser extent) | | |
| Cd²⁺ | Na, Ca | Na/Ca exchanger | Both - depending |
| | (Cl, K to a lesser | | on the |
| | extent) | | circumstances |
| Cefadroxil | H | H/peptide transport | Inhibit |
| CGP 37157 | Na, Ca | Na/Ca exchanger | Inhibit |
| Chalcone | H, K | H/K-ATPase | Inhibit |
| Charybdotoxin | K | Ca-activated K | Inhibit |
| | | channels, BK | |
| Charybdotoxin | K | Ca-activated K | Inhibit |
| | | channels, IK | |
| Charybdotoxin | K | Kv channels | Inhibit |
| Chloroquine | K | K-ATP channels | Inhibit |
| | (Tight junctions | | |
| | to a lesser extent) | | |
| Chlorpropamide | K | K-ATP channels | Inhibit |
| Chromanol 293B | K | KCNQ1 (KvLQT1) | Inhibit |
| | (Cl to a lesser | | |
| | extent) | | |
| Ciclazindol | K | K-ATP channels | Inhibit |
| Ciguatoxin | Na | Na channel kinetics | Promote |
| Cilnidipine | Ca | Cav channels | Inhibit |
| Cilnidipine | Ca | N- and P/Q-type Ca | Inhibit |
| | | channels | |
| Cismethrin | Na | NaV channels | Inhibit |
| Clofilium | K | KCNH2 (ERG) | Inhibit |
| Clofilium | K | KCNQ1 (KvLQT1) | Inhibit |
| Clotrimazole | K | Ca-activated K | Inhibit |
| | | channels, IK | |
| Clotrimazole | K | KCNQ1 (KvLQT1) | Inhibit |
| Co²⁺ | Cl | Ca-activated Cl | Inhibit |
| | | channels | |
| Co²⁺ | Ca | L- and N-Type Ca | Inhibit |
| | | channels | |
| Co²⁺ | Na/Ca | Na/Ca exchanger | Inhibit |
| Concanamycin | H | H-ATPase (V-type H- | Inhibit |
| | | ATPase) | |
| Conotoxin GVIA | Ca | N-type Ca channels | Inhibit |
| (w-Conotoxin | | | |
| GVIA) | | | |
| Conotoxin MVIIC | Ca | N- and P/Q-type Ca | Inhibit |
| (w-Conotoxin | | channels | |
| MVIIC) | | | |
| Conotoxin PVIIA | K | Kv channels | Inhibit |
| (k-Conotoxin | | | |
| PVIIA) | | | |
| Conotoxin SMIIIA | Na | Na channels | Inhibit |
| (m-Conotoxin | | | |
| SMIIIA) | | | |
| Conotoxin ViTx | K | K channels | Inhibit |
| Correolide | K | Kv channels | Inhibit |
| CP 339818 HCl | K | Kv channels | Inhibit |
| Cromakalim | K | K-ATP channels | Promote |
| Cs⁺ | K | K channels | Inhibit |
| Cs⁺ | K | Kv channels | Inhibit |
| Cu²⁺ | | Aquaporin-3 | Inhibit |
| Cu²⁺ | Na, Ca | Na/Ca exchanger | Inhibit |
| Cu²⁺ | Na, K | Na/K-ATPase | Inhibit |
| Cu²⁺ | | tight junctions | Promote |
| CyanoGuanidine | K | K-ATP channels | Promote |
| Cyclopiazonic Acid | Ca | SERCA | Inhibit |
| Cyfluthrin | Na | NaV channels | Inhibit |
| Cyhalothrin | Na | NaV channels | Inhibit |
| Cypermethrin | Na | NaV channels | Inhibit |
| Cyphenothrin | Na | Na channel kinetics | Inhibit |
| Darodipine | Ca | Cav channels | Inhibit |
| DCCD | H | H-ATPase (V-type H- | Inhibit |
| | | ATPase) | |
| Decanoic acid | K | K-ATP channels | Inhibit |
| Decarbamylsaxitoxin | Na | Na channels | Inhibit |
| Deltamethrin | Na | Na channel kinetics | Inhibit |
| Dendrotoxin (a | K | Kv channels | Inhibit |
| Dendrotoxin) | | | |
| Dendrotoxin (b | K | Kv channels | Inhibit |
| Dendrotoxin) | | | |
| Dendrotoxin (d | K | K inward rectifier | Inhibit |
| Dendrotoxin) | | | |
| Dequalinium | K | Ca-activated K | Inhibit |
| dichloride | | channels, SK | |
| DHS-1 | K | Ca-activated K | Promote |
| (Dehydrosoyasaponin- | | channels, BK | |
| 1) | | | |
| Diazepam | Cl | Cl channels | Promote |
| Diazoxide | K | K-ATP channels | Promote |
| Dibucaine | K | K-ATP channels | Inhibit |
| Dichlorphenamide | H, HCO₃ | Carbonic Anhydrase | Inhibit |
| Dideoxyforskolin | Cl | Cl channels | Inhibit |
| DIDS | Cl | Cl channels | Inhibit |
| | (H, K to a lesser | | |
| | extent) | | |
| DIDS | K | KCNQ1 (KvLQT1) | Promote |
| | (Cl, H to a lesser | | |
| | extent) | | |
| Diethylstilbestrol | H | ATP Synthase (F- | Inhibit |
| (DES) | | type H-ATPase) | |
| Diethylstilbestrol | H | H-ATPase (V-type H- | Inhibit |
| (DES) | | ATPase) | |
| Dihydropyridines | Ca | Cav channels | Inhibit |
| Diltiazem (D-cis | Ca | Cav channels | Inhibit |
| Diltiazem-HCl) | | | |
| Diltiazem (L-cis- | Ca | CNG channels | |
| diltiazem) | | | |
| Diltiazem (L-cis- | K | Cav channels | Inhibit |
| diltiazem) | | | |
| Dimethadione | K | K channels | Inhibit |
| Dimethylamiloride | Na, Ca | Na/Ca exchanger | Inhibit |
| | (H to a lesser | | |
| | extent) | | |
| Dimethylamiloride | Na, H | Na/H antiporter, | Inhibit |
| | (Ca to a lesser | (NHE) | |
| | extent) | | |
| Diphenylbutylpiperidine | Ca | L- and T-type Ca | Inhibit |
| (DPBP) | | Channels | |
| Dofetilide | K | KCNH2 (ERG) | Inhibit |
| DPMX | Cl | CFTR | Promote |
| E-4031 | K | KCNH2 (ERG) | Inhibit |
| EDTA | Ca | Aqueous | Inhibit |
| | | compartments | |
| Efaroxan | K | K-ATP channels | Inhibit |
| EGTA | Ca | Aqueous | Inhibit |
| | | compartments | |
| Eicosapentanoic | | tight junctions | Inhibit |
| acid (EPA) | | | |
| EIPA | Na, H | Na/H antiporter, | Inhibit |
| (Ethylisopropyl | | (NHE) | |
| amiloride) | | | |
| Ellagic acid | H, K | H/K-ATPase | Inhibit |
| Englitazone | K | K-ATP channels | Inhibit |
| Enprofylline | Cl | CFTR | Promote |
| Ergotoxin | K | KCNH2 (ERG) | Inhibit |
| Erythrosin 5′- | Ca | SERCA | Inhibit |
| isothiocyanate | | | |
| Erythrosine B | H | H-ATPase (V-type H- | Inhibit |
| | | ATPase) | |
| Esomeprazole | H, K | H/K-ATPase | Inhibit |
| Estradiol (11-b- | K | KCNQ1 (KvLQT1) | Inhibit |
| Estradiol) | | | |
| Estradiol (17-b- | Na, H | NHE | Inhibit |
| Estradiol) | | | |
| Ethacrynic acid | Cl | Cl channels | Inhibit |
| Ethacrynic acid | Cl | Cl-ATPase | Inhibit |
| Ethoxyzolamide | H | Carbonic Anhydrase | Inhibit |
| FCCP | H | Membrane | Inhibit |
| Fe²⁺ | Na, Ca | Na/Ca exchanger | Inhibit |
| | (K to a lesser | | |
| | extent) | | |
| Fe²⁺ | Na, K | Na/K-ATPase | Inhibit |
| | (Ca to a lesser | | |
| | extent) | | |
| Felodipine | Ca | Cav channels | Inhibit |
| Fenofibrate | K | K-ATP channels | Inhibit |
| Fenpropathrin | NA | NaV channels | Inhibit |
| Fenvalerate | Na | NaV channels | Inhibit |
| Flecainide acetate | Na | Na channels | Inhibit |
| Flunarizine | Ca | Ca channels | Inhibit |
| dihydrochloride | (Na to a lesser | | |
| | extent) | | |
| Flunarizine | Na | Na channels | Inhibit |
| dihydrochloride | (Ca to a lesser | | |
| | extent) | | |
| FPL 64176 | Ca | Cav channels | Promote |
| FR177995 | H | H-ATPase (V-type H- | Inhibit |
| | | ATPase) | |
| Furosemide | Na, K, Cl | Na/K/Cl cotransporter | Inhibit |
| Fusicoccin | H, K | H-ATPase (V-type H- | Promote |
| | | ATPase) | |
| Gabapentin HCl | Ca | Cav channels | Inhibit |
| Gaboon viper | K | K inward rectifier | Inhibit |
| venom | | | |
| Gadolinium | Ca | Ca-activated Cl | Inhibit |
| | | channels | |
| Gadolinium | | Mechano-sensitive | Inhibit |
| | | channels | |
| Gallopamil | Ca | Ca-ATPase | Inhibit |
| Glibenclamide | Ca | CFTR | Inhibit |
| | (K to a lesser | | |
| | extent) | | |
| Glibenclamide | K | K-ATP channels | Inhibit |
| (Glyburide) | (Ca to a lesser | | |
| | extent) | | |
| Glipizide | K | K-ATP channels | Inhibit |
| Glycyrrhetinic acid | GJC | Gap Junctions | Inhibit |
| (18-b- | | | |
| Glycyrrhetinic acid) | | | |
| Gonyautoxin II | Na | Na channels | Inhibit |
| Gonyautoxin III | Na | Na channels | Inhibit |
| Gramicidin A | K | Membrane | |
| Grayanotoxin | Na | Na channel kinetics | Promote |
| Guanethidine | K | K-ATP channels | Inhibit |
| Guanidine | Na, H | NHE | Inhibit |
| methanesulfonate | | | |
| Haloperidol | K | KCNH2 (ERG) | Inhibit |
| Halothane | K | Ca-activated K | Inhibit |
| | | channels | |
| Halothane | K | K inward rectifier | Inhibit |
| Halothane | K | K Tandem Pore | Promote |
| | | channels | |
| Hanatoxin-1 | K | Kv channels | Inhibit |
| Hanatoxin-2 | K | Kv channels | Inhibit |
| Heptanol | GJC | Gap Junctions | Inhibit |
| HMR-1098 | K | K-ATP channels | Inhibit |
| HMR-1556 | K | KCNQ1 (KvLQT1) | Inhibit |
| HMR-1556 | Ca | Cav channels | Inhibit |
| HOCl | | tight junctions | Inhibit |
| HOE642 | Na, H | NHE | Inhibit |
| (cariporide) | | | |
| HOE694 | Na, H | NHE | Inhibit |
| Hongotoxin | K | Kv channels | Inhibit |
| Hydroxydecanoate | K | K-ATP channels | Inhibit |
| (5- | | (mitochondrial) | |
| Hydroxydecanoate) | | | |
| Hydroxyzine | K | KCNH2 (ERG) | Inhibit |
| Iberiotoxin | K | Ca-activated K | Inhibit |
| | | channels, BK | |
| IBMX | Cl | CFTR | Promote |
| Ibutilide | K | KCNH2 (ERG) | Inhibit |
| indanyloxyacetic | Cl | Cl channels | Inhibit |
| acid (IAA) | | | |
| Iodo-resiniferatoxin | Ca | TRPV1 | Inhibit |
| Ionomycin | Ca | Membrane | |
| Isoflurane | K | K Tandem Pore | Promote |
| | | channels | |
| Isradipine | Ca | Cav channels | Inhibit |
| Kaliotoxin | K | Ca-activated K | Inhibit |
| | | channels, BK | |
| Kaliotoxin | K | Kv channels | Inhibit |
| KB-R7943 | Na, Ca | Na/Ca exchanger | Inhibit |
| mesylate | | | |
| Kurtoxin | Ca | T-type Ca channels | Inhibit |
| Lamotrigine | Ca | Ca channels | |
| | (Na, K to a lesser | | |
| | extent) | | |
| Lamotrigine | K | KCNH2 (ERG) | Inhibit |
| | (Na, Ca to a | | |
| | lesser extent) | | |
| Lamotrigine | K | Kv channels | Promote |
| | (Na, Ca to a | | |
| | lesser extent) | | |
| Lamotrigine | Na | NaV channels | Inhibit |
| | (Ca, K to a lesser | | |
| | extent) | | |
| Lansoprazole | H, K | H/K-ATPase | Inhibit |
| Lanthanum | Ca | Ca channels | Inhibit |
| Lanthanum | Cl | Ca-activated Cl | Inhibit |
| | | channels | |
| Levocromakalim | K | K-ATP channels | Promote |
| Lidocaine | K | K-ATP channels | Inhibit |
| Lidocaine n-ethyl | Na | Na channels | Inhibit |
| bromide | | | |
| Lindane | | Gap Junctions | Inhibit |
| Linoleoylamide | K | KCNH2 (ERG) | Inhibit |
| Linopirdine | K | Ca-activated K | Inhibit |
| | | channels | |
| Linopirdine | K | KCNQ2/3 | Inhibit |
| (specific to 2/3) | | | |
| Linopirdine | K | Kv channels | Inhibit |
| dihydrochloride | | | |
| Lobatamide C | H | H-ATPase (V-type H- | Inhibit |
| | | ATPase) | |
| Lobatamide C A15 | H | H-ATPase (V-type H- | Inhibit |
| | | ATPase) | |
| Lobatamide C A16 | H | H-ATPase (V-type H- | Inhibit |
| | | ATPase) | |
| Lobatamide C A6 | H | H-ATPase (V-type H- | Inhibit |
| | | ATPase) | |
| Lonidamine | Cl | CFTR | Inhibit |
| Loperamide | Ca | Cav channels | Inhibit |
| LY83583 | Ca | CNG channels | Inhibit |
| LY97241 | K | KCNH2 (ERG) | Inhibit |
| Margatoxin | K | Kv channels | Inhibit |
| MaxiPost | K | Ca-activated K | Promote |
| | | channels, BK | |
| Mefenamic acid | K | KCNQ1 (KvLQT1) | Promote |
| Mefloquine | K | K-ATP channels | Inhibit |
| Meglitinide | K | K-ATP channels | Inhibit |
| Mepivacaine | K | K-ATP channels | Inhibit |
| Methazolamide | H, HCO3 | Carbonic Anhydrase | Inhibit |
| Methoxy-verapamil | Ca | Cav channels | Inhibit |
| Mexiletine | K | K-ATP channels | Inhibit |
| Mg²⁺ | Na, Ca | Na/Ca exchanger | Inhibit |
| Mibefradil | Ca | L- and T-type Ca | Inhibit |
| | | channels | |
| Minoxidil | K | K-ATP channels | Promote |
| MK-499 | K | KCNH2 (ERG) | Inhibit |
| Mn²⁺ | Cl | Ca-activated Cl | Inhibit |
| | (Na, Ca to a | channels | |
| | lesser extent) | | |
| Mn²⁺ | Ca | Na/Ca exchanger | Inhibit |
| | (Na, Cl to a lesser | | |
| | extent) | | |
| Mn²⁺ | Na | Na/Ca exchanger | Inhibit |
| | (Ca, Cl to a lesser | | |
| | extent) | | |
| Moclobemide | H | Aqueous | Promote |
| | | compartments | |
| Monensin | Na | Membrane | Inhibit |
| Nateglinide | K | K-ATP channels | Inhibit |
| NBD | H | ATP Synthase (F- | Inhibit |
| (chloronitrobenzoxadiazole) | | type H-ATPase) | |
| NBD | H | H-ATPase (V-type H- | Inhibit |
| (chloronitrobenzoxadiazole) | | ATPase) | |
| Neosaxitoxin | Na | Na channels | Inhibit |
| N-Ethylmaleimide | H | H-ATPase (V-type H- | Inhibit |
| (NEM) | | ATPase) | |
| Nicardipine | Ca | L-type Ca channels | Inhibit |
| Nicardipine | Ca | N- and P/Q-type Ca | Inhibit |
| | | channels | |
| NiCl₂ | Na, Ca | Na/Ca exchanger | Inhibit |
| NiCl₂ | Ca | T-type Ca channels | Inhibit |
| Nicorandil | K | K-ATP channels | Promote |
| | | (mitochondrial) | |
| Nifedipine | | KCNG channels | Inhibit |
| Nifedipine | Ca | Cav channels | Inhibit |
| Niflumic acid | Cl | Ca-activated Cl | Inhibit |
| | | channels | |
| Nigericin | K | Membrane | |
| Niguldipine HCl | Ca | Cav channels | Inhibit |
| [(R)(−) isomer] | | | |
| Niguldipine HCl | Ca | Cav channels | Inhibit |
| [(S)(+) isomer] | | | |
| Nilvadipine | Ca | Cav channels | Inhibit |
| Nimodipine | Ca | Cav channels | Inhibit |
| Nisoldipine | Ca | Cav channels | Inhibit |
| Nitrendipine | K | Ca-activated K | Inhibit |
| | (Ca to a lesser | channels, IK | |
| | extent) | | |
| Nitrendipine | Ca | Cav channels | Inhibit |
| | (K to a lesser | | |
| | extent) | | |
| Norepinephrine | Ca | Ca-ATPase | Promote |
| Noxiustoxin | K | Kv channels | Inhibit |
| NPPB (PAA) | Cl | Cl channels | Inhibit |
| NS004 | K | Ca-activated K | Promote |
| | | channels, BK | |
| NS1608 | K | Ca-activated K | Promote |
| | | channels, BK | |
| NS1619 | K | Ca-activated K | Promote |
| | | channels, BK | |
| Ochratoxin A | Ca | Ca-ATPase | Inhibit |
| Oleamide | K | KCNH2 (ERG) | Inhibit |
| Oligomycin | H | ATP Synthase (F- | Inhibit |
| | (Na, K to a lesser | type H-ATPase) | |
| | extent) | | |
| Oligomycin | H | H-ATPase (V-type H- | Inhibit |
| | (Na, K to a lesser | ATPase) | |
| | extent) | | |
| Oligomycin | Na, K | Na/K-ATPase | Inhibit |
| | (H to a lesser | | |
| | extent) | | |
| Omeprazole | H, K | H/K-ATPase | Inhibit |
| Orthovanadate | Cl | Cl transport | Inhibit |
| | (Na, K, H to a | | |
| | lesser extent) | | |
| Ouabain | Na, K | Na/K-ATPase | Inhibit |
| P1075 | K | K-ATP channels | Promote |
| | | (sarcolemmal) | |
| PAA (NPPB) | Cl | Cl channels | Inhibit |
| Palytoxin (PTX) | Na, K | Na/K-ATPase | Inhibit |
| Pantoprazole | H | H/K-ATPase | Inhibit |
| Paspalicine | K | Ca-activated K | Inhibit |
| | | channels, BK | |
| Paspalinine | K | Ca-activated K | Inhibit |
| | | channels, BK | |
| Paspalitrem A | K | Ca-activated K | Inhibit |
| | | channels, BK | |
| Paspalitrem C | K | Ca-activated K | Inhibit |
| | | channels, BK | Inhibit |
| Paxilline | K | Ca-activated K | Inhibit |
| | | channels, BK | |
| Penfluridol | Ca | T-type Ca channels | |
| Penitrem A | K | Ca-activated K | Inhibit |
| | | channels, BK | |
| Pentoxifylline | Cl | CFTR | Promote |
| pentylenetetrazol | K | K-ATP channels | Promote |
| pentylenetetrazol | K | Kv channels | Both - depending |
| | | | on the |
| | | | circumstances |
| Permethrin | Na | NaV channels | Inhibit |
| Phenothrin | Na | Na channel kinetics | Inhibit |
| Phentolamine | K | K-ATP channels | Inhibit |
| Phenylalkylamines | Ca | Cav channels | Inhibit |
| PHM | K | K-ATP channels | Inhibit |
| (phentolamine | | | |
| mesylate) | | | |
| PI(4,5)P2 | K | K inward rectifier | Promote |
| PI(4,5)P2 | K | K-ATP channels | Inhibit |
| PI(4,5)P2 | K | KCNH2 (ERG) | Promote |
| Pinacidil | K | K-ATP channels | Promote |
| PKF 217-744 | K | K-ATP channels | Promote |
| Pompilidotoxin (b- | Na | Na channels | Promote |
| Pompilidotoxin; | | | |
| wasp toxin) | | | |
| Prenylamine | Ca | Ca-ATPase | Inhibit |
| Procaine | K | K-ATP channels | Inhibit |
| | (Na to a lesser | | |
| | extent) | | |
| Procaine | Na | NaV channels | Inhibit |
| | (K to a lesser | | |
| | extent) | | |
| Prodigiosin | H | H/K-ATPase | Inhibit |
| Prodigiosin | H | H-ATPase (V-type H- | Inhibit |
| | | ATPase) | |
| Propranolol | K | K-ATP channels | Inhibit |
| Prostaglandin J2 | K | K-ATP channels | Inhibit |
| (15-deoxy- | | | |
| Delta12,14- | | | |
| Prostaglandin J2) | | | |
| Pumaprazole | H | H/K-ATPase | Inhibit |
| Pyrethroids | Na | Na channel kinetics | |
| Quercetin | H, K | H/K-ATPase | Inhibit |
| Quinidine | K | KCNH2 (ERG) | Inhibit |
| Quinine | K | K-ATP channels | Inhibit |
| QX 222 | Na | Na channels | Inhibit |
| QX 314 | Na | NaV channels | Inhibit |
| Rabeprazole | H, K | H/K-ATPase | Inhibit |
| repaglinide | K | K-ATP channels | Inhibit |
| Retigabine | K | KCNQ | Promote |
| Rilmakalim | K | K-ATP channels | Promote |
| Riluzole | Ca | Ca channels | Inhibit |
| | (Na to a lesser | | |
| | extent) | | |
| Riluzole HCl | Na | Na channels | Inhibit |
| | (Ca to a lesser | | |
| | extent) | | |
| Riodipine | Ca | Cav channels | Inhibit |
| (Ryosidine) | | | |
| RP 66471 | K | K-ATP channels | Promote |
| rTamapin | K | KCNN2 | Inhibit |
| Ruthenium red | Ca | Ca translocators | Inhibit |
| | | (many) | |
| Saxitoxin (IIa (—OH) | Na | Na channels | Inhibit |
| STX) | | | |
| Saxitoxin (PSP) | Na | Na channels | Inhibit |
| Saxitoxinol (a- | Na | Na channels | Inhibit |
| saxitoxinol) | | | |
| Saxitoxinol (b- | Na | Na channels | Inhibit |
| saxitoxinol) | | | |
| SB-242784 | H | H-ATPase (V-type H- | Inhibit |
| | | ATPase) | |
| SCA40 | K | Ca-activated K | Promote |
| | | channels, BK | |
| SCH28080 | H, K | H/K-ATPase | Inhibit |
| Scorpion toxins | Na | Na channel kinetics | Promote |
| (American b) | | | |
| Scorpion toxins | Na | Na channel kinetics | Promote |
| (North African a) | | | |
| Scyllatoxin | K | Ca-activated K | Inhibit |
| | | channels, SK | |
| Sea Anemone | Na | Na channel kinetics | Promote |
| toxins | | | |
| Sevoflurane | K | Ca-activated K | Promote |
| | (Ca to a lesser | channels, BK | |
| | extent) | | |
| Sevoflurane | K | K Tandem Pore | Promote |
| | (Ca to a lesser | channels | |
| | extent) | | |
| Sevoflurane | Ca | Cav channels | Inhibit |
| SG 209 | K | K channels | Promote |
| SNX-111 | Ca | N-type Ca channels | Inhibit |
| SNX-482 | Ca | R-type Ca channels | Inhibit |
| sodium caprate | | tight junctions | Inhibit |
| Sophalcone | H, K | H/K-ATPase | Inhibit |
| Sophoradin | H, K | H/K-ATPase | Inhibit |
| Sotalol | K | KCNH2 (ERG) | Inhibit |
| SR 33805 oxalate | Ca | Cav channels | Inhibit |
| Sr²⁺ | Na, Ca | Na/Ca exchanger | Inhibit |
| Streptolysin O | | Membrane | Inhibit |
| Strophanthidin | Na, K | Na/K-ATPase | Inhibit |
| TAC-101 | | Gap Junctions | Promote |
| Tamoxifen | Cl | Cl channels | Inhibit |
| TEA | K | Ca-activated K | Inhibit |
| | | channels, BK | |
| TEA | K | KCNQ | Inhibit |
| TEA | K | Kv channels | Inhibit |
| (Tetraethylammonium) | | | |
| Tedisamil | K | K channels | Inhibit |
| Tefluthrin | Na | NaV channels | Neg |
| Tenidap | K | K inward rectifier | Promote |
| Tenidap | Na | Na/HCO3 co- | Inhibit |
| | | transporter | |
| Terfenadine | K | KCNH2 (ERG) | Inhibit |
| Terikalant | K | K inward rectifier | Inhibit |
| Tertiapin-Q (bee | K | K inward rectifier | Inhibit |
| venom toxin) | | | |
| Tetracaine | | CNG channels | Inhibit |
| Tetracaine | K | K-ATP channels | Inhibit |
| Tetrahydroberberine | K | Kv channels | Inhibit |
| (THB) | | | |
| Tetramethrin | Na | Na channel kinetics | |
| Tetrandrine | K | Ca-activated K | Inhibit |
| | | channels, BK | |
| Tetrodotoxin (TTX) | Na | Na channels | Inhibit |
| Thapsigargin | Ca | SERCA | Inhibit |
| Theophylline | Cl | CFTR | Promote |
| Tiludronate (skelid) | H | H-ATPase (V-type H- | Inhibit |
| | | ATPase) | |
| Tityustoxin-Ka | K | Kv channels | Inhibit |
| TMB-8 | K | K-ATP channels | Inhibit |
| Tolazamide | K | K-ATP channels | Inhibit |
| Tolbutamide | K | K-ATP channels | Inhibit |
| TPEN | Zn, Cu, Fe | Other | Inhibit |
| TRAM-34 | K | Ca-activated K | Inhibit |
| | | channels, IK | |
| Tributyl Tin (TBT) | Cl, OH | Membrane | |
| Tricaine | Na | NaV channels | Inhibit |
| Trifluorperazine | Ca | Calmodulin | Inhibit |
| Trimethadione | K | K inward rectifier | Inhibit |
| Troglitazone | K | K-ATP channels | Inhibit |
| U-37883 | K | K-ATP channels | Inhibit |
| U-37883A | K | K-ATP channels | Inhibit |
| UCL 1684 | K | Ca-activated K | Inhibit |
| | | channels, SK | |
| Valinomycin | H, K | Membrane | |
| Vanadate | Na, K | Na/K-ATPase | Inhibit |
| | (Cl and H to a | | |
| | lesser extent) | | |
| Verapamil HCl | K | KCNH2 (ERG) | Inhibit |
| Verapamil HCl | Ca | Cav channels | Inhibit |
| | (K to a lesser | | |
| | extent) | | |
| Veratridine | Na | Na channel kinetics | Promote |
| Verruculogen | K | Ca-activated K | Inhibit |
| | | channels, BK | |
| Vinpocetine | Na | NaV channels | Inhibit |
| Way-123398 | K | KCNH2 (ERG) | Inhibit |
| WIN 17317-3 | K | Kv channels | Inhibit |
| Xanthoangelol | H, K | H/K-ATPase | Inhibit |
| XE 991 | K | KCNQ1 (KvLQT1), | Inhibit |
| dihydrochloride | | KCNQ1 | |
| | | (KvLQT1) + minK, | |
| | | KCNQ2 + 3 | |
| Y-26762 | K | K-ATP channels | Promote |
| YS-035 | K | K channels | Inhibit |
| Zatebradine | K | Kv channels | Inhibit |
| ZD6169 | K | K-ATP channels | Promote |
| ZD7288 | K | HCN channels | Inhibit |
| | (Na to a lesser | | |
| | extent) | | |
| ZD7288 | Na | HCN channels | Inhibit |
| | (K to a lesser | | |
| | extent) | | |
| ZD7288 | Ca | T-type Ca channels | Inhibit |
| Zetekitoxin AB | Na | NaV channels | Inhibit |
| ZM 181,037 | K | K-ATP channels | Inhibit |
| ZM 226600 | K | K-ATP channels | Promote |
| (Anilide tertiary | | | |
| carbinols) | | | |
| Zn | H, Na | NHE | Inhibit |
| ZnCl₂ | Na, Ca | Na/Ca exchanger | Inhibit |

TABLE 2

| Compound | Concentration | Target and function | Reference |
|---|---|---|---|
| Amiloride | 2.5 mM | Inhibitor of Na⁺/H⁺ | (Harris and Fliegel, |
| | | antiporter | 1999, International |
| | | | Journal of Molecular |
| | | | Medicine 3: 315-21) |
| 4-Aminopiridine | 2.6 mM | Blocker of Kv | (Abraham et al., |
| | | channels | 2003, Acta Biol Hung |
| | | | 54: 63-78) |
| 9-anthracene- | 2.5 μM | Inhibitor of Cl⁻ | (Yarar et al., 2001, J |
| carboxylic acid | | channels | Soc Gynecol Investig |
| | | | 8: 206-9) |
| Benzamil | 10 μM | Inhibitor of epithelial | (Taguchi et al., 2005, |
| | | sodium channels | Biochem Biophys |
| | | | Res Commun 327: |
| | | | 915-9) |
| Diazoxide | 10 μM | Opener of K⁺_{ATP} | (D'Hahan et al., |
| | | channels | 1999, Proceedings of |
| | | | the National |
| | | | Academy of |
| | | | Sciences of the |
| | | | United States of |
| | | | America 96: 12162-7) |
| EIPA | 50 μM | Inhibitor of Na⁺/H⁺ | (Pizzonia et al., |
| | | antiporter | 1996, Journal of |
| | | | Neuroscience |
| | | | Research 44: 191-8) |
| EM12 | 0.7 mM | Inducer of gap- | (Onat et al., 2001, |
| | | junctional | Biochem Pharmacol |
| | | communication | 62: 1081-6) |
| Gadolinium chloride | 10 μM | Blocker of mechano- | (Krasznai et al., |
| | | sensitive channels | 2003, Cell Motil |
| | | | Cytoskeleton 55: |
| | | | 232-43) |
| Glibenclamide | 1.44 mM | Inhibitor of K⁺_{ATP} | (Quayle et al., 1997, |
| | | channels | Physiol Rev 77: |
| | | | 1165-232) |
| 18-β-Glycyrrhetinic | 26.5 μM | Blocker of gap- | (Davidson and |
| acid | | junctional | Baumgarten, 1988, |
| | | communication | Journal of |
| | | | Pharmacology & |
| | | | Experimental |
| | | | Therapeutics 246: |
| | | | 1104-7) |
| Heptanol | Dilution of | Blocker of gap- | (Deleze and Herve, |
| | 1 × 10⁻⁵ | junctional | 1983, J Membr Biol |
| | | communication | 74: 203-15; Takens- |
| | | | Kwak et al., 1992, |
| | | | American Journal of |
| | | | Physiology 262: |
| | | | C1531-8) |
| Lanthanum chloride | 10 μM | Blocker of Ca⁺⁺ | (Nathan et al., 1988, |
| | | channels | Journal of General |
| | | | Physiology 91: 549-72) |
| Loperamide | 0.2 mM | Ca⁺⁺ channel blocker | (Harper et al., 1997, |
| | | | Proc Natl Acad Sci USA |
| | | | 94: 14912-7) |
| Ouabain | 16 μM | Inhibitor of Na⁺/K⁺- | (Liu, 2005, Front |
| | | ATPase | Biosci 10: 2056-63) |
| PPADS | 50 μM | PX27 channels | (Ziganshin et al., |
| | | blocker | 1993, Br J |
| | | | Pharmacol 110: |
| | | | 1491-5) |
| Quinidine | 50 μM | Blocker of slow | (Yao et al., 1996, J |
| | | delayed K⁺ rectifier | Pharmacol Exp Ther |
| | | | 279: 856-64) |
| SCH28080 | 0.12 mM | Inhibitor of H⁺/K⁺- | (Vagin et al., 2002, |
| | | ATPase | Biochemistry 41: |
| | | | 12755-62) |
| Suramin | 100 μM | Blocker of Cl⁻, Ca⁺⁺ | (Bachmann et al., |
| | | channels | 1999, Naunyn |
| | | | Schmiedebergs Arch |
| | | | Pharmacol 360: 473-6; |
| | | | Emmick et al., |
| | | | 1994, J Pharmacol |
| | | | Exp Ther 269: 717-24) |
| Tetraethylammonium | 1 mM | Blocker of Ca⁺⁺- | (Shen et al., 1994, |
| | | activated K⁺ | Pflugers Arch 426: |
| | | channels | 440-5) |
| THB | 7.3 mM | Inhibitor of voltage- | (Wu and Jin, 1996, |
| | | gated K⁺ channels | Neurosci Lett 207: |
| | | | 155-8 |
| Concanamycin | 150 nM | Inhibitor of V-ATPase | (Drose et al., 1993, |
| | | | Biochemistry 32: |
| | | | 3902-6; Woo et al., |
| | | | 1996, Biological & |
| | | | Pharmaceutical |
| | | | Bulletin 19: 297-9) |

### Description of Table 2: Table of Reagents.

This table lists compounds that were tested for their ability to specifically inhibit regeneration while permitting normal primary tail development, wound healing, and general embryogenesis. The particular concentration of compound used in this study are provided. However, concentration used may vary depending on the biological process and organisms being evaluated. The major targets are listed for each drug, but many of these compounds also interact with other transporters. Any target of a reagent is thus ruled out of further consideration when a given compound does not affect regeneration, and in these cases broader specificity is a benefit because it allows a greater number of candidates to be filtered out. This screen implicated the V-ATPase transporter that we subsequently validated molecularly and characterized.

TABLE 3

| | | Regeneration | | | | | | |
|---|---|---|---|---|---|---|---|---|
| | | Index | | Treated/ | Statistical | | | |
| Experiment | N | Control | Treated | Control | Test | Statistic | | Probability |
| Regeneration in | 226 | 216 | 49 | 23% | MWU | U = 11.628 | Z = 11.357 | p << 0.001 |
| Concanamycin | | | | | | | | |
| PMA-expressing | 127 | 75 | 226 | 301% | KW + DQ | H = 81.486 | Q = 4.672 | p < 0.01 |
| Regeneration in | | | | | | | | |
| Concanamycin | | | | | | | | |
| Regeneration in | 81 | 187 | 144 | 77% | MWU | U = 990.5 | Z = 2.926 | p = 0.002 |
| Palytoxin | | | | | | | | |
| YCHE78-expressing | 66 | 265 | 194 | 73% | KW + DQ | H = 100.232 | Q = 3.556 | p < 0.01 |
| Regeneration | | | | | | | | |
| PMA-expressing | 103 | 15 | 42 | 280% | MWU | U = 1638.5 | Z = 13.005 | p << 0.001 |
| Regeneration of | | | | | | | | |
| Refractory Tail | | | | | | | | |

### Description of Table 3: Primary Data and Statistical Analysis for Regeneration Assays

Effect of the inhibitor of V-ATPase on tail regeneration is expressed as a “Regeneration Index” (RI) computed for each dish of embryos (see Methods).

TABLE 4

| | 24 hpa | | 48 hpa | |
|---|---|---|---|---|
| Treatment | Individual counts | Average | Individual counts | Average |
| Control | 81 | 125 | 68 | 53 |
| | 131 | | 49 | |
| | 88 | | 45 | |
| | 113 | | 37 | |
| | 184 | | 65 | |
| | 136 | | 43 | |
| | 95 | | 10 | |
| | 171 | | 108 | |
| Concanamycin- | 79 | 51 | 39 | 49 |
| exposed | 26 | | 58 | |
| | 45 | | 51 | |
| | 53 | | 46 | |

### Description of Table 4:: Analysis of Medial Region Proliferation

Cells positive for the H3P proliferation marker were counted in control and concanamycin-exposed larvae in a square region, with each side equal to the dorso-ventral height of the tail, located immediately posterior to the amputation plane.

In control amputated larvae, by 48 hpa, the number of proliferating cells in the mid-flank drops by a factor of 2.3. In contrast, in concanamycin-exposed larvae, the number does not change significantly between 24 and 48 hpa. It is important to note that there are two separate V-ATPase dependent events here. First, there is a general and immediate (by 24 hpa) 3-fold reduction in the number of proliferating cells after concanamycin exposure. This mild reduction of mitosis does not impact general development and is far smaller than the 10:1 reduction in proliferation in the regeneration bud. Thus, while the V-ATPase may be involved in proliferation in general, it is absolutely central to the up-regulation of proliferation in the bud.

Secondly, the normal down-regulation of proliferation at 48 hours does not occur when the V-ATPase is inhibited. This reduction is initiated by regeneration (is not local to the flank), and is V-ATPase-dependent. A model whereby the V-ATPase inhibition maintains proliferating cells locally in the flank is ruled out by the general negative effect of concanamycin on proliferation (opposite to the maintenance observed) and the fact that a local model would predict a further 3-fold reduction of the proliferating cell numbers in the flank, which is not observed. ANOVA analysis (N=24, F=11.02, p=0.0002) indicates a significant difference in the number of H3P-positive cells in the flank at 24 vs. 48 hours due to V-ATPase inhibition.

TABLE 5
Non-limiting examples of iontransporters are provided below. The nucleotide sequences,
amino acid sequences, and other materials provided in these accession numbers are
hereby incorporated by reference in their entirety

| | DDBJ/EMBL/GeneBank No. (nucleotide |
|---|---|
| Ion Transporter | and amino acid sequences.) |
| TRPV1 | DQ898279 (human); NM_001001445 |
| | (mouse) |
| CaV P/Q type (CACNA1A) | NM_000068 (human); NM_007578 |
| | (mouse) |
| Ca-ATPase (ATP2B1) | NM_001001323 (human); NM_001016839 |
| | (frog) |
| Na/Ca exchanger (SLC8A1) | NM_021097 (human); NM_011406 |
| | (mouse) |
| KCNQ1 (KvLQT1) | NM_000218 (human); NM_008434 |
| | (mouse) |
| KCNH2 (ERG) | NM_000238 (human); NM_013569 |
| | (mouse) |
| KCNK1 | NM_002245 (human); NM_001011490 |
| | (frog) |
| KATP (Kir6.2) | NM_000891 (human); NM_008425 |
| | (mouse) |
| ROMK | NM_000220 (human); NM_019659 |
| | (mouse) |
| V-type H-ATPase | NM_005177, (for all subunits see |
| | Beyenbach et al., J Exp Biol. 2006 |
| | Feb; 209(Pt 4): 577-89, incorporated by |
| | reference herein) |
| Na/H exchanger (NHE-1) | S68616 (human); U51112 (mouse) |
| F-type H-ATPase (PMA1) | X03534 (Saccharomyces cerevisiae) |
| NaV (SCN5A) | NM_000335 (human); NM_021544 |
| | (mouse) |
| Na dependent anion exchanger (SLC9A1) | NM_003047 (human); NM_016981 |
| | (mouse) |

We claim:

1. A method of promoting regeneration, comprising
(a) providing a population of naturally regenerating cells;
(b) determining the membrane potential or pH range permissive for regeneration in said population of regenerating cells;
(c) providing a population of non-regenerating cells;
(d) determining the membrane potential or pH range of said population of non-regenerating cells; and
(e) contacting the population of non-regenerating cells with an agent that modulates ion flux mediated by a class of ion transporter proteins, which agent modifies the membrane potential or pH of one or more cells in the population of non-regenerating cells to the range permissive for regeneration as determined in step (b); thereby promoting regeneration of one or more cells in the population of cells.

2. A method of promoting regeneration, comprising
(a) determining the membrane potential or pH range permissive for regeneration in a population of regenerating cells;
(b) providing a population of non-regenerating cells;
(c) determining the membrane potential or pH range of said population of non-regenerating cells; and
(d) contacting the population of non-regenerating cells with an agent that modulates ion flux mediated by a class of ion transporter proteins, which agent modifies the membrane potential or pH of one or more cells in the population of non-regenerating cells to the range permissive for regeneration as determined in step (b); thereby promoting regeneration of one or more cells in the population of cells.

3. The method of claim 1, wherein step (b) comprises
(i) providing a population of regenerating cells;
(ii) contacting said population of cells with an agent which is a voltage sensitive agent that produces a detectable signal; and
(iii) measuring the detectable signal to calculate an average membrane potential or pH of said population of cells during regeneration.

4. The method of claim 1, wherein steps (a) and (b) are repeated for multiple types of naturally regenerating cells and the average membrane potential or pH range are used in step (e).

5. The method of claim 1, wherein step (d) comprises
(i) providing a population of non-regenerating cells;
(ii) contacting said population of cells with an agent which is a voltage sensitive agent that produces a detectable signal; and
(iii) measuring the detectable signal to calculate an average membrane potential or pH of said population of cells.

6. The method of claim 1, wherein the method is an in vitro method and the population of non-regenerating cells are in culture.

7. The method of claim 1, wherein the method is an in vitro method and the population of non-regenerating cells are in a preparation of tissue in culture.

8. The method of claim 1, wherein the agent inhibits ion flux mediated by the class of transporter proteins.

9. The method of claim 1, wherein the agent promotes ion flux mediated by the class of transporter proteins.

10. The method of claim 1, wherein the agent is an ion channel protein or a nucleotide construct that encodes an ion channel protein.

11. The method of claim 10, wherein the ion transporter protein is a hyperpolarizing transporter.

12. The method of claim 10, wherein the ion transporter protein is a depolarizing transporter.

13. The method of claim 10, wherein the ion transporter protein is an H⁺ pump.

14. The method of claim 13, wherein the H⁺ pump is a V-ATPase H⁺ pump.

15. A method of promoting regeneration, comprising
(a) providing a population of cells of known membrane potential or pH; and
(b) contacting the population of cells with an agent that modulates ion flux mediated by a class of ion transporter proteins, which agent modifies the membrane potential or pH of one or more cells in the population of cells to a range permissive for regeneration; thereby promoting regeneration of one or more cells in the population of cells.

16. The method of claim 15, wherein contacting the population of cells with the agent promotes dedifferentiation.

17. The method of claim 16, wherein the dedifferentiated cells are cultured for a time sufficient to allow proliferation.

18. The method of claim 15, wherein the membrane potential range permissive for regeneration is between −70 mV and 30 mV.

19. A method of promoting regeneration, comprising
(a) providing a population of cells; and
(b) contacting the population of cells with an agent that increases ion flux mediated by a V-ATPase H⁺ pump, which agent promotes relative hyperpolarization of cell membranes of one or more cells in the population of cells, thereby promoting regeneration of one or more cells in the population of cells.

20. The method of claim 19, wherein contacting the population of cells with the agent promotes dedifferentiation.

21. The method of claim 20, wherein the dedifferentiated cells are cultured for a time sufficient to allow proliferation.

22. The method of claim 19, wherein the agent is a nucleotide construct encoding the V-ATPase H⁺ pump.

23. A method of promoting dedifferentiation, comprising
(a) determining the membrane potential or pH range permissive for dedifferentiation in a population of dedifferentiated cells;
(b) providing a population of differentiated cells;
(c) determining the membrane potential or pH range of said population of differentiated cells; and
(d) contacting the population of differentiated cells with an agent that modulates ion flux mediated by a class of ion transporter proteins, which agent modifies the membrane potential or pH of one or more cells in the population of differentiated cells to the range permissive for dedifferentiation as determined in step (b); thereby promoting dedifferentiation of one or more cells in the population of cells.

24. A method of inhibiting dedifferentiation, comprising
(a) determining the membrane potential or pH range permissive for dedifferentiation in a population of dedifferentiated cells;
(b) providing a population of cells;
(c) determining the membrane potential or pH range of said population of cells; and
(d) contacting the population of cells with an agent that modulates ion flux mediated by a class of ion transporter proteins, which agent modifies the membrane potential or pH of one or more cells in the second population of cells out of the range permissive for dedifferentiation as determined in step (b); thereby inhibiting dedifferentiation of one or more cells in the population of cells.

25. The method of claim 23, further comprising culturing the population of cells, which population of cells includes one or more dedifferentiated cells, wherein said culturing promotes regeneration.

26. The method of claim 23, wherein the compound inhibits ion flux mediated by the class of transporter proteins.

27. The method of claim 23, wherein the compound promotes ion flux mediated by the class of transporter proteins.

28. A method of identifying progenitor cells, comprising
(a) contacting a population of cells with an agent, which agent is a voltage sensitive agent that produces a detectable signal in response to a depolarized cell membrane;
(b) identifying, in the population of cells, one or more cells having a membrane potential of greater than or equal to −20 mV,
wherein the one or more cells having a membrane potential of greater than or equal to −20 mV are identified as candidate progenitor cells.

29. A method of identifying progenitor cells, comprising
(a) contacting a population of cells with an agent, which agent is a pH sensitive agent that produces a detectable signal;
(b) identifying, in the population of cells, one or more cells which have an intracellular pH of less than or equal to 6.7,
wherein the one or more cells having an intracellular pH of less than or equal to 6.7 are identified as candidate progenitor cells.

30. The method of claim 28, wherein the method is an in vitro method and the population of cells is in culture.

31. The method of claim 28, wherein the method is an in vivo method and the cells are in an animal.

32. The method of claim 28, wherein the candidate progenitor cells have a membrane potential greater than or equal to −20 mV and less than or equal to 30 mV.

33. The method of claim 28, further comprising
contacting the population of cells with a pH sensitive agent that produces a detectable signal; and
identifying, in the population of cells, one or more cells with an intracellular pH of less than or equal to 6.7,
wherein the one or more cells having both a membrane potential of greater than or equal to −20 mV and an intracellular pH of less than or equal to 6.7 are candidate progenitor cells.

34. The method of claim 28, further comprising
(c) separating candidate progenitor cells from the population of the cells.

35. The method of claim 28, further comprising
(d) culturing candidate progenitor cells separated in (c) to produce a population of cells enriched for candidate progenitor cells.

36. The method of claim 28, wherein the detectable signal is a fluorescent signal.

37. A method of identifying progenitor cells, comprising
(a) contacting a population of cells with a first agent, which first agent is a voltage sensitive agent that produces a detectable signal in response to a depolarized cell membrane;
(b) contacting the population of cells with a second agent, which second agent is a pH sensitive agent that produces a detectable signal;
(c) identifying, in the population of cells, one or more cells having a membrane potential of greater than or equal to −20 mV, and an intracellular pH of less than or equal to 6.7,
wherein the one or more cells having both a membrane potential of greater than or equal to −20 mV and an intracellular pH of less than or equal to 6.7 are identified as candidate progenitor cells.

38. The method of claim 37, wherein the population of cells is contacted simultaneously with the first agent and the second agent.

39. The method of claim 37, wherein the population of cells is contacted sequentially with the first agent and the second agent.

40. The method of claim 37, further comprising
(d) separating candidate progenitor cells from the population of the cells.

41. The method of claim 40, further comprising
(e) culturing candidate progenitor cells separated in (d) to produce a population of cells enriched for candidate progenitor cells.

42. A method of separating progenitor cells from an animal or tissue, comprising
(a) contacting an animal or tissue with an agent, which agent is a voltage sensitive agent that produces a detectable signal in cells in response to a depolarized cell membrane;
(b) identifying, in the animal or tissue, one or more cells having a membrane potential of greater than or equal to −20 mV,
wherein the one or more cells having a membrane potential of greater than or equal to −20 mV are identified as candidate progenitor cells;
(c) removing identified candidate progenitor cells, thereby separating candidate progenitor cells from the animal or tissue.

43. A method of separating progenitor cells from an animal or tissue, comprising
(a) contacting an animal or tissue with an agent, which agent is a pH sensitive agent that produces a detectable signal;
(b) identifying, in the animal or tissue, one or more cells with an intracellular pH of less than or equal to 6.7,
wherein the one or more cells having an intracellular pH of less than or equal to 6.7 are identified as candidate progenitor cells;
(c) removing identified candidate progenitor cells, thereby separating candidate progenitor cells from the animal or tissue.

44. The method of claim 42, further comprising
contacting the animal or tissue with an agent, which agent is a pH sensitive agent that produces a detectable signal; and
identifying, in the animal or tissue, one or more cells with an intracellular pH of less than or equal to 6.7,
wherein the one or more cells having both a membrane potential of greater than or equal to −20 mV and an intracellular pH of less than or equal to 6.7 are identified as candidate progenitor cells.

45. The method of claim 42, wherein removing identified candidate progenitor cells comprises dissecting out candidate progenitor cells, thereby removing candidate progenitor cells from the animal or tissue.

46. The method of claim 42, wherein removing identified candidate progenitor cells comprises
dissociating the animal or tissue; and
sorting candidate progenitor cells, thereby separating candidate progenitor cells from the animal or tissue.

47. The method of claim 46, wherein sorting the candidate progenitor cells comprises an automated method of sorting candidate progenitor cells based on the detectable signal.

48. A method for identifying a candidate class of ion transporter proteins which mediate ion flux during a particular biological process, comprising
(a) providing a population of cells for measuring a particular biological process;
(b) contacting the population of cells with a first compound that modulates ion flux mediated by a first class of ion transporter proteins;
(c) measuring the particular biological process in the population of cells in the presence of the compound versus the absence of the compound;
(d) determining whether the compound which modulates ion flux mediated by the first class of ion transporter proteins changes the particular biological process in the population of cells, thereby identifying a candidate class of ion transporters which may mediate ion flux during the particular biological process;
(e) providing a second population of cells for measuring said particular biological process;
(f) contacting the second population of cells with a second compound that modulates ion flux mediated by a second class of ion transporter proteins, which second class of ion transporter proteins comprises a subset of the first class of ion transporter proteins;
(g) measuring the particular biological process in the population of cells in the presence of the second compound versus the absence of the second compound; and
(h) determining whether the second compound which modulates ion flux mediated by the second class of ion transporter proteins changes the particular biological process in the population of cells,
thereby identifying a class of ion transporters which mediate ion flux during the particular biological process.

49. The method of claim 48, wherein the compound inhibits ion flux mediated by the class of transporter proteins.

50. The method of claim 48, wherein the compound promotes ion flux mediated by the class of transporter proteins.

51. The method of claim 48, wherein the particular biological process is selected from cell proliferation, cell differentiation, apoptosis, cell survival, cell migration, regeneration, or dedifferentiation.

52. The method of claim 48, wherein measuring the particular biological process comprises measuring a change in gene expression, a change in protein expression, or a change in morphology.

53. The method of claim 48, wherein both the first compound and the second compound change the particular biological process, thereby identifying a candidate class of ion transporter proteins which mediate ion flux during the particular biological process, and which candidate class of ion transporter proteins is a subset of the first class of ion transporter proteins.

54. The method of claim 48, furthering comprising
providing a population of cells for measuring said particular biological process;
inhibiting expression or activity of an ion transporter protein, which ion transporter protein is a member of the candidate class of ion transporter proteins which mediate the particular biological process;
measuring the particular biological process in the population of cells; and
determining whether inhibition of the expression or activity of said ion transporter protein changes the particular biological process in the population of cells,
thereby identifying an ion transporter protein which mediates ion flux during the particular biological process.

55. The method of claim 54, wherein inhibiting the expression or activity of the ion transporter protein comprises
contacting the population of cells with an agent that specifically inhibits the expression or activity of the ion transporter protein and does not substantially inhibit the expression or activity of other ion transporter proteins that are a member of the candidate class of ion transporter proteins.

56. A method for modulating a particular biological process, comprising
(a) identifying a candidate class of ion transporter proteins which may mediate ion flux during a particular biological process according to the method of claim 48;
(b) contacting a population of cells with a compound that modulates the expression or activity of one or more ion transporters proteins of the candidate class of ion transporter proteins; thereby modulating the particular biological process.


SEQUENCE LISTING (52 sequences)

SEQ ID NO: 1 - DNA, 2520 bp, Homo sapiens
atgaagaaatggagcagcacagacttgggggcagctgcggacccactccgaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctacacgccctggtggaggtggccgacaacacggccgacaacacgaagtttgtgacgagcatgtacaatgagattctgatcctgggggccaaactgcacccgacgctgaagctggaggagctcaccaacaagaagggaatgacgccgctggctctggcagctgggaccgggaagatcggggtcttggcctatattctccagcgggagatccaggagcccgagtgcaggcacctgtccaggaagttcaccgagtgggcctgcgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtgaacagctacagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctgtgtaattctcacctacatcctcctgctcaacatgctcatcgccctcatgggtgagactgtcaacaagatcgcacaggagagcaaggacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 2 - protein, 839 aa, Homo sapiens [sequence omitted]

SEQ ID NO: 3 - DNA, 2520 bp, Mus musculus
atggagaaatgggctagcttagactcggatgaatctgagcccccagcccaagagaactcctgcccggaccctccagacagagaccctaactccaagccgcctccagccaagccccacatctttgctaccaggagtcgcacccggctttttgggaagggtgactcagaagaggcctctcccatggactgcccttatgaggaaggcgggctggcctcctgccctatcatcaccgtcagctctgttgtcactctccagaggtctgtggatggacctacctgtctcaggcagacatcccaggactctgtctccactggtgttgagacgcccccaaggctctatgatcgcaggagcatcttcgacgctgtggctcagagcaactgccaggagctggagagcctgctgtccttcctgcagaagagcaagaagcgcctgactgacagcgagttcaaagacccagagacgggaaagacctgtctgctcaaagccatgctcaatctgcacaatgggcagaacgacaccattgctctgctcctggacattgcccggaagacagatagcctgaagcagtttgtcaatgccagctacacagacagctactacaagggccagacagcattacacattgccattgaaaggcggaacatggcactggtgaccctcttggtggagaatggagcagatgtccaggctgctgctaacggggacttcttcaagaaaaccaaagggaggcctggcttctactttggtgagctgcccctgtccctggctgcgtgcaccaaccagctggccattgtgaagttcctgctgcagaactcctggcagcctgcagacatcagtgcacgggattcggtgggcaacacggtgctgcacgcccttgtggaggtggcagataacacagctgacaacaccaagttcgtgacaaacatgtacaacgagatcctgatcctgggggccaaactccaccccacactgaagctagaagaactcaccaacaagaaggggcttacaccgctggctctggctgccagcagtgggaagattggggtcttggcctacattctccagagggagatccacgaaccagagtgccggcacctgtccaggaagttcactgaatgggcctatgggcccgtgcactcctccctttatgacctgtcctgcattgacacctgtgagaagaattcagtgctggaggtgatcgcctacagtagcagtgagacccccaaccgccacgacatgcttctcgtggagcccttgaaccgactcctgcaggacaagtgggacagatttgtcaagcgcatcttctacttcaacttcttcgtctactgcttgtatatgatcatcttcaccacggctgcttactatcggcctgtggaaggcttgcccccctataagctgaataacaccgttggggactatttccgtgtcactggagagatcctgtctgtgtcaggaggagtctacttcttcttccgagggatccagtatttcctgcagaggcgaccatccctcaagagtttgtttgtggacagctacagtgagatacttttctttgtacagtcactgttcatgctggtgtctgtggtactgtacttcagccatcgcaaggagtatgtggcttccatggtgttctccctggccatgggctggaccaacatgctctactacacccgaggattccagcagatgggcatctatgctgtcatgattgagaagatgatcctcagagacctgtgtcggtttatgttcgtctacctcgtgttcttgtttggattttccacagccgtagtgacactgatcgaggatgggaagaataactcactgcctgtggagtccccaccacacaagtgtcggggatctgcctgcaggccaggtaactcttacaacagcctgtattccacatgtctggagctgttcaagttcaccatcggcatgggtgacctggagttcaccgagaactatgacttcaaggctgtcttcatcatcctgttactggcctatgtgattctcacctacatcctcctgctcaacatgctcattgctctcatgggcgagactgtcaacaagattgcacaagagagcaagaacatctggaagctgcagcgagccatcaccatcctggatacagagaagagtttcctgaagtgcatgaggaaggccttccgctccggcaagctgctgcaggtggggttcacgccggacggcaaggatgacttccggtggtgcttcagggtggatgaggtgaactggactacctggaacaccaacgtgggcatcatcaacgaggacccaggcaactgtgagggcgtcaagcgcaccctgagcttctccctgcggtcaggccgagtttcagggagaaactggaagaactttgccctggttccccttctgagggacgcaagcactcgagataggcatagcacccagccggaagaagttcagctgaagcactatacgggatcccttaagccagaggatgctgaggtcttcaaggattccatggccccaggggagaaatga

SEQ ID NO: 4 - protein, 839 aa, Mus musculus
MEKWASLDSDESEPPAQENSCPDPPDRDPNSKPPPAKPHIFATRSRTRLFGKGDSEEASPMDCPYEEGGLASCPIITVSSVVTLQRSVDGPTCLRQTSQDSVSTGVETPPRLYDRRSIFDAVAQSNCQELESLLSFLQKSKKRLTDSEFKDPETGKTCLLKAMLNLHNGQNDTIALLLDIARKTDSLKQFVNASYTDSYYKGQTALHIAIERRNMALVTLLVENGADVQAAANGDFFKKTKGRPGFYFGELPLSLAACTNQLAIVKFLLQNSWQPADISARDSVGNTVLHALVEVADNTADNTKFVTNMYNEILILGAKLHPTLKLEELTNKKGLTPLALAASSGKIGVLAYILQREIHEPECRHLSRKFTEWAYGPVHSSLYDLSCIDTCEKNSVLEVIAYSSSETPNRHDMLLVEPLNRLLQDKWDRFVKRIFYFNFFVYCLYMIIFTTAAYYRPVEGLPPYKLNNTVGDYFRVTGEILSVSGGVYFFFRGIQYFLQRRPSLKSLFVDSYSEILFFVQSLFMLVSVVLYFSHRKEYVASMVFSLAMGWTNMLYYTRGFQQMGIYAVMIEKMILRDLCRFMFVYLVFLFGFSTAVVTLIEDGKNNSLPVESPPHKCRGSACRPGNSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGFTPDGKDDFRWCFRVDEVNWTTWNTNVGIINEDPGNCEGVKRTLSFSLRSGRVSGRNWKNFALVPLLRDASTRDRHSTQPEEVQLKHYTGSLKPEDAEVFKDSMAPGEK

SEQ ID NO: 5 - DNA, 7807 bp, Homo sapiens
cgcaccctccttccgcccctccttctccggggtcagccaggaagatgtcccgagctgctatccccggctcggcccgggcagccgccttctgagcccccgacccgaggcgccgagccgccgccgcccgatgggctgggccgtggagcgtctccgcagtcgtagctccagccgccgcgctcccagccccggcagcctcagcatcagcggcggcggcggcggcggcggcggcgtcttccgcatcgttcgccgcagcgtaacccggagccctttgctctttgcagaatggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggaatggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggagggaccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttgcggtcggattcattataaggatatgtacagtttattacgagtaatatctccccctctcggcttaggcaagaaatgtcctcatagggttgcttgcaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcaggcggtggccaggccgggccgggcggccaccagcggccctcggaggtacccaggccccacggccgagcctctggccggagatcggccgcccacggggggccacagcagcggccgctcgcccaggatggagaggcgggtcccaggcccggcccggagcgagtcccccagggcctgtcgacacggcggggcccggtggccggcatctggcccgcacgtgtccgaggggcccccgggtccccggcaccatggctactaccggggctccgactacgacgaggccgatggcccgggcagcgggggcggcgaggaggccatggccggggcctacgacgcgccaccccccgtacgacacgcgtcctcgggcgccaccgggcgctcgcccaggactccccgggcctcgggcccggcctgcgcctcgccttctcggcacggccggcgactccccaacggctactacccggcgcacggactggccaggccccgcgggccgggctccaggaagggcctgcacgaaccctacagcgagagtgacgatgattggtgctaagcccgggcgagg

SEQ ID NO: 6 - protein, 2261 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAACGRIHYKDMYSLLRVISPPLGLGKKCPHRVACKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQ

SEQ ID NO: 7 - DNA, 7926 bp, Mus musculus
ggggtcaccgcctctgccggcatgtcccgagccgcgctccccggctcggccgggccgtcccgctcgtgagccccccgagctgaggccgggcgccgccgcccgatgggctgggccgtggagcgtctccgcagtcgcaggtgcagccgccgtccccacagcccggccagcctgagcagcggcggcggtggtggtggtggtggtggcggtggccgtggcttccgcagcgctcgccgcagcaacagcagccgccgcgtaacccgaggccctttgctctttgcagaatggcccgctttggagacgagatgccgggccgctacggcgcaggcggaggaggctcagggccggccgccggggtggtcgtgggcgccgcgggcggccgaggagccgggggcagccggcagggcgggcagcccggagcgcagaggatgtacaagcagtcgatggcgcagagagcgcggaccatggccctctacaaccccatccctgtccgccagaactgcctcacggtcaaccgctccctgttcctcttcagtgaagacaacgtggtgagaaaatacgccaaaaagatcactgaatggcctcccttcgagtacatgatcctggccaccatcattgctaactgcatcgtcctggccctggagcagcacctccctgatgatgacaagacacccatgtctgagcggctggatgacacggaaccatacttcattggaatcttctgttttgaggctggaattaagatcgtggctcttggctttgccttccacaaaggctcctacctgaggaatggctggaacgtcatggactttgtcgtggtgctaacaggcatcttggccactgtcgggacggagtttgacctacggacactgagggcggttcgtgtgctgcggccactcaagctggtgtctggaatcccaagtttacaagtcgtcctgaagtcaatcatgaaggcgatgatccctctgctgcagatcggcctcctcctgttttttgcaatccttatttttgcaatcatagggttagaattttatatggggaaatttcataccacctgctttgaagaggggacagacgacatccagggtgagtcgccagctccgtgtgggacagaggagcctgcccgcacctgccccaacgggaccaaatgtcagccgtactgggaagggcccaacaacggcatcactcagttcgacaacatcctgtttgctgtgctcactgttttccagtgcatcaccatggaaggctggactgatctcctctacaatagcaacgatgcctcagggaacacttggaactggttgtacttcatccccctcatcatcatcggctccttttttatgctgaaccttgttctgggtgtgctgtctggggagtttgccaaagaaagggaacgtgtggagaaccggagagcttttctgaagctcagaagacaacagcagattgaacgtgagctcaatggatacatggagtggatctcaaaagcagaagaggtgattcttgcggaggacgagacagacgtggagcagaggcacccttttgatggagctcttcggagagctactctgaagaaaagcaagacagacctgctcaaccctgaggaggccgaggaccagctggctgacatcgcgtctgtggggtctcccttcgccagagccagcatcaaaagtgccaagctggagaactcgactttttttcacaagaaggagagaagaatgcgtttctacatccgccgcatggtcaaaactcaggccttctactggaccgtgctcagtctggtagccctcaacacgctgtgcgtcgccattgtccactacaaccagcccgagtggctctccgacttcctctactatgcagaattcattttcttaggactctttatgtccgaaatgtttataaaaatgtacgggctcgggacgcggccttacttccactcttccttcaactgctttgactgtggggtcatcattgggagcatctttgaagtcatctgggctgtcatcaaacctggtacatcctttggaatcagtgtgttacgagctctcaggttattgcgaattttcaaagtcacaaagtactgggcatctctcagaaacctggttgtctccctcctcaactccatgaagtccatcataagtctgttgttcctccttttcctgttcatcgttgtctttgccctcctggggatgcagctgttcggtggccaattcaattttgatgaggggactccgcccaccaacttcgacacttttccagcagcaataatgactgtgtttcagatcctgactggcgaggattggaatgaggtcatgtatgatgggatcaagtctcaggggggcgtgcagggcggcatggtgttctccatctacttcatcgtcctcaccctcttcgggaactacaccctgctgaacgtgttcttggccatcgcagtggacaacctggccaatgcccaggagctcaccaaggatgagcaagaagaggaagaggcagccaatcagaagctggctctacagaaagccaaggaggtagcagaagtgagtcccctgtctgcagccaacatgtccatagccgtgaaggagcagcagaagaaccagaagcctaccaagtcggtgtgggagcagcgcaccagcgagatgcgcaagcagaacctgctggccagccgcgaggcgctgtacggggacgcggctgagcgctggcccaccccctacgcgcgcccgctgcggcccgacgtgaagactcacttggaccggccgcttgtggtggacccgcaggagaaccgtaacaacaacaccaacaagagccgtgcgccagaagcgctgcgcccgaccgcgcggccccgcgaaagcgcgcgcgaccccgacgcgcggcgcgcctggcccggcagccctgagcgcgcccctggacgagagggcccgtatgggcgcgagagcgagcctcagcagcgcgagcacgcgccgccccgcgagcacgcaccctgggacgcggacaccgagcgcgccaaagccggggacgcgccccgccgccacacgcaccgacctgtggcggagggcgagcctcgccgccaccacgcccgccgccggcccggggacgaacccgacgacaggccagagcgcaggccacgtccccgcgacgccactaggccggcccgcgctgcagacggcgaaggcgacgatggggagcgcaagcggcgacaccgacacgggccgccggcccacgacgacagggagcgcagacaccgacgcagaaaagagaaccagggctctggggtccccgtgtctggtcccaacttgtccaccaccaggccaatccagcaggacctgggccgccaggacctgccgctggctgaggatcttgacaacatgaagaataacaagctggccaccggggaacctgccagtccccacgacagcctgggccacagcggccttccccccagccccgccaaaatcgggaacagcaccaaccctggtcccgccttggccaccaatccccagaacgctgccagccgcaggacgcccaacaatccgggcaacccgtccaaccccggcccccccaagactcccgagaacagccttatcgtcaccaaccccagcagcacccagcccaactcagcgaagactgccaggaaacccgagcacatggcggtggagatccccccagcctgcccgcccctcaaccacaccgtggtccaagtaaacaaaaacgccaacccagacccactgccaaagaaagaggaagagaagaaagaggaagaggaggcagaccccggggaggacggcccaaagcccatgccgccctacagctccatgttcatcctctccaccaccaacccccttcgccggctgtgccattacatcctgaacctgcgctacttcgagatgtgcatcctcatggtcattgccatgagcagcatcgctctggccgccgaggacccggtgcagcccaacgcaccccgcaacaatgtgctgcgatattttgactatgtttttacaggcgtgtttacctttgagatggtgatcaagatgatcgacctgggcctcgtcttgcatcagggggcctatttccgtgacctatggaacattctggacttcatagtggtcagtggggccctggtggcctttgccttcactggcaatagcaaaggaaaggacattaacaccattaagtctctccgagtcctccgggtgctacgacctctaaagaccatcaagcggctgccaaagctgaaggccgtgtttgactgcgtggtgaactcgctcaagaacgtcttcaacatcctcattgtgtacatgctgttcatgttcatcttcgctgtggtagccgtgcagctcttcaagggcaagttcttccactgcacggacgagtccaaggagttcgagagagactgtcgaggcaagtacctcctgtacgagaagaacgaagtgaaggcccgggaccgcgagtggaagaagtacgagttccactacgacaacgtgctctgggctctgctcacacttttcacagtgtccacaggagagggctggccacaggtcctcaagcactcagtggatgctacttttgagaaccagggccccagccccgggtaccgcatggagatgtccatcttctacgtggtgtactttgtagtgtttcccttcttctttgtcaatatcttcgtggccttgatcatcatcacctttcaggagcagggagacaagatgatggaagagtacagtttggagaaaaatgagagggcctgcatcgactttgccatcagtgccaagccgctgaccaggcacatgccccagaacaagcagagcttccagtatcgaatgtggcagttcgtcgtgtccccaccctttgagtacaccatcatggccatgatcgctctcaacaccatcgtgctaatgatgaagttctacggagcctcggtggcctatgaaaacgcccttcgcgtgttcaacattgtcttcacctccctcttctctctcgaatgtgtgctcaaagtcatggcttttgggattctgaattatttccgcgatgcctggaacatcttcgactttgtgactgttctgggcagcatcacagacatcctcgtcaccgagtttgggaataacttcatcaacctgagctttctccgcctcttccgtgctgccagactcatcaaactcctccgccagggttacaccatccgcattctcctctggactttcgtgcagtccttcaaggccctaccttatgtctgtctgctgatcgccatgctcttcttcatctacgccatcatcgggatgcaggtgtttggcaacatcggcattgacggggaagacgaggacagcgacgaggatgaattccaaatcacggagcacaataacttccggaccttcttccaagcgctcatgcttctcttccggagcgccacaggagaagcgtggcacaacatcatgctctcctgcctcagcgggaagccgtgcgataagaactctgggatcctcacggcagactgcggcaacgagttcgcgtacttctactttgtctcgttcatcttcctgtgctcgtttctgatgctgaatctctttgtcgctgtcatcatggacaacttcgagtacctcacccgagattcttccatcctgggcccccatcacttggatgaatacgtgcgtgtctgggcagagtatgaccctgctgcctgcggccgcattcactataaggacatgtacagtttattgcgagtaatatcgccccctctcggcttaggcaagaaatgtcctcatagggttgcttgcaagagactcctgcggatggacctgccggtagcagatgacaacacagttcacttcaactccaccctgatggctctgatccgaaccgcccttgatatcaaaattgccaagggtggagctgacaagcagcagatggatgcagagcttcgcaaggagatgatggccatttggcccaacctgtctcagaagaccctggatctgctggtcacacctcacaagtccacggacctgacagtgggtaagatctacgcagccatgatgatcatggagtattaccggcagagcaaggccaagaaactgcaggccatgcgagaggagcagaaccggacaccactcatgttccagcgcatggagcctccatcaccaacacaggagggaggacccagccagaatgcccttccctccactcagctggacccaggaggaggcctgatggctcacgaaggcggcatgaaggagagcccgtcctgggtgacccagcgggcccaagagatgttccagaagactggcacctggagcccagagcgagggccacccatcgacatgcctaacagccagcccaactcccagtctgtggagatgcgagaaatgggtactgatggctactcagacagcgaacactacctccccatggaaggccagaccagggccgcctccatgccccgcctcccagcagagaaccagaggagaaggggccggccacgtggaaatgacctcagtaccatctctgataccagccccatgaagcgctcagcctctgtgctgggacccaaagcccggcgactggatgactactcactagagcgggtaccgcctgaggagaaccaacggtaccaccaacgtcgccgggaccgtggccaccgcacctctgagcgctctctgggccgatacactgatgtggacacaggtttggggacagatctgagcatgaccacccaatcgggtgacctgccctccaaagatcgggaccaggaccggggccgacccaaggaccggaagcatcggccacaccaccaccaccaccaccatcatcatcatcccccggcccccgaccgggatcgctacgcacaggagcggccggacaccggccgggcgcgggcccgggagcagcgctggtcccgctcgcccagcgaaggtcgggagcacacgacacacagacagggcagtagttccgtcagtggaagcccagccccctctacgtccggcaccagcacgccgcggcgcggccgccgccagctcccccagaccccctgcaccccgcggccgcttgtgtcctactcgcccgctccgcgcaggcctgcggcgcgcaggatggcgggtcccgcagcgccccctggcggctcgccgcggggttgccgccgtgcgccgcgctggcctgcgcatgcgcctgagggcccgcggccgcgcggtgccgactacaccgagcccgacagcccgcgcgagccgccagggggcgcgcacgatcccgcgccgcggtcgcccaggactccgcgcgccgcgggctgcgcgtcaccgcgccacggccgcaggctgcccaatggttactacgcggggcacggcgcgccgcggccgcgcacagcccgcaggggcgcgcacgacgcgtacagcgagagcgaggacgactggtgctaagccgccccgccccccgcagcgcccgtaaagtgcgggcgcactgcaaatgataaacaataacgtttttaaaaaagaaaacccgggagaaaaacaaaaatagcttctattgatgagttttatcgtctcgattgaatctttcgttttcccggtgaatgaaaccgaggtggctgcaagccggaaggacggctgcacgccccacacacttctcagacccacgggagtgcttgcgggccacgccaaacttattactgcctgcagagatcaactacaagaaaacaacactaacagtttttaaaaggacaaaaaaaaattgagaaaagttttttttctgatatttggtcctgcttgaaataacaaaagaaaaaagaaaaaattaccaccatcactgattcccttgcttctttttttcctttatctttttaaaaattttttttcctttttttcctatcttgtttgaaaaacgtgggcttgggactgtgaaatattgcatgacattaaaaagaacaaaaaaataataaaaacgaaaaaacttgaatcc

SEQ ID NO: 8 - protein, 2368 aa, Mus musculus
MARFGDEMPGRYGAGGGGSGPAAGVVVGAAGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIVALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDVEQRHPFDGALRRATLKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPTKSVWEQRTSEMRKQNLLASREALYGDAAERWPTPYARPLRPDVKTHLDRPLVVDPQENRNNNTNKSRAPEALRPTARPRESARDPDARRAWPGSPERAPGREGPYGRESEPQQREHAPPREHAPWDADTERAKAGDAPRRHTHRPVAEGEPRRHHARRRPGDEPDDRPERRPRPRDATRPARAADGEGDDGERKRRHRHGPPAHDDRERRHRRRKENQGSGVPVSGPNLSTTRPIQQDLGRQDLPLAEDLDNMKNNKLATGEPASPHDSLGHSGLPPSPAKIGNSTNPGPALATNPQNAASRRTPNNPGNPSNPGPPKTPENSLIVTNPSSTQPNSAKTARKPEHMAVEIPPACPPLNHTVVQVNKNANPDPLPKKEEEKKEEEEADPGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFERDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDGEDEDSDEDEFQITEHNNFRTFFQALMLLFRSATGEAWHNIMLSCLSGKPCDKNSGILTADCGNEFAYFYFVSFIFLCSFLMLNLFVAVIMDNFEYLTRDSSILGPHHLDEYVRVWAEYDPAACGRIHYKDMYSLLRVISPPLGLGKKCPHRVACKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQNRTPLMFQRMEPPSPTQEGGPSQNALPSTQLDPGGGLMAHEGGMKESPSWVTQRAQEMFQKTGTWSPERGPPIDMPNSQPNSQSVEMREMGTDGYSDSEHYLPMEGQTRAASMPRLPAENQRRRGRPRGNDLSTISDTSPMKRSASVLGPKARRLDDYSLERVPPEENQRYHQRRRDRGHRTSERSLGRYTDVDTGLGTDLSMTTQSGDLPSKDRDQDRGRPKDRKHRPHHHHHHHHHHPPAPDRDRYAQERPDTGRARAREQRWSRSPSEGREHTTHRQGSSSVSGSPAPSTSGTSTPRRGRRQLPQTPCTPRPLVSYSPAPRRPAARRMAGPAAPPGGSPRGCRRAPRWPAHAPEGPRPRGADYTEPDSPREPPGGAHDPAPRSPRTPRAAGCASPRHGRRLPNGYYAGHGAPRPRTARRGAHDAYSESEDDWC

SEQ ID NO: 9 - DNA, 6950 bp, Homo sapiens
ggccaaaggtcaagatacttctctgggaaatgttgctgctgatgctgctttacaaagtcatacaatgagtgtttggtttaagaaagattttcatacttaaaagattttcatcttggaaatacatcaagtgaaaattaaattcttttgggaaacattttccttctgatatattatacttgtaatgggcgacatggcaaacaactcagttgcttacagtggtgtgaaaaactctttgaaggaagctaatcatgatggagactttggaattacgctcgcagagctgcgggctctcatggagctcaggtccacagatgcattacgaaaaatacaggaaagctatggagatgtctatggaatttgcaccaaattgaaaacatctcccaatgaaggtttaagtggaaaccctgcagatttagaaagaagagaagcagtgtttggaaagaattttatacctcctaaaaagccaaaaacctttcttcaattagtatgggaagcattacaagatgtcactttaattatattagaaattgcagccatagtatcattgggcctttctttttatcagcctccagaaggggataatgcactttgtggagaagtttctgttggggaggaagaaggtgaaggtgaaactggttggattgaaggagctgcaatcctcttgtctgtagtgtgtgtggtgttagtaacagctttcaatgactggagtaaggaaaaacagtttagaggtttgcagagccgaattgaacaagaacagaagttcactgtcatcaggggtggtcaggtcattcagatacctgtagctgacattactgttggagatattgctcaagtgaaatatggtgatcttcttccagctgacggcatacttattcaaggcaacgatcttaaaattgatgaaagctcattgactggtgaatcagatcatgttaaaaagtctttagataaggatcccttacttctatcaggtactcatgtaatggaaggctctggaagaatggtagttacagctgtaggtgtaaattctcaaactggaattatctttaccttacttggagctggaggtgaagaggaagagaagaaagatgagaagaaaaaggaaaagaaaaataagaaacaagatggagctattgagaatcgcaacaaagcaaaagcccaggatggtgcagccatggaaatgcagccattgaagagtgaagaaggtggagatggtgatgaaaaagataaaaagaaagcaaatttgccaaaaaaggaaaaatctgttttacaagggaaacttacaaaactggctgttcagattggcaaagcaggtctgttgatgtctgccatcacagttatcattctagtattatattttgtcattgacaccttctgggttcagaaaagaccatggcttgctgagtgcacaccaatttatatacaatactttgtgaagttcttcattattggagttacagttttagtggtcgcagtgccagaaggtcttccacttgcagtcacgatctcactggcttattcagtcaaaaaaatgatgaaagataataacttagtaaggcatctggatgcttgtgaaaccatgggaaatgctacagctatttgttcagataaaacaggaactttgacaatgaacagaatgacagtcgttcaagcttacataaatgaaaaacattataaaaaggttcctgaaccagaagctattccaccaaatattttgtcctatcttgtaacaggaatttctgtgaattgtgcttatacatcaaaaatattgccaccagagaaagagggtggattacctcgtcacgttggtaataaaactgaatgtgccttgttgggacttcttttggatttaaaacgggattatcaggatgttagaaatgaaataccagaagaagcactgtacaaagtctacaccttcaattctgttaggaagtccatgagtactgtcctgaaaaattcagatggaagttatcgaatattcagcaagggtgcatctgagataattctgaaaaagtgtttcaaaatcttgagtgctaatggtgaggcaaaagtattcagaccaagggaccgtgatgatattgtaaaaactgtgattgaaccgatggcatcagaaggcttgagaaccatatgtcttgcattcagagattttccagcaggagaaccagaaccagagtgggataatgaaaatgatattgtcaccggccttacatgcattgctgttgtggggattgaagatcctgtgagacctgaggtgccagatgcaattaaaaagtgtcagagggctggaattactgtgcggatggtcactggtgataatattaatactgctcgggccattgctaccaaatgtggtattttacatcctggggaagattttctgtgcctagaaggtaaagattttaacagaagaatacgaaatgaaaaaggagagattgagcaagagaggatagacaagatttggccaaaacttcgagtacttgcaagatcatctcctactgataagcatacactggttaaaggtataattgacagcactgtctcagaccaacgccaggttgtagctgtaactggtgatggtacaaatgatggcccagcactaaagaaagcagatgttggatttgcaatgggtattgctggaactgatgtagctaaagaagcatccgatattattctcacagatgacaactttacaagcattgttaaagcagttatgtggggacgaaatgtctatgacagcatctcaaaattccttcagttccaacttactgttaatgtagtagcagtgattgttgcttttacgggcgcctgcattactcaagactcaccgcttaaggctgtgcagatgctgtgggtaaacctcataatggatacactcgcttccctggctctggcaacggaaccacccactgagtctctcttgcttcggaaaccttatggtagaaataagcctctcatctcacgtacaatgatgaagaatattttgggtcatgcattctatcaacttgtagtagtctttacactcttatttgctggagaaaagttttttgacattgatagtggaagaaatgctcctttgcatgctcctccttcagaacattatactattgtttttaatacctttgtgctgatgcaacttttcaacgaaataaatgcccggaaaattcatggtgaaagaaatgtattcgaaggaatctttaacaatgccatcttctgcacaattgttttaggcacttttgtggtacagataataattgtgcagtttggtggaaaacctttcagttgttcagaactttcaatagaacagtggctatggtcaatattcctaggaatgggaacattactctggggccagcttatttcaacaattccaactagccgtttaaaattcctcaaagaagctggtcatggaacacaaaaggaagaaatacctgaggaggaattagcagaggatgttgaagagattgatcacgctgaaagggagttgcggcgtggccaaatcttgtggtttagaggtctgaacagaatccaaacacagatggatgtagtgaatgctttccagagtggaagttccattcagggggctctaaggcggcaaccctccatcgccagccagcatcatgatgtaacaaatatttctacccctacacatgtagtgttttcctcttctactgcttctactactgtggggtattcgagtggtgaatgcatttcgtagttctttatatgaagggttagaaaaaccggaatcaagaagttcgattcacaactttatgacacatcctgagtttaggatagaagattcagagcctcatatcccccttattgatgacactgatgccgaagatgatgctcctacaaaacgtaactccagtcctccaccctctcccaacaaaaataacaatgctgttgacagtggaattcaccttacaatagaaatgaacaagtctgctacctcttcatccccaggaagcccactacatagtttggaaacatcactctgattgtaagctgaatgttaacacactagctgcattgtaaagaaacaaattgaaactgggtcttttcacatattgtgatggacaagctagtattcttgtctttggacttcaacagaagacacacttgtacgaatgtagatttatttttttaaaaaaaaaaaagcaaagctttctgccagactgagggtgctttttggggggtgggagaaatgaactgacagataaacagtaactcagcgtaagtgacctgtggatcatcagtagtacccaagcatggtcctgaacagtgtattagcagagctttagtttatctcagtcctggatgggagagagagggaggagtaaaactgggcaagacaaagagccctggatcgttgaattgatagtttaatttctgctgttggctgttaataatttggattatttatgtttataaatgatacagatctgtttacaaggtttgtagatactttttttgttcctgttcatagatgggaagcttccttataactgatgcagagaaaaattagtccttcaaatactgctgtattttcaggaaaaaaaaaaaaaggggtgtttctattgaaactgtactaaatttttgcctacaatttaccttgttaaatattgtagataaattgctaatactaatagccaaatagataacactaatgctttgtttaaaaaaacgaaaaaaacaaaacaaaacaaaaaaacatgagcagtggtgttctaatgaagttatgacatctgtcctaattagtttcttaaatacatttactacttaatggttttgaagcaactgtttaatttttaaaatttttgaaaacttgctaataaccttttgttcagaatttagtagattgtttaatgcaggacatcaactacataatgaaagatgcttgaaatgcttttgttatttcaggcaaatcaaattaaatcagactatcaaactacaagagaaccttagtcttttttgtttttgtctaaacatgttagtttagtgatgtcttggtgaactgtgagtgaactgtattgatttttctaatctttagagaccttcacacagcatgagggctgttggcattttgaaaaaggttaataagtagaagcatacatgttttccttttgtttttgaaacttgtttgtaaacataaataaatatgcccttttattaaataaattcatggcaatgtttttttaaaagacatttcagcttctttactcagttctgcatatacagtgcatcagtctgttctctttgaccacaattggaatacatttcaagatggtaacagccccatgaagtaccgctttaattccttgctggtaatctaatagaatcagattaagaaaattaacacaattttaccatttgacatactcttcattgaatttagtttgtctaaaatagtctcacaggtaaaaaacataacaacaatgaccaccattgtctttgatacatttattttaattcttaagtggccaaataacagattaggtaaaatgagattacatgaaaaggttctaaaatgatatatttagatgcaataattaaccatatgctagaatgcccacctctaagccaattggattctttagctctctttttctttccttcatctaccttcttctcttttttgcctctgtctttaccctttagaaactggcatgtgttagggcccagatttcaccagatacacaatcctcaggctgacataaagtttacgaggactgctactataactgagagtgctgaccaagcaaagaaacatttagttgatggtgcttgatatttttggtgggtgggggaaatctttttctcaccatcaaatctttgcagtaatcctctgcaggtcttagaaacacattggctctgtcagcatttgtaattgtacatttattcatcaggtatttatagtgaatcatactgtaaaaaagaggtagcatgatatgctggaaagcatattagactaggagtcaagacctggtttctagtcttggttttgccattaatgtgctgcatgatcttgggaaaatcatttaccctaaataagggtggtggactatcttgaagaaccctttcaactctaaaaattgaccctatcaactggagagcccacaaggcatagggaatagagaaaagggaaatgttttcagttttcttttttattttggttatgcttaaatatgaatgtaattaccaaaagatttagaagtgcatgtgctttggaggatttgtattgagcttttacagtattcatttttcaactcaaggcaatggctttctacaccaactctaatccataaacgggtcttatgacatctatgaagtagtagcaagacatgcttagtgtgtatttctctctttgagacactgtaatttctaccagaaatttccagagcattatgtaggtagaaaaaaatgcaagcaagctgttaaagatcttggatcccattatatagtatgtatagctgaaatctgtaattcaatcactttttctcttttatcctctaaccaaaaaattgtttaattttgcatcccaaatgtttttaatctttgtatattttttaaaaatccttttctcctcatcattgccttttttgtggttgtaaatagacttacttgcactttgaagatgagttactccttgtcatcttacaaatatgtgatatggtaattttcataacagatgtcagttttgaaccaagaattggtgatttgtttataagaaaaaaactggcttcatttctgtgaaattgctctttgaaaatttctttttacacgtgtaagccaactgagataccgtgatggtgttgatttctttcaatgatgcttaccatctattttagccactgagccttttattatttgtctatttgtaaagtttatttgtcttaactcatttaataaatatactgtttatctgtttctgaaaaaaaaaaaaaaaaaaa

SEQ ID NO: 10 - protein, 1176 aa, Homo sapiens
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHVVFSSSTASTTVGYSSGECIS

SEQ ID NO: 11 - DNA, 2670 bp, Xenopus tropicalis
ccccgggcttgagggtgcattatcataaagacttgtagttcttttgtcagattttattagccatacatgactggtgtgagtgagatgtctacctgcctgcatcttcaatacacttgagagtttctaagtgctacataatcctccgtagctttcatcagttttatttctgttagctgctctataaaagtgcctttttctctttgctttttatttattgtagatgggatggtcctttctcttgttctctctctctcttgctgagcaagctgtcacaatctctgattccttgcagatggatgtagtgaatgctttccaaagtggaagttccattcagggggctttaaggcggcaaccctccatcgccagccagcatcatgatgtaacaaatatttctacccctacacatgtagtgttttcctcctctactgcttctactactgtggggtgtgagtgtgtgttcctaagtgcatgaaattaacacatcctaattcacatacctaacgtttctcattttttcctcagtaatcaatctcattgttcaggctgctcataaagcttccccttcacaaggataaaaagtcctgtgttgtttatatatttttttccatagaaggctattgaactggttggaatggtagcaaatgcaagttttgaaaaatgtctgcaaccctgcacgcttttatttataagtatattgctgcgtttatgtatatatttgaaagttgattgtgctgctgtaaattttcgagtatataccgtatttttttatgcagaaggcatgatgtttggctttgggaatattttacacttatatatcttagtttacacatttctatttagtgtaggcaaccacataaaggataaatcctttacagtcattttaaattttcttgtgactactaggatatggactagctaactattgacatagttggttgtaaatgtgaccatgggatggcagaagacatctagacaaatactctattccttttctctaaaagtggaaatctatgctcagagagagatccttcccaattgccttccctacattctctccatgaccatgcttcgctgtgacataatcccaggcagtttagctttatggtctatgaagtggatgtgattattgttaggttgtgtgtttttggtagcaaaggtcccatttatgagaactaaattcacatgaggcaaaacttgtgctagaaaaagggttttgctaaggaaagctgatatttgccttccttagcaatgctctccagcaagtgtttgccctaaagaatctccttctgtgtgctattactattagaacttaatataatgtaatcgcaaatttgaacagcgttgctatacaagaaagcagagcacgaataacactttatcttgggcttactatccacaaaaatgtctatttctatccacagaattacttctactacttcagctgttgttgaaactagtgtttagccaaaattaattttatataccgtatgttttatgttggcattgttggttgtgagtgaggcatgtggattctagtaaacaggatggcaacctgataggaacatataaaaccactctaagaacagttcatgcaccgggttgtatgtcactcaaaacatataaagtttcaaggttttccaacatgtatagtttaaataaatatatccatatggcctacaaattcatagggagtacttcatttactacatgttgtgagctacatctaccactgaagcccctgatctacttgtatatagctgtaatgtgattttgttcatgcaagaaaaatatgcacatagatcactttctagcttgctaattgttcggcgatttgatcaattagtcttaacattttggcaccagacaatgttatatggaggtttttagtttctatggtgctctcatagacaaacccttcttcttttgaggggaaaacctgctgtctggcagatattggtgctggtaagcagctttggaaagctgagtaataccagtacaagtgttgagttttctacctgagttattaagggcaccaacctaatgctagataaaaataccaatggtttgttacccattataaattgcggcaagacatgttgtttaggctttccaggtcagcagattaacatagaataagacaaaaacaaatatcaaggcctgttgctgcaagttatcttacacacttgctcttatctattcttctctaagtactattcttgtgttgtatacttgtattagcagtctctctctctatttggtggataacaacaatccaaatgcaaatttggtattaaaggtttgaccacaattgtttttttttatttgaagattgaactgtgtttaaacaaatgtttttttttccccttagggttccagattattttaaaaatgaaaaaagtgatgtttaacaatgtacttttacattttccaggttattaacttttgattccagcatcaagtatatttaattaaacttctatatagtcttatctttgccaaaaatgcacttagtaatcatgtctgttagcgctcacattagggatgcattaaacctatcgtttttggattcgggcaaataccaaatcttctacactagatttgtttgagtacttaactgaaccctatttttcgtattcaagtttgctataagccaaaaaaaaaaaaaaaaaa

SEQ ID NO: 12 - protein, 59 aa, Xenopus tropicalis
MDVVNAFQSGSSIQGALRRQPSIASQHHDVTNISTPTHVVFSSSTASTTVGCECVFLSA

SEQ ID NO: 13 - DNA, 3250 bp, Homo sapiens
ccggaggattttgaggacacttgtggagagctcgaattccagaatgatgaaattgttaggttgtgacagttggaagtgtcatgtacaacatgcggcgattaagtctttcacccaccttttcaatgggatttcatctgttagttactgtgagtctcttattttcccatgtggaccatgtaattgctgagacagaaatggaaggagaaggaaatgaaactggtgaatgtactggatcatattactgtaagaaaggggtgattttgcccatttgggaaccccaagacccttcttttggggacaaaattgctagagctactgtgtattttgtggccatggtctacatgtttcttggagtctctatcatagctgatcggttcatgtcctctatagaagtcatcacatctcaagaaaaagaaataaccataaagaaacccaatggagagaccaccaagacaactgtgaggatctggaatgaaacagtttctaacctgaccttgatggccctgggatcttctgctcctgagattctcctttcagtaattgaagtgtgtggccataacttcactgcaggagacctcggtcctagcaccatcgtgggaagtgctgcattcaatatgttcatcattattgcactctgtgtttatgtggtgcctgacggagagacaaggaagattaagcatttgcgtgtcttctttgtgacagcagcctggagcatctttgcctacacctggctttacattattttgtctgtcatatctcctggtgttgtggaggtctgggaaggtttgcttactttcttcttctttcccatctgtgttgtgttcgcttgggtagcggataggagacttctgttttacaagtatgtctacaagaggtatcgagctggcaagcagagggggatgattattgaacatgaaggagacaggccatcttctaagactgaaattgaaatggacgggaaagtggtcaattctcatgttgaaaatttcttagatggtgctctggttctggaggtggatgagagggaccaagatgatgaagaagctaggcgagaaatggctaggattctgaaggaacttaagcagaagcatccagataaagaaatagagcaattaatagaattagctaactaccaagtcctaagtcagcagcaaaaaagtagagcattttatcgcattcaagctactcgcctcatgactggagctggcaacattttaaagaggcatgcagctgaccaagcaaggaaggctgtcagcatgcacgaggtcaacactgaagtgactgaaaatgaccctgttagtaagatcttctttgaacaagggacatatcagtgtctggagaactgtggtactgtggcccttaccattatccgcagaggtggtgatttgactaacactgtgtttgttgacttcagaacagaggatggcacagcaaatgctgggtctgattatgaatttactgaaggaactgtggtgtttaagcctggtgatacccagaaggaaatcagagtgggtatcatagatgatgatatctttgaggaggatgaaaatttccttgtgcatctcagcaatgtcaaagtatcttctgaagcttcagaagatggcatactggaagccaatcatgtttctacacttgcttgcctcggatctccctccactgccactgtaactatttttgatgatgaccacgcaggcatttttacttttgaggaacctgtgactcatgtgagtgagagcattggcatcatggaggtgaaagtattgagaacatctggagctcgaggaaatgttatcgttccatataaaaccatcgaagggactgccagaggtggaggggaggattttgaggacacttgtggagagctcgaattccagaatgatgaaattgtcaaaacaatatcagtcaaggtaattgatgatgaggagtatgagaaaaacaagaccttcttccttgagattggagagccccgcctggtggagatgagtgagaagaaagccctgttattgaatgagcttggtggcttcacaataacaggaaaatacctgtttggccaacctgtcttcaggaaggttcatgctagagaacatccgattctctctactgtaatcaccattgcagacgaatatgatgacaagcagccactgaccagcaaagaggaagaggagaggcgcattgcagaaatggggcgccccatcctgggagagcacaccaagttggaagtgatcattgaagaatcctatgaattcaagagtactgtggacaaactcattaagaagacaaacctggcccttgtggttgggactaacagctggagagaacagttcattgaagctatcactgtcagtgctggggaagatgatgacgacgatgaatgtggggaagagaagctgccctcctgtttcgattacgtgatgcactttctgactgtgttctggaaggtcctgtttgccttcgtcccccctactgaatactggaatggctgggcgtgtttcattgtctccatcctcatgattggcctactgacagctttcattggagacctggcttcccactttggctgcaccattggcctgaaagattctgtgactgcagtcgtgttcgtcgcacttggaacatcagtgccagacacatttgccagcaaagtggcagccacccaggaccagtatgcagacgcctccataggtaacgtcacgggcagcaacgcggtgaatgtcttcctgggaatcggtgtggcctggtccatcgctgccatctaccacgcagccaatggggaacagttcaaagtgtcccctggcacactagctttctctgtcactctcttcaccatttttgctttcatcaatgtgggggtgctgctgtatcggcggaggccagaaatcggaggtgagctgggtgggccccggactgccaagctcctcacatcctgcctctttgtgctcctatggctcttgtacattttcttctcctccctggaggcctactgccacataaaaggcttctaaaggaactatcagatatagtaaatttatatatatacatatatatacataaaaattatgtataatggacagaggaaactgacatttgtcatgttcacttacctgctgatggaatccagcttcaagagcatactctgtactagggccgaagtaaaaaaccatcacctcccattcccaggggcatcatcatgttcaacaaggcatggaggcagggccatctttgcagctcagtctagaagggctgcactctc

SEQ ID NO: 14 - protein, 973 aa, Homo sapiens
MYNMRRLSLSPTFSMGFHLLVTVSLLFSHVDHVIAETEMEGEGNETGECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSTIVGSAAFNMFIIIALCVYVVPDGETRKIKHLRVFFVTAAWSIFAYTWLYIILSVISPGVVEVWEGLLTFFFFPICVVFAWVADRRLLFYKYVYKRYRAGKQRGMIIEHEGDRPSSKTEIEMDGKVVNSHVENFLDGALVLEVDERDQDDEEARREMARILKELKQKHPDKEIEQLIELANYQVLSQQQKSRAFYRIQATRLMTGAGNILKRHAADQARKAVSMHEVNTEVTENDPVSKIFFEQGTYQCLENCGTVALTIIRRGGDLTNTVFVDFRTEDGTANAGSDYEFTEGTVVFKPGDTQKEIRVGIIDDDIFEEDENFLVHLSNVKVSSEASEDGILEANHVSTLACLGSPSTATVTIFDDDHAGIFTFEEPVTHVSESIGIMEVKVLRTSGARGNVIVPYKTIEGTARGGGEDFEDTCGELEFQNDEIVKTISVKVIDDEEYEKNKTFFLEIGEPRLVEMSEKKALLLNELGGFTITGKYLFGQPVFRKVHAREHPILSTVITIADEYDDKQPLTSKEEEERRIAEMGRPILGEHTKLEVIIEESYEFKSTVDKLIKKTNLALVVGTNSWREQFIEAITVSAGEDDDDDECGEEKLPSCFDYVMHFLTVFWKVLFAFVPPTEYWNGWACFIVSILMIGLLTAFIGDLASHFGCTIGLKDSVTAVVFVALGTSVPDTFASKVAATQDQYADASIGNVTGSNAVNVFLGIGVAWSIAAIYHAANGEQFKVSPGTLAFSVTLFTIFAFINVGVLLYRRRPEIGGELGGPRTAKLLTSCLFVLLWLLYIFFSSLEAYCHIKGF

SEQ ID NO: 15 - DNA, 3344 bp, Mus musculus
ggatcgggcaggtcagccttcagagctggtcggtttctcatccagcccatgcagacagagacggcttgacagaggttggagcagttggaagtcttattgtacaacatgcttcgattaagtctcccacccaatgtttcaatgggatttcgtctggtagctctggtggctctcttgttttcccatgttgaccatataactgcagatacagaggcagaaacaggaggaaatgaaaccactgaatgtactggctcatattactgtaagaaaggggtgatcttgcccatttgggaaccccaagacccatcttttggggacaaaattgctagagcaactgtgtattttgtggccatggtctacatgttccttggagtttctattattgcagaccggtttatgtcctctatagaggtcatcacctctcaagagaaagaaataacgataaagaaaccgaatggagagaccaccaagacgacggtgagaatctggaacgagactgtgtcgaacctgaccttgatggccctgggatcttctgctcctgagattctcctgtcagtcattgaagtgtgcggccataacttcaccgcaggggacctgggtcccagcaggatcgtgggaagtgctgcctttaacatgttcatcataatcgcactctgtgtttacgtggtccctgatggagagacaaggaagatcaagcatctgcgtgtgttctttgtgacagcagcctggagcatctttgcctatacctggctttatataatcttgtctgtcagctctcctggagttgtggaggtctgggaaggcttgcttactttcttcttctttcccatctgcgttgtgttcgcgtgggtagcagacaggcggcttctcttttacaagtatgtctacaagcggtacagggccggcaagcagagggggatgatcattgaacatgaaggagacagaccagcttccaaaactgaaatcgaaatggatgggaaagtggtcaactctcatgttgacaatttcttagatggggctctggttttggaagttgatgagagggaccaagatgatgaggaagccaggcgtgagatggcaaggattctgaaggaacttaagcagaagcatcctgagaaagaaattgagcaattaatagaattagccaactaccaggtcctaagtcaacagcagaaaagccgagcattttacaggattcaagctactcgcctgatgaccggagctggcaacatcttgaagaggcacgcagctgatcaagcaaggaaggctgtcagtatgcatgaagtcaacatggaaatggctgaaaacgacccagtcagtaagatcttctttgagcaaggaacataccagtgtctagagaactgtggtactgtggccctcaccattatgcgcagagggggcgacttgagcaccactgtgtttgttgacttcaggacagaagacggcacagccaatgctgggtctgattatgaattcacggaagggactgtgatcttcaaaccaggggagacccagaaggaaatcagagttggcatcattgatgatgatatctttgaagaagatgaaaacttccttgtgcatcttagcaatgtcagagtctcttcagatgtttcagaagatggcatactagaatccaatcacgcttcttcaattgcttgtcttgggtcacccagcactgccaccataaccatttttgataatgaccatacaggcatctttacatttgaggaacccgtgactcacgtgagcgagagcattggcatcatggaggtgaaggttttgagaacctctggagctcgaggaaatgttatcattccctacaaaactattgaaggcacagcccgaggtggaggggaagactttgaggacacctgtggagtgccggaattccagaatgatgaaatagtcaaaacaatatcagtcaaggtaataatcgatgacgaggagtatgagaaaaacaagaccttcattgagattggagagccccgtctggtggagatgagtgagaagaaagccctgttgttgaatgagcttggtggcttcacattaacaggaaaagagatgtatggccaacctatcttcaggaaggtccatgctagagatcatccgattccctctactgtaatcaccatctcagaggaatatgatgacaagcagccactgaccagcaaagaagaccaggagaggcgcattgcggaaatggggcgccccatcctaggcgagcacaccaagctggaggtgatcatcgaagagtcttacgaattcaagagcactgtggacaaactcattaagaagacgaacctggcccttgtggtggggaccaacagctggagagagcagtttatcgaagccatcactgtcagcgctggggaacgtgacgatgatgatgaatatggggaggagaagctgccctcctgttttgattacgtgatgcactttctcacagtgttctggaaggttctgtttgccttcgtcccacctacagaatactggaatggctgggcctgcttcattgtctccatcctcatgatcggcctactgaccgccttcattggagacctggcttcccactttggctgcaccattggtctgaaagattccgtgactgccgttgtgtttgttgctcttggaacctcggtgccagacacatttgccagcaaagtagcagctacccaggaccagtatgcagatgcgtctataggcaatgtcactggaagcaatgctgtgaatgtcttcctgggaatcggcgtggcctggtccattgctgccatctgccatgcggccaacggggaacagttcaaagtgtcccccggcacgctagctttctctgtcactctcttcactatttttgctttcatcaacgtgggggtgctgctgtatcggcggaggccagaaataggagccatcttcggaatgtcaataattctgtagtgaatttcacagagaacttcccaagaacgtcttgctgttggtcagtttcaagttcaagctttttggtgtgtttgccttttcagtcttcatgctgtagggaaaatctgatctggatcttcagggttcttaattattatgtgtaaatgatttcatttgatttgtttgattatttttattttgtgtgtattgtgcacggctttagggggacaggcacactcggtgctgttcctgacaattggtacacattattgacacagaaaacaaaaacaaaaatcaataaagtttctaactgtttgtgatttcatcaccagtggcacagcatgttctgtactgtggaatgtccctctcctcactgtcacctacttcattttcagttgtttgttttcatgctctgaagaaataaagaccaaaaaggc

SEQ ID NO: 16 - protein, 940 aa, Mus musculus
MLRLSLPPNVSMGFRLVALVALLFSHVDHITADTEAETGGNETTECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSRIVGSAAFNMFIIIALCVYVVPDGETRKIKHLRVFFVTAAWSIFAYTWLYIILSVSSPGVVEVWEGLLTFFFFPICVVFAWVADRRLLFYKYVYKRYRAGKQRGMIIEHEGDRPASKTEIEMDGKVVNSHVDNFLDGALVLEVDERDQDDEEARREMARILKELKQKHPEKEIEQLIELANYQVLSQQQKSRAFYRIQATRLMTGAGNILKRHAADQARKAVSMHEVNMEMAENDPVSKIFFEQGTYQCLENCGTVALTIMRRGGDLSTTVFVDFRTEDGTANAGSDYEFTEGTVIFKPGETQKEIRVGIIDDDIFEEDENFLVHLSNVRVSSDVSEDGILESNHASSIACLGSPSTATITIFDNDHTGIFTFEEPVTHVSESIGIMEVKVLRTSGARGNVIIPYKTIEGTARGGGEDFEDTCGVPEFQNDEIVKTISVKVIIDDEEYEKNKTFIEIGEPRLVEMSEKKALLLNELGGFTLTGKEMYGQPIFRKVHARDHPIPSTVITISEEYDDKQPLTSKEDQERRIAEMGRPILGEHTKLEVIIEESYEFKSTVDKLIKKTNLALVVGTNSWREQFIEAITVSAGERDDDDEYGEEKLPSCFDYVMHFLTVFWKVLFAFVPPTEYWNGWACFIVSILMIGLLTAFIGDLASHFGCTIGLKDSVTAVVFVALGTSVPDTFASKVAATQDQYADASIGNVTGSNAVNVFLGIGVAWSIAAICHAANGEQFKVSPGTLAFSVTLFTIFAFINVGVLLYRRRPEIGAIFGMSIIL

SEQ ID NO: 17 - DNA, 3262 bp, Homo sapiens
gcggcggggctggcagcagtggctgcccgcactgcgcccgggcgctcgccttcgctgcagctcccggtgccgccgctcgggccggccccccggcaggccctcctcgttatggccgcggcctcctccccgcccagggccgagaggaagcgctggggttggggccgcctgccaggcgcccggcggggcagcgcgggcctggccaagaagtgccccttctcgctggagctggcggagggcggcccggcgggcggcgcgctctacgcgcccatcgcgcccggcgccccaggtcccgcgccccctgcgtccccggccgcgcccgccgcgcccccagttgcctccgaccttggcccgcggccgccggtgagcctagacccgcgcgtctccatctacagcacgcgccgcccggtgttggcgcgcacccacgtccagggccgcgtctacaacttcctcgagcgtcccaccggctggaaatgcttcgtttaccacttcgccgtcttcctcatcgtcctggtctgcctcatcttcagcgtgctgtccaccatcgagcagtatgccgccctggccacggggactctcttctggatggagatcgtgctggtggtgttcttcgggacggagtacgtggtccgcctctggtccgccggctgccgcagcaagtacgtgggcctctgggggcggctgcgctttgcccggaagcccatttccatcatcgacctcatcgtggtcgtggcctccatggtggtcctctgcgtgggctccaaggggcaggtgtttgccacgtcggccatcaggggcatccgcttcctgcagatcctgaggatgctacacgtcgaccgccagggaggcacctggaggctcctgggctccgtggtcttcatccaccgccaggagctgataaccaccctgtacatcggcttcctgggcctcatcttctcctcgtactttgtgtacctggctgagaaggacgcggtgaacgagtcaggccgcgtggagttcggcagctacgcagatgcgctgtggtggggggtggtcacagtcaccaccatcggctatggggacaaggtgccccagacgtgggtcgggaagaccatcgcctcctgcttctctgtctttgccatctccttctttgcgctcccagcggggattcttggctcggggtttgccctgaaggtgcagcagaagcagaggcagaagcacttcaaccggcagatcccggcggcagcctcactcattcagaccgcatggaggtgctatgctgccgagaaccccgactcctccacctggaagatctacatccggaaggccccccggagccacactctgctgtcacccagccccaaacccaagaagtctgtggtggtaaagaaaaaaaagttcaagctggacaaagacaatggggtgactcctggagagaagatgctcacagtcccccatatcacgtgcgaccccccagaagagcggcggctggaccacttctctgtcgacggctatgacagttctgtaaggaagagcccaacactgctggaagtgagcatgccccatttcatgagaaccaacagcttcgccgaggacctggacctggaaggggagactctgctgacacccatcacccacatctcacagctgcgggaacaccatcgggccaccattaaggtcattcgacgcatgcagtactttgtggccaagaagaaattccagcaagcgcggaagccttacgatgtgcgggacgtcattgagcagtactcgcagggccacctcaacctcatggtgcgcatcaaggagctgcagaggaggctggaccagtccattgggaagccctcactgttcatctccgtctcagaaaagagcaaggatcgcggcagcaacacgatcggcgcccgcctgaaccgagtagaagacaaggtgacgcagctggaccagaggctggcactcatcaccgacatgcttcaccagctgctctccttgcacggtggcagcacccccggcagcggcggcccccccagagagggcggggcccacatcacccagccctgcggcagtggcggctccgtcgaccctgagctcttcctgcccagcaacaccctgcccacctacgagcagctgaccgtgcccaggaggggccccgatgaggggtcctgaggaggggatggggctgggggatgggcctgagtgagaggggaggccaagagtggccccacctggccctctctgaaggaggccacctcctaaaaggcccagagagaagagccccactctcagaggccccaataccccatggaccatgctgtctggcacagcctgcacttgggggctcagcaaggccacctcttcctggccggtgtgggggccccgtctcaggtctgagttgttaccccaagcgccctggcccccacatggtgatgttgacatcactggcatggtggttgggacccagtggcagggcacagggcctggcccatgtatggccaggaagtagcacaggctgagtgcaggcccaccctgcttggcccagggggcttcctgaggggagacagagcaacccctggaccccagcctcaaatccaggaccctgccaggcacaggcagggcaggaccagcccacgctgactacagggccgccggcaataaaagcccaggagcccatttggagggcctgggcctggctccctcactctcaggaaatgctgacccatgggcaggagactgtggagactgctcctgagcccccagcttccagcaggagggacagtctcaccatttccccagggcacgtggttgagtggggggaacgcccacttccctgggttagactgccagctcttcctagctggagaggagccctgcctctccgcccctgagcccactgtgcgtggggctcccgcctccaacccctcgcccagtcccagcagccagccaaacacacagaaggggactgccacctccccttgccagctgctgagccgcagagaagtgacggttcctacacaggacaggggttccttctgggcattacatcgcatagaaatcaataatttgtggtgatttggatctgtgttttaatgagtttcacagtgtgattttgattattaattgtgcaagcttttcctaataaacgtggagaatcacaggctgggctgggcactgctctcaccttggttcctggggcatccatggggtctctcacagacaggacccctgcagttcccctggaagcagtgcccaggtggctgtggaataggaacgctaaaaaaaaaaaaaaaaa

SEQ ID NO: 18 - protein, 676 aa, Homo sapiens [sequence omitted]

SEQ ID NO: 19 - DNA, 3052 bp, Mus musculus
cccgtggctgggctggcagcagtggctacccgcgctgcgccctgcgctctgcgctgccttcacctcagctccgaggagcagcggggtcaggggtcctgtctggccatggacacggcctcgtccccgcccagtgctgaaaggaagcgcgcgggttggagccgcctgctaggcgcccggcggggcagcgcggtggtcaagaagtgtcccttctcactggagctggccgaaggtggccctgagggcagcacggtctatgcgcccatcgcgccaaccggagcccccgggctcgcgccccccatgtcgaccccagtgtcgcccgccccggcccctgcagacctcggcccacgtccgcgggtgagccttgacccgcgggtctccatctacagtgcgcgccgcccgctgctggcgcgcacccacatccagggccgagtctacaacttcctcgagcgccccacgggttggaagtgtttcgtgtaccacttcaccgtcttcctcattgttctggtctgcctcatcttcagtgtcctgtccactattgagcagtatgccgctctggccaccgggaccctcttctggatggagattgtccttgtggtgttctttgggacagaatatgtggtccgcctctggtctgcaggctgccgcagcaagtacgtgggcatctggggccggctacgttttgcccggaagcccatttccatcattgacctcatcgtggttgtagcctctatggttgtcctctgcgtgggttccaaaggacaagtgttcgccacatcagctatcaggggtatccgcttccttcagatcctgcggatgctgcatgtcgatcgccaggggggtacctggaggctcctgggctctgtagtcttcattcaccgccaggagctgatcaccaccctgtacattggctttctgggccttatcttctcctcctactttgtctacttggctgagaaagatgcggtgaacgagtccggccgcatcgagtttggcagctacgcagatgctctgtggtggggggtggtcacagtcactaccattggctacggggataaggtacctcagacgtgggttgggaagaccatcgcctcctgtttctctgtcttcgccatatccttctttgcactcccagcggggatacttggctctgggttcgcgctgaaggtccagcagaagcagaggcagaagcacttcaaccggcagatcccagctgcagcctcactcatccagactgcatggaggtgctatgccgctgagaaccctgactcagccacttggaagatctatgtccggaagcctgctcggagtcacacgcttctgtcccccagccccaaacctaaaaagtctgtcatggtaaagaagaagaagttcaagctggataaggataatgggatgagtcctggagagaagatgttcaatgttcctcacatcacttatgatcccccagaggataggaggccagaccatttctccattgatggctatgacagctcagtaaggaagagccctacactgctggaagtaagcacaccccatttcttgagaacaaacagctttgcagaggacctggacctggaaggggagacactgctgacccccatcacccatgtgtcacagctgcgggatcaccatcgggccaccatcaaggtcatcaggcgcatgcagtactttgtagccaagaagaaattccagcaagcacggaagccctacgacgtgcgagatgtcatcgagcagtactcccagggccacctgaaccttatggtgcgcattaaagaactacagagaaggctggatcagtccattgggaagccatctttgttcatccccatctcagaaaagagcaaagaccgtggcagtaacaccatcggtgcccgtctgaacagggtggaagacaaggtgacacaactggaccagagactggtgatcatcacagacatgctccaccagctgctgtccatgcaacaaggtggtccaacctgcaacagcaggtcacaagttgtagccagcaatgaaggtggctccatcaaccctgagctcttcctacccagcaacagcctgcccacctacgaacaactgactgtgccccagacaggccctgatgagggttcctgaaggagctcagaagggaggtccagggtaaaccccccacaggtctgccccacctccctctcaacaggggcacacccacctggttctcacccccttgaaggcctgatatgacagcctagctccctaaggccccgaaacccatgggccaagccactggcctggatctggatatcaccaacacccctctgcccagccagtgggaactagaacttgtctgggcatggggttcctctcaggcccctatcaagatgttgatgctgctcttctgatggctggaacatagggatggggagtatatgtgtaaagcaggatgcagctcaggcagagggaagcccatccaggtatgccaagtggcttcctaaagggaaacaggaagagtctggggtttccaccctcagccctgaatccagagaccatcagggaacgtgcagggcaagacaggcccggcccacactaactgcaggctgtagcccagtgggagctcagcaggctagagcctgactccttcattctctagtgatgttgaccacaacaagatactagcgggattttcacaccccccccaaaggatatcccaagcaagggattgtccactgtccaccctagccaggtatggactgacaacttctcccctaaagtgaaaagcagggccctcaagccctatgagccccacatgtgcagcctgcctccttcttgctggtcagaggtgctggaccaatgctcagactccagcctctttcacgccacattgaagttaggggcatcaccgattcttccaggattggttccttgggcttgggcaccacgcctcacagaaatcaagataatttctggtgattcagactcttgtgttttaatgaatttcgtagtatgatcttgattgagagacttttccaaataaagttgggacgtcccaggttaaaaaaaaaaaaaaaaaaaaaaa

SEQ ID NO: 20 - protein, 668 aa, Mus musculus
MDTASSPPSAERKRAGWSRLLGARRGSAVVKKCPFSLELAEGGPEGSTVYAPIAPTGAPGLAPPMSTPVSPAPAPADLGPRPRVSLDPRVSIYSARRPLLARTHIQGRVYNFLERPTGWKCFVYHFTVFLIVLVCLIFSVLSTIEQYAALATGTLFWMEIVLVVFFGTEYVVRLWSAGCRSKYVGIWGRLRFARKPISIIDLIVVVASMVVLCVGSKGQVFATSAIRGIRFLQILRMLHVDRQGGTWRLLGSVVFIHRQELITTLYIGFLGLIFSSYFVYLAEKDAVNESGRIEFGSYADALWWGVVTVTTIGYGDKVPQTWVGKTIASCFSVFAISFFALPAGILGSGFALKVQQKQRQKHFNRQIPAAASLIQTAWRCYAAENPDSATWKIYVRKPARSHTLLSPSPKPKKSVMVKKKKFKLDKDNGMSPGEKMFNVPHITYDPPEDRRPDHFSIDGYDSSVRKSPTLLEVSTPHFLRTNSFAEDLDLEGETLLTPITHVSQLRDHHRATIKVIRRMQYFVAKKKFQQARKPYDVRDVIEQYSQGHLNLMVRIKELQRRLDQSIGKPSLFIPISEKSKDRGSNTIGARLNRVEDKVTQLDQRLVIITDMLHQLLSMQQGGPTCNSRSQVVASNEGGSINPELFLPSNSLPTYEQLTVPQTGPDEGS

SEQ ID NO: 21 - DNA, 3900 bp, Homo sapiens
ccatgggctcaggatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagaaaa

SEQ ID NO: 22 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 23 - DNA, 4235 bp, Mus musculus
gaattcccgggagcccggcgctggagcgggcggagcgcggcgcggggacgaggtgcagccgggcccgtggcaccaccagcccgggtcgacagacaggtgtgccggcgccgcagctccagcagcttcaggcccggcccagggtccggagccggcagccactaagcagagcgggaatcccgcgccctcggtccagttccggccccgcccgcactcagcttggccgcggggtgcggagaccacggcccggccaggccacccgaagcctagtgctgggccgggccgggccagggtgggtgggggcccgcccggcccgcccatgggctcaggatgccggtgcggaggggccacgtcgcgccgcagaacaccttcctcgacaccatcatccgcaagtttgagggccagagccgcaagttcatcatcgctaacgcgcgcgtagagaactgcgctgtcatctactgcaacgacggcttctgcgaactgtgtggctactcgcgggccgaggtgatgcagcggccctgcacctgcgatttcctgcatgggccgcgcacgcagcgccgtgccgccgcgcagatcgcgcaggccctgctgggcgcagaggagcgcaaagtggagatcgccttctaccggaaagatgggagctgcttcctgtgtctggtggatgtggtacccgtgaagaatgaagatggggctgtgatcatgttcatcctcaactttgaagtagtgatggagaaggacatggtagggtccccggctcatgacaccaaccacaggggcccctctaccagctggctagcttctggccgggccaagactttccgcctgaagctgcctgccttgttggcactgacggccagggaatcatctgtgcggacaggaagcatgcgcagtgctggagcccctggggcggtggtggtggatgtggacctgacgccggcagcacccagcagtgagtccctggccttggatgaggtttctgccatggacaaccatgtggcagggcttgggcctgcagaagagaggcgagcactggtgggcccggggtctgcttcaccagtagccagcatccgaggccctcacccatcgccacgagctcagagccttaaccctgatgcctcaggttccagctgcagcctggcccggacacgctcccgtgagagctgcgctagcgtgcgccgcgcatcatccgcggatgacattgaggcaatgcgggctggagcgctgccccctccgccccgccatgcaagcacaggggccatgcaccccctgcgcagtggcctgcttaactccacctcagactctgaccttgtacgctaccgaaccattagcaagataccccaaatcactctcaactttgtggacctcaaaggcgaccctttcctggcttcacccaccagtgaccgggagattatagcacccaagataaaagagcggacccacaatgtcaccgagaaggtcacccaggtcctgtccttgggagcagatgtgctgcctgagtataagctgcaggccccaagaatccaccgctggaccatcctccactacagccccttcaaggcggtgtgggactggctcatcctgctgctggtcatctacacggcagtcttcacaccgtactcggccgccttcctgctgaaggagactgaagatgggtcccaagcccctgactgtggctacgcctgccagcccctgactgtagtggacctcatcgtggacatcatgttcattgtggacatccttatcaatttccgtaccacctatgttaatgccaacgaggaggtggttagccatcctggccgcattgctgtgcactacttcaagggctggtttctcattgacatggtggctgccatccccttcgacctgctcatctttggctctggctccgaggagctgatcgggctgctgaaaactgcgaggctgctgcggctggtgcgcgtggctcggaagctggaccgctactcggagtacggggcagcagtgctcttcctgctcatgtgcaccttcgccctcattgcgcactggttggcctgcatctggtacgcgatcggcaacatggaacagcctcacatggactcacacatcggctggctgcacaacttgggcgaccagatcggcaagccctacaacagcagcggcctgggaggcccctccatcaaggacaagtatgtcacagcgctctacttcaccttcagcagcctcaccagcgtgggctttggcaatgtctctcccaacaccaactcagagaagatcttctccatctgtgtcatgctcattggctccctcatgtatgccagcatcttcggcaacgtgtccgccatcatccagcggctgtactcgggcacggcccgctaccacacacagatgctccgggtgcgggagtttatccgctttcaccagattcccaacccattacgccagcgcctcgaggagtacttccagcatgcttggtcctacaccaatggcatcgacatgaacgcggtgctgaagggcttccctgagtgcctccaggctgacatctgcctacacctgaaccgctcgctgctgcagcattgcaagccattccgaggggccactaaaggctacctgagggcgctggctatgaagttcaagaccacacatgcaccaccaggggacacactagtgcacgccggggacctgcttactgccctctacttcatctccaggggctccatcgagatcctgcggggtgatgtcgtggtggccatcttggggaagaacgacatctttggagagcctctgaacctatatgcccgtcctggaaagtccaatggggatgtgcgggccctcacgtactgtgacctgcacaagatccatcgtgatgacttgctggaggtgttggacatgtatcccgagttctcagatcacttttggtctagcctggagatcaccttcaaccttcgagataccaacatgattcctggctccccaggcagtgcagaactagagagcggctttaacaggcaacgcaagcgcaaactgtcattccgcaggcgtacagacaaggacacagagcagccaggggaggtgtcagccctggggcaaggccctgcccgagttgggccagggccgagttgccggggacagccgggagggccatggggggagagcccatccagcggcccttccagcccagagagcagtgaagatgagggccctggtcgcagctccagtcccctccgcctggtgcccttctccagccccaggcctccgggagaccccccaggtggggagcctttgacagaggatggggagaaaagtgacacctgtaatcccttgtcaggtgccttctctggggtgtccaatattttcagcttctgggggaacagtcgggggcgccagtaccaggagttgcctcgatgccctgcccctgcccccagcctcctcaacatccccttgtctagccctggtcggcgatcccggggtgatgtggagagcaggctggacgcactccagagacagctgaacaggctggaaacccggctaagtgcagacatggccactgtcctacagctgctacagaggcaaatgaccctggtccctcctgcctacagtgctgtaaccacccctgggcccggccccacttccgcatcccctttgttgcctgtcggccctgtccccactctcaccctggactcgctttctcaggtttcccagttcgtggcgtttgaggagcttcctgcaggagccccagagctcccccaagatggccccactcgacgcctctccctgcccggccagctgggggcccttacctcccagcccctgcacagacatggctcagatccaggcagttagtgatgctgcccactgtggacatgtggctcaccctgggttcagggcattacctgaccgctctccttggagaccctggtttggaggggagacctgtgcagaagcacctccatccccacccttgggaccagcatctcctcctgcaataccctgggccccactgggaggggcaggacagagctgggcagtggatgggggtctgtggtccccccactgccctgggggcattagctggtctaactgcccggaggcaccggccctgggccttaggcacctcaaggacttttctgctatttactgctcttattgttaaggataataattaaggatcatatgaataattaatgaagatgctgatgactatgaataataaataattatcctgaggagactccagggtgctggttagcttcaaaaaaaaa

SEQ ID NO: 24 - protein, 1162 aa, Mus musculus
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPSTSWLASGRAKTFRLKLPALLALTARESSVRTGSMRSAGAPGAVVVDVDLTPAAPSSESLALDEVSAMDNHVAGLGPAEERRALVGPGSASPVASIRGPHPSPRAQSLNPDASGSSCSLARTRSRESCASVRRASSADDIEAMRAGALPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEDGSQAPDCGYACQPLTVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSHIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGYLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSAELESGFNRQRKRKLSFRRRTDKDTEQPGEVSALGQGPARVGPGPSCRGQPGGPWGESPSSGPSSPESSEDEGPGRSSSPLRLVPFSSPRPPGDPPGGEPLTEDGEKSDTCNPLSGAFSGVSNIFSFWGNSRGRQYQELPRCPAPAPSLLNIPLSSPGRRSRGDVESRLDALQRQLNRLETRLSADMATVLQLLQRQMTLVPPAYSAVTTPGPGPTSASPLLPVGPVPTLTLDSLSQVSQFVAFEELPAGAPELPQDGPTRRLSLPGQLGALTSQPLHRHGSDPGS

SEQ ID NO: 25 - DNA, 1901 bp, Homo sapiens
gggcaggaagacggcgctgcccggaggagcggggcgggcgggcgcgcgggggagcgggcggcgggcgggagccaggcccgggcgggggcgggggcggcggggccagaagaggcggcgggccgcgctccggccggtctgcggcgttggccttggctttggctttggcggcggcggtggagaagatgctgcagtccctggccggcagctcgtgcgtgcgcctggtggagcggcaccgctcggcctggtgcttcggcttcctggtgctgggctacttgctctacctggtcttcggcgcagtggtcttctcctcggtggagctgccctatgaggacctgctgcgccaggagctgcgcaagctgaagcgacgcttcttggaggagcacgagtgcctgtctgagcagcagctggagcagttcctgggccgggtgctggaggccagcaactacggcgtgtcggtgctcagcaacgcctcgggcaactggaactgggacttcacctccgcgctcttcttcgccagcaccgtgctctccaccacaggttatggccacaccgtgcccttgtcagatggaggtaaggccttctgcatcatctactccgtcattggcattcccttcaccctcctgttcctgacggctgtggtccagcgcatcaccgtgcacgtcacccgcaggccggtcctctacttccacatccgctggggcttctccaagcaggtggtggccatcgtccatgccgtgctccttgggtttgtcactgtgtcctgcttcttcttcatcccggccgctgtcttctcagtcctggaggatgactggaacttcctggaatccttttatttttgttttatttccctgagcaccattggcctgggggattatgtgcctggggaaggctacaatcaaaaattcagagagctctataagattgggatcacgtgttacctgctacttggccttattgccatgttggtagttctggaaaccttctgtgaactccatgagctgaaaaaattcagaaaaatgttctatgtgaagaaggacaaggacgaggatcaggtgcacatcatagagcatgaccaactgtccttctcctcgatcacagaccaggcagctggcatgaaagaggaccagaagcaaaatgagccttttgtggccacccagtcatctgcctgcgtggatggccctgcaaaccattgagcgtaggatttgttgcattatgctagagcaccagggtcagggtgcaaggaagaggcttaagtatgttcatttttatcagaatgcaaaagcgaaaattatgtcactttaagaaatagctactgtttgcaatgtcttattaaaaaacaacaaaaaaagacacatggaacaaagaagctgtgaccccagcaggatgtctaatatgtgaggaaatgagatgtccacctaaaattcatatgtgacaaaattatctcgaccttacataggaggagaatacttgaagcagtatgctgctgtggttagaagcagattttatacttttaactggaaactttggggtttgcatttagatcatttagctgatggctaaatagcaaaatttatatttagaagcaaaaaaaaaaagcatagagatgtgttttataaataggtttatgtgtactggtttgcatgtacccacccaaaatgattatttttggagaatctaagtcaaactcactatttataatgcataggtaaccattaactatgtacatataaagtataaatatgtttatattctgtacatatggtttaggtcaccagatcctagtgtagttctgaaactaagactatagatattttgtttcttttgatttctctttatactaaagaatccagagttgctacaataaaataaggggaataataaacttgagagtgaataaccat

SEQ ID NO: 26 - protein, 336 aa, Homo sapiens
MLQSLAGSSCVRLVERHRSAWCFGFLVLGYLLYLVFGAVVFSSVELPYEDLLRQELRKLKRRFLEEHECLSEQQLEQFLGRVLEASNYGVSVLSNASGNWNWDFTSALFFASTVLSTTGYGHTVPLSDGGKAFCIIYSVIGIPFTLLFLTAVVQRITVHVTRRPVLYFHIRWGFSKQVVAIVHAVLLGFVTVSCFFFIPAAVFSVLEDDWNFLESFYFCFISLSTIGLGDYVPGEGYNQKFRELYKIGITCYLLLGLIAMLVVLETFCELHELKKFRKMFYVKKDKDEDQVHIIEHDQLSFSSITDQAAGMKEDQKQNEPFVATQSSACVDGPANH

SEQ ID NO: 27 - DNA, 1589 bp, Xenopus tropicalis
caaggagtgaggcagccaataacgactttgagggcaaagggcagacatacaccttgctgaacacaccagcaactcccacggcaaacacaagtggtcctttctctgtggggccgccagtcgttgcttgttgtgacctttgctacctgagaagggaggaaaccgacgtcacaatgtacaggagacactgccacatcagctctccgttcccagagagacgctatgttcccctcgcagagccgtgaattttccataacataatcaggaaatgatgctccaagccctggccaccaacccctgcctgcggctgatccaacggaaccagacatgctggtgctttgtcctcttgatgttgggctacctgctcttccttctgataggggcagccatattctcagcggtggagctgccccacgaacacgttctccgggaggagctgctggacttaaagcatcgctacctgcaggagaatgagtgcctgagcgaggagaggctggagagcttcctcagccgagtactggaggccagcaattacggggtgtccatgctgaacaatgtctctgggaaccccaactgggacttcacctctgccctgttctttgttagcacggtgctctccactacagggtatgggcacacagtgccgctctctaacgcagggaagaccttctgtatcatttactctatcatcgggatccctctgacactgctcctgttcactgcgctggtgcagcggatcatggtgcacgtgacccacaggcccatttcctacttccacctccgctgggggtacaacaagcagactgtggccgtcgttcacgcgctggtcattgggtttgtggccattttgtgctttttcctcataccggccgcaattttctcggctctcgaagatgattggaatttcctagagtcgttttacttctgcttcatttctttgagcaccatcggcctgggagactatgtgcccgcggaggggcaaaaccagaggtaccgccagctgtacaagttcggcatcacttgttacctgatattgggcctcattgtgatgcttgtggttctggagactttctgtgagctgcaggggctgaagaaattccgaaaaatgttctacaggaagaaaatgaaagagggagaccaattaaacatcatagaacacgatcagctgaccctcgcctccatttcagagcaagcggcgtccttaaaggaggaacaaatgctggacgaacactctgtgcccgatggcccactaaatagtgattagtaggacagactggcgctcgttatggatcccattaattatatatactgtctcattcttcattaaaggagagggtctgattaaaaagaacttatggcgtgacaaggacgggggtgttccgatcccatgtgcagttgacctgcattttatttttattgaactttttatttcctagtgatgtctttgtttgagtggttgctagcgtcactttccctagcaacatacctaagggaaatgaatgaataggaactagagcagagaaatgaaagagaaagtgattagcaataaaagcctcaccactaaaaaaaaaaaaaaaaaaaaaaaaaaaagg

SEQ ID NO: 28 - protein, 330 aa, Xenopus tropicalis
MLQALATNPCLRLIQRNQTCWCFVLLMLGYLLFLLIGAAIFSAVELPHEHVLREELLDLKHRYLQENECLSEERLESFLSRVLEASNYGVSMLNNVSGNPNWDFTSALFFVSTVLSTTGYGHTVPLSNAGKTFCIIYSIIGIPLTLLLFTALVQRIMVHVTHRPISYFHLRWGYNKQTVAVVHALVIGFVAILCFFLIPAAIFSALEDDWNFLESFYFCFISLSTIGLGDYVPAEGQNQRYRQLYKFGITCYLILGLIVMLVVLETFCELQGLKKFRKMFYRKKMKEGDQLNIIEHDQLTLASISEQAASLKEEQMLDEHSVPDGPLNSD

SEQ ID NO: 29 - DNA, 5397 bp, Homo sapiens
gcgcactggagccctggccagcgcgcagccttcccggcgccggcgggctgggtcttgggaattctggtttgctttggctcactcgctttttacaaaccactggatcttacatgcctctgtaccccccacttccactccatgtccccatgctcctgcgccagcaacaggacatgttctctggatgtcagctgagtcattaaagtaactctgcatgtcagtagacagaccttggtagaaccacaaggctcccagagacacccatctctcctcatttttttggtgtgtgtgtcttcaccgaacattcaaaactgtttctccaaagcgttttgcaaaaactcagactgttttccaaagcagaagcactggagtccccagcagaagcgatgggcagtgtgcgaaccaaccgctacagcatcgtctcttcagaagaagacggtatgaagttggccaccatggcagttgcaaatggctttgggaacgggaagagtaaagtccacacccgacaacagtgcaggagccgctttgtgaagaaagatggccactgtaatgttcagttcatcaatgtgggtgagaaggggcaacggtacctcgcagacatcttcaccacgtgtgtggacattcgctggcggtggatgctggttatcttctgcctggctttcgtcctgtcatggctgttttttggctgtgtgttttggttgatagctctgctccatggggacctggatgcatccaaagagggcaaagcttgtgtgtccgaggtcaacagcttcacggctgccttcctcttctccattgagacccagacaaccataggctatggtttcagatgtgtcacggatgaatgcccaattgctgttttcatggtggtgttccagtcaatcgtgggctgcatcatcgatgctttcatcattggcgcagtcatggccaagatggcaaagccaaagaagagaaacgagactcttgtcttcagtcacaatgccgtgattgccatgagagacggcaagctgtgtttgatgtggcgagtgggcaatcttcggaaaagccacttggtggaagctcatgttcgagcacagctcctcaaatccagaattacttctgaaggggagtatatccctctggatcaaatagacatcaatgttgggtttgacagtggaatcgatcgtatatttctggtgtccccaatcactatagtccatgaaatagatgaagacagtcctttatatgatttgagtaaacaggacattgacaacgcagactttgaaatcgtggtcatactggaaggcatggtggaagccactgccatgacgacacagtgccgtagctcttatctagcaaatgaaatcctgtggggccaccgctatgagcctgtgctctttgaagagaagcactactacaaagtggactattccaggttccacaaaacttacgaagtccccaacactcccctttgtagtgccagagacttagcagaaaagaaatatatcctctcaaatgcaaattcattttgctatgaaaatgaagttgccctcacaagcaaagaggaagacgacagtgaaaatggagttccagaaagcactagtacggacacgccccctgacatagaccttcacaaccaggcaagtgtacctctagagcccaggcccttacggcgagagtcggagatatgactgactgattccttctctggaatagttactttacaacacggtctgttggtcagaggcccaaaacagttatacagatgacggtactggtcaagatgggtcaagcaagcggccacaagggactgaggcaagcacaatggtttcaaagaaagactgtaagctccatgattagcataaagcactaaccatgtctccatgtgacccgatggcacatagatgttgtagaataagttatgggtttttatgttttgttttgtgtttttccaaaacttgaacttgcaggcaagccttggttgggtatttgatttatccagaatgcttctctttagggaacaaggatgtttttaatggcataacaaaggcaagactctgccttaatttttgaaaagctgctaactacatgaacacaaatgtgtatttttgttgcagtgtagttttccttttgtgtaattttaaagtcagtgttgaattttattgaaagctcatgatgcgcttcaaagtggcaagtatttggctattaactgccaaaacaagagcctgattttttgaggccagtaattcgtttgctagaattgattttttttctctctctctttgttacataagggcattatgtaacactagccgaatggtagcctctgggttgttgtttttttcttttcctccatgatgttaatgggttatctcaaattttaagttaaactacctaaaataaataccaaagataatgcatatttttgcacagtggagcttacacttaaaagaaaacaaagccccatgggctgccttgaaatcaagagacaataactttgaacctcagcaagaccttgaaccgccggttcattttgcaccttattcagaaaatagagcatcatactcaccgagtctagtcagtgtagtgcttttaaaaattttgtcctttcatgtaacttttttattttaagaggaagaagaagaaaggggcacacacacacaataccgacgtctatcctttcctgctaggcagtgctggccaggctcatgtgtagtgtgcgagatggtgatgtactcttatatttttctgggcttttccttttgcacattccaaaattcatttcataagacaagatcttcataggacctccttggcatcctggcattctcaaaactgagccatccagcatgaaagataaatgggtttaaacccttgctgctgaatttattgcctggactgtcaggacatcaccagcccaccttcaccttagggaagatgccacacctggcctccacacttgctcttctgatcagtctgtctggattgagtcctacagtgtcagatagggcggcaaatgccaaagcagggaaacagggaggtgtggacaagccagtttgatgcagcacttcagatcaagtgcttaggaaggagaggaaacttgccttttttatggcagaggatagtaatgaaaatgtctcagtattttagggtcaatgagagccataaaaatataacataatcacaagtaaaggagataatggtctaaaacagctatttcccttttctgtgtgcatacttatgactgaatgtgagctaagcattttctcctgtggagccctagagcaggttactaaggaaggacacattgttttccagaagcctcccctgcctggctgactgccttgctagaaacataattttttttttctcactgaagctcaataatggaactctttttttttttttttttaatttaaagttccctatttgtgaattctgggattactgacttttctttttaattggagtctcaaaatcaactctcttatggtattatatctctgtatgccattaaaaaacagcttgttctagaatcatgtattttgtaaactgatgtttgtgatggtctctggttcttgaacagccatatctgaatgccgtgcctgcaaaactatgacaatttttgctgttttcagccttcagatttgatggcttgggaaactgaggtgttattttcaatgaaacaaagaaagagatgttaagcaagtggttgttttagatccaaatgtaaaggcaggtttgggaaggtgtttaaagagttggaggaattggggattgagttgtaaagaaaacttacagaagaggcaacaatttggttcttgacagtgagaggatattgagggcttcagctgctgctattatgatgttttgcaaaggaaaataatcaaaccaaagagtattcagtgatatgtaaattaaatgaagatacagtggagaatgggggtgaccacaaaagaggctccccctaaacacacagtgctgccacttaaaaagacttgagaaatttgaaagggggtgggtatggggggggcaagaaagagggagggaaatctttcaacttatttctgaaaaagagaaaaaaatataaaatttctggtgcacaggtttgttttttcaagaaaattttgcagaagctatgtttttaaagtgtacattttataaagtttatcagatattttcatatttaaagccaaatgtaaatagaggtctgtaaagaaaaataattgccatagaaagtataatttcagtgcagtaatttctgagagctagtacctatatgctaccggttagcatggttttagcaaatatataccagccttataaggttcgtattgctatgttcttctgttatttatttcagcatggactgttcatttgaaacctttttctagttattagcgttttaacagttacaagctttaaatggcaattttttttttttttttttttttttttttttttttgtcaagagccaagacacaggtaatgcacgacattgattgctgcattttaccttcaaaatatttgtccttattgactgggtctccttaattaatgtacacatgtcattagaatgcagacggaggggactcaccatgaatatctggggttgattcccagatgtgtgttgcttctctattgcaagcagattccctgttggatttacttcggatttattcccttttaaagaatttttgcccatatctggaagggcactatatttttgggaggagccatagattcctggttatcctatttttaaacaaaatgtagacaaagtgaactctattttgattattgagaaaggagtagttttctatccctctaagagtatacttgaatcagacattttaaggatgtcactatggcactgttgtcatttccaaattcctagaaaagtttgttttactttgtttttattctgttaatgcattctttcttctctttacttcctttcttaccagtacactcctatctcaactctgtttatttgatgagttctgtcccgtaaatcatatttcccttacaattaataaatgtcacttcatattttataataaaccactcagtaaaagcaaaagcttgtcctgagaagtagagtgagttctttttcactctgtgtctaataatgttaaggtgggaaaaaaaaaagtgtggcatagctacctgcccatccccaaccctcagcaaagtagaatctcttttctggtaattttgggtttccgctctgggctctggcaagttgaacaatcctagccattgacaatcgtgatagttattattttcccatttgctgtctttttgtatctaaagtcttcctattgtactgcacaaaccatggattgtacatatttttatatattatgtcttattttattatttctaaataaaaaaattaaaaattgaaaaaaaaaaaaa

SEQ ID NO: 30 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 31 - DNA, 4274 bp, Mus musculus
gagagcgcactggagccctggccagcgcgcagccttcccagcaccggcaagcagggtcttgggaattctcacttgcttcggctcattctctttcacaaaaaccactggatcttacatgcttctgtaatccccacttccactccatgtccccatgatcctgtaccagcaacaggacaagttctctggatgtcagctgagttactaaggtaactttgctggtcaaaagaaccccaaggttctcggaagcatccatctctcctcattaataaatatatatattaattatatatatatataattttttttggtgtgtcttcaccgaacattcaaaactgtttcttctaagggttttgcaaaaactcagactgttttctaaagcagaaacactggcgtccccagcggaagcaatgggcagtgtgagaaccaaccgctacagcatcgtctcttcggaggaagatggcatgaagctggccactatggcagttgccaatggctttgggaatggcaagagtaaagtccatacccgacaacagtgcaggagccgctttgtgaagaaagatggtcattgcaatgttcagtttatcaacgtgggtgagaagggacagaggtacctggcagacatctttactacctgtgtcgacatccgctggaggtggatgctggttatcttctgtcttgccttcgtgctctcctggctgttctttggctgtgtgttttggttgatagccctgctccatggggatctagatacttctaaagtgagcaaagcatgcgtgtcagaggtcaacagcttcacggctgccttcctcttctccatcgagacccagacaaccattggctatggtttcaggtgtgtgacagacgagtgcccaattgctgtcttcatggtggtattccagtcaatcgtaggctgcatcattgacgccttcatcattggtgcagtcatggcgaagatggcaaagccaaagaagagaaatgagactcttgtcttcagtcacaatgctgtgattgccatgagggatggcaaactctgcttgatgtggagagtgggtaaccttcgaaagagccaccttgtggaagctcatgtccgggcacagcttctcaaatctaggatcacttcagaaggggagtatatccctttggaccagatagacatcaatgttggttttgatagtggaattgaccgcatatttctagtgtcccccatcactatcgttcacgaaatagatgaagacagccctttatatgacttgagtaagcaggacattgacaatgcagactttgaaattgttgtcatactggaaggcatggtggaggcgactgccatgacaactcaatgccggagttcgtatctggccaatgaaattctctggggtcaccgctatgagccagtgctctttgaagagaaacactactataaagtagactattcaagattccataagacttatgaagtacctaacacccccctttgtagtgccagagacttagcagagaagaaatacatcctttcaaatgcaaattcattttgctatgaaaatgaagttgccctaacaagcaaagaggaagaggaggatagtgagaacggagtcccagagagcacaagcacagactcacctcctggcatagatctccacaaccaggcaagcgtacctctagagcccaggcccttaaggcgagaatcggagatatgactggctgattccgtctttggaatacttactttgctacacagcctgacgttggtcagaggtccgagacagttatacagaccatggtactggtcgagaggtgggtgaaagcaagcagccacaagagactaaggctagcacaaaggtttcaaggaaagactaagctggatgactgatgtaaagtgctttgcaggcctccaagagacatgatggcacatatctgttgtagtataagttatggggtttttaatgtattgttttgtgtttttacaaaacttgaatatgcaggcaagcctcagtttgggtacatgacttacctggaatgtttctctttaggggaacaagagtgattttaatggcataacacaggcaagactctgccttaattttttgaaaagctgctaactacatgaacacgaactgtatttttgttgcagtgtagtttatcttttacataacgttaagacgtcagtgttgagcattgttgaaagcgcacagtgtgctttaaagcatcaagtatttggctattaactgccaaaaatgaaagcctgattttctgaggccagtaatttgtttgctaaaaattgatctctctgtctacctgtcagtctctgtctctgtctctctctcgttatctctctctctctctctctctctctctctctcattacataatagcattatataacactagccaaatggtagcctctgggttttatacttttcttttccacaatgctaataggttatctcaaactttcagttagacgaccttaaatgaataccaaagataatgcacattgttttgtttttgtttttgtttttttctttttctttttttttctttcctttttttttctttttttgcacatggaggttatatttttttaaaaagaaacaaaggctcctgcacagattgtcttggaaatccagagacaccatctttgaacttcagcagggtgtgaagcagtccgttcatttttgcactatagtctgaaaagaacaccaaagtctgatcaatgtagtgtgttttcgagtctgttcttttgtgcttctttcttatttggaggggaggagggaagcataggtcacagttacatatgcactataatcaatatttttttctttccctttttctgagcttttctctcttagcacattccaaagttggttccataagatcagatcttggttgaactttgttggtaacctggcaccttccaaactgtcacccagcagggaagatgaacagatttcaaattttgcagctgactcatagccttgactgccagggcatttctagctcagtgtcacattaagaaagatgtctggcctctagtcttgtccttctaaggtctctgcagagatagagagagagagagagagagagagagagagagagagagagagagagagagactttcaatgcagcaatccactttacaggtgagagggaagtttcctttttcatgacagagggtaagagggactgtttcagtattttaggtcttagtgagccatataaacacaacataattacaagctaaggagttaagagtctaaagggttatttctctctctgtaaacattcatgattaatgccagtgggtcattgtctcttggaaagtctcagagcacgttgccaaggactcattattttctagaagcctcttttacctattcaccttcctttataaaaacctccctccctttcccaaacactagctcaatcattcaattctccctccctttgtttctcttggttttaatgttctctacttgtaaaccctggggctgctgagttcctttttagttggcgtctcaaaatcaagcctctaatggtattacaccctaatatgccattaaaaatcagcttgttctagaaccatgtattttatttaggttgatgtttgtgccggtctgtggcttgtgagcctctgtatctggatacagtgcctgtaagaaacgttgacaatttctgctgttttcaactttctaagtcaatggatttgaaaactgttggtgttatggaaatcaagagagaagcgtcgagaaagtgtacatctgggttttaaggcagggttgcaagttgcttaaagagtttgctgggttggggacagggagaagtcacaggagagaacacagactggcttctgacagtgtgaggctatggaaggcttcagatgctgctattaaggtgatttgcaaagtaaagtaatcaaaccaaagagtattcagtggaatgtcaatcaaatgaagatgcagtaaagagtgggggtgacaacatggactgaccccaaacacaaaaagacttgagttatttgaaaagagggagggggtgggtacagagacaagaaaagtgggaggggaaatctttcaattttttttatctttatttgcttaaaaagaagaaaccatctaaaatttctggtgcatagatttgtttttcgtttgtttgtttgttcgttttttttctttttgtttttcaaggaaaattttgcagaagcgacatttttaaagtgtacattttataaagtttatcagatattttcatatttaaagccaaatgtaaatagaaatctg

SEQ ID NO: 32 - protein, 428 aa, Mus musculus [sequence omitted]

SEQ ID NO: 33 - DNA, 2332 bp, Homo sapiens
gtctgcatagaaagaccaacaaccagcaccacttccttgctttttccagccatgaatgcttccagtcggaatgtgtttgacacgttgatcagggtgttgacagaaagtatgttcaaacatcttcggaaatgggtcgtcactcgcttttttgggcattctcggcaaagagcaaggctagtctccaaagatggaaggtgcaacatagaatttggcaatgtggaggcacagtcaaggtttatattctttgtggacatctggacaacggtacttgacctcaagtggagatacaaaatgaccattttcatcacagccttcttggggagttggtttttctttggtctcctgtggtatgcagtagcgtacattcacaaagacctcccggaattccatccttctgccaatcacactccctgtgtggagaatattaatggcttgacctcagcttttctgttttctctggagactcaagtgaccattggatatggattcaggtgtgtgacagaacagtgtgccactgccatttttctgcttatctttcagtctatacttggagttataatcaattctttcatgtgtggggccatcttagccaagatctccaggcccaaaaaacgtgccaagaccattacgttcagcaagaacgcagtgatcagcaaacggggagggaagctttgcctcctaatccgagtggctaatctcaggaagagccttcttattggcagtcacatttatggaaagcttctgaagaccacagtcactcctgaaggagagaccattattttggaccagatcaatatcaactttgtagttgacgctgggaatgaaaatttattcttcatctccccattgacaatttaccatgtcattgatcacaacagccctttcttccacatggcagcggagacccttctccagcaggactttgaattagtggtgtttttagatggcacagtggagtccaccagtgctacctgccaagtccggacatcctatgtcccagaggaggtgctttggggctaccgttttgctcccatagtatccaagacaaaggaagggaaataccgagtggatttccataactttagcaagacagtggaagtggagacccctcactgtgccatgtgcctttataatgagaaagatgttagagccaggatgaagagaggctatgacaaccccaacttcatcttgtcagaagtcaatgaaacagatgacaccaaaatgtaacagtggcttttcaacgggagtaaagcaaagtctctaaagctcctagtacctagaagcattatgaagcagtcaacaatttaggggtacgaaagtaggatgagagccttcaaagtctaccagcacaaagacccctgagccccgcaattgtgatcccacaagacatgcatctccacaaggctactgtattagaacgtgcaatgcatttatatgaaactggtgtatggaagacataggtgctctcttgaaatcttaaatatgattatttgagctcatataaggtggattggagcagataaaattatcaaaagtttcatgaacaggccaaacaaaatattttttaaagtttccttaaagaagttatgaactttagaaaggatcaggggacaataataatctcattttgattctactgataagaatgactccacttttaatgtggacttttactcatggaaaaattgtctcctaatttggggagatgaaccaaccaatcaatgacaagaaaacgcttacacaaagaacaatttgaggctctaagcttctcatgtggtacgtttagacagaggctaaatctgcacactagaatcttgatgataccttcctgcaagacagaatgctttagttaaaagtggtgatgatatttctttcaatctgtattggatggcttaaagggctataaatctgtttataaagagcatttcctgctcttcgaagacagcaatgaggagttggaaggtgcaaagtcagtagagaagggaatgtatcattaatgcacctgagaagaaacagtttcatgtgttcctccacctagagtttgtactggaatgctatttctaaagaagaagtgggaaagagagaggaatgggatggagccccacagtcagaatgttactatgtctttctttccctgacagcccatcttcctaaaagggaccagcttatggaaggctcgaccttgaggggaaagttttactgtgaaagtcttcttcagatccccacctgcatcattccgaatgtgtcctggaaaaaaactggtactcaaagctgcttaggaatcaaaatgttttcagtgtgttgattaatatagtaaatttctgaaactgtg

SEQ ID NO: 34 - protein, 391 aa, Homo sapiens
MNASSRNVFDTLIRVLTESMFKHLRKWVVTRFFGHSRQRARLVSKDGRCNIEFGNVEAQSRFIFFVDIWTTVLDLKWRYKMTIFITAFLGSWFFFGLLWYAVAYIHKDLPEFHPSANHTPCVENINGLTSAFLFSLETQVTIGYGFRCVTEQCATAIFLLIFQSILGVIINSFMCGAILAKISRPKKRAKTITFSKNAVISKRGGKLCLLIRVANLRKSLLIGSHIYGKLLKTTVTPEGETIILDQININFVVDAGNENLFFISPLTIYHVIDHNSPFFHMAAETLLQQDFELVVFLDGTVESTSATCQVRTSYVPEEVLWGYRFAPIVSKTKEGKYRVDFHNFSKTVEVETPHCAMCLYNEKDVRARMKRGYDNPNFILSEVNETDDTKM

SEQ ID NO: 35 - DNA, 3102 bp, Mus musculus
tgttggtttaaaccacacaactccactcttgagttaaccattgaaagctaatgcaagcaagcatcattgtgaggcttaagattcattaaggtgggcctcaaagaagtcggcctttgcacagacaaaactgaacagcaccactcacttgctttgcccagcatggatgcttcagatcggaggtgtatgtgcagagtgctgatcagggcactgacagaaaggatgttcaaacatcttcgaagatggtttgtcactcacatatttgggcgttctcggcaacgagcaaggttggtctccaaagatggaaggtgtaacatcgagtttggcaatgtagatgcacagtcgaggtttatattctttgtggatatctggacaactgtacttgacctgaaatggaggtacaaaatgaccgtgttcatcacagccttcttggggagttggtttctctttggtctcctgtggtatgtcgtagcctatgttcataaggatctcccagagttctacccacctgacaaccgtactccttgtgtggagaacattaatggcatgacatcagcctttctgttttctctagagacccaagtgaccataggttacggattcaggtttgtgacagaacagtgtgccactgccatttttctgcttatcttccagtctattcttggagtgatcatcaattctttcatgtgtggtgccatattagccaagatctctagacccaaaaaacgtgcaaagaccattacattcagcaagaatgcggtgatcagcaaacgtggggggaagctctgtctcctcatccgagtagcaaatcttaggaaaagccttctgattggcagtcacatatatggtaagcttctgaagactaccatcacacctgaaggagagaccattattttggatcagaccaatataaactttgtagttgatgctggcaatgaaaatttgttcttcatttccccactgacaatctaccacattattgaccacaacagccctttcttccacatggcggcagaaactctttcccaacaggacttcgagttggttgtctttttagatggcacagtagaatccaccagtgcaacctgccaagtccgcacatcatacatcccagaagaggtgctttggggttaccgttttgttcccatcgtatccaagaccaaggaagggaaataccgagtggatttccataactttggtaagacggtggaagtggagacccctcattgtgccatgtgcctctataatgagaaagatgccagggccaggatgaagagaggctatgacaaccctaactttgtcttgtcagaagttgatgaaacagacgacacccaaatgtagcagtggcttttctacctacaaagagtctctcaaggacctaagtgctggctgtgttcagaagcatcctgcagttgggggactgaaagcaggatgagaacccaagaagtctaccagcagagtcaccccctcagccccatggctatgattctagaagactcgtagctctaaagtctgcagcagtaagcatacggtggaagcatgtgaatgaacctggcgtgtggaagccaaaaggagctcacttggatcttcactctgactgtgtaagctcgtacagtgttgatggaaacaaagtcatccattgacatgtactggagaaaagttaggagctttagaaaacttcaggagctagccatatttcctgtttgattctatggatgagaaagatatcacattttatcttaaagtagacttttatcaacggaaaatctgtcttctgagttgggaagtgagccaaccagtcactgacaataagagcctgtcatacacagaatcactaaaggctctaacttcttaagctctgctgtttgaagcctttgtctgagtctgggtccatgtttcagaaggggtaaggtgggcatccagtgactgcacctttctcaacccaaggtacagaagaacagaaagcctcaaccaatttcataactaactcagacgctgcagcccatataggaatggacctgaatgatttccggtggtgcaatcaatggaggccaagcccattccttagactaaatgaacattctcttgcaaaggagagtgtttgagctaaaagtgttggtgaagaggctgaagagatgacattctttgtaagttcatgctgcgcacgcatgaagacctgggtctgaacctccaacaatcacatacaaagcttgacttagtaggatgctccccctccccaatcctagtactggggaggtagggataggaggatgtctcaagttttctggacagctgaatgggtgaggtaccagttcagggagagactacaaaaataaggtgaaggggctggcgggaagactctggttaaaagcaccgtggctgatcttccagagaacctaggtttggttcctggcacagtacttcataactatctataactccagtttcagggcatctgatgctcttttctggcccctatgagcaccaggtgcacatgcgagcaaaacactatacacatacgattaaaaagataaggtagagaatgactgaagaagaaatcttacgtcaacttctgatctacacacacacacacacacacacacacacacacacacagagcatagtgatgtttgtttcagtattcagtgggtaggttatagggccacaggtctggtaaaagaggttttattccttcctctctcaagatggtgctgaggactgggaaagcagtcagcctggagtgtgtatgatgctaacaccgaatggttcttgtgtttctctgactagtggttgaactgcaattctaggtatgaaggagaaaggaacgagatacagcaccacagtcaggatgctgctgctttcttgtctctaaaggccatccctacatcttccaaagggaacaagttagcaggagaatttacttcggaggaaggttctaatgtcttgtttgttcatttccacatctgcatcattctgatttcaccttgggggaaaaaaagtagaattcaaagctgttcaaaaatcaaaatatcattttagtatgtgattaataaatttctgaaactgtatgagcaaaaaaaaaaaaaaaaaaaaaaaaaaaaaaaa

SEQ ID NO: 36 - protein, 372 aa, Mus musculus
MFKHLRRWFVTHIFGRSRQRARLVSKDGRCNIEFGNVDAQSRFIFFVDIWTTVLDLKWRYKMTVFITAFLGSWFLFGLLWYVVAYVHKDLPEFYPPDNRTPCVENINGMTSAFLFSLETQVTIGYGFRFVTEQCATAIFLLIFQSILGVIINSFMCGAILAKISRPKKRAKTITFSKNAVISKRGGKLCLLIRVANLRKSLLIGSHIYGKLLKTTITPEGETIILDQTNINFVVDAGNENLFFISPLTIYHIIDHNSPFFHMAAETLSQQDFELVVFLDGTVESTSATCQVRTSYIPEEVLWGYRFVPIVSKTKEGKYRVDFHNFGKTVEVETPHCAMCLYNEKDARARMKRGYDNPNFVLSEVDETDDTQM

SEQ ID NO: 37 - DNA, 4187 bp, Homo sapiens
ggcggcgcaggggcggggctttacggacgcaagcacgtcgaagcgctgctcctggagccgcggagggtgcgggtttggctgcggtggtttctgtggcggttgctgtggcggagtttggaggttggagagaaatccaggtactcactagactggtaccttctgccaccatgggggagcttttccggagtgaagaaatgacactggcccagctttttctacagtcagaggctgcttattgttgtgtcagtgaattaggagaacttggaaaggttcagtttcgtgacttaaatccagatgtgaatgttttccaacggaaatttgtgaatgaagttagaagatgtgaagaaatggatcgaaagcttcgatttgttgagaaagagataagaaaagctaacattccgattatggacaccggtgaaaacccagaggttcccttcccccgggacatgattgacttagaggccaattttgagaagattgaaaatgaactgaaggaaatcaacacaaaccaggaagctctgaagagaaacttcctggaactgaccgaattaaaatttatacttcgcaaaactcagcaattttttgatgagatggcggatccagacttgttggaagagtcctcatccctcttggagccaagtgagatgggaagaggcactcctttaagacttggcttcgtggctggtgtcattaaccgggagcgcatccctacttttgagcgcatgctttggcgggtatgccggggaaatgtgttcctgcgacaggctgaaatcgagaaccccctggaggatcctgtgactggcgactacgtgcacaagtctgtgtttatcattttcttccaaggcgatcagctgaaaaacagagtcaagaaaatctgtgaagggttccgagcctcactctatccctgtcctgagacaccacaggagaggaaggaaatggcttctggagtgaataccaggattgatgatctccaaatggttctgaatcaaacggaggatcaccgccagagggttctgcaggcagctgctaagaacatccgtgtctggttcatcaaagtgcggaagatgaaggccatctatcacaccctgaacctgtgcaacatagatgtgactcagaaatgcttgattgcagaggtctggtgccctgtcaccgaccttgactccatccagtttgcactcagaaggggcacggaacacagtggttccactgtaccttccattttgaacaggatgcagacaaaccagactcccccaacctataacaaaaccaacaagtttacctatggctttcagaacatagtagatgcttatggaattggaacttaccgagagataaatccagctccgtatactattatcacgttcccttttctatttgctgtgatgtttggagacttcggtcatggcattttaatgaccctttttgctgtgtggatggtactgagggagagccggatcctttcccagaagaatgagaatgagatgtttagcactgtgttcagtggtcgatacattattttattgatgggtgtgttctccatgtacactggcctcatctacaatgattgcttttccaagtctcttaatatctttgggtcatcctggagtgtacggccgatgtttacttataattggactgaagagacgcttcgggggaaccctgttctacagctgaacccagccctccctggagtgtttggtggaccatacccttttggcattgatccaatttggaacattgctaccaataaactgacgttcttgaactcctttaagatgaagatgtctgttatccttggtatcatccatatgctgtttggagtcagcctgagtctgttcaaccatatctatttcaagaagcccctgaatatctactttggatttattcctgaaataatcttcatgacctctttgtttggctatttggttatccttattttttacaagtggacggcctatgatgctcatacctctgagaatgcaccaagccttctgatccatttcataaacatgttcctcttttcctacccagagtctggttattcaatgttgtattctggacagaaaggaattcagtgtttcctggtagtggttgcactactgtgtgtaccttggatgctgctgtttaaaccattggtccttcgccgtcagtatttgaggagaaagcatttgggaactctcaactttggtgggatcagggtgggcaacggaccgacagaggaggatgctgagattattcagcatgaccagctctccacccactcagaggacgcagacgagtttgactttggggacaccatggtccaccaggccatccacaccatcgagtactgcctgggctgcatctccaacactgcctcctacttgcggctctgggccctcagcctcgctcatgcgcagctgtctgaggtgctttggaccatggtgatccacatcggcctgagcgtgaagagcttggcgggaggtttggtgctgttcttcttcttcactgcctttgccaccctgaccgtggccatcctcctgatcatggagggcctctcggcctttctccacgcactgcgcttacactgggttgagttccagaataaattctacagcgggaccggtttcaagttcttacccttctccttcgagcatattcgggaagggaagtttgaagagtgagtccctgtgagggccgtgtgccccatgctaccctccccgcctccctccacagtgatcagctgtgcctctctgcctgttggttgtgatctgtgggcaccagctcattcgtgtcaccctgtctgtgagtcatttagatagaatagtcctccttgggtctcccaccacccctagctttgtgtgtagtgtagtgattttctggctgtcactcatactcactgggcaccagccttgccctcttagcctccatccatccagacagcccttcccacctcctggtggtgagccagtctgcattcccacgccatcccaaagccctttcatcttccccgtgcattgtagatggaaggagcacccatgccattcacatctagactttgagttccctgcatctgccaccgtagtttctagcaggagtagtggggggagtaatacagattcttccctagaaggggacactggtaacatgtcccactcttggattagcaggggtgggtccaggaagatgatatttgcgtcttttgcccacccccctggcattcagctggacccaactaggccatcatgagtggcttctccctgtcatccccaggggtcataggatatctacaccgcctttctgaccccaccctgcactcccatcctttcctctctccccgttcatgccctgcactacatagcacagccgggatgcttggaacagaggccttggctgctccgcagtgcacagggcttccctctctcggggttggcttcttcccaggccttgcatgggccctgcccacaagcacaccctcaggccgagggtgcagactgatgctcttccctgatggagaccctgagatcttccccacccccaatcatgatgtcttcagtgtgggactggggtcctcttggttctgcctgcagcctgcctggctccgcccctagtgccccctcctcaccacactggccccaggtctcaggaggggtgtcctgggcagggaaggtcagtgtcactgatggtttgctgtttggaagccattggcagggctgccgtgcatgtggctgtgagggctgcacagtcctgccaaggggcttcctccttgtcaccccgaaccttgtaatcgtgtgctggcgtggcagccctggctaagttaatccccaccgctttcagtggtagaaagaattccctgagtgggccaggctggtgccctcctcctaccctggcttttctgagtgagctgcctggagccctcatcccctctcccaggctgggctggccctgggcggggccactgtgtgctggcccactgtgacctgacccgaccttgtgcagcccccctgccctggtgtcctgggttttcgtgatgatctttgctctgtttccagtggggtttgaagcagagttcagggaaccctgcccaaggtcctcctgttcagacattcctatgttgaataaagtatgtttgacttccccggaaaaaaaaaaaaaaaaaaaaaaaaaaaaaaaaaaaaaaaaaaaaaaaaaaaaaaaaaaaaaaaaaa

SEQ ID NO: 38 - protein, 831 aa, Homo sapiens
MGELFRSEEMTLAQLFLQSEAAYCCVSELGELGKVQFRDLNPDVNVFQRKFVNEVRRCEEMDRKLRFVEKEIRKANIPIMDTGENPEVPFPRDMIDLEANFEKIENELKEINTNQEALKRNFLELTELKFILRKTQQFFDEMADPDLLEESSSLLEPSEMGRGTPLRLGFVAGVINRERIPTFERMLWRVCRGNVFLRQAEIENPLEDPVTGDYVHKSVFIIFFQGDQLKNRVKKICEGFRASLYPCPETPQERKEMASGVNTRIDDLQMVLNQTEDHRQRVLQAAAKNIRVWFIKVRKMKAIYHTLNLCNIDVTQKCLIAEVWCPVTDLDSIQFALRRGTEHSGSTVPSILNRMQTNQTPPTYNKTNKFTYGFQNIVDAYGIGTYREINPAPYTIITFPFLFAVMFGDFGHGILMTLFAVWMVLRESRILSQKNENEMFSTVFSGRYIILLMGVFSMYTGLIYNDCFSKSLNIFGSSWSVRPMFTYNWTEETLRGNPVLQLNPALPGVFGGPYPFGIDPIWNIATNKLTFLNSFKMKMSVILGIIHMLFGVSLSLFNHIYFKKPLNIYFGFIPEIIFMTSLFGYLVILIFYKWTAYDAHTSENAPSLLIHFINMFLFSYPESGYSMLYSGQKGIQCFLVVVALLCVPWMLLFKPLVLRRQYLRRKHLGTLNFGGIRVGNGPTEEDAEIIQHDQLSTHSEDADEFDFGDTMVHQAIHTIEYCLGCISNTASYLRLWALSLAHAQLSEVLWTMVIHIGLSVKSLAGGLVLFFFFTAFATLTVAILLIMEGLSAFLHALRLHWVEFQNKFYSGTGFKFLPFSFEHIREGKFEE

SEQ ID NO: 39 - DNA, 4516 bp, Homo sapiens
ctcggcgcgcctggacccctgcccctctctgggtggagaagctcccggccgcttcccggtttcactccttctcagcctgggctcccagcccctctctccttttcctggactggctctcacccccttcggtccccttcctttagctcaggctccctaccccttcctttagcccacagcccagagtcccagctcctcagtcactttcctcagccaaaggtcccagccttccttcttcctttcctttgcactatccctatcctgccccttcctctatccctagggctcagtttcccacatccgtcctcccccttcccaggcccggagttccagaccttttggtctcctttcgtggtcgttcctgggtccttgccccctttccccactttggagttccagattgcaaacccagcctccctccacccccagaaaattgcttccatggaaatgcctctctaaaacatgaacttttcctagagactacgccagtctctcttcccacttgctgaccctttgctacctatgtgcccggttttactctcatttgggtaaggtcgaggctggctctggaagcagcaccatggttctgcggtctggcatctgtggcctctctccacatcggatcttcccttccttactcgtggtggttgctttggtggggctgctgcctgttctcaggagccatggcctccagctcagcccaactgccagcaccattcgaagctcagagccaccacgagaacgctcgattggggatgtcaccaccgctccaccggaggtcaccccagagagccgccctgttaatcattccgtcactgatcatggcatgaagccgcgcaaggcctttccagtcctgggcatcgactacacacacgtgcgcacccccttcgagatctccctctggatccttctggcctgcctcatgaagataggtttccatgtgatccccactatctcaagcatcgtcccggagagctgcctgctgatcgtggtggggctgctggtggggggcctgatcaagggtgtaggcgagacaccccccttcctgcagtccgacgtcttcttcctcttcctgctgccgcccatcatcctggatgcgggctacttcctgccactgcggcagttcacagaaaacctgggcaccatcctgatctttgccgtggtgggcacgctgtggaacgccttcttcctgggcggcctcatgtacgccgtgtgcctggtgggcggtgagcagatcaacaacatcggcctcctggacaacctgctcttcggcagcatcatctcggccgtggaccccgtggcggttctggctgtctttgaggaaattcacatcaatgagctgctgcacatccttgtttttggggagtccttgctcaatgacgccgtcactgtggtcctgtatcacctctttgaggagtttgccaactacgaacacgtgggcatcgtggacatcttcctcggcttcctgagcttcttcgtggtggccctgggcggggtgcttgtgggcgtggtctacggggtcatcgcagccttcacctcccgatttacctcccacatccgggtcatcgagccgctcttcgtcttcctctacagctacatggcctacttgtcagccgagctcttccacctgtcaggcatcatggcgctcatagcctcaggagtggtgatgcgcccctatgtggaggccaacatctcccacaagtcccacaccaccatcaaatacttcctgaagatgtggagcagcgtcagcgagaccctcatcttcatcttcctcggcgtctccacggtggccggctcccaccactggaactggaccttcgtcatcagcaccctgctcttctgcctcatcgcccgcgtgctgggggtgctgggcctgacctggttcatcaacaagttccgtatcgtgaagctgacccccaaggaccagttcatcatcgcctatgggggcctgcgaggggccatcgccttctctctgggctacctcctggacaagaagcacttccccatgtgtgacctgttcctcactgccatcatcactgtcatcttcttcaccgtctttgtgcagggcatgaccattcggcccctggtagacctgttggctgtgaagaaaaagcaagagacgaagcgctccatcaacgaagagatccacacacagttcctggaccaccttctgacaggcatcgaagacatctgtggccactacggtcaccaccactggaaggacaagctcaaccggtttaataagaaatatgtgaagaagtgtctgatagctggcgagcgctccaaggagccccagctcattgccttctaccacaagatggagatgaagcaggccatcgagctggtggagagcgggggcatgggcaagatcccctctgccgtctccaccgtctccatgcagaacatccaccccaagtccctgccttccgagcgcatcctgccagcactgtccaaggacaaggaggaggagatccgcaaaatcctgaggaacaacttgcagaagaccaggcagcggctgcggtcctacaacagacacacgctggtggcagacccctacgaggaagcctggaaccagatgctgctccggaggcagaaggcccggcagctggagcagaagatcaacaactacctgacggtgccagcccacaagctggactcacccaccatgtctcgggcccgcatcggctcagacccactggcctatgagccgaaggaggacctgcctgtcatcaccatcgacccggcttccccgcagtcacccgagtctgtggacctggtgaatgaggagctgaagggcaaagtcttagggttgagccgggatcctgcaaaggtggctgaggaggacgaggacgacgatgggggcatcatgatgcggagcaaggagacttcgtccccaggaaccgacgatgtcttcacccccgcgcccagtgacagccccagctcccagaggatacagcgctgcctcagtgacccaggcccacaccctgagcctggggagggagaaccgttcttccccaaggggcagtaacgccagggccagcaggcagcgcctgtcccctcacagactcttccaccagagcaggggctgctgggggctccccttgcccttcctgacccggattggccctgcccctccccctaccgcatggcagctgggcccacagcccccaccccagcacagctcctcccctgccgcctcccgggaagcatcctccccaccagagctgcctccccaatccatttggcagaactgctggggctggtgaggccggccctgcccctccctagatccaggcttctcccggacctggactagggcctcggaggctcctccctctgcctcatcctcctcctcattcagaccaatcttagtttctaaccaaagagtctctggctcagctgtggtcccacccaggaagggagggagctgaggcctcccttgagtaggccctgctttatcaggggacaaaccaggggtaccaggcacatggctgggggagggactgctgacccaccaaggtctcacactcctcctgccagctctgtcaccctggccaccacccaacctatccttactcagagctgcgggctgagggcatctctgagtgtctctgcctggagcaggggtggtttctacggtgacagtgacgtgactcagagcttttcgaactgtgctcccacggggaccactgggcccctcaggggaagctgctaggggaaggactggccgtggctccagaatgtgctgcctttttaagttttgtttgttcacactcctatatatgattgtttgcacagagggcgctcctgtttttaaaacattttgaaaacccctggctgaacagtgctctgcctctaactccctcctcacactccagaattacccttcctcatctgtgcctgtctgtccaacccctcccccacgtctctctgcctgctgggctcttaactgttgctcgaagactgtgacatcagaagtaactcccactcctaatcaagagtctctccagcctcacagatgctggcctcttggcacctgcctagctcttgggcctgacctccagtcctgctggcctgctcttacttcccccaccctgggtttggcccctggaacctttcccttgtgtgtaccacaccctgcctgctgtggagcccattgtggaggcggtgggggggagaaggcctcccctgaggatcccctgtcccctggggctggtggattgggcagaatcctgggcccccagagacctttgcccacacacactccttccccttgtccctggggcactcccccaggattgtgcaatagtcagagtgtccctttttgcaggggactgggccatgggtcctcggcccatctgtccatcctcctctccatgcaagtgctgtttgggcaggagtcaccatgcaagggtgacatcgacaaccacgtaccaagccaccgcagctgctgccactctgctgcctgtacagaagaaactgaatctttttcatattctaataaatcaatgtgagtttt

SEQ ID NO: 40 - protein, 815 aa, Homo sapiens
MVLRSGICGLSPHRIFPSLLVVVALVGLLPVLRSHGLQLSPTASTIRSSEPPRERSIGDVTTAPPEVTPESRPVNHSVTDHGMKPRKAFPVLGIDYTHVRTPFEISLWILLACLMKIGFHVIPTISSIVPESCLLIVVGLLVGGLIKGVGETPPFLQSDVFFLFLLPPIILDAGYFLPLRQFTENLGTILIFAVVGTLWNAFFLGGLMYAVCLVGGEQINNIGLLDNLLFGSIISAVDPVAVLAVFEEIHINELLHILVFGESLLNDAVTVVLYHLFEEFANYEHVGIVDIFLGFLSFFVVALGGVLVGVVYGVIAAFTSRFTSHIRVIEPLFVFLYSYMAYLSAELFHLSGIMALIASGVVMRPYVEANISHKSHTTIKYFLKMWSSVSETLIFIFLGVSTVAGSHHWNWTFVISTLLFCLIARVLGVLGLTWFINKFRIVKLTPKDQFIIAYGGLRGAIAFSLGYLLDKKHFPMCDLFLTAIITVIFFTVFVQGMTIRPLVDLLAVKKKQETKRSINEEIHTQFLDHLLTGIEDICGHYGHHHWKDKLNRFNKKYVKKCLIAGERSKEPQLIAFYHKMEMKQAIELVESGGMGKIPSAVSTVSMQNIHPKSLPSERILPALSKDKEEEIRKILRNNLQKTRQRLRSYNRHTLVADPYEEAWNQMLLRRQKARQLEQKINNYLTVPAHKLDSPTMSRARIGSDPLAYEPKEDLPVITIDPASPQSPESVDLVNEELKGKVLGLSRDPAKVAEEDEDDDGGIMMRSKETSSPGTDDVFTPAPSDSPSSQRIQRCLSDPGPHPEPGEGEPFFPKGQ

SEQ ID NO: 41 - DNA, 4126 bp, Mus musculus
ttcccttgcccgtggtccagcctatccggcccttcccctctcttcaggacgcagtttccccgatttccttctctcagcctgaagttctagatcctttcggtttcctccttagtccctccaaggtacttaacccctttttgcagtttggactttgagattgtgaatctctagaaaacgaaaatacttctctaaaacatgaacttttcctagagattgccccagtctcaccccatctgcaggactccttgctacctatgtcccaggtcttacacgcacttcgctaagggctgaggcctcctaggctgggggatcagtatgatgcttcggtggtccggcgtctggggatttcatccacctcggatcttcccctccttgctggtggtggttgccttggtgggactgctacctgttctcaggagccacggcctccagcacagccctactgccagcaccatcagaggttctgaaccaccccgggaacgctcaattggggatgtcaccacggcaccatcagagcctctccatcgcccagatgaccacaatttgaccaacttaatcattgaacatggcggtaagccatctcggaaggccttcccagtcctggacattgactaccctcacgtgcgcacaccctttgagatctccctctggattctcctggcctgcctcatgaagataggtttccatgtgatccccaccatctcaagcatcgtcccggagagctgcctgctgatcgtagtggggctgctggtggggggcctgatcaagggtgtcggagagacgccccccttcctgcaatcagacgtcttcttcctcttcctgctgccacccatcatcctggatgcaggctacttcctgcctctgcggcagttcacggagaacctgggcaccatcctgatctttgctgtggtgggcacactgtggaatgcgttcttcctgggtggcctcctgtacgccgtgtgcctggtgggcggcgagcagatcaacaacattggcctgctggacaccctgctctttggcagcatcatctccgccgtggaccccgtggctgtgctggccgtcttcgaggagatccacatcaacgaactgctacacatccttgtcttcggggagtccctgctcaacgatgccgtcactgtggtcctctatcacctctttgaggaatttgccagctatgactctgtgggcatctcggacatcttcctcggcttcctgagcttcttcgtggtggccctcggtggggtgtttgtgggcgtggtctacggggtcatcgcggctttcacctcccgatttacctcccacatccgggtcatcgagccgctcttcgtcttcctctacagctacatggcttacctgtcagctgagctcttccacctgtccggaatcatggccctcatcgcctcaggagtagtgatgcgcccctatgtggaggccaacatctcccacaaatcccacaccaccatcaagtacttcctgaagatgtggagcagcgtcagtgagaccctcatcttcatcttcctcggcgtctccactgtggccggctcccaccagtggaactggaccttcgtcatcagcaccctgctcttctgcctcatcgcccgggtgctgggtgtcctggtcctgacctggttcatcaacaagttccgcattgtcaagctgacccccaaggaccagttcatcattgcctatgggggcctgcgaggggccatcgccttctcgctgggctacctcctggacaagaagcacttccccatgtgtgacctgttcctcaccgccatcatcaccgtcatctttttcaccgtctttgtgcagggcatgaccattcggcccctggtcgacctgttggccgtgaagaaaaagcaagaaacaaagcgctccatcaacgaggagatccacacacagttcctggaccaccttctgacaggcatcgaggacatctgtggtcattatggccaccatcactggaaggacaagctcaaccgttttaataagaagtatgtgaagaagtgcctgatagcaggagagcgctccaaagagccccagctcattgccttctaccacaagatggagatgaagcaggccattgagctggtggagagcggaggcatgggcaagatcccatctgctgtctccactgtctctatgcagaacatccaccccaaggctgtgacttcagaccgcatcttgccagcactgtccaaggacaaggaggaagagatccgcaaaatcttgaggagcaatctgcagaaaacccggcaacggctgcggtcctataacagacacacgctggtggccgacccctacgaggaagcctggaaccagatgctgctccggaggcagaaggcccggcagctggagcagaagatcaccaactacctgacagtaccggcccacaagctggactcgcccaccctgtctcgggcccgcataggctcagacccactggcctatgaacccaaggcagacctgcctgtcatcaccattgacccggcctccccacagtcccctgagtctgtggacctggtgaacgaggagttgaagggcaaggtcctagggctaaaccggggtcccagggtgactccggaggaggaggaggaagatgaggatggcatcatcatgattcggagcaaggagccctcatccccaggcaccgacgacgtcttcacccctggatcaagtgacagccccagctcccagagaatacaacgctgcctcagtgacccaggcccccaccctgagcctggggagggagagcctttcatccccaaaggacagtagagccaggctagtgggtgtcccacagactctcccacgagagtgggggctgggcgctcctcttgtctcctgacctggattggctctgcccccttcctccgcatggcagctgggcccacagcccccactgcagcacggcttgcccctgcctcctgggaagggtcctctcctacagaactgccttcccaatccatccatttggcggatggctgggctgctgaggcaggccccgccccatctctagatccaggctctcccatagtggaaccagggctcctcaaggccctccttccctccagcccctcctcatccagaccaactctccatccttgatcatcatttctaaccaaagagccactggctccactgtggtcccagcaggaaaggagggagctgagccttccgtggccgtgactgggcccctgtatcagaggacagcaggcctgtggctgggaagcagacccgtgacccatagtagagctgcccacggcagcgccacggttctcccgctagcctcggtgctctggccgccacccgagctgtccttactcagagctgcgggctgagggcgtctctgagcttctctgctgggcgctggggcacattcaacagagacgtgattggggtgatgaggaccactgggccttagtggggcttacacgggagggactgtccgaggctctggaaagtgtcctcttcttacacttggcttatttgcacctcactgtaagtatgattctgtttgcacagaggcactcttgttttcagaccctttgagtcccccatgccagtcagagctctgccctttccccaccttcccttaaatggaacctctgctgctgggggcccttgcccacagcaccgattctgatgtggcccaacctgtcatgactcaagctcaccagcagggggcctcccggtacctcccttgctcttgggcctgcttttacttctgttggccagttctaccaccaccccttatctggaccactctctttcctgcattctgtagagtagaaagtactggacccccctagaggcctttgctcacaggcacacactccctaccgccaccatggctggggcaagcccatgggtatctgcccaacccctgggcactccctccccaggatcgtgcaataatagtcagagggtccctcccaggggatggggccatgggtgcatggcccagctgtctcctctccatgtaagtgctgttttgggcaagagttgccaggcagggtgacatcgacaaccatgtacaaagccaccgcagctgctgccgctctgccgcctgtacaaaggaaactgaacctttttcatattctaataaatcaacgtgagtttttga

SEQ ID NO: 42 - protein, 820 aa, Mus musculus
MMLRWSGVWGFHPPRIFPSLLVVVALVGLLPVLRSHGLQHSPTASTIRGSEPPRERSIGDVTTAPSEPLHRPDDHNLTNLIIEHGGKPSRKAFPVLDIDYPHVRTPFEISLWILLACLMKIGFHVIPTISSIVPESCLLIVVGLLVGGLIKGVGETPPFLQSDVFFLFLLPPIILDAGYFLPLRQFTENLGTILIFAVVGTLWNAFFLGGLLYAVCLVGGEQINNIGLLDTLLFGSIISAVDPVAVLAVFEEIHINELLHILVFGESLLNDAVTVVLYHLFEEFASYDSVGISDIFLGFLSFFVVALGGVFVGVVYGVIAAFTSRFTSHIRVIEPLFVFLYSYMAYLSAELFHLSGIMALIASGVVMRPYVEANISHKSHTTIKYFLKMWSSVSETLIFIFLGVSTVAGSHQWNWTFVISTLLFCLIARVLGVLVLTWFINKFRIVKLTPKDQFIIAYGGLRGAIAFSLGYLLDKKHFPMCDLFLTAIITVIFFTVFVQGMTIRPLVDLLAVKKKQETKRSINEEIHTQFLDHLLTGIEDICGHYGHHHWKDKLNRFNKKYVKKCLIAGERSKEPQLIAFYHKMEMKQAIELVESGGMGKIPSAVSTVSMQNIHPKAVTSDRILPALSKDKEEEIRKILRSNLQKTRQRLRSYNRHTLVADPYEEAWNQMLLRRQKARQLEQKITNYLTVPAHKLDSPTLSRARIGSDPLAYEPKADLPVITIDPASPQSPESVDLVNEELKGKVLGLNRGPRVTPEEEEEDEDGIIMIRSKEPSSPGTDDVFTPGSSDSPSSQRIQRCLSDPGPHPEPGEGEPFIPKGQ

SEQ ID NO: 43 - DNA, 5069 bp, Saccharomyces cerevisiae
aagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgactgatacatcatcctcttcatcatcctcttcagcatcttctgtttcagctcatcagccaactcaagaaaagcctgctaagacttacgatgacgctgcatctgaatcttctgacgatgacgatatcgatgctttaatcgaagaactacaatctaatcacggtgtcgacgacgaagacagtgataacgatggtccagttgccgccggtgaagctagaccagttccagaagaatatttacaaactgacccatcttacggtttaacttccgatgaagttttgaaaagaagaaagaagtacggtttgaatcaaatggctgacgaaaaagaatctttagtcgttaagttcgttatgtttttcgtcggtccaattcaattcgttatggaagccgctgctattttggctgccggtttgtccgattgggtcgatttcggtgttatctgtggtttgttaatgttaaacgctggtgttggtttcgttcaagaattccaagctggttctattgtcgatgaattgaaaaagactttggctaacactgctgttgttatcagagacggtcaattggttgaaattccagccaacgaagtcgtcccaggtgatattttgcaattggaagatggtactgttatcccaactgatggtcgtattgtcactgaagactgtttcttgcaaatcgatcaatctgctattactggtgaatctttggctgtcgacaaacattacggtgaccaaactttctcttcttccactgttaagagaggtgaaggtttcatggttgttaccgctaccggtgacaacactttcgttggtagagctgctgctttggttaacaaagccgctggtggtcaaggtcatttcactgaagttttgaacggtattggtattatcttattggttttggtcattgccactttgttgttggtctggactgcttgtttctacagaaccaacggtattgttagaatcttgagatacactctaggtattactattattggtgtcccagtcggtttgccagctgtcgttaccaccactatggccgtcggtgctgcttacttggctaagaaacaagccattgttcaaaagttgtctgctattgaatccttggctggtgtcgaaatcttgtgttccgacaaaaccggtactttgaccaagaacaagttgtccttgcacgaaccatacactgttgaaggtgtttctccagacgacttgatgttgactgcttgtttggctgcttccagaaagaagaagggtttggatgctattgataaggctttcttgaagtctttgaagcaatatccaaaggctaaggacgctttgaccaagtacaaggttttggaattccatccatttgaccctgtctccaagaaggttactgccgttgtcgaatctccagaaggtgaaagaattgtttgtgttaagggtgctccattattcgtcttgaagactgtcgaagaagatcacccaatcccagaagatgtccatgaaaactacgaaaacaaggttgctgaattggcttctagagggttccgtgctttaggtgttgctagaaagagaggtgaaggtcactgggaaatcttgggtgttatgccatgtatggatccaccaagagacgatactgctcaaactgttagcgaagctagacacttgggtctaagagttaagatgttaactggtgatgctgttggtattgctaaggaaacttgtagacaattgggtttgggtaccaacatttacaacgctgaaagattaggtctaggtggcggtggtgacatgccaggttctgaattagctgattttgttgaaaatgccgatggtttcgctgaagttttcccacaacataaatacagagttgttgaaatcttgcaaaacagaggttacttggttgctatgactggtgatggtgttaacgatgctccatctttgaagaaggctgatactggtattgctgtcgaaggtgctactgatgctgccagatctgctgctgatattgttttcttggctcctggtctatctgctattattgatgctttgaagacctccagacaaatttttcacagaatgtactcttacgttgtttaccgtattgctttgtctctacatttggaaatcttcttgggtctatggattgctattttggataactctttggacattgatttgattgttttcatcgctattttcgctgatgttgctactttggctattgcttacgataatgctccttactctccaaagcccgttaaatggaacctaccaagattatggggtatgtctattattttgggcatagttttagctataggttcttggattaccttgactactatgttcttaccaaagggtggtattatccaaaacttcggtgctatgaacggtattatgttcttgcaaatttccttgactgaaaactggttgattttcattaccagagctgctggtccattctggtcttctatcccatcctggcaattggctggtgccgtcttcgctgtcgacatcatcgctaccatgtttaccttattcggttggtggtctgaaaactggactgatattgttactgtcgtccgtgtctggatctggtctatcggtatcttctgtgttttgggtggtttctactacgaaatgtccacttctgaagcctttgacagattgatgaacggtaagccaatgaaggaaaagaagtctaccagaagtgtcgaagacttcatggctgctatgcaaagagtctctactcaacacgaaaaggaaacctaatcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagttattttatccacacacacaccttaaaatttttagattaaatggcatcaactcttagcttcacacacacacacacaccgaagctggttgttttatttgatttgatataattggtttctctggatggtactttttctttctttttctttcttggttattttccattttaaaatatgaaacgcacacaagtcataattattctaatagagcacaattcacaacacgcacatttcaactttaatatttttttagaaacactttatttagtctaattcttaatttttaatatatataatgcacacacactaatttattcattaattttttattgagtaggatttgaaaatatttggtatctttgcaagatgtttgtatagagggacaaagaatcgtctttattatggtcaaggctttacgtcataatagttcctgcccagctcttctataatactttaaagatctcttctcgtttgctccatttggaagtctcgcttacgtttatgcgcccatacagacactcaagatacacacttacatgaacgtatacaaatttactaacactacttgaaaatatgaaccacagtacatcatattaagacgtagtattcgatgattgaaggccgcctccgcgaatacctttactgattttgccggttaatcgcatcgaaatttcttcgtcacaagaaagcaaacaaatcgccaggccattctaccagtttccttttcttatgaagatgtaaaagctactaaccgcacattactctagatgactcagtttagtctgaccttctatactataactaccctggcgctatgatgatgagcggttcttttattgcggaaacgaaaattccgggaccggcgaaatttgcccggttttgtcgtaaccggcttcatgagtcggcttcaatagtagttgaatacttatttaaacagcagaacttaactcactcatcacgctgtttccgctgaattttctcaaaatatctaagcagtcaacaaatataaagaatattgaaatttgacagttttttgtcgctatcgatttttattatttgctgttttaaatcatggtttacactccatccaagggtccaagaactctttacgataaggtttttgatgcacatgttgtccatcaagatgaaaatggttcctttttgttgtatatcgacagacacttggttcatgaagtcacctctccacaagctt

SEQ ID NO: 44 - protein, 918 aa, Saccharomyces cerevisiae [sequence omitted]

SEQ ID NO: 45 - DNA, 8526 bp, Homo sapiens
agacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatgaaaaaaaaaaaaaaaaaaaaaaaaaa

SEQ ID NO: 46 - protein, 2015 aa, Homo sapiens [sequence omitted]

SEQ ID NO: 47 - DNA, 8452 bp, Mus musculus
ggagcccgagcggagcggcggagccgagacggcggcggcgcccgtgggatgtggggatcgcgcccccggggccgctgagcctgcgcccgctgccccaagccctacgccgaaccgagcccgcgccgcgctgcagccgcccacccctaggcgcgggccggggactagcagcttccttccaggcagcctgaggagagcctgtgcccccagaagcaggatgagaagatggcaaacttcctgttacctcggggcaccagcagcttccgtaggttcacccgggagtcactggcggccatcgagaagcgcatggctgaaaaacaagcccgtggttcggccacctcacaggagagccgtgagggcctgccagaggaggaggctccccggccccagctggacctacaggcctccaaaaagctgccagatctctatggcaacccaccccgagagctcattggggagcccctggaagacctggaccctttctatagtacccagaagaccttcatcgtgctgaataagggcaaaaccatcttccggttcagtgccaccaatgccttgtacgtcctcagccccttccacccggtgcgcagagcggctgtgaagattttggtacattcgctcttcagcatgctcatcatgtgcaccatcctaaccaactgtgtgttcatggcacaacacgaccctccgccttggaccaaatatgttgagtacaccttcaccgccatctacacctttgagtctctggtcaagattctagctcgaggcttctgcctgcatgcgttcaccttcctccgggacccgtggaactggctagacttcagtgtgatcgtcatggcgtatgtatcagagaatataaagctaggcaatttgtcggctcttcgaactttcagagtcctgagagctctgaaaacgatttcagttattccaggcctgaagacaatcgtgggagccctaatccagtctgtgaagaagctagccgatgtgatggtccttactgtcttctgcctcagtgtctttgccctcattggcctgcagctcttcatgggcaacctgaggcacaagtgcgtgcgtaactttactgagctcaatggcaccaatggttccgtggaggccgatggcatagtctggaactctctggacgtctacctcaatgacccagccaattacctactcaagaatggcaccacggatgtgttgctgtgtgggaacagctctgatgccgggacatgtcctgaaggctaccggtgcctgaaggcaggtgagaaccctgaccacggttacaccagcttcgactccttcgcctgggctttccttgcgctcttccgcctgatgacacaggactgctgggaacgtctataccagcagaccctgaggtccgcaggaaagatctacatgatcttcttcatgcttgtcatattcctgggctccttctaccttgtgaacttgatcctggctgtggtggccatggcttacgaggagcaaaaccaagccaccatcgccgagacggaagagaaggagaagcgtttccaggaggccatggagatgctcaagaaggaacacgaggctctcaccatcaggggcgtggatactgtgtcccgtagctctctggagatgtctcctttggccccagtaaccaaccatgagagaaggagcaagaggaggaaacgactgtcttcagggacagaggatggcggagatgacaggctccccaagtcggactcagaagacggtcccagagcattgaatcagctcagcctcacccatgggctcagccggacatccatgaggccccgctccagccgagggagcattttcacattccggcgacgggaccaaggctccgaggcagattttgcagatgacgagaacagtacagcaggagagagcgagagccaccgcacatcgctgctggtgccctggcccctgcgccgtcccagcacccaagggcagcccggctttggaacctcagctcctggtcatgtcctcaatggcaaaagaaacagcaccgtggactgcaatggggtggtttccttgctgggggcaggggacgcagaggccacctctccagggagccaccttctccgccctatagtgctggacagacccccagacacgaccacgccgtcagaggagccgggtgggccccagatgctgacaccgcaggctccatgtgcagatggctttgaggagcccggggcacggcaacgggcactcagcgccgtcagtgtcctcaccagcgctctggaagagttggaggagtcccaccgtaagtgtccaccatgctggaatcgctttgcccagcattacctcatctgggagtgctgcccactctggatgtccatcaagcagaaggtgaagttcgtggtcatggacccgtttgctgacctcaccatcaccatgtgcatcgtgctcaacacgctcttcatggctctggagcactacaacatgacagccgagtttgaggagatgctgcaggtcggaaacttggtcttcacgggaatcttcacggcagagatgaccttcaagatcatcgcccttgacccatactactacttccagcagggctggaatatcttcgacagcatcatcgtcatcctcagcctcatggagctgggcctgtctcgcatgggcaacctgtctgtgctgcgttccttccgtctgctgcgggtcttcaagctggccaaatcatggcccaccctgaacacactcatcaagatcatcgggaactctgttggtgccctggggaacctgaccctggtgctggccatcatcgtcttcatcttcgccgtggtgggtatgcagctcttcggcaagaactactcagagctgaggcaccgaatcagcgactctggcctgctgccccgctggcacatgatggacttcttccacgccttcctcatcatcttccgcatcctctgtggagagtggatcgagaccatgtgggactgcatggaggtgtctgggcagtcgctgtgcttgctggtcttcctgctggtcatggtcattggcaaccttgtggtcctgaatctcttcttggccttgctgctcagctccttcagcgcagacaacctcacagcccctgacgaggatggggagatgaacaacctccagctggccctggctcgcatccaaaggggccttcgctttgtcaagcggaccacctgggacttctgctgcgggctcctgcggcggcgacctaagaagcccgcggctcttgccacccacagccagctgcccagctgtatcgccgctcccaggtccccaccacccccagaggtggagaaggcgcccccagcccgcaaggaaacacggttcgaggaagacaagcggccgggccagggcacccctggggataccgagcctgtgtgtgttcccatcgcagtggctgagtcagacactgatgaccaggaagaggatgaggagaacagccttggcacggaggaggaagagtccagcaaacagcaggaatcccaagttgtgtctggtggccacgagcccccccaggagcccagggcctggagccaggtgtcagagaccacatcctctgaagccgaggccagtacgtctcaggcagactggcagcaagagcgggaagcggagccccgggccccggggtgcggtgagactcccgaggacagttactccgagggtagcaccgctgacatgaccaacaccgcagacctcctggagcaaatcccagacctcggggaggacgtcaaggacccagaggactgcttcactgaaggctgcgtccgacgttgtccttgctgcatggtggacacgacgcaggccccagggaaggtctggtggagactgcgcaagacctgctaccgcatagtggagcacagctggttcgagactttcatcatcttcatgatcctgctcagcagcggagcgctggccttcgaggacatctacctggaggagcggaagaccatcaaggttctgctggagtacgccgacaagatgttcacctatgtctttgtgttggagatgctgctcaagtgggtggcctacggcttcaagaagtacttcaccaacgcctggtgctggctggacttcctaattgtggacgtctcactggtcagcctcgtggccaacaccttaggctttgccgagatgggtcccatcaagtccctgaggacactgcgtgcacttcgacccctgagggctttgtcacgattcgagggcatgagggtggtggtcaacgcgctggtgggcgccatcccctccatcatgaatgtcctcctcgtctgcctcatcttctggctcatcttcagcatcatgggcgtgaacctcttcgccgggaagttcggtaggtgcatcaaccagaccgaaggggacctgcctctgaactacaccattgtgaacaacaagagcgagtgtgagtccttcaacgtgactggagagttgtactggaccaaggtgaaagtcaactttgacaacgtgggggctgggtacctggccctgctgcaggtggcgacatttaaaggctggatggacatcatgtatgcggctgtggactccagagggtatgaagagcagccgcagtgggaggacaacctctacatgtacatctacttcgtcgtcttcatcatcttcggctccttcttcaccctcaacctgtttattggtgtcatcattgataacttcaaccagcagaagaaaaagttagggggccaggacatcttcatgacggaggagcagaagaagtactacaatgccatgaagaagctgggctccaagaaaccccagaagcccatcccacggcctctgaacaagtaccagggtttcatattcgacattgtgaccaagcaggccttcgatgttaccatcatgttcctcatctgtttaaacatggtgaccatgatggtagagacagatgatcagagccctgaaaaagtcaacatcttggccaagatcaacctgctcttcgtggccatcttcacaggagagtgtattgtcaagatggctgccctgcgccactattacttcaccaacagctggaacatcttcgatttcgtggttgtcatcctctccatcgttggcactgtcctctccgacatcatccagaagtacttcttctccccaacgctcttccgtgtcatccgtctggccaggatcggccgcatcctcaggctgatccgaggagccaaggggattcgcacgttgctcttcgccctcatgatgtccctgcccgccctcttcaacatcggcctcctcctcttcctcgtcatgttcatctactccatcttcggcatggccaactttgcctacgtcaagtgggaggctggcatcgacgacatgttcaacttccagacctttgccaacagcatgctgtgccttttccagatcaccacgtcggctggctgggatggcctcctcagccccatcctcaacacaggacccccctactgtgaccccaacctgcccaacagcaatggctccagggggaactgtgggagcccagcagtgggcatcctcttcttcaccacctacatcatcatctccttcctcatcgtggtcaacatgtacattgccatcatcctggagaacttcagtgtggccacagaagagagcacagagcccctgagcgaggatgacttcgacatgttctatgagatctgggagaagttcgacccggaggccacccagttcattgagtatttggccctgtccgactttgccgatgccctgtctgagccactccgcatcgccaagcccaaccagataagcctcatcaatatggacctgcccatggtgagcggggaccgcatccactgtatggacatcctgttcgccttcaccaagagggtgctcggggagtctggggagatggatgccctgaagatccagatggaggagaaattcatggcggccaatccttccaagatctcctatgagcccatcaccaccaccctgcggcgaaaacatgaggaggtgtcggccacggtcatccagcgggccttccggaggcacctcctgcagcgctcggtgaagcatgcttccttcctcttccgccagcaagcgggcagcagtggcctctctgatgaggatgcccccgagcgagagggactcattgcctacatgatgaatgagaacttctcccggcgcagcggtcccctctccagctcttccatctcctccacgtccttcccaccgtcctatgacagcgtcacgagggccaccagtgataacctcccagtgcgtgcgtcggactacagccgcagcgaagatcttgcagacttccctccatctccagatagggaccgagagtctatagtgtgaacctggccacccgtccggccaggacgtgccctgttgtatcctgacaaaccttaagcagttgaaaccagtgtcggctaaggccttcctagctctggaggtgacaggggaacctccaggtggaggaaccacacggcagagttctgtgtgtctgtgttgaagctagaggcggctagagcaaccctggctggtagaggcctggcacaccctagtagccgggtctggccaggcagcgccgcatggctctgaacagcaactctgtccccctctgaggtgaaacgggaaacacactgtacatgttgtgaacaagctttcataggtttattatatttgatatttttttactcgagcaaagaacatctttttccatgaacatctgcagcagttcacacggcctcttcctaaccctgagcaggtgtcaatggagaactctgctctcagagctaacgtggaatcttcgcacccaaggggagggttgtggttcctgtcacctgggctgaggagccaggaaggcctaagcacttcactcacacacgcgagtgggcgagtgcgtgtgcctgtgcacacttcacacactggggcgggacataggccgcagggacccagggtcgcctcaggcaaatgtggttggtcggtggctccaaaggcgagaggtttttcttcctggccccccagtgtgggtttcttcctgtgaactcactctgtgcccctgttgtccacaggattccagtctcctgttctcagtcaggcttcgcttgaggtcagtgctgtaaaaattgaaagcttccagagagctgccctggtctcccttcctctcctgtaagagtattaatgttggggcccaagggctcgaggaaggcagcctcgctcacagccaggcacgtagcttgcttctgagatgtggcaggccctgtcccaaggccaagggaggagaaggctgtcccacgtgggccgctgaactgtgtccagctgtggcagcaaagggagcggtctccatttcccaggctcctctggggtctctgcctctggccgtgtgtcaaggagatgcccactggggctccaaccgtcttcagacactgtggaagtgggagggtagcctggccctgtctaggagaaaggccctgcgtggtcaggcctggttctgtgacctggaaggtgacaagccatccatcccccatgggagaggagacagtgtggcatctgggcccagcccagggtctgactatcctaggcgtctcctgagaaggctttttcagggaaaacaaacaaaccgattttataagtccaatcatccccgtgacctcctcagctgctgacaatcctatttagcatatgcaaatcttttattgtagaggaccagaccatcgctgctgaagggacaggctggtggagcctgagcaggcaggggcttggctgcccaagtctagctctccagcagagccaccctcggctgggggcacccacctcacaggccacctcagtggcgcctgcccactgcgggctggctgctcctacctacctcgccgagctgtcggagggctggacgctcctggcagtgctgaaggagggggcactgcgggcaagtcaagtattatgtttcttcttgtcacctcagttcccttggtggcagctggctcccgccccacctgcccctggcggcttatccaatgtccccacggagtccggtgtggggggtttggttctaactgtcctggtgatgcctctctgagtggaaatcttatttttgtagatctgtactttgctctcgaggcttggagagatgtgtgtccctccctggcactcacttcaggccgcatgggcaggaatgtggcctggggctggcagggttggcagggctgccatcccagctggccggaaagaggctgtggtttcgaatatgcagatggccttggagccttaagacaggtgccgggctggctgcagacagggtggctgaggggaggggcaatcgagtcccgagtccttggcttagcatggcctccttttctagaactgcacagaggggaggagagagtagccggtagacggaaggagggaggggcatgcgtacatgtcaggtgttgtttgaggtaaatgagcacttctcactaagcttcgtgtataaatggaatacattatttttcaaacaaacgaataaatggcttacacg

SEQ ID NO: 48 - protein, 2020 aa, Mus musculus
MANFLLPRGTSSFRRFTRESLAAIEKRMAEKQARGSATSQESREGLPEEEAPRPQLDLQASKKLPDLYGNPPRELIGEPLEDLDPFYSTQKTFIVLNKGKTIFRFSATNALYVLSPFHPVRRAAVKILVHSLFSMLIMCTILTNCVFMAQHDPPPWTKYVEYTFTAIYTFESLVKILARGFCLHAFTFLRDPWNWLDFSVIVMAYVSENIKLGNLSALRTFRVLRALKTISVIPGLKTIVGALIQSVKKLADVMVLTVFCLSVFALIGLQLFMGNLRHKCVRNFTELNGTNGSVEADGIVWNSLDVYLNDPANYLLKNGTTDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVTNHERRSKRRKRLSSGTEDGGDDRLPKSDSEDGPRALNQLSLTHGLSRTSMRPRSSRGSIFTFRRRDQGSEADFADDENSTAGESESHRTSLLVPWPLRRPSTQGQPGFGTSAPGHVLNGKRNSTVDCNGVVSLLGAGDAEATSPGSHLLRPIVLDRPPDTTTPSEEPGGPQMLTPQAPCADGFEEPGARQRALSAVSVLTSALEELEESHRKCPPCWNRFAQHYLIWECCPLWMSIKQKVKFVVMDPFADLTITMCIVLNTLFMALEHYNMTAEFEEMLQVGNLVFTGIFTAEMTFKIIALDPYYYFQQGWNIFDSIIVILSLMELGLSRMGNLSVLRSFRLLRVFKLAKSWPTLNTLIKIIGNSVGALGNLTLVLAIIVFIFAVVGMQLFGKNYSELRHRISDSGLLPRWHMMDFFHAFLIIFRILCGEWIETMWDCMEVSGQSLCLLVFLLVMVIGNLVVLNLFLALLLSSFSADNLTAPDEDGEMNNLQLALARIQRGLRFVKRTTWDFCCGLLRRRPKKPAALATHSQLPSCIAAPRSPPPPEVEKAPPARKETRFEEDKRPGQGTPGDTEPVCVPIAVAESDTDDQEEDEENSLGTEEEESSKQQESQVVSGGHEPPQEPRAWSQVSETTSSEAEASTSQADWQQEREAEPRAPGCGETPEDSYSEGSTADMTNTADLLEQIPDLGEDVKDPEDCFTEGCVRRCPCCMVDTTQAPGKVWWRLRKTCYRIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSECESFNVTGELYWTKVKVNFDNVGAGYLALLQVATFKGWMDIMYAAVDSRGYEEQPQWEDNLYMYIYFVVFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKVNILAKINLLFVAIFTGECIVKMAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPNLPNSNGSRGNCGSPAVGILFFTTYIIISFLIVVNMYIAIILENFSVATEESTEPLSEDDFDMFYEIWEKFDPEATQFIEYLALSDFADALSEPLRIAKPNQISLINMDLPMVSGDRIHCMDILFAFTKRVLGESGEMDALKIQMEEKFMAANPSKISYEPITTTLRRKHEEVSATVIQRAFRRHLLQRSVKHASFLFRQQAGSSGLSDEDAPEREGLIAYMMNENFSRRSGPLSSSSISSTSFPPSYDSVTRATSDNLPVRASDYSRSEDLADFPPSPDRDRESIV

SEQ ID NO: 49 - DNA, 4516 bp, Homo sapiens; identical to SEQ ID NO: 39

SEQ ID NO: 50 - protein, 815 aa, Homo sapiens; identical to SEQ ID NO: 40

SEQ ID NO: 51 - DNA, 4606 bp, Mus musculus
atcagtccgctaccgcgggacccggggctgtgtcaggtctggaagtggagctggagctgaccgaaggagctgccgctgggccggggtggaacacagctctgcaccggggtcttaaggaggggtgtgttgctgctggctcactgcgagccgcctccagcctgcgccgaggcaccttcccgccaggccgtctgcctctgtcttccgcctccctccatttccccggaatctcagcccggcgcggctggaccctggctctcctgtgggtgggaaagccccagcctttttcctgctatactcctttatcccaggctggccttccagccttctctcgcctttccctgcggagtgggtctcacccttttccgtctttagcccaggctccttacccctcccctttagccgagttccagcaccctggtgattttccttagccaaaggtcctatccttcttcctcctttcccttgcccgtggtccagcctatccggcccttcccctctcttcaggacgcagtttccccgatttccttctctcagcctgaagttctagatcctttcggtttcctccttagtccctccaaggtacttaacccctttttgcagtttggactttgagattgtgaatctctagaaaacgaaaatacttctctaaaacatgaacttttcctagagattgccccagtctcaccccatctgcaggactccttgctacctatgtcccaggtcttacacgcacttcgctaagggctgaggcctcctaggctgggggatcagtatgatgcttcggtggtccggcgtctggggatttcatccacctcggatcttcccctccttgctggtggtggttgccttggtgggactgctacctgttctcaggagccacggcctccagcacagccctactgccagcaccatcagaggttctgaaccaccccgggaacgctcaattggggatgtcaccacggcaccatcagagcctctccatcgcccagatgaccacaatttgaccaacttaatcattgaacatggcggtaagccatctcggaaggccttcccagtcctggacattgactaccctcacgtgcgcacaccctttgagatctccctctggattctcctggcctgcctcatgaagataggtttccatgtgatccccaccatctcaagcatcgtcccggagagctgcctgctgatcgtagtggggctgctggtggggggcctgatcaagggcgtcggagagacgccccccttcctgcaatcagacgtcttcttcctcttcctgctgccacccatcatcctggatgcaggctacttcctgcctctgcggcagttcacggagaacctgggcaccatcctgatctttgctgtggtgggcacactgtggaatgcgttcttcctgggtggcctcctgtacgccgtgtgcctggtgggcggcgagcagatcaacaacattggcctgctggacaccctgctctttggcagcatcatctctgccgtggaccctgtggctgtgctggccgtcttcgaggagatccacatcaacgaactgctacacatccttgtcttcggggagtccctgctcaacgatgccgtcactgtggtcctctatcacctctttgaggaatttgccagctatgactctgtgggcatctcggacatcttcctcggcttcctgagcttcttcgtggtggccctcggtggggtgtttgtgggcgtggtctacggggtaatcgcagctttcacctcccgatttacctctcacatccgggtcatcgagccgctcttcgtcttcctctacagctacatggcttacctgtcagctgagctcttccacctgtccggaatcatggccctcatcgcctcaggagtagtgatgcgcccctatgtggaggccaacatctcccacaaatcccacaccaccatcaagtacttcctgaagatgtggagcagcgtcagtgagaccctcatcttcatcttcctcggcgtctccactgtggccggctcccaccagtggaactggaccttcgtcatcagcaccctgctcttctgcctcatcgcccgggtgctgggtgtcctggtcctgacctggttcatcaacaagttccgcattgtcaagctgacccccaaggaccagttcatcattgcctatgggggcctgcgaggggccatcgccttctcgctgggctacctcctggacaagaagcacttccccatgtgtgacctgttcctcaccgccatcatcaccgtcatctttttcaccgtctttgtgcagggcatgaccattcggcccctggtcgacctgttggccgtgaagaaaaagcaagaaacaaagcgctccatcaacgaggagatccacacacagttcctggaccaccttctgacaggcatcgaggacatctgtggtcattatggccaccatcactggaaggacaagctcaaccgttttaataagaagtatgtgaagaagtgcctgatagcaggagagcgctccaaagagccccagctcattgccttctaccacaagatggagatgaagcaggccattgagctggtggagagcggaggcatgggcaagatcccatctgctgtctccactgtctctatgcagaacatccaccccaaggctgtgacttcagaccgcatcttgccagcactgtccaaggacaaggaggaagagatccgcaaaatcttgaggagcaatctgcagaaaacccggcaacggctgcggtcctataacagacacacgctggtggccgacccctacgaggaagcctggaaccagatgctgctccggaggcagaaggcccggcagctggagcagaagatcaccaactacctgacagtaccggcccacaagctggactcgcccaccctgtctcgggcccgcataggctcagacccactggcctatgaacccaaggcagacctgcctgtcatcaccattgacccggcctccccacagtcccctgagtctgtggacctggtgaacgaggagttgaagggcaaggtcctagggctaaaccggggtcccagggtgactccggaggaggaggaggaagatgaggacggcatcatcatgattcggagcaaggagccctcatccccaggcaccgacgacgtcttcacccctggatcaagtgacagccccagctcccagagaatacaacgctgcctcagtgacccaggcccccaccctgagcctggggagggagagcctttcatccccaaaggacagtagagccaggctagtgggtgtcccacagactctcccacgagagtgggggctgggcgctcctcttgtctcctgacctggattggctctgcccccttcctccgcatggcagctgggcccacagcccccactgcagcacggcttgcccctgcctcctgggaagggtcctctcctacagaactgccttcccaatccatccatttggcggacggctgggctgctgaggcaggccccgccccatctctagatccaggctctcccatagtggaaccagggctcctcaaggccctccttccctccagcccctcctcatccagaccaactctccatccttgatcatcatttctaaccaaagagccactggctccactgtggtcccagcaggaaaggagggagctgagccttccgtggccgtgactgggcccctgtatcagaggacagcaggcctgtggctgggaaacagacccgtgacccatagcagagctgcccacggcagcgccacggttctcccgctagcctcggtgctctggccaccacccgagctgtccttactcagagctgcgggctgagggcgtctctgagcttctctgctgggcgctggggcacattcgacagtgacatgattggggtgatgaggaccactgggccttagtggggcttacacgggagggactgtccgaggcgctggaaagtgtcctctacttacacttggcttatttgcacctcactgtaagtatgattctgtttgcacagaggcactcttgttttcagaccctttgagtcccccatgccagtcagagctctgccctttccccctcgtccttcctcgtgtctaacaccgtcccttaaatggaacctctgctgctgggggcccttgcccacagcaccgattctgatgtggcccaacctgtcatgactcaagctcaccagcagggggcctcctggtacctcctttgctcttgggcctgcttttacttctgttggccagttctaccaccaccccttatctggaccactctctttcctgcattctgtagagtagaaagtactggaccccctagaggcctttgctcacaggcacacactccctaccgccaccatggctggggccaagcccatgggtatctgcccaacccctgggcactccctccccaggatcgtgcaataatagtcagagggtccctcccaggggatggggccatgggtgcatggcccagctgtctcctctccatgcaagtgctgttttgggcaggagttgccaggcagggtgacatcgacaaccatgtacaaagccaccgcagctgctgccgctctgccgcctgtacaaaggaaactgaacctttttcatattctaataaatcaatgtgagtttttgat

SEQ ID NO: 52 - protein, 820 aa, Mus musculus; identical to SEQ ID NO: 42